(19) United States
(12) Patent Application Publication
(10) Pub. No.: US 2024/0116909 A1
(43) Pub. Date: Apr. 11, 2024

(54) NOVEL COMPOUNDS USEFUL AS STING AGONISTS AND USES THEREOF

(71) Applicant: Jacobio Pharmaceuticals Co., Ltd., Beijing (CN)

(72) Inventors: Yang YE, Beijing (CN); Xiuwei LI, Beijing (CN); Guiqun YANG, Beijing (CN); Fashun YAN, Beijing (CN); Yanping WANG, Beijing (CN); Wei LONG, Beijing (CN)

(21) Appl. No.: 18/284,897

(22) PCT Filed: Mar. 29, 2022
(86) PCT No.: PCT/CN2022/083557

§ 371 Date: Sep. 29, 2023

(30) Foreign Application Priority Data
Jan. 12, 2021 (WO) PCT/CN2022/071458
Mar. 30, 2021 (WO) PCT/CN2021/083783
Apr. 29, 2021 (WO) PCTCN2021/090945
May 11, 2021 (WO) PCT/CN2021/128942
Aug. 9, 2021 (WO) PCT/CN2021/111479
Nov. 11, 2021 (WO) PCT/CN2021/130097
Dec. 16, 2021 (WO) PCT/CN2021/138806
Dec. 30, 2021 (WO) PCT CN2021/143043

(51) Int. Cl. C07D 409/12; C07D 495/04; C07D 519/00; C07D 495/14; C07D 471/04

(52) U.S. Cl. CPC C07D 409/12; C07D 495/04; C07D 519/00; C07D 495/14; C07D 471/04

(57) ABSTRACT

Compounds of general Formula I, II, III, IV, V and their pharmaceutically acceptable salts, that may be useful as inductors of type I interferon production, specifically as STING active agents, are provided. Also provided are synthesis, compositions, and uses of such compounds.

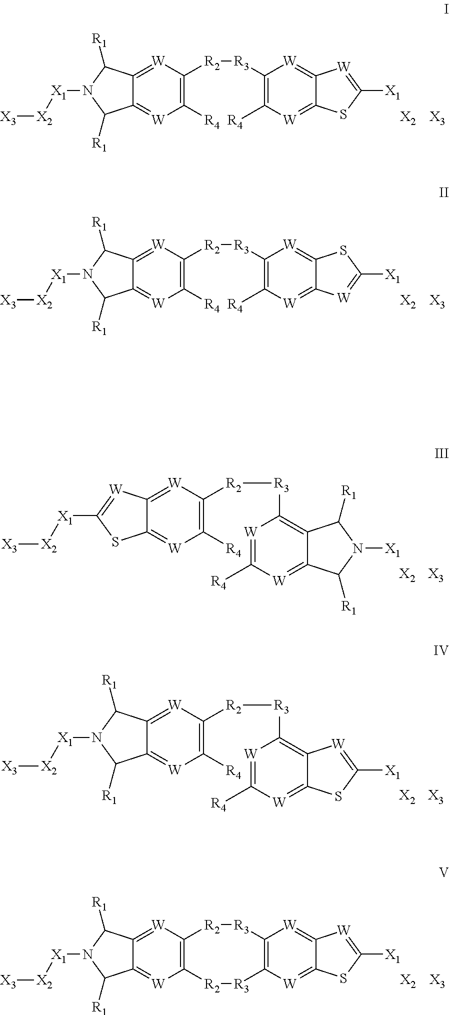

### CROSS REFERENCE TO RELATED APPLICATIONS

The application claims the benefit of PCT application Ser. No. PCT/CN2021/083783 filed on Mar. 30, 2021; PCT application Ser. No. PCT/CN2021/090945 filed on Apr. 29, 2021; PCT application Ser. No. PCT/CN2021/111479 filed on Aug. 9, 2021; PCT application Ser. No. PCT/CN2021/128942 filed on Nov. 5, 2021; PCT application Ser. No. PCT/CN2021/130097 filed on Nov. 11, 2021; PCT application Ser. No. PCT/CN2021/138806 filed on Dec. 16, 2021; PCT application Ser. No. PCT/CN2021/143043 filed on Dec. 30, 2021; and PCT application Ser. No. PCT/CN2022/071548 filed on Jan. 12, 2022. The entire contents of these applications are incorporated herein by reference in their entirety.

### TECHNICAL FIELD

The present invention relates to compounds and derivatives thereof and that may be useful as STING (Stimulator of Interferon Genes) agonists that activate the STING pathway. The present invention also relates to compositions comprising such compounds, processes for the synthesis of such compounds, and uses of such compounds to induce immune responses, to induce STING-dependent type I interferon production, and/or to treat a cell proliferation disorder, such as cancer.

### BACKGROUND ART

The immune system has evolved to recognize and neutralize different types of threats in order to maintain the homeostasis of the host, and it is generally broken down into two arms: adaptive and innate. The adaptive immune system is specialized to recognize as foreign those antigens not naturally expressed in the host and to mount an anti-antigen response through the coordinated actions of many leukocyte subsets. The hallmark of adaptive immune responses is their ability to provide “memory” or long-lasting immunity against the encountered antigen. While this specific and long-lasting effect is critical to host health and survival, the adaptive immune response requires time to generate a full-blown response.

The innate immune system compensates for this time delay and is specialized to act quickly against different insults or danger signals. It provides the first line of defense against bacteria, viruses, parasites and other infectious threats, but it also responds strongly to certain danger signals associated with cellular or tissue damage. The innate immune system has no antigen specificity but does respond to a variety of effector mechanisms. Opsonization, phagocytosis, activation of the complement system, and production of soluble bioactive molecules such as cytokines or chemokines are all mechanisms by which the innate immune system mediates its response. By responding to these damage-associated molecular patterns (DAMPs) or pathogen-associated molecular patterns (PAMPs) described above, the innate immune system is able to provide broad protection against a wide range of threats to the host.

Free cytosolic DNA and RNA are among these PAMPs and DAMPs. It has recently been demonstrated that the main sensor for cytosolic DNA is cGAS (cyclic GMP-AMP synthase). Upon recognition of cytosolic DNA, cGAS catalyzes the generation of the cyclic-dinucleotide 2′3′-cGAMP, an atypical second messenger that strongly binds to the ER-transmembrane adaptor protein STING. A conformational change is undergone by cGAMP-bound STING, which translocates to a perinuclear compartment and induces the activation of critical transcription factors IRF-3 and NF-κB. This leads to a strong induction of type I interferons and production of pro-inflammatory cytokines such as IL-6, TNF-α and IFN-γ.

The importance of type I interferons and pro-inflammatory cytokines on various cells of the immune system has been very well established. In particular, these molecules strongly potentiate T-cell activation by enhancing the ability of dendritic cells and macrophages to uptake, process, present and cross-present antigens to T-cells. The T-cell stimulatory capacity of these antigen-presenting cells is augmented by the up-regulation of critical co-stimulatory molecules, such as CD80 or CD86. Finally, type I interferons can rapidly engage their cognate receptors and trigger the activation of interferon-responsive genes that can significantly contribute to adaptive immune cell activation.

From a therapeutic perspective, type I interferons are shown to have antiviral activities by directly inhibiting human hepatitis B virus and hepatitis C virus replication, and by stimulating immune responses to virally infected cells. Compounds that can induce type I interferon production are used in vaccines, where they act as adjuvants, enhancing specific immune responses to antigens and minimizing side effects by reducing dosage and broadening the immune response.

In addition, interferons, and compounds that can induce interferon production, have potential use in the treatment of human cancers. Such molecules are potentially useful as anti-cancer agents with multiple pathways of activity. Interferons can inhibit human tumor cell proliferation directly and may be synergistic with various approved chemotherapeutic agents. Type I interferons can significantly enhance anti-tumor immune responses by inducing activation of both the adaptive and innate immune cells. Finally, tumor invasiveness may be inhibited by interferons by modulating enzyme expression related to tissue remodeling.

In view of the potential of type I interferons and type I interferon-inducing compounds as anti-viral and anti-cancer agents, there remains a need for new agents that can induce potent type I interferon production. With the growing body of data demonstrating that the cGAS-STING cytosolic DNA sensory pathway has a significant capacity to induce type I interferons, the development of STING activating agents is rapidly taking an important place in today's anti-tumor therapy landscape.

Thus, there remains a need in the art for developing compounds with novel structure, good biological activity and high druggability, and methods for preventing or treating disease and disorders, such as cancer. The compounds of the present invention fulfill this need.

### SUMMARY OF INVENTION

The present invention relates to novel compounds useful as STING agonists and for the treatment of cell proliferation disorders. The present disclosure includes compounds of general formula (I), compounds of general formula (II), compounds of general formula (III), compounds of general formula (IV), compounds of general formula (V), and pharmaceutically acceptable salts thereof. These compounds and their pharmaceutically acceptable salts may be useful as agents to induce immune responses, to induce STING-dependent type I interferon production, and/or to treat a cell proliferation disorder.

The present invention relates to novel compounds of general structure as Formula I, II, III, IV, V, or a pharmaceutically acceptable salt:

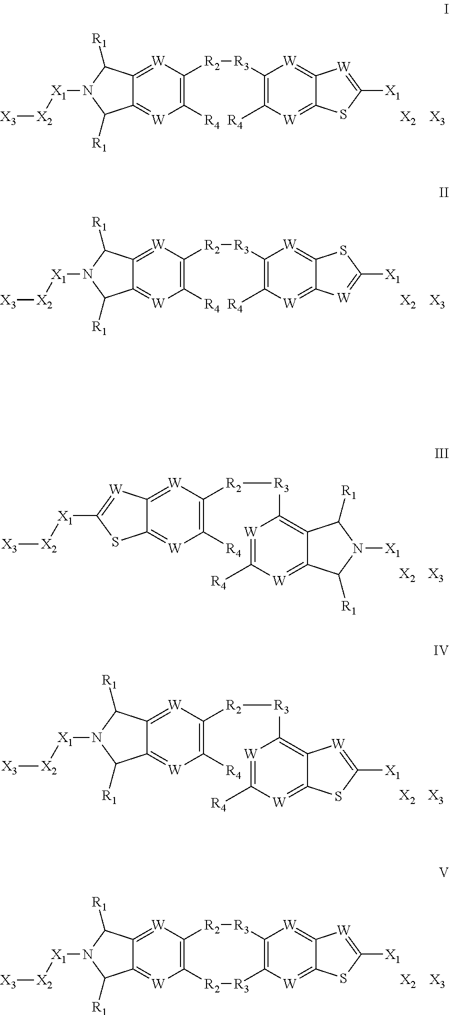

wherein
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl- T_{b}-, -Tₐ-C(═O)-T_{b}-, -Tₐ-C(═CH₂)-T_{b}-, -Tₐ-C(═O)—C(═O)-T_{b}-, -Tₐ-C(═O)(C₁-C₆alkyl)-C(═O)-T_{b}-, -Tₐ-C(═O)—C₃-C₁₂cycloakyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R₄ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring; and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from (C(R⁸)₂)₍₁₋₃₎, —NR⁸(C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂,

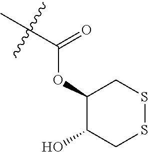 [SMILES: *C(=O)O[C@H]1CSSC[C@@H]1O]

and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂OR⁶, P(═O)(R⁶)₂, C₁-C₆alkyl, C₂-C₆ alkenyl, C₂-C₆ alkynyl, C₃-C₈ cycloalkyl, C₆-C₁₀aryl, 3-8 membered heterocycloalkyl, or 3-10 membered heteroaryl.

In some embodiments of Formula I,

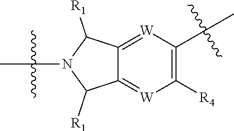

is independently selected from the group consisting of

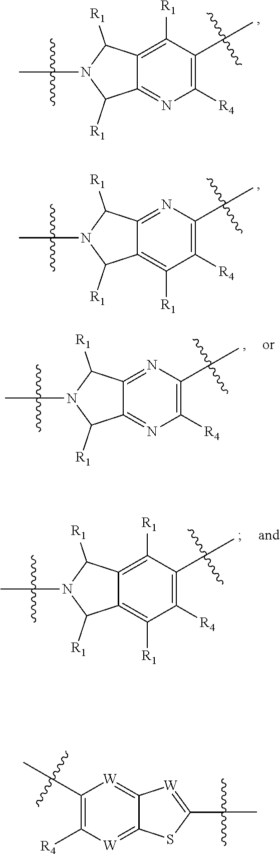

is independently selected from the group consisting of

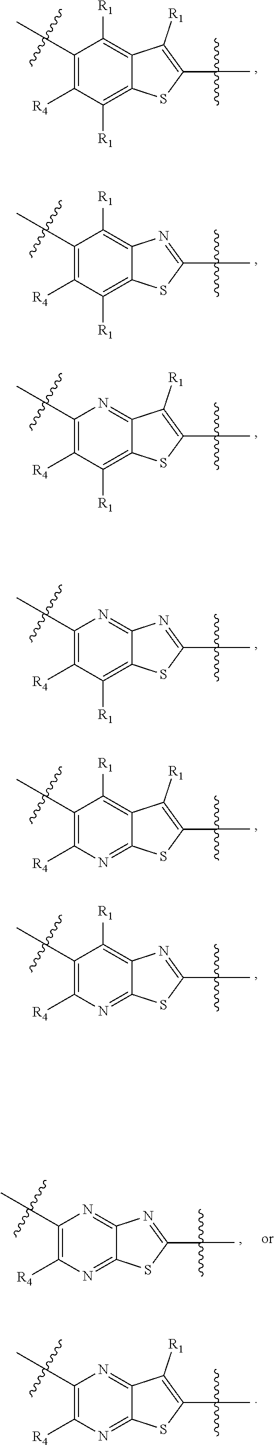

In some embodiments of Formula I,

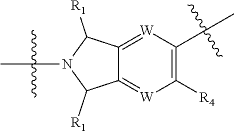

is independently selected from the group consisting of

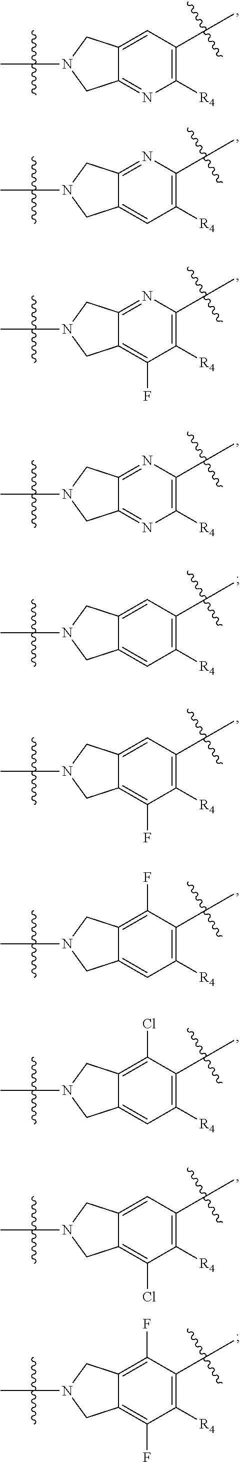

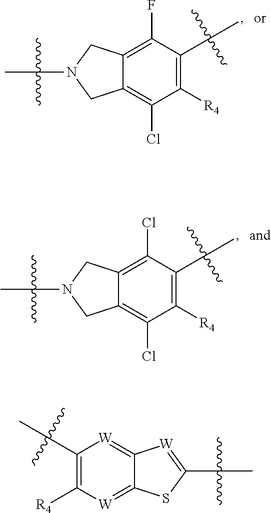

is independently selected from the group consisting of

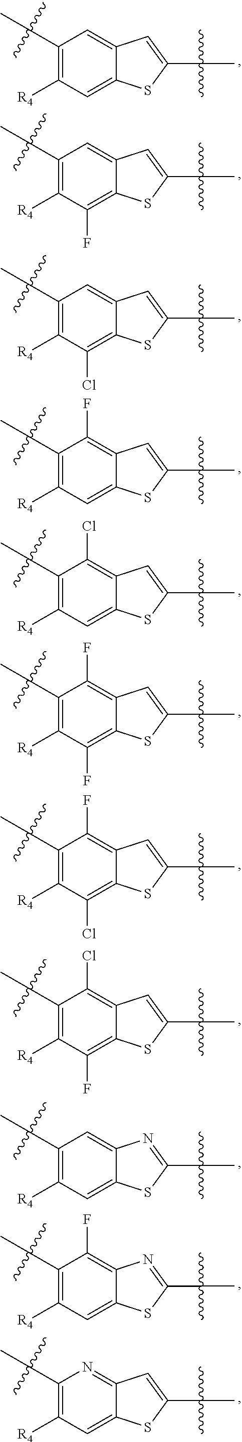

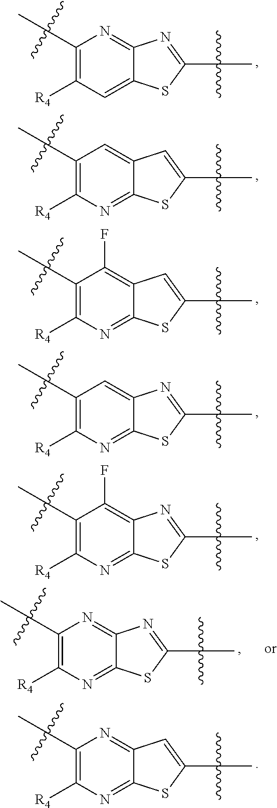

In some embodiments of Formula I, the compound is of Formula I-1:

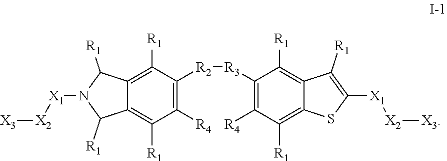

In some embodiments of Formula II,

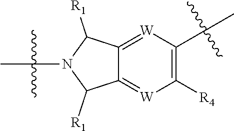

is independently selected from the group consisting of

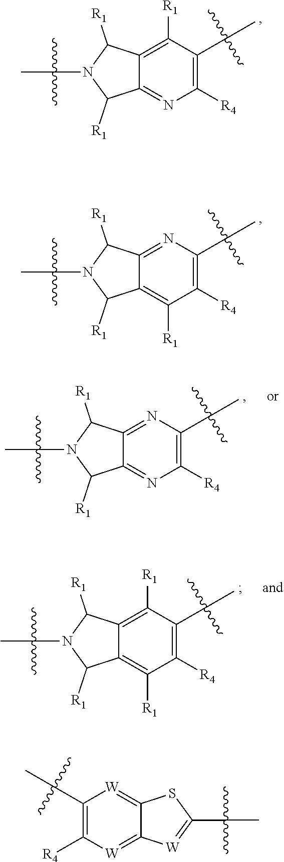

is independently selected from the group consisting of

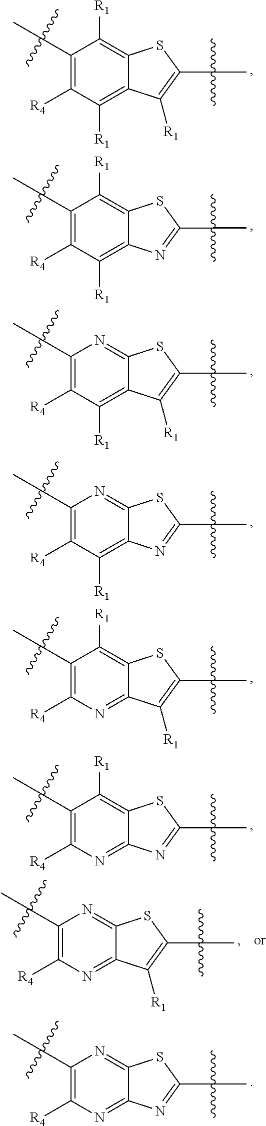

In some embodiments of Formula II,

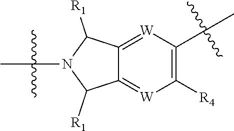

is independently selected from the group consisting of

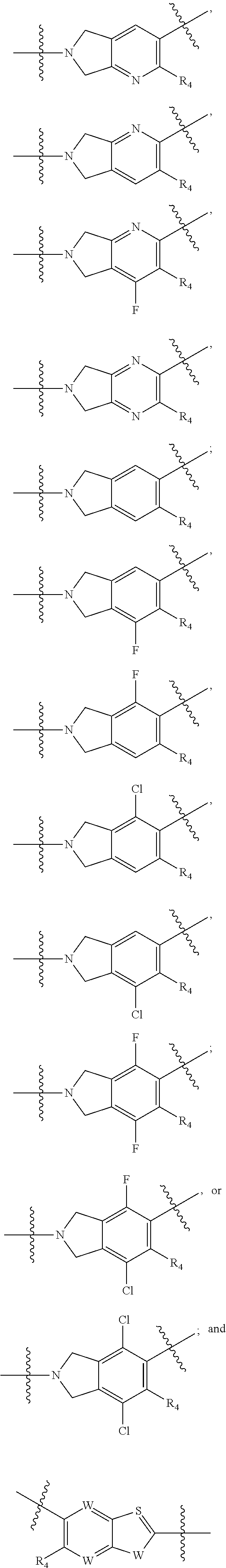

is independently selected from the group consisting of

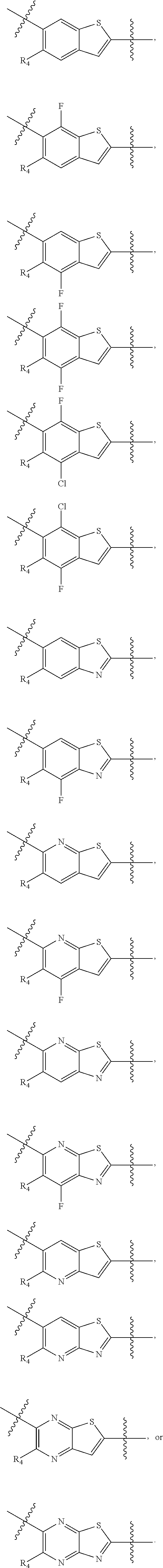

In some embodiments of Formula III,

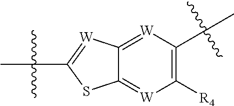

is independently selected from the group consisting of

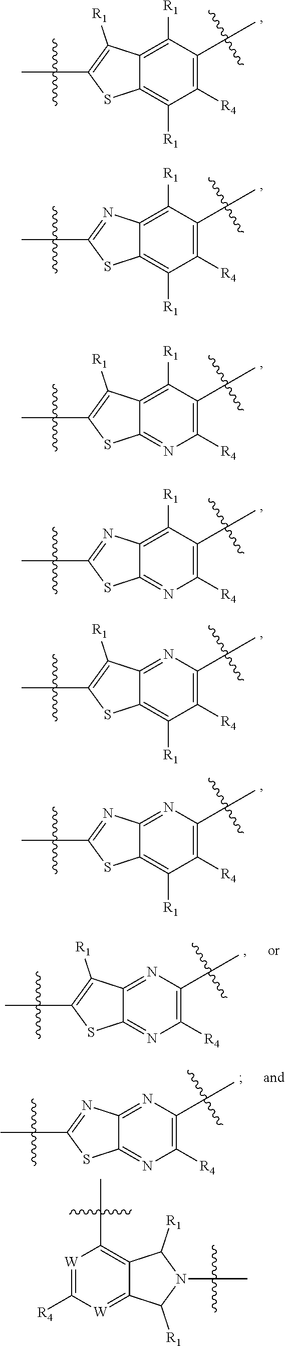

is independently selected from the group consisting of

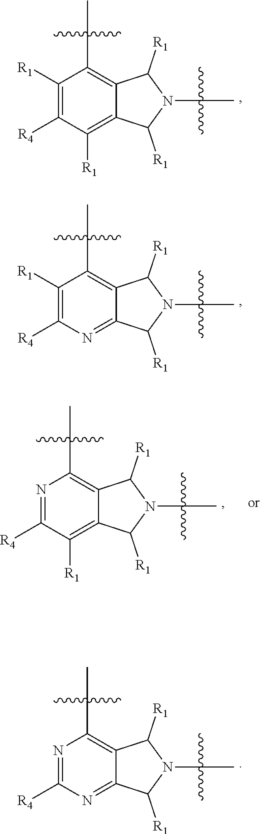

In some embodiments of Formula III,

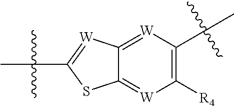

is independently selected from the group consisting of

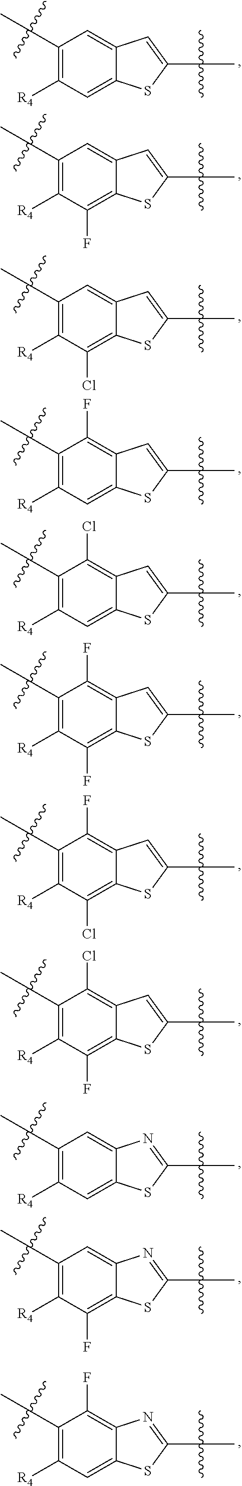

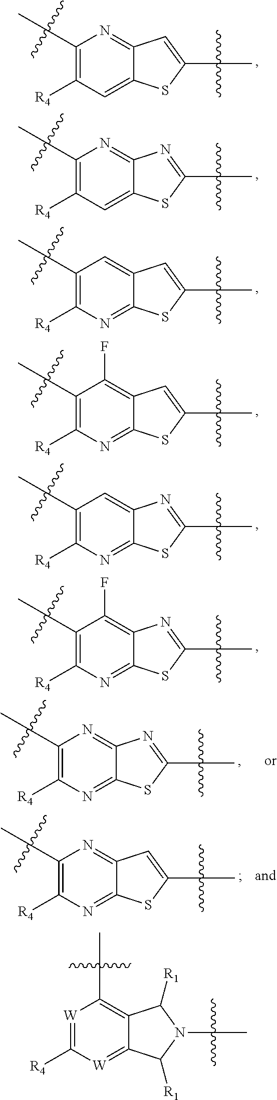

is independently selected from the group consisting of

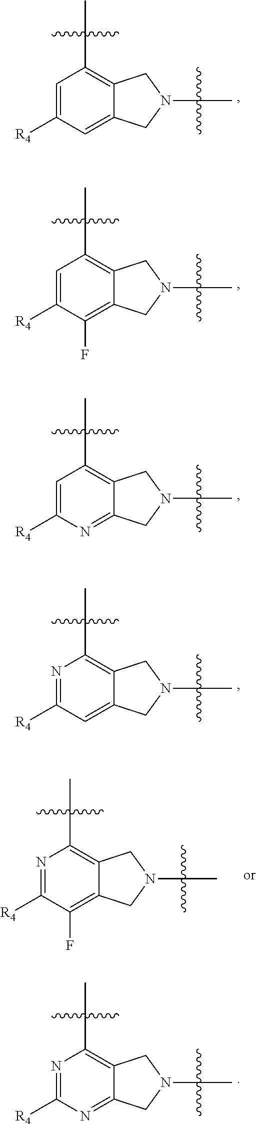

In some embodiments of Formula IV,

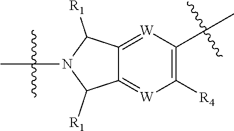

is independently selected from the group consisting of

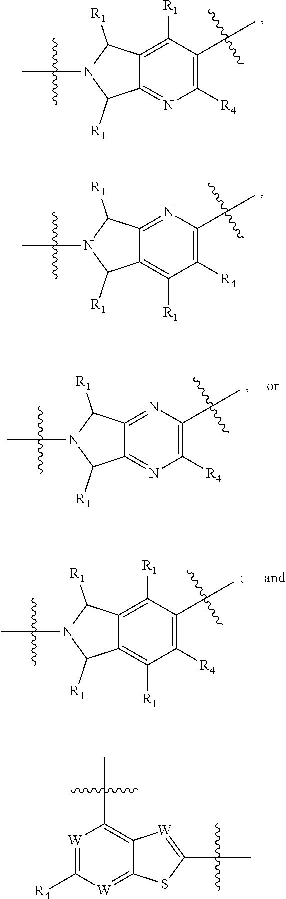

is independently selected from the group consisting of

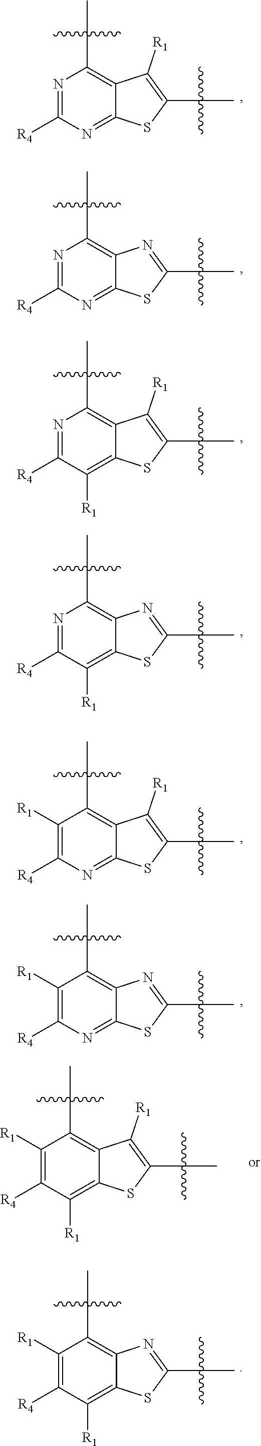

In some embodiments of Formula IV,

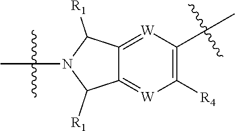

is independently selected from the group consisting of

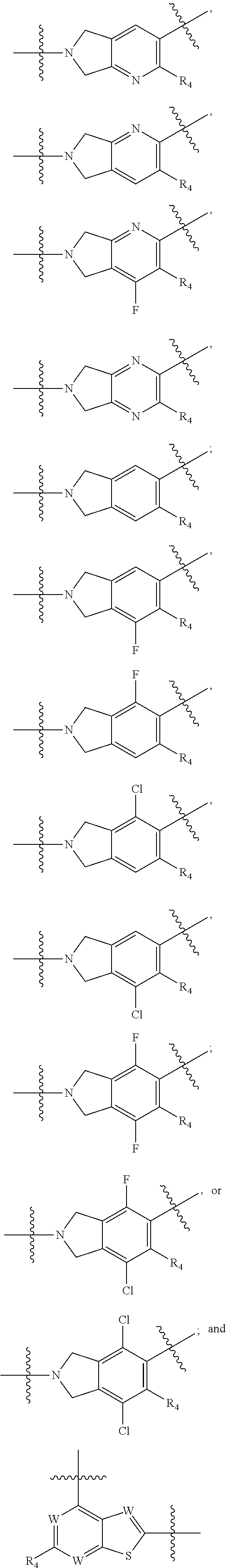

is independently selected from the group consisting of

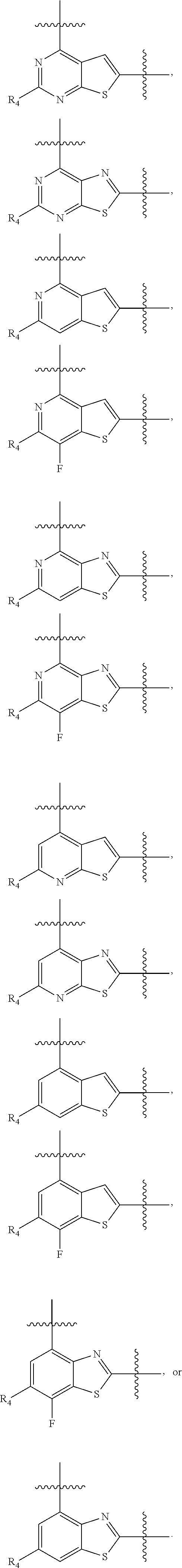

In some embodiments of Formula V,

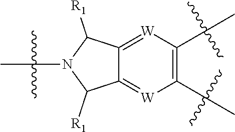

is independently selected from the group consisting of

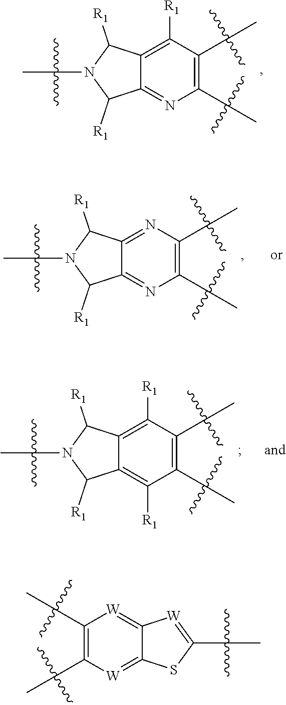

is independently selected from the group consisting of

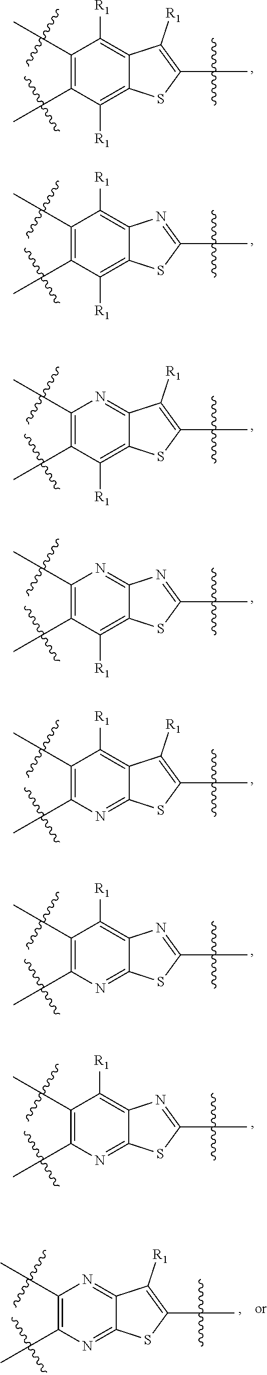

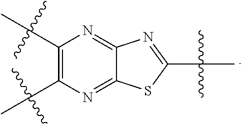 [SMILES: *c1nc2nc(*)c(*)nc2s1]

In some embodiments of Formula V,

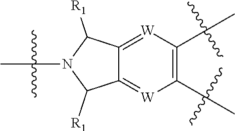

is independently selected from the group consisting of

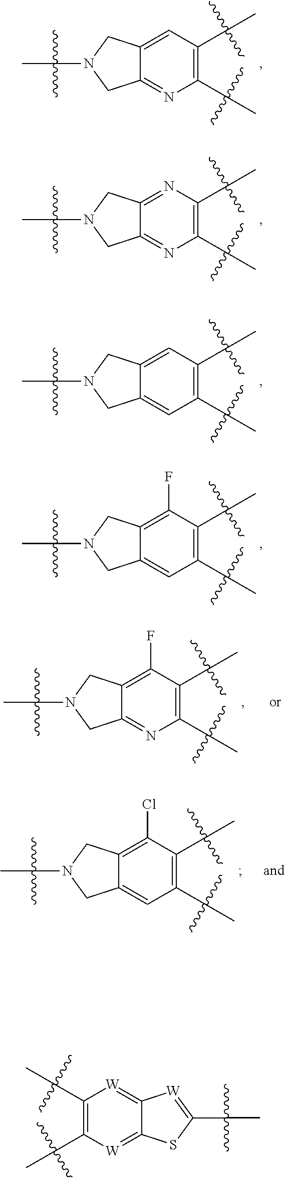

is independently selected from the group consisting of

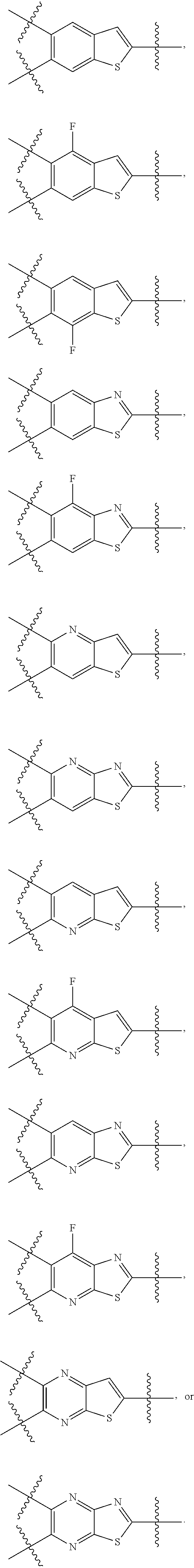

In some embodiments of Formula I, I-1, II, III, IV or V, each W is independently is CR₁.

In some embodiments of Formula I, I-1, II, III, IV or V, each W is independently is CH or CF.

In some embodiments of Formula I, I-1, II, III, IV or V, each W is independently is N.

In some embodiments of Formula I, I-1, II, III, IV or V, each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂.

In some embodiments of Formula I, I-1, II, III, IV or V, each R₁ is independently selected from the group consisting of H, deuterium, halogen, C₁-C₃ alkyl, CN and C₁-C₃ haloalkyl.

In some embodiments of Formula I, I-1, II, III, IV or V, wherein each R₁ is independently selected from the group consisting of H, deuterium, halogen, CN and C₁-C₃ alkyl.

In some embodiments of Formula I, I-1, II, III, IV or V, wherein each R₁ is independently selected from the group consisting of H, deuterium, F, Cl, Br, CN and methyl.

In some embodiments, each R₁ independently is hydrogen or halogen.

In some embodiments, each R₁ independently is hydrogen or F.

In some embodiments, each R₁ independently is hydrogen or CN.

In some embodiments, each R₁ independently is deuterium.

In some embodiments of Formula I, I-1, II, III, IV or V, R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)- T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)- T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs. wherein in -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and
   each Rs independently is H, deuterium or C₁-C₆ alkyl optionally substituted with one or more halogen.

In some embodiments of Formula I, I-1, II, III, IV or V, R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)- T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)- T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs; and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and each Rs independently is H, deuterium or C₁-C₆ alkyl optionally substituted with one or more halogen.

In some embodiments of Formula I, I-1, II, III, IV or V, R₂ and R₃ are independently selected from O—(C₁-C₄ alkylene or haloalkylene)-C₂-C₆alkenyl, C₁-C₅ alkylene or haloalkylene, (C₁-C₄ alkylene or haloalkylene)-N(R⁶), and N(R⁶)—(C₁-C₄ alkylene or haloalkylene)-C₂-C₆alkenyl, —C₀₋₆alkyl-NH—C₀₋₆alkyl-, —C₀₋₆alkyl-N(C₁₋₆alkyl)-C₀₋₆alkyl-, —C₀₋₆alkyl-O—C₀₋₆alkyl-, —C₀₋₆alkyl-PEGₙ-O—C₀₋₆alkyl, —C₀₋₆alkyl-S—S—C₀₋₆alkyl, —C₀₋₆alkyl-S—S—S—C₀₋₆alkyl, —C₀₋₆ alkyl-C₂-C₆alkenyl-C₀₋₆ alkyl-, —C₀₋₆ alkyl-C₂-C₆alkynyl-C₀₋₆ alkyl-, —C₀₋₆ alkyl-C(═O)—C₀₋₆ alkyl-, —C₀₋₆ alkyl-C(═CH₂)—C₀₋₆alkyl-, —C₀₋₆ alkyl-C(═O)—C(═O)—C₀₋₆alkyl-, —C₀₋₆ alkyl-C(═S)—C₀₋₆alkyl-, —C₀₋₆ alkyl-S(═O)₂—C₀₋₆alkyl-, —C₀₋₆alkyl-S(═O)—C₀₋₆alkyl-, —C₀₋₆ alkyl-P(═O)(—OH)—C₀₋₆alkyl-, —C₀₋₆ alkyl-C₃-C₁₂ cycloalkyl-C₀₋₆alkyl-, —C₀₋₆ alkyl-C₆-C₁₂ aryl-C₀₋₆alkyl-, —C₀₋₆ alkyl-(3- to 12-membered heterocyclyl)-C₀₋₆ alkyl-, —C₀₋₆ alkyl-(5- to 12-membered heteroaryl)-C₀₋₆ alkyl-, —C₀₋₆alkyl-O-(5- to 12-membered heteroaryl)-O—C₀₋₆alkyl-, —C₀₋₆alkyl-O—C(═O)—NH—C₀₋₆alkyl-, —C₀₋₆alkyl-O—C(═O)—C₀₋₆alkyl-, —C₀₋₆alkyl-NH—C(═O)—C₀₋₆alkyl-, —OC(═O)—O—, —NH—C(═O)—NH—, or —NH—C(═S)—NH—; wherein the C₂-C₆ alkenyl, C₂-C₆alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

In some embodiments of Formula I, I-1, II, III, IV or V, R₂ and R₃ are independently selected from -Tₐ-C₂-C₆ alkenyl-T_{b}-, -Tₐ-C(═O)-T_{b}-, -Tₐ-PEGₙ-O-T_{b}, -Tₐ-S—S-T_{b}, -Tₐ-S—S—S-T_{b}, -Tₐ-C(═CH₂)-T_{b}-, or -Tₐ- (C₆-C₁₂ aryl)-T_{b}-, wherein the C₂-C₆alkenyl or C₆-C₁₂ aryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are —N(Rs)-, —O—, —(C₁-C₆ alkyl)-O—, or —O—(C₁-C₆ alkyl)-O—; wherein the C₁-C₆alkyl it optionally substituted with one or more halogen; each Rs independently is H, deuterium or C₁-C₆ alkyl optionally substituted with one or more halogen.

In some embodiments of Formula I, I-1, II, III, IV or V, wherein R₂-R₃ is selected from

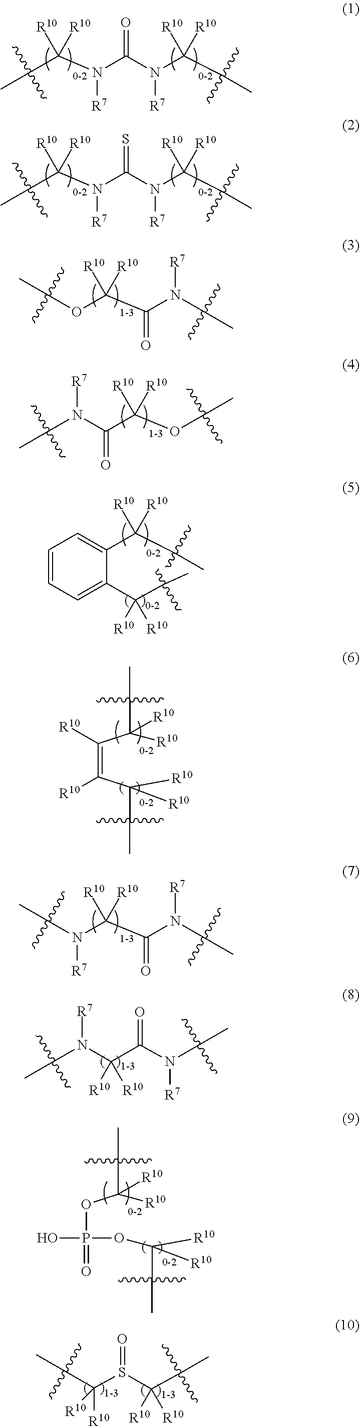

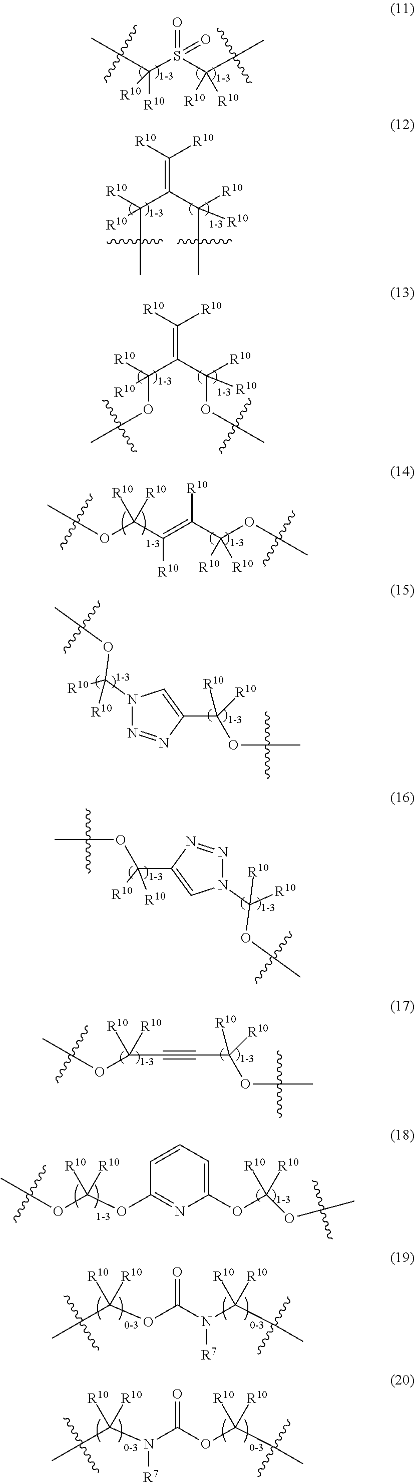

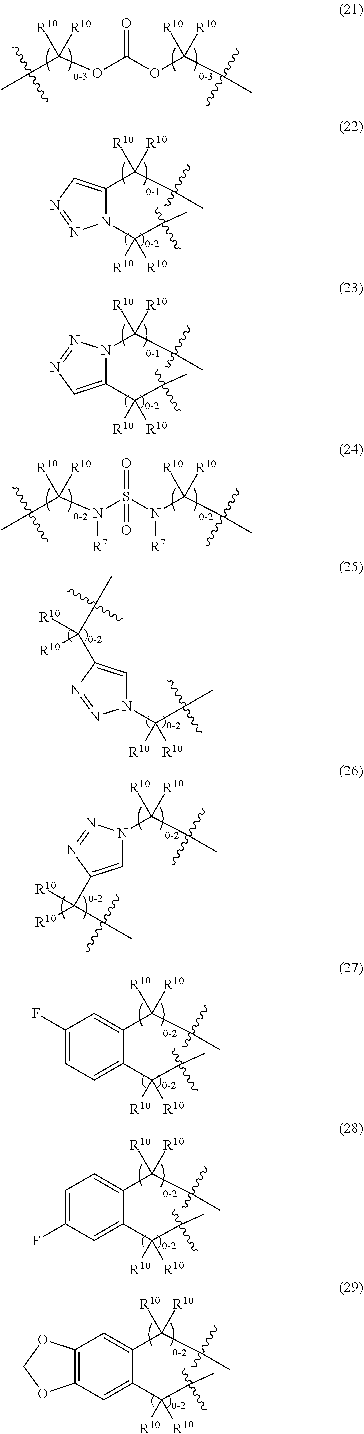

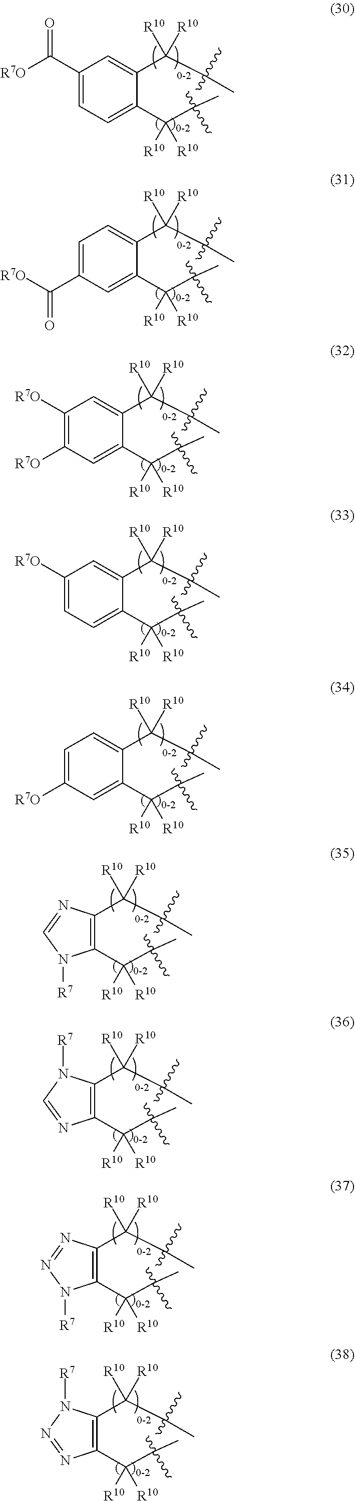

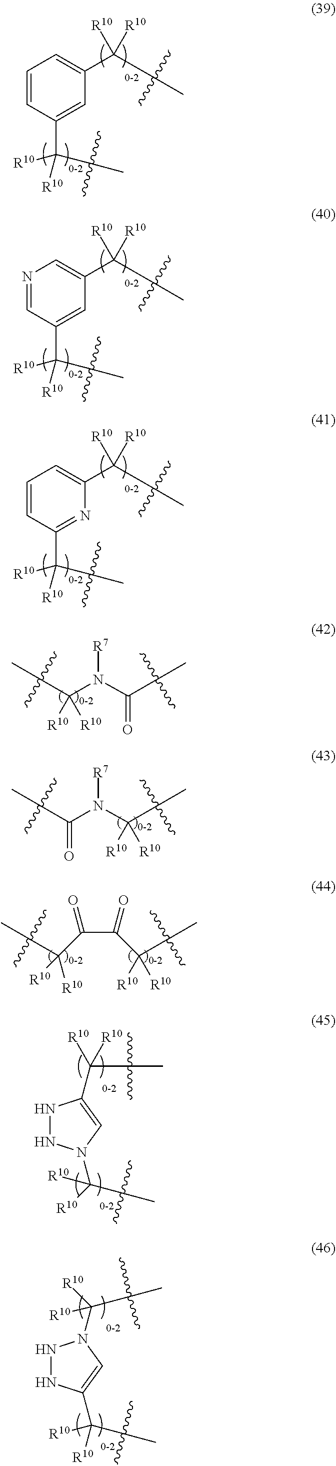

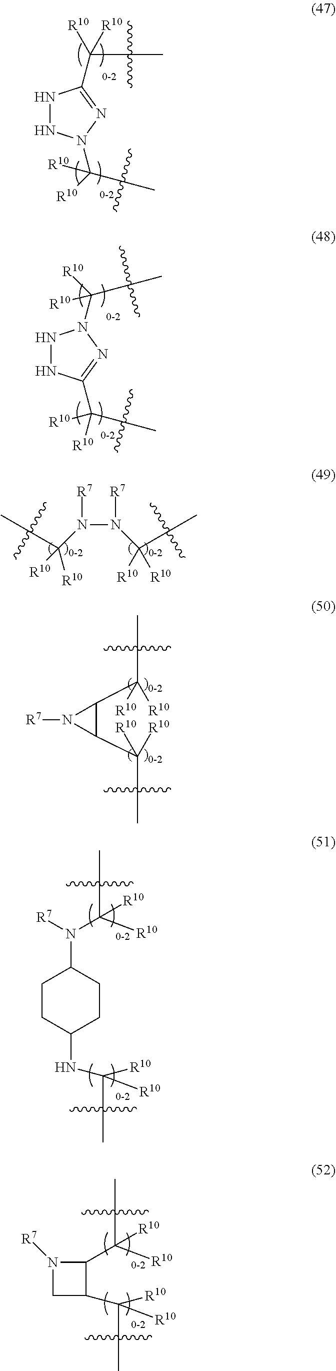

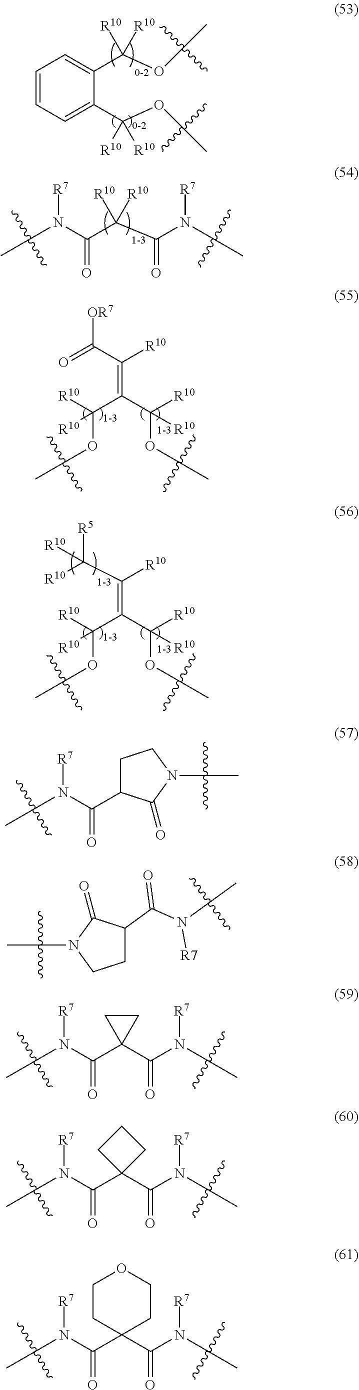

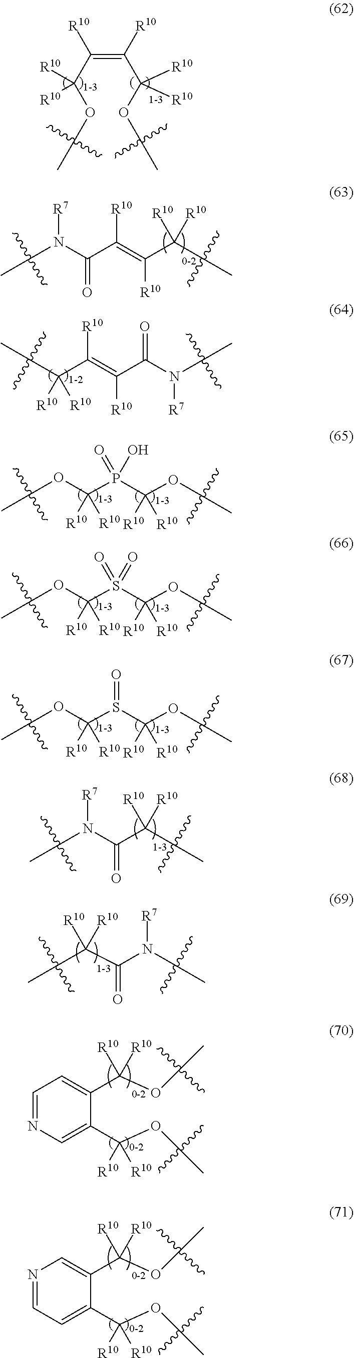

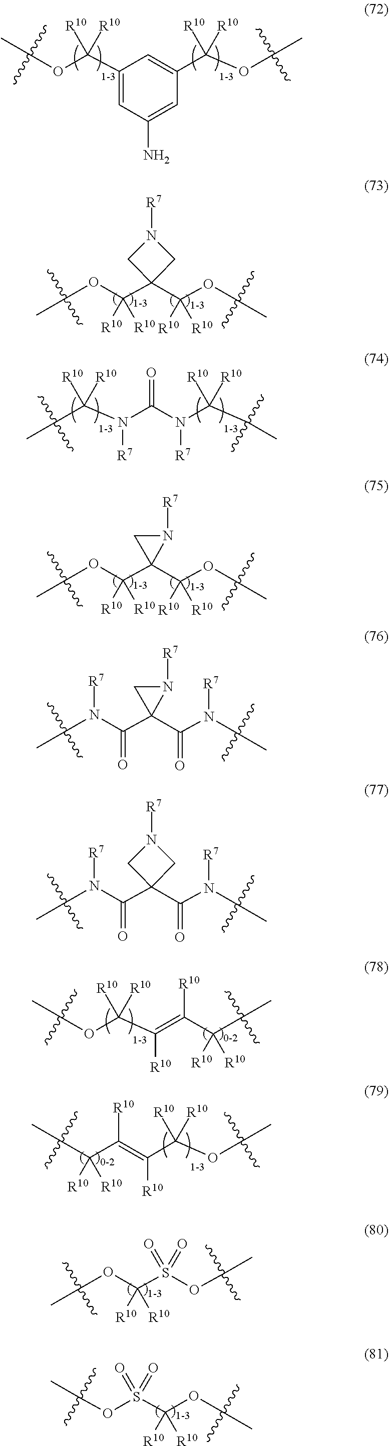

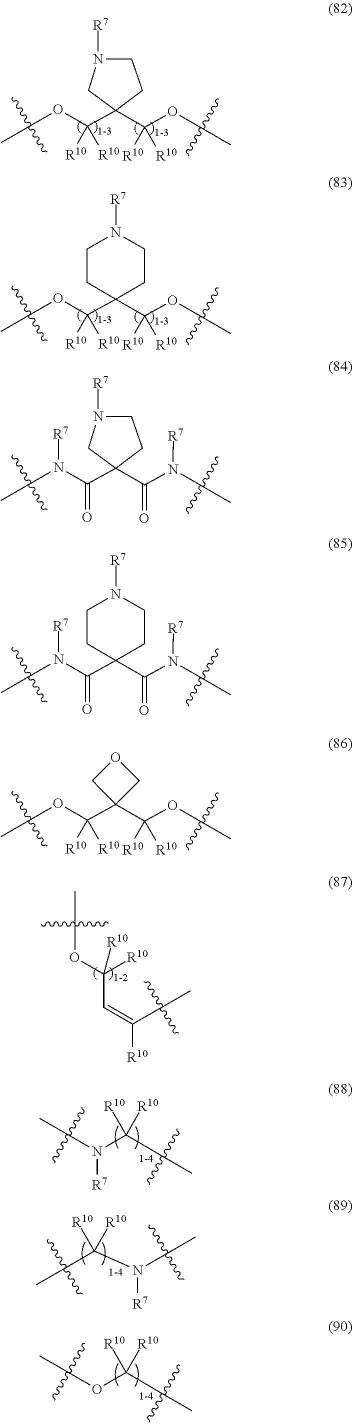

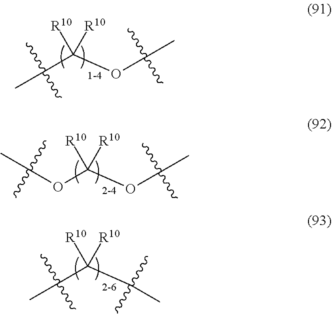

wherein:
each Rₛ is independently —OR⁷, NR⁷ or —C(O)OR⁷;
each R⁷ is independently hydrogen, deuterium or C₁₋₂ alkyl; and
each R¹⁰ is independently hydrogen, deuterium, C₁₋₂alkyl or halogen.

In some embodiments of Formula I, I-1, II, III, IV or V, wherein each R⁷ is independently hydrogen, deuterium or methyl.

In some embodiments, each R¹⁰ is independently hydrogen, deuterium, methyl or fluorine.

In some embodiments, one R¹⁰ is hydrogen, and the other R¹⁰ is methyl or fluorine.

In some embodiments, one R¹⁰ is deuterium, and the other R¹⁰ is methyl or fluorine.

In some embodiments of Formula I, I-1, II, III, IV or V, wherein R₂-R₃ is selected from the group consisting of —(CH₂)₂₋₈—, —O(CH₂)₁₋₇—, —O(CH₂)₁₋₆O—, —OCH₂CH(CH₃)CH₂O—, —OCH(CH₃)CH₂CH(CH₃)O—,

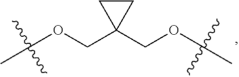 [SMILES: *OCC1(CO*)CC1]

—NH(CH₂)₁₋₇—, —(CH₂)₁₋₆NH(CH₂)₁₋₆—, —(CH₂)₁₋₆N(CH₃)(CH₂)₁₋₆—, —NH(CH₂)₁₋₆O—, —NH—CO—NH—, —N(CH₃)CO—NH—,

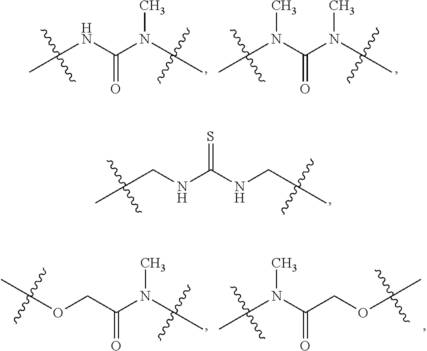

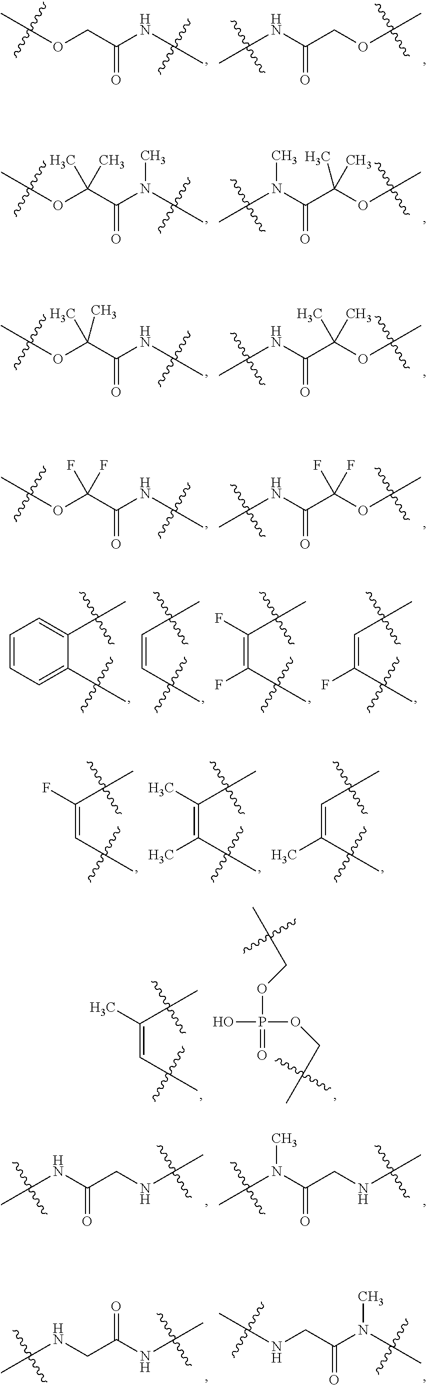

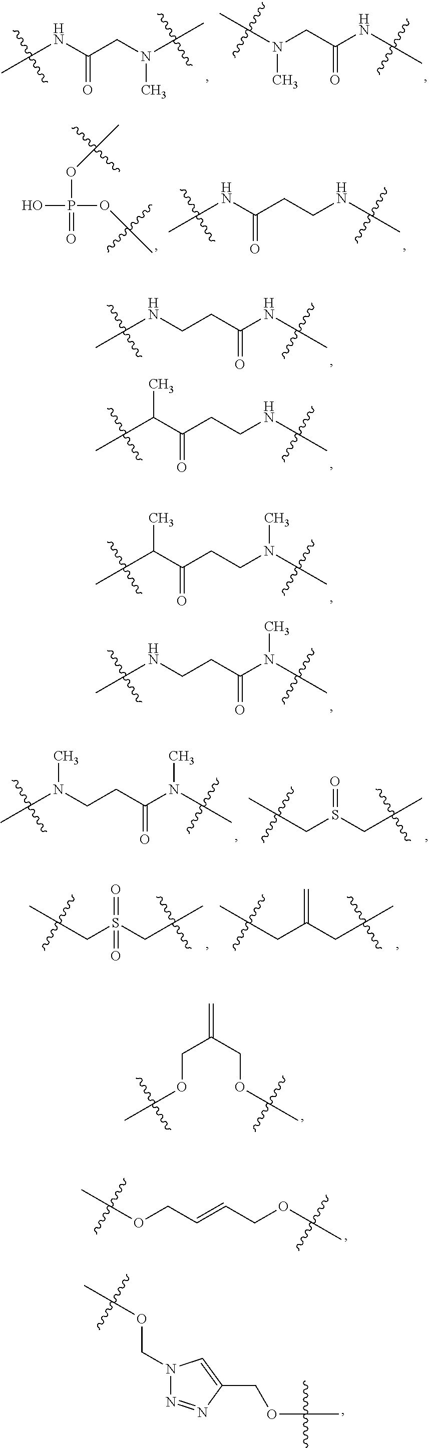

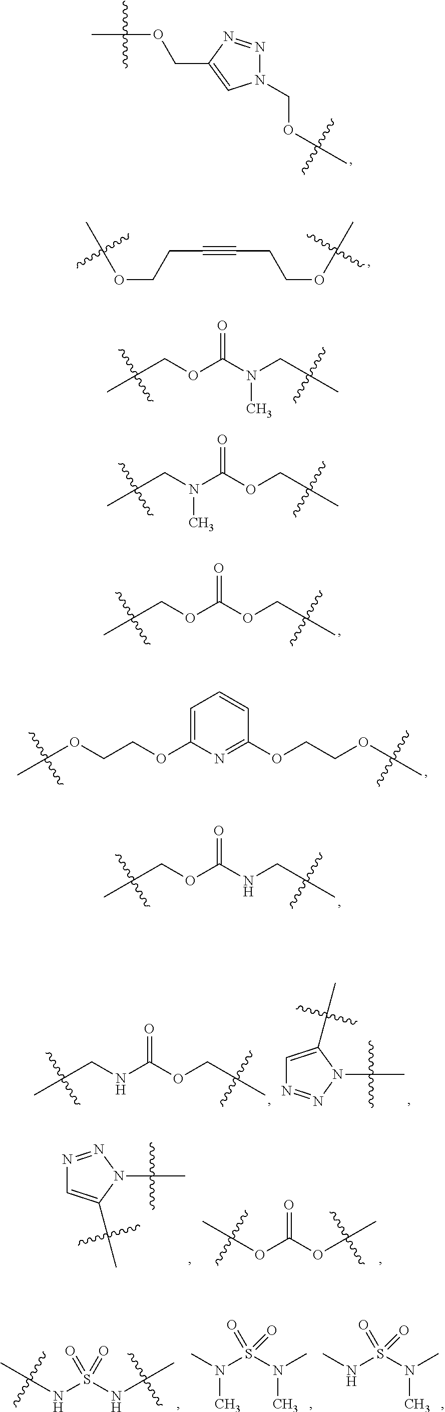

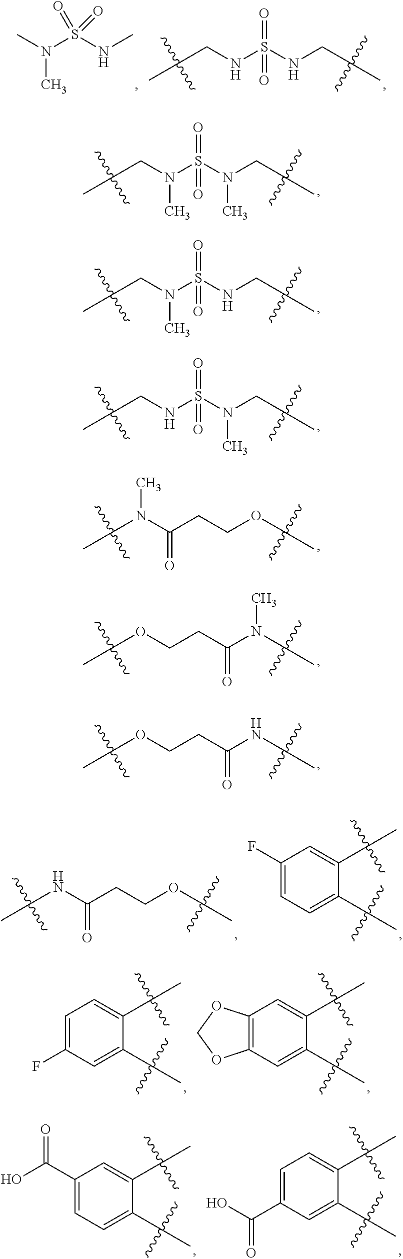

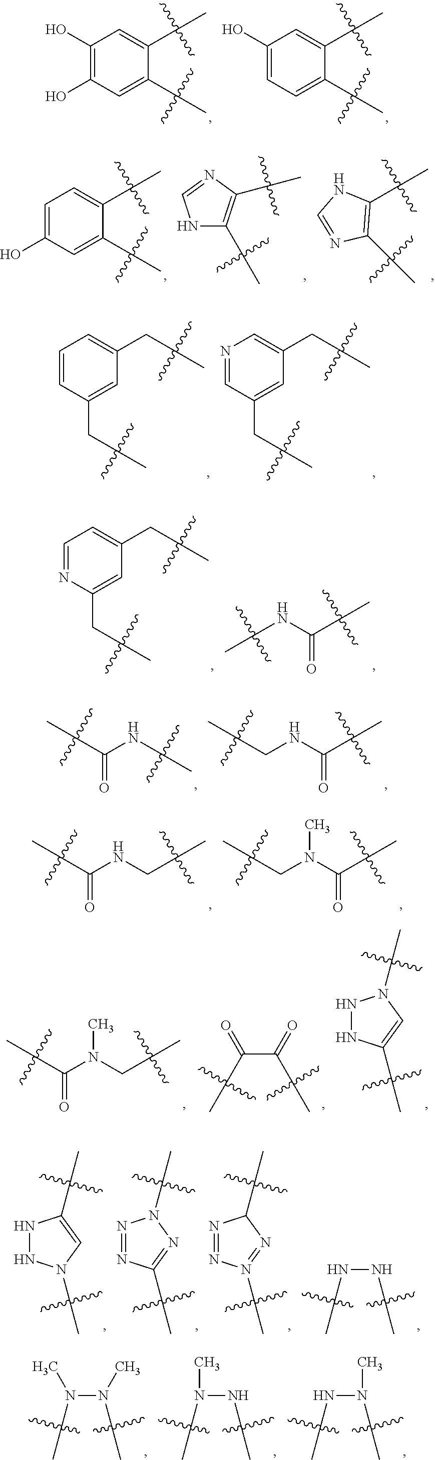

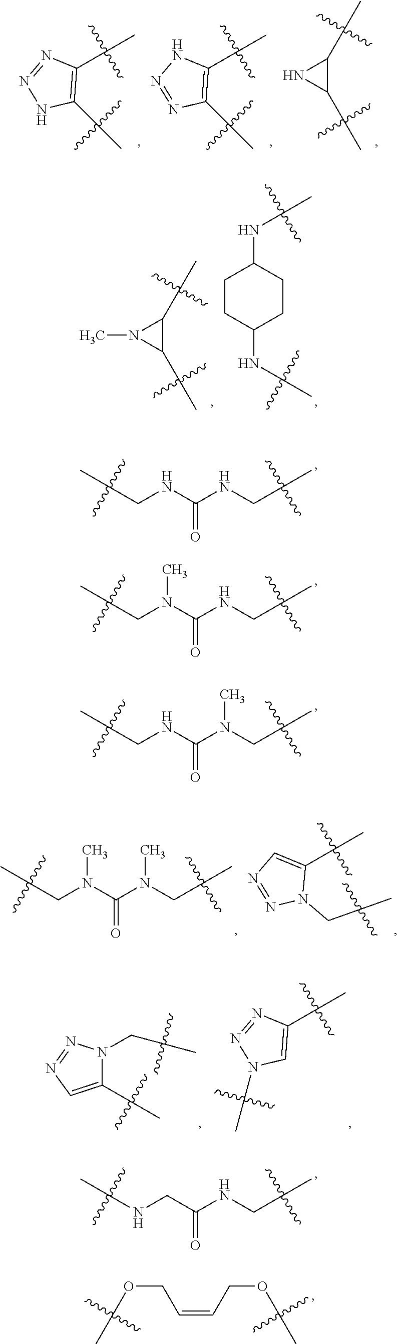

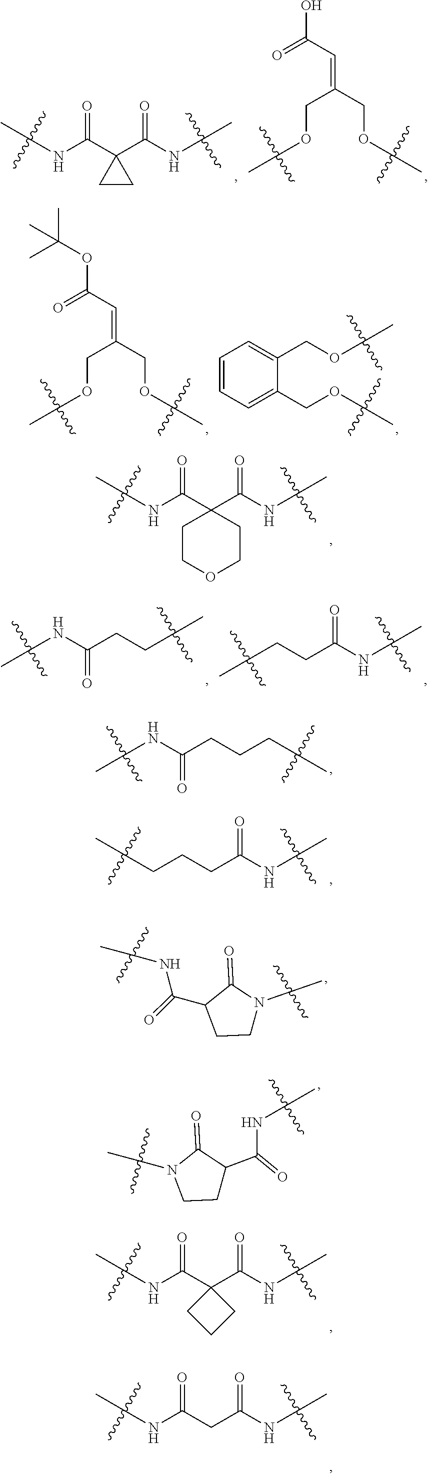

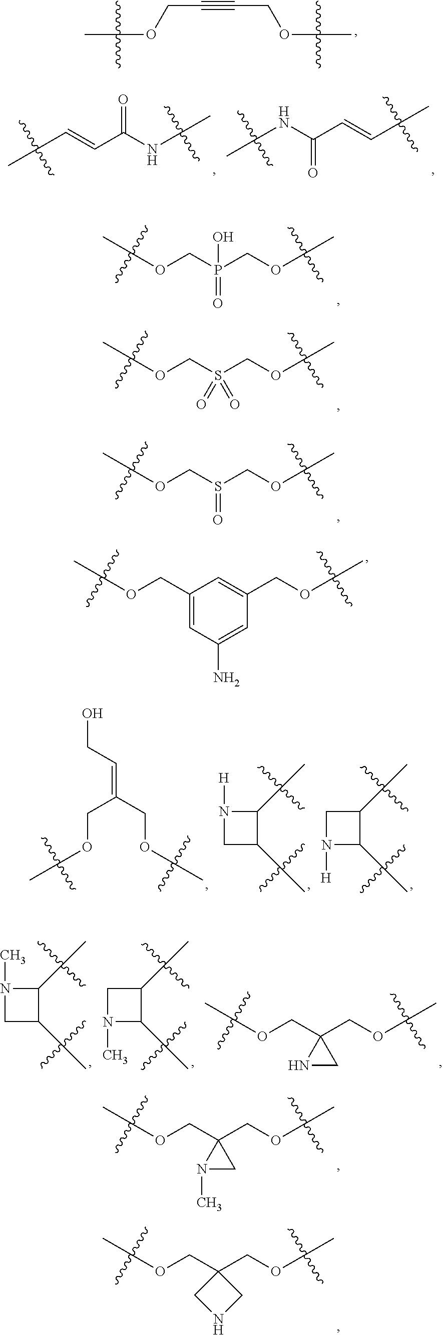

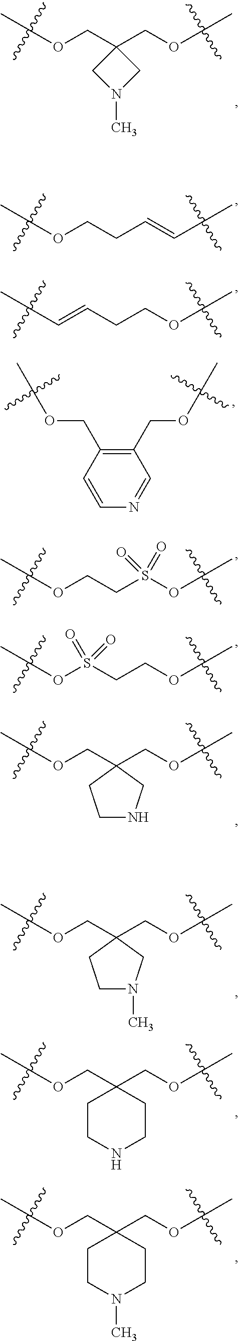

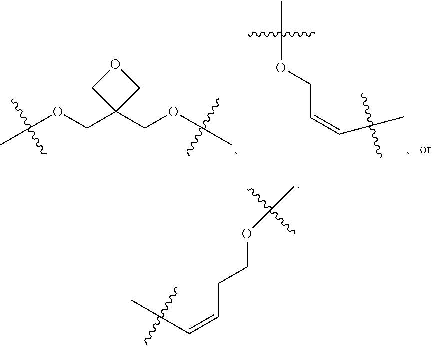 [SMILES: *OCC1(CO*)COC1 ; */C=C\CO* ; */C=C\CCO*]

In some embodiments of Formula I, I-1, II, III, IV or V, wherein R₂-R₃ is selected from the group consisting of —(CH₂)₂—, —(CH₂)₃—, —(CH₂)₄—, —(CH₂)₅—, —O(CH₂)₂—, —O(CH₂)₃—, —O(CH₂)₄—, —O(CH₂)₂O—,

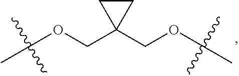 [SMILES: *OCC1(CO*)CC1]

—O(CH₂)₃O—, —O(CH₂)₄O—, —OCH₂CH(CH₃)CH₂O—, —OCH(CH₃)CH₂CH(CH₃)O—, —O(CH₂)₄O—, —O(CH₂)₅O—, —NH(CH₂)₂—, —NH(CH₂)₃—, —NH(CH₂)₄—, —(CH₂)₂NH—, —(CH₂)₃NH—, —(CH₂)₄NH—, —CH₂NHCH₂—, —CH₂N(CH₃)CH₂—, —NH(CH₂)₃O—, —NH—CO—NH—,

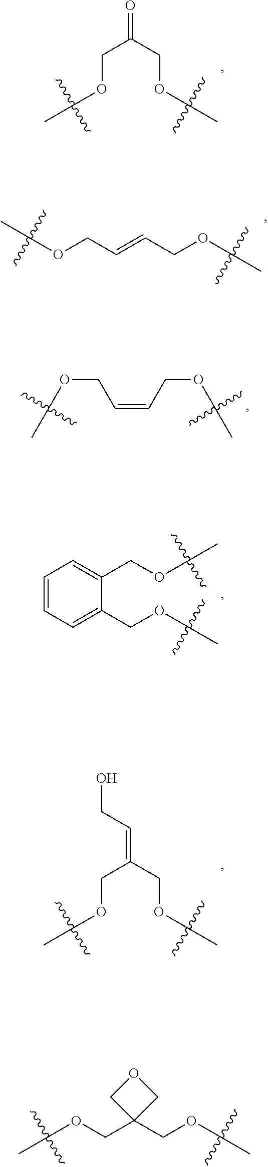 [SMILES: *OCC(=O)CO* ; *OC/C=C/CO* ; *OC/C=C\CO* ; *OCc1ccccc1CO* ; *OCC(=CCO)CO* ; *OCC1(CO*)COC1]

### or —N(CH₃)CONH—.

In some embodiments of Formula I, I-1, II, III or IV, each R₄ is independently selected from the group consisting of H, deuterium, F, Cl, Br, I, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶).

In some embodiments of Formula I, I-1, II, III or IV, each R₄ is independently selected from the group consisting of H, deuterium, F, Cl, Br, I, OH, C₁-C₃ alkyl, C₁-C₃ haloalkyl, OC₁-C₃ alkyl, OC₁-C₃ haloalkyl, C₂-C₃ alkenyl, C₂-C₃alkynyl, and N(R⁶)₂.

In some embodiments of Formula I, I-1, II, III or IV, each R₄ independently is selected from the group consisting of H, deuterium, Br, Cl, OH, CH₃, CH₂CH₃, CH═CH₂, C≡CH, OCH₃, OCFH₂, OCF₂H, OCF₃, and N(R⁶)₂.

In some embodiments of Formula I, I-1, II, III or IV, each R₄ independently is selected from the group consisting of H, deuterium, Br, OH, CH₃, CH₂CH₃, CH═CH₂, C≡CH, OCH₃, NH₂ and NHCH₃.

In some embodiments of Formula I, I-1, II, III, IV or V, each R⁶ is independently selected from the group consisting of —H, deuterium, —F, —Cl, —Br, —I, —NH₂, —CN, —OH, —N₃, —NO₂, carboxyl, C₁-C₃alkyl, C₁-C₃alkoxy, C₂-C₄alkenyl, C₂-C₄alkynyl, 6-membered aryl, 7-membered aryl, 8-membered aryl, 5-membered heteroaryl, 6-membered heteroaryl, 7-membered heteroaryl, 8-membered heteroaryl, 5-membered heterocyclic ring, 6-membered heterocyclic ring, 7-membered heterocyclic ring, 8-membered heterocyclic ring, 5-membered carbocyclic ring, 6-membered carbocyclic ring, 7-membered carbocyclic ring, or 8-membered carbocyclic ring; and each of which is independently optionally substituted with deuterium, —F, —Cl, —Br, —I, —NH₂, —CN, —OH, —NO₂, carbonyl, ═O, oxo, carboxyl, C₁-C₃alkoxy, or C₁-C₃alkyl; and each of the heteroaryl and heterocyclic ring contains 1 or 2 heteroatoms selected from N, O or S.

In some embodiments of Formula I, I-1, II, III, IV or V, each R⁶ is independently selected from the group consisting of —H, deuterium, —F, —Cl, —Br, —I, —NH₂, —CN, —OH, —N₃, —NO₂, carboxyl, methyl, ethyl, propyl, isopropyl, methoxy, ethoxy, propoxy, isopropoxy, ethylene, 6-membered aryl, 5-membered heteroaryl, 6-membered heteroaryl, 5-membered heterocyclic ring, 6-membered heterocyclic ring, 5-membered carbocyclic ring, or 6-membered carbocyclic ring; and each of which is independently optionally substituted with deuterium, —F, —Cl, —Br, —I, —NH₂, —CN, —OH, —NO₂, carbonyl, ═O, oxo, carboxyl, methyl, ethyl, propyl, isopropyl, methoxy, ethoxy, propoxy or isopropoxy; and each of the heteroaryl and heterocyclic ring contains 1 or 2 heteroatoms selected from N, O or S.

In some embodiments of Formula I, I-1, II, III, IV or V, each R⁶ is independently selected from the group consisting of H, deuterium, —F, —Cl, —Br, —I, —NH₂, —CN, —OH, methyl, ethyl, propyl, isopropyl, methoxy, ethoxy, propoxy, isopropoxy, CH2F, —CHF2,

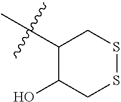 [SMILES: *C1CSSCC1O]

### and —CF3.

In some embodiments of Formula I, I-1, II, III, IV or V, each X₁ is independently selected from the group consisting of C═O, —CH₂—, —CHF—, and —CF₂—.

In some embodiments of Formula I, I-1, II, III, IV or V, each X₁ is selected from the group consisting of C═O and —CH₂—.

In some embodiments of Formula I, I-1, II, III, IV or V, each X₁ is C═O.

In some embodiments of Formula I, I-1, II, III, IV or V, each X₂ is independently selected from (C(R⁸)₂)₍₁₋₃₎, wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle.

In some embodiments, each X₂ independently is —(C(R⁸)₂)₁₋₃—, wherein each R⁸ independently is hydrogen, deuterium, halogen, C₁-C₆ alkyl, CN, OR⁶, N(R⁶)₂, or C₃-C₆ cycloalkyl; wherein the C₁-C₆ alkyl is optionally substituted with one or more halogen, OR⁶, or N(R⁶)₂.

In some embodiments, each X₂ independently is —(C(R⁸)₂)₁₋₃—, wherein at least two R⁸, together with the one or more atoms to which they are attached, form C₃-C₆ cycloalkyl or 3- to 6-membered heterocycloalkyl.

In some embodiments, each X₂ independently is —C(R⁸)₁₋₃—.

In some embodiments, each X₂ independently is —(CH₂)₁₋₃—.

In some embodiments, each X₂ independently is —C(R⁸)₂—.

In some embodiments, each X₂ independently is —CH₂—.

In some embodiments, each X₂ independently is —C(R⁸)₂C(R⁸)₂—.

In some embodiments, each X₂ independently is —CH₂CH₂—.

In some embodiments, each X₂ independently is —C(R⁸)2C(R⁸)₂C(R⁸)₂—.

In some embodiments, each X₂ independently is —CH₂CH₂CH₂—.

In some embodiments of Formula I, I-1, II, III, IV or V, each X₂ is CH₂CHR⁸, where R⁸ is selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl.

In some embodiments of Formula I, I-1, II, III, IV or V, each X₂ is CH₂CHR⁸, wherein R⁸ is selected from the group consisting of H, deuterium, CH₃, CH₂OH, CH₂CH₃, CH₂CH₂CH₃, CH(CH₃)₂, CH₂OCH₃, and cyclopropyl.

In some embodiments of Formula I, I-1, II, III, IV or V, each X₂ is CHR⁸CHR⁸, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on different carbon atoms are taken together, along with the atoms to which they are attached, to form a 3- to 6-membered fused ring.

In some embodiments of Formula I, I-1, II, III, IV or V, each X₂ is CHR⁸CHR⁸, where each R⁸ is independently selected from the group consisting of H, deuterium and C₁-C₃ alkyl, and optionally the 2 R⁸ on different carbon atoms are taken together, along with the atoms to which they are attached, to form a 3- to 6-membered fused ring.

In some embodiments of Formula I, I-1, II, III, IV or V, each X₂ is CH₂C(R⁸)₂, where each R⁸ is independently selected from the group consisting of H, deuterium, C₁-C₃alkyl, C₁-C₃ alkyl substituted by OH, C₁-C₃ alkyl substituted by OC₁-C₃ alkyl, and C₃-C₆ cycloalkyl, and optionally the 2 R⁸ on a single carbon atom are taken together, along with the atoms to which they are attached, to form a 3- to 6-membered spirocycle.

In some embodiments of Formula I, I-1, II, III, IV or V, each X₂ is CH₂C(R⁸)₂, where each R⁸ is independently selected from the group consisting of H, deuterium and C₁-C₃ alkyl, and optionally the 2 R⁸ on a single carbon atom are taken together, along with the atoms to which they are attached, to form a 3- to 6-membered spirocycle.

In some embodiments of Formula I, I-1, II, III, IV or V, each X₃ is independently selected from the group consisting of COOR⁶, C(O)SR⁶, C(S)OR⁶, SO₂R⁶, C(O)N(R⁹)₂,

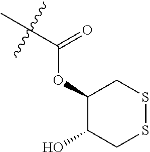 [SMILES: *C(=O)O[C@H]1CSSC[C@@H]1O]

### and CN.

In some embodiments of Formula I, I-1, II, III, IV or V, each X₃ is independently selected from the group consisting of COOR⁶, SO₂R⁶, C(O)N(R⁹)₂,

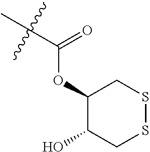 [SMILES: *C(=O)O[C@H]1CSSC[C@@H]1O]

### and CN.

In some embodiments of Formula I, I-1, II, III, IV or V, each X₃ is independently selected from the group consisting of COOR⁶, C(O)N(R⁹)₂,

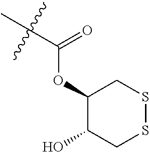 [SMILES: *C(=O)O[C@H]1CSSC[C@@H]1O]

### and CN.

In some embodiments of Formula I, I-1, II, III, IV or V, each X₃ is independently selected from the group consisting of COOH, COOCH₃, CONH₂, CONH—SO₂—N(CH₃)₂, CONH—SO₂—CH₃,

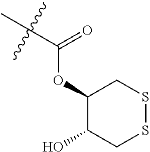 [SMILES: *C(=O)O[C@H]1CSSC[C@@H]1O]

### and CN.

In some embodiments of Formula I, I-1, II, III, IV or V, each R⁹ is independently selected from the group consisting of H, deuterium, COOR⁶, and SO₂R⁶.

In some embodiments of Formula I, I-1, II, III, IV or V, each R⁹ is independently H or deuterium, preferably H.

In some embodiments, the compound is of Formula Ia, IIa, IIIa, IVa or Va:

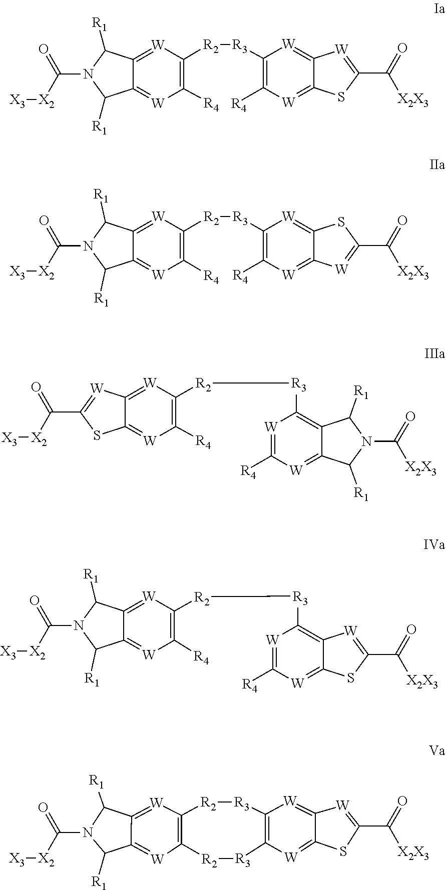

or a pharmaceutically acceptable salt thereof.

In some embodiments, the compound is of Formula Ib, IIb, IIIb, or IVb:

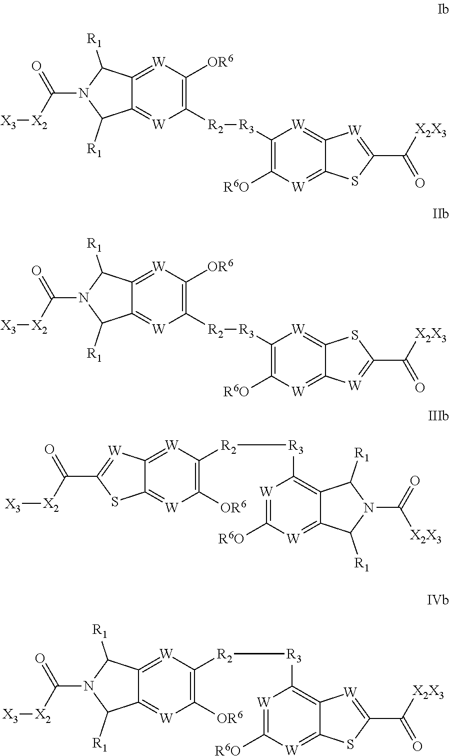

or a pharmaceutically acceptable salt thereof.

In some embodiments, the compound is of Formula Ic, IIc, IIIc, IVc or Vc:

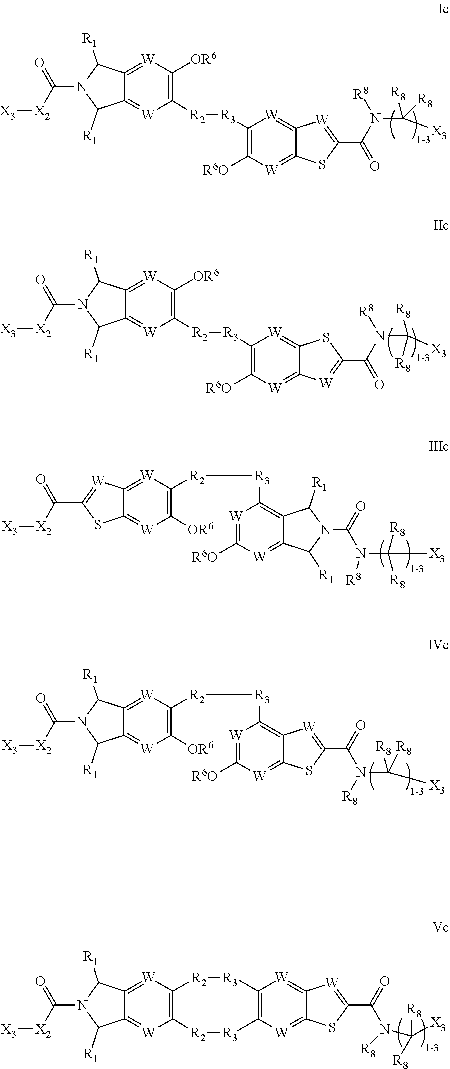

or a pharmaceutically acceptable salt thereof.

In some embodiments of the preceding Formulae defined herein, each heterocyclic ring group and each carbocyclic ring group includes single ring, spiral ring, bridge ring, fused ring and various combinations of spiral ring, bridge ring and/or fused ring.

In some embodiments of the preceding Formulae defined herein, the compound is selected from the compounds described in Table 1 and pharmaceutically acceptable salts thereof.

In some embodiments of the preceding Formulae defined herein, the compound is the compounds described in Table 1.

TABLE 1

| | |
|---|---|
| 1. | 4-(5-(3-((2-(3-carboxypropanoyl)-6-methoxybenzo[b]thiophen-5-yl)oxy)propoxy)-6- |
| | methoxyisoindolin-2-yl)-4-oxobutanoic acid |
| 2. | 4-(5-(3-((2-(3-carboxypropanoyl)-4-chloro-6-methoxyisoindolin-5-yl)oxy)propoxy)-4- |
| | fluoro-6-methoxybenzo[b]thiophen-2-yl)-4-oxobutanoic acid |
| 3. | 4-(5-(3-((2-(3-carboxypropanoyl)-4-chloro-6-methoxybenzo[b]thiophen-5-yl)oxy)propoxy)- |
| | 4-fluoro-6-methoxyisoindolin-2-yl)-4-oxobutanoic acid |
| 4. | 4-(5-(3-((2-(3-carboxypropanoyl)-4-fluoro-6-methoxybenzo[b]thiophen-5-yl)oxy)propoxy)- |
| | 4-fluoro-6-methoxyisoindolin-2-yl)-4-oxobutanoic acid |
| 5. | sodium |
| | (S)-4-(5-(3-((2-((S)-3-carboxylatobutanoyl)-6-methoxybenzo[b]thiophen-5-yl)oxy)pro- |
| | poxy)-6-methoxyisoindolin-2-yl)-2-methyl-4-oxobutanoate |
| 6. | sodium |
| | (S)-4-(5-(3-((2-((S)-3-carboxylatobutanoyl)-4-fluoro-6-methoxybenzo[b]thiophen-5-yl)oxy) |
| | propoxy)-6-methoxyisoindolin-2-yl)-2-methyl-4-oxobutanoate |
| 7. | sodium |
| | 4-(5-(3-((2-(3-carboxylatopropanoyl)-6-hydroxybenzo[b]thiophen-5-yl)oxy)propoxy)-6- |
| | methoxyisoindolin-2-yl)-4-oxobutanoate |
| 8. | sodium |
| | 4-(5-(3-((2-(3-carboxylatopropanoyl)-6-methoxyisoindolin-5-yl)oxy)propoxy)-6- |
| | methoxythieno[3,2-b]pyridin-2-yl)-4-oxobutanoate |
| 9. | 4-(5-(3-((6-bromo-2-(3-carboxypropanoyl)benzo[b]thiophen-5-yl)oxy)propoxy)-6-methoxy |
| | isoindolin-2-yl)-4-oxobutanoic acid |
| 10. | 4-(5-(3-((2-(3-carboxypropanoyl)-4-fluoro-6-methoxyisoindolin-5-yl)oxy)propoxy)-6- |
| | methoxybenzo[b]thiophen-2-yl)-4-oxobutanoic acid |
| 11. | 4-(5-(3-((6-(3-carboxypropanoyl)-3-methoxy-6,7-dihydro-5H-pyrrolo[3,4-b]pyridin-2- |
| | yl)oxy)propoxy)-6-methoxythieno[3,2-b]pyridin-2-yl)-4-oxobutanoic acid |
| 12. | 4-(5-(3-((2-(4-ethoxy-4-oxobutanoyl)-6-methoxybenzo[b]thiophen-5-yl)oxy)propoxy)- |
| | 6-methoxyisoindolin-2-yl)-4-oxobutanoic acid |
| 13. | sodium |
| | 4-(5-(3-((2-(3-carboxylatopropanoyl)-5-methoxythieno[2,3-b]pyridin-6-yl)oxy)propoxy)-6- |
| | methoxyisoindolin-2-yl)-4-oxobutanoate |
| 14. | 4-(5-(3-((2-(3-carboxypropanoyl)-4-chloro-6-methoxyisoindolin-5-yl)oxy)propoxy)-6- |
| | methoxybenzo[b]thiophen-2-yl)-4-oxobutanoic acid |
| 15. | 4-(5-(3-((2-(3-carboxypropanoyl)-5-methoxythieno[3,2-b]pyridin-6-yl)oxy)propoxy)-6- |
| | methoxyisoindolin-2-yl)-4-oxobutanoic acid |
| 16. | 4-(5-((2-(((2-(3-carboxypropanoyl)-4-chloro-6-methoxyisoindolin-5-yl)oxy)meth- |
| | yl)allyl)oxy)-4-fluoro-6-methoxybenzo[b]thiophen-2-yl)-4-oxobutanoic acid |
| 17. | (S)-4-(5-(3-((2-((S)-3-carboxybutanoyl)-4-fluoro-6-methoxybenzo[b]thiophen-5- |
| | yl)oxy)propoxy)-4-fluoro-6-methoxyisoindolin-2-yl)-2-methyl-4-oxobutanoic acid |
| 18. | 4-(5-(3-((2-((2-carboxyethyl)carbamoyl)-6-methoxybenzo[b]thiophen-5-yl)oxy)propoxy)-6- |
| | methoxyisoindolin-2-yl)-4-oxobutanoic acid |
| 19. | 4-(5-((2-(((2-(3-carboxypropanoyl)-6-methoxybenzo[b]thiophen-5-yl)oxy)meth- |
| | yl)allyl)oxy)-6-methoxyisoindolin-2-yl)-4-oxobutanoic acid |
| 20. | 4-(5-(3-((2-(3-carboxypropanoyl)-4-fluoro-6-methoxybenzo[b]thiophen-5-yl)oxy)propoxy)- |
| | 6-methoxyisoindolin-2-yl)-4-oxobutanoic acid |
| 21. | trans-2-(5-(3-((2-(3-carboxypropanoyl)-4-fluoro-6-methoxyisoindolin-5-yl)oxy)propoxy)-6- |
| | methoxybenzo[b]thiophene-2-carbonyl)cyclopropane-1-carboxylic acid |
| 22. | 4-(5-(3-((2-(3-carboxybutanoyl)-6-methoxybenzo[b]thiophen-5-yl)oxy)propoxy)-6- |
| | methoxyisoindolin-2-yl)-2-methyl-4-oxobutanoic acid |
| 23. | (S)-4-(5-(3-((2-((S)-3-carboxybutanoyl)-6-methoxybenzo[b]thiophen-5-yl)oxy)propoxy)-6- |
| | methoxyisoindolin-2-yl)-2-methyl-4-oxobutanoic acid |
| 24. | (S)-4-(5-(3-((2-(3-carboxypropanoyl)-6-methoxyisoindolin-5-yl)oxy)propoxy)-6-meth- |
| | oxybenzo[b]thiophen-2-yl)-2-methyl-4-oxobutanoic acid |
| 25. | (S)-4-(5-(3-((2-(3-carboxypropanoyl)-6-methoxybenzo[b]thiophen-5-yl)oxy)propoxy)-6- |
| | methoxyisoindolin-2-yl)-2-methyl-4-oxobutanoic acid |
| 26. | (S)-4-(5-(2-((2-((S)-3-carboxybutanoyl)-6-methoxybenzo[b]thiophen-5-yl)oxy)ethoxy)-6- |
| | methoxyisoindolin-2-yl)-2-methyl-4-oxobutanoic acid |
| 27. | (S)-4-(5-(4-((2-((S)-3-carboxybutanoyl)-6-methoxybenzo[b]thiophen-5-yl)oxy)butoxy)-6- |
| | methoxyisoindolin-2-yl)-2-methyl-4-oxobutanoic acid |
| 28. | (S)-4-(5-((5-((2-((S)-3-carboxybutanoyl)-6-methoxybenzo[b]thiophen-5- |
| | yl)oxy)pentyl)oxy)-6-methoxyisoindolin-2-yl)-2-methyl-4-oxobutanoic acid |
| 29. | (S)-4-(5-(4-(2-((S)-3-carboxybutanoyl)-6-methoxybenzo[b]thiophen-5-yl)butoxy)-6- |
| | methoxyisoindolin-2-yl)-2-methyl-4-oxobutanoic acid |
| 30. | (S)-4-(5-(3-((2-((S)-3-carboxybutanoyl)-6-methoxybenzo[b]thiophen-5-yl)amino)propoxy)- |
| | 6-methoxyisoindolin-2-yl)-2-methyl-4-oxobutanoic acid |
| 31. | (S)-4-(5-(4-((2-((S)-3-carboxybutanoyl)-6-methoxybenzo[b]thiophen-5-yl)amino)butyl)-6- |
| | methoxyisoindolin-2-yl)-2-methyl-4-oxobutanoic acid |
| 32. | (S)-4-(5-(4-(2-((S)-3-carboxybutanoyl)-6-methoxybenzo[b]thiophen-5-yl)butyl)-6- |
| | methoxyisoindolin-2-yl)-2-methyl-4-oxobutanoic acid |
| 33. | (S)-4-(5-(5-(2-((S)-3-carboxybutanoyl)-6-methoxybenzo[b]thiophen-5-yl)pentyl)-6- |
| | methoxyisoindolin-2-yl)-2-methyl-4-oxobutanoic acid |
| 34. | (2S)-4-(5-((4-((2-((S)-3-carboxybutanoyl)-6-methoxybenzo[b]thiophen-5-yl)oxy)pentan-2- |
| | yl)oxy)-6-methoxyisoindolin-2-yl)-2-methyl-4-oxobutanoic acid |
| 35. | (S)-4-(5-(3-((2-((S)-3-carboxybutanoyl)-6-methoxybenzo[b]thiophen-5-yl)oxy)-2,2- |
| | dimethylpropoxy)-6-methoxyisoindolin-2-yl)-2-methyl-4-oxobutanoic acid |
| 36. | (S)-4-(5-((1-(((2-((S)-3-carboxybutanoyl)-6-methoxybenzo[b]thiophen-5-yl)oxy)methyl)cy- |
| | clopropyl)methoxy)-6-methoxyisoindolin-2-yl)-2-methyl-4-oxobutanoic acid |
| 37. | 4-(5-(3-((2-(3-carboxypropanoyl)-6-methoxyisoindolin-5-yl)oxy)propoxy)-6-methoxy- |
| | benzo[b]thiophen-2-yl)-2,2-dimethyl-4-oxobutanoic acid |
| 38. | 4-(5-(3-((2-(3-carboxypropanoyl)-6-methoxybenzo[b]thiophen-5-yl)oxy)propoxy)-6- |
| | methoxyisoindolin-2-yl)-2,2-dimethyl-4-oxobutanoic acid |
| 39. | 4-(5-(3-((2-(3-carboxy-3-methylbutanoyl)-6-methoxybenzo[b]thiophen-5-yl)oxy)propoxy)- |
| | 6-methoxyisoindolin-2-yl)-2,2-dimethyl-4-oxobutanoic acid |
| 40. | (S)-4-(5-((4-(2-((S)-3-carboxybutanoyl)-6-methoxybenzo[b]thiophen-5-yl)butyl)amino)-6- |
| | methoxyisoindolin-2-yl)-2-methyl-4-oxobutanoic acid |
| 41. | (S)-4-(5-((3-((2-((S)-3-carboxybutanoyl)-6-methoxybenzo[b]thiophen-5-yl)oxy)pro- |
| | pyl)amino)-6-methoxyisoindolin-2-yl)-2-methyl-4-oxobutanoic acid |
| 42. | (S)-4-(5-(4-((2-((S)-3-carboxybutanoyl)-6-methoxybenzo[b]thiophen-5-yl)oxy)butyl)-6- |
| | methoxyisoindolin-2-yl)-2-methyl-4-oxobutanoic acid |
| 43. | (S)-4-(5-(3-((2-((S)-3-carboxybutanoyl)-4-fluoro-6-methoxybenzo[b]thiophen-5- |
| | yl)oxy)pro-poxy)-6-methoxyisoindolin-2-yl)-2-methyl-4-oxobutanoic acid |
| 44. | (S)-4-(5-(3-((2-((S)-3-carboxybutanoyl)-4-chloro-6-methoxybenzo[b]thiophen-5- |
| | yl)oxy)propoxy)-6-methoxyisoindolin-2-yl)-2-methyl-4-oxobutanoic acid |
| 45. | (S)-4-(5-(3-((4-bromo-2-((S)-3-carboxybutanoyl)-6-methoxybenzo[b]thiophen-5- |
| | yl)oxy)propoxy)-6-methoxyisoindolin-2-yl)-2-methyl-4-oxobutanoic acid |
| 46. | (S)-4-(5-(3-((2-((S)-3-carboxybutanoyl)-6-methoxybenzo[d]thiazol-5-yl)oxy)propoxy)- |
| | 6-methoxyisoindolin-2-yl)-2-methyl-4-oxobutanoic acid |
| 47. | (S)-4-(5-(3-((2-((S)-3-carboxybutanoyl)-6-hydroxybenzo[b]thiophen-5-yl)oxy)propoxy)-6- |
| | methoxyisoindolin-2-yl)-2-methyl-4-oxobutanoic acid |
| 48. | (S)-4-(5-(3-((2-((S)-3-carboxybutanoyl)-4,7-difluoro-6-methoxybenzo[b]thiophen-5- |
| | yl)oxy)propoxy)-6-methoxyisoindolin-2-yl)-2-methyl-4-oxobutanoic acid |
| 49. | (S)-4-(5-(3-((2-((S)-3-carboxybutanoyl)-4,7-dichloro-6-methoxybenzo[b]thiophen-5- |
| | yl)oxy)propoxy)-6-methoxyisoindolin-2-yl)-2-methyl-4-oxobutanoic acid |
| 50. | (2S)-4-(5-(3-((2-((S)-3-carboxybutanoyl)-6-methoxy-3-methylisoindolin-5-yl)oxy)pro- |
| | poxy)-6-methoxybenzo[b]thiophen-2-yl)-2-methyl-4-oxobutanoic acid |
| 51. | (S)-4-(5-(3-((2-((S)-3-carboxybutanoyl)-4-fluoro-6-methoxyisoindolin-5-yl)oxy)propoxy)- |
| | 6-methoxybenzo[b]thiophen-2-yl)-2-methyl-4-oxobutanoic acid |
| 52. | (S)-4-(5-(3-((2-((S)-3-carboxybutanoyl)-4-chloro-6-methoxyisoindolin-5-yl)oxy)propoxy)- |
| | 6-methoxybenzo[b]thiophen-2-yl)-2-methyl-4-oxobutanoic acid |
| 53. | (S)-4-(5-(3-((4-bromo-2-((S)-3-carboxybutanoyl)-6-methoxyisoindolin-5-yl)oxy)propoxy)- |
| | 6-methoxybenzo[b]thiophen-2-yl)-2-methyl-4-oxobutanoic acid |
| 54. | (S)-4-(5-(3-((2-((S)-3-carboxybutanoyl)-6-hydroxyisoindolin-5-yl)oxy)propoxy)-6- |
| | methoxybenzo[b]thiophen-2-yl)-2-methyl-4-oxobutanoic acid |
| 55. | (S)-4-(6-(3-((2-((S)-3-carboxybutanoyl)-6-methoxybenzo[b]thiophen-5-yl)oxy)propoxy)-4- |
| | fluoro-5-methoxyisoindolin-2-yl)-2-methyl-4-oxobutanoic acid |
| 56. | (S)-4-(6-(3-((2-((S)-3-carboxybutanoyl)-6-methoxybenzo[b]thiophen-5-yl)oxy)propoxy)-4- |
| | chloro-5-methoxyisoindolin-2-yl)-2-methyl-4-oxobutanoic acid |
| 57. | (S)-4-(5-(3-((7-bromo-2-((S)-3-carboxybutanoyl)-6-methoxyisoindolin-5-yl)oxy)propoxy)- |
| | 6-methoxybenzo[b]thiophen-2-yl)-2-methyl-4-oxobutanoic acid |
| 58. | (S)-4-(5-(3-((2-((S)-3-carboxybutanoyl)-4,7-difluoro-6-methoxyisoindolin-5-yl)oxy)pro- |
| | poxy)-6-methoxybenzo[b]thiophen-2-yl)-2-methyl-4-oxobutanoic acid |
| 59. | (S)-4-(5-(3-((2-((S)-3-carboxybutanoyl)-6-methoxybenzo[b]thiophen-5-yl)oxy)propoxy)-7- |
| | chloro-4-fluoro-6-methoxyisoindolin-2-yl)-2-methyl-4-oxobutanoic acid |
| 60. | (S)-4-(5-(3-((2-((S)-3-carboxybutanoyl)-4,7-dichloro-6-methoxyisoindolin-5-yl)oxy)pro- |
| | poxy)-6-methoxybenzo[b]thiophen-2-yl)-2-methyl-4-oxobutanoic acid |
| 61. | (S)-4-(5-(3-((2-((S)-3-carboxybutanoyl)-4-chloro-6-methoxybenzo[b]thiophen-5- |
| | yl)oxy)propoxy)-4-fluoro-6-methoxyisoindolin-2-yl)-2-methyl-4-oxobutanoic acid |
| 62. | (S)-4-(5-(3-((2-((S)-3-carboxybutanoyl)-4-chloro-6-methoxyisoindolin-5-yl)oxy)propoxy)- |
| | 4-fluoro-6-methoxybenzo[b]thiophen-2-yl)-2-methyl-4-oxobutanoic acid |
| 63. | (S)-4-(5-(3-((2-((S)-3-carboxybutanoyl)-4-chloro-6-methoxybenzo[b]thiophen-5- |
| | yl)oxy)propoxy)-4-chloro-6-methoxyisoindolin-2-yl)-2-methyl-4-oxobutanoic acid |
| 64. | (S)-4-(6-(3-((2-((S)-3-carboxybutanoyl)-7-fluoro-6-methoxybenzo[b]thiophen-5- |
| | yl)oxy)propoxy)-4-fluoro-5-methoxyisoindolin-2-yl)-2-methyl-4-oxobutanoic acid |
| 65. | (S)-4-(6-(3-((2-((S)-3-carboxybutanoyl)-7-chloro-6-methoxybenzo[b]thiophen-5- |
| | yl)oxy)propoxy)-4-fluoro-5-methoxyisoindolin-2-yl)-2-methyl-4-oxobutanoic acid |
| 66. | (S)-4-(5-(3-((2-((S)-3-carboxybutanoyl)-7-chloro-6-methoxyisoindolin-5-yl)oxy)propoxy)- |
| | 7-fluoro-6-methoxybenzo[b]thiophen-2-yl)-2-methyl-4-oxobutanoic acid |
| 67. | (S)-4-(5-(3-((2-((S)-3-carboxybutanoyl)-4,7-difluoro-6-methoxyisoindolin-5- |
| | yl)oxy)propoxy)-7-chloro-6-methoxybenzo[b]thiophen-2-yl)-2-methyl-4-oxobutanoic acid |
| 68. | (S)-4-(5-(3-((2-((S)-3-carboxybutanoyl)-4,7-difluoro-6-methoxyisoindolin-5-yl)oxy)pro- |
| | poxy)-7-fluoro-6-methoxybenzo[b]thiophen-2-yl)-2-methyl-4-oxobutanoic acid |
| 69. | (S)-4-(5-(3-((2-((S)-3-carboxybutanoyl)-4-chloro-7-fluoro-6-methoxyisoindolin-5- |
| | yl)oxy)propoxy)-7-fluoro-6-methoxybenzo[b]thiophen-2-yl)-2-methyl-4-oxobutanoic acid |
| 70. | (S)-4-(6-(3-((2-((S)-3-carboxybutanoyl)-4,7-difluoro-6-methoxybenzo[b]thiophen-5- |
| | yl)oxy)propoxy)-4-fluoro-5-methoxyisoindolin-2-yl)-2-methyl-4-oxobutanoic acid |
| 71. | (S)-4-(6-(3-((2-((S)-3-carboxybutanoyl)-7-chloro-4-fluoro-6-methoxybenzo[b]thiophen- |
| | 5-yl)oxy)propoxy)-4-fluoro-5-methoxyisoindolin-2-yl)-2-methyl-4-oxobutanoic acid |
| 72. | (S)-4-(6-(3-((2-((S)-3-carboxybutanoyl)-4,7-dichloro-6-methoxybenzo[b]thiophen-5- |
| | yl)oxy)propoxy)-4-fluoro-5-methoxyisoindolin-2-yl)-2-methyl-4-oxobutanoic acid |
| 73. | (S)-4-(5-(3-((2-((S)-3-carboxybutanoyl)-4-chloro-7-fluoro-6-methoxyisoindolin-5- |
| | yl)oxy)propoxy)-7-chloro-6-methoxybenzo[b]thiophen-2-yl)-2-methyl-4-oxobutanoic acid |
| 74. | (S)-4-(5-(3-((2-((S)-3-carboxybutanoyl)-4,7-dichloro-6-methoxyisoindolin-5-yl)oxy)pro- |
| | poxy)-7-fluoro-6-methoxybenzo[b]thiophen-2-yl)-2-methyl-4-oxobutanoic acid |
| 75. | (S)-4-(5-(3-((2-((S)-3-carboxybutanoyl)-7-chloro-4-fluoro-6-methoxyisoindolin-5- |
| | yl)oxy)propoxy)-7-fluoro-6-methoxybenzo[b]thiophen-2-yl)-2-methyl-4-oxobutanoic acid |
| 76. | (S)-4-(6-(3-((2-((S)-3-carboxybutanoyl)-4,7-difluoro-6-methoxybenzo[b]thiophen-5- |
| | yl)oxy)propoxy)-4-chloro-5-methoxyisoindolin-2-yl)-2-methyl-4-oxobutanoic acid |
| 77. | (S)-4-(6-(3-((2-((S)-3-carboxybutanoyl)-4-chloro-7-fluoro-6-methoxybenzo[b]thiophen- |
| | 5-yl)oxy)propoxy)-4-chloro-5-methoxyisoindolin-2-yl)-2-methyl-4-oxobutanoic acid |
| 78. | (S)-4-(5-(3-((2-((S)-3-carboxybutanoyl)-4-fluoro-6-hydroxyisoindolin-5-yl)oxy)propoxy)- |
| | 4-fluoro-6-methoxybenzo[b]thiophen-2-yl)-2-methyl-4-oxobutanoic acid |
| 79. | (S)-4-(5-(3-((2-((S)-3-carboxybutanoyl)-4-chloro-6-methoxybenzo[b]thiophen-5- |
| | yl)oxy)propoxy)-4-fluoro-6-hydroxyisoindolin-2-yl)-2-methyl-4-oxobutanoic acid |
| 80. | (S)-4-(5-(3-((2-((S)-3-carboxybutanoyl)-4-chloro-6-hydroxyisoindolin-5-yl)oxy)propoxy)- |
| | 4-fluoro-6-methoxybenzo[b]thiophen-2-yl)-2-methyl-4-oxobutanoic acid |
| 81. | (S)-4-(5-(3-((2-((S)-3-carboxybutanoyl)-4-chloro-6-hydroxyisoindolin-5-yl)oxy)propoxy)- |
| | 4-chloro-6-methoxybenzo[b]thiophen-2-yl)-2-methyl-4-oxobutanoic acid |
| 82. | (S)-4-(6-(3-((2-((S)-3-carboxybutanoyl)-6-methoxybenzo[b]thiophen-5-yl)oxy)propoxy)-4- |
| | fluoro-5-hydroxyisoindolin-2-yl)-2-methyl-4-oxobutanoic acid |
| 83. | (S)-4-(5-(3-((2-((S)-3-carboxybutanoyl)-7-fluoro-6-hydroxyisoindolin-5-yl)oxy)propoxy)- |
| | 7-fluoro-6-methoxybenzo[b]thiophen-2-yl)-2-methyl-4-oxobutanoic acid |
| 84. | (S)-4-(6-(3-((2-((S)-3-carboxybutanoyl)-7-chloro-6-methoxybenzo[b]thiophen-5- |
| | yl)oxy)propoxy)-4-fluoro-5-hydroxyisoindolin-2-yl)-2-methyl-4-oxobutanoic acid |
| 85. | (S)-4-(5-(3-((2-((S)-3-carboxybutanoyl)-7-chloro-6-hydroxyisoindolin-5-yl)oxy)propoxy)- |
| | 7-fluoro-6-methoxybenzo[b]thiophen-2-yl)-2-methyl-4-oxobutanoic acid |
| 86. | (S)-4-(5-(3-((2-((S)-3-carboxybutanoyl)-4,7-difluoro-6-methoxyisoindolin-5-yl)oxy)pro- |
| | poxy)-4-chloro-7-fluoro-6-methoxybenzo[b]thiophen-2-yl)-2-methyl-4-oxobutanoic acid |
| 87. | (S)-4-(5-(3-((2-((S)-3-carboxybutanoyl)-4-chloro-7-fluoro-6-methoxybenzo[b]thiophen-5- |
| | yl)oxy)propoxy)-4-chloro-7-fluoro-6-methoxyisoindolin-2-yl)-2-methyl-4-oxobutanoic acid |
| 88. | (S)-4-(5-(3-((2-((S)-3-carboxybutanoyl)-4,7-difluoro-6-methoxybenzo[b]thiophen-5- |
| | yl)oxy)propoxy)-4, 7-difluoro-6-methoxyisoindolin-2-yl)-2-methyl-4-oxobutanoic acid |
| 89. | (S)-4-(5-(3-((2-((S)-3-carboxybutanoyl)-4,7-difluoro-6-methoxyisoindolin-5-yl)oxy)pro- |
| | poxy)-7-chloro-4-fluoro-6-methoxybenzo[b]thiophen-2-yl)-2-methyl-4-oxobutanoic acid |
| 90. | (S)-4-(5-(3-((2-((S)-3-carboxybutanoyl)-4,7-dichloro-6-methoxybenzo[b]thiophen-5- |
| | yl)oxy)propoxy)-4,7-difluoro-6-methoxyisoindolin-2-yl)-2-methyl-4-oxobutanoic acid |
| 91. | (S)-4-(5-(3-((2-((S)-3-carboxybutanoyl)-4,7-dichloro-6-methoxybenzo[b]thiophen-5- |
| | yl)oxy)propoxy)-4-chloro-7-fluoro-6-methoxyisoindolin-2-yl)-2-methyl-4-oxobutanoic acid |
| 92. | (S)-4-(5-(3-((2-((S)-3-carboxybutanoyl)-4-chloro-7-fluoro-6-methoxyisoindolin-5- |
| | yl)oxy)propoxy)-7-chloro-4-fluoro-6-methoxybenzo[b]thiophen-2-yl)-2-methyl-4- |
| | oxobutanoic acid |
| 93. | (S)-4-(5-(3-((2-((S)-3-carboxybutanoyl)-4,7-dichloro-6-methoxyisoindolin-5-yl)oxy)pro- |
| | poxy)-4-chloro-7-fluoro-6-methoxybenzo[b]thiophen-2-yl)-2-methyl-4-oxobutanoic acid |
| 94. | (S)-4-(5-(3-((2-((S)-3-carboxybutanoyl)-4-chloro-7-fluoro-6-methoxybenzo[b]thiophen- |
| | 5-yl)oxy)propoxy)-7-chloro-4-fluoro-6-methoxyisoindolin-2-yl)-2-methyl-4- |
| | oxobutanoic acid |
| 95. | (2S,2′S)-4,4′-(7,8,11,13,17,18-hexahydro-6H,12H,16H-thieno[2″,3″:4′,5′]benzo[1′,2′:9,10] |
| | [1,4,8,11]tetraoxacyclotetradecino[2,3-f]isoindole-2,12-diyl)bis(2-methyl-4-oxobutanoic |
| | acid) |
| 96. | (S)-4-(6-methoxy-5-(3-((6-methoxy-2-((S)-3-methyl-4-(methylsulfonamido)-4- |
| | oxobutanoyl)isoindolin-5-yl)oxy)propoxy)benzo[b]thiophen-2-yl)-2-methyl-4- |
| | oxobutanoic acid |
| 97. | (S)-4-(5-(3-((2-((S)-4-((N,N-dimethylsulfamoyl)amino)-3-methyl-4-oxobutanoyl)- |
| | 6-methoxyisoindolin-5-yl)oxy)propoxy)-6-methoxybenzo[b]thiophen-2-yl)-2-methyl- |
| | 4-oxobutanoic acid |
| 98. | (S)-4-(5-(3-((2-(3-cyanopropanoyl)-6-methoxyisoindolin-5-yl)oxy)propoxy)-6- |
| | methoxybenzo[b]thiophen-2-yl)-2-methyl-4-oxobutanoic acid |
| 99. | 4-(5-(3-((2-(3-cyanopropanoyl)-6-methoxybenzo[b]thiophen-5-yl)oxy)propoxy)-6-methoxy |
| | isoindolin-2-yl)-4-oxobutanenitrile |
| 100. | (S)-4-(5-(3-((2-(3-cyanopropanoyl)-6-methoxybenzo[b]thiophen-5-yl)oxy)propoxy)-6- |
| | methoxyisoindolin-2-yl)-2-methyl-4-oxobutanoic acid |
| 101. | (1R,2R)-2-(5-(3-((2-((S)-3-carboxybutanoyl)-6-methoxyisoindolin-5-yl)oxy)propoxy)- |
| | 6-methoxybenzo[b]thiophene-2-carbonyl)cyclobutane-1-carboxylic acid |
| 102. | (1R,2R)-2-(5-(3-((2-((1R,2R)-2-carboxycyclobutane-1-carbonyl)-6-methoxybenzo[b]thio- |
| | phen-5-yl)oxy)propoxy)-6-methoxyisoindoline-2-carbonyl)cyclobutane-1-carboxylic acid |
| 103. | (1R,2R)-2-(5-(3-((2-((S)-3-carboxybutanoyl)-6-methoxybenzo[b]thiophen-5-yl)oxy)pro- |
| | poxy)-6-methoxyisoindoline-2-carbonyl)cyclobutane-1-carboxylic acid |
| 104. | (1R,2R)-2-(5-(3-((2-((S)-3-carboxybutanoyl)-6-methoxyisoindolin-5-yl)oxy)propoxy)- |
| | 6-methoxybenzo[b]thiophene-2-carbonyl)cyclopropane-1-carboxylic acid |
| 105. | (1R,2R)-2-(5-(3-((2-((1R,2R)-2-carboxycyclopropane-1-carbonyl)-6-methoxyben- |
| | zo[b]thiophen-5-yl)oxy)propoxy)-6-methoxyisoindoline-2-carbonyl)cyclopropane-1- |
| | carboxylic acid |
| 106. | (1R,2R)-2-(5-(3-((2-((S)-3-carboxybutanoyl)-6-methoxybenzo[b]thiophen-5-yl)oxy)pro- |
| | poxy)-6-methoxyisoindoline-2-carbonyl)cyclopropane-1-carboxylic acid |
| 107. | (S)-N-(N,N-dimethylsulfamoyl)-4-(5-(3-((2-((S)-4-((N,N-dimethylsulfamoyl)amino)- |
| | 3-methyl-4-oxobutanoyl)-6-methoxybenzo[b]thiophen-5-yl)oxy)propoxy)-6- |
| | methoxyisoindolin-2-yl)-2-methyl-4-oxobutanamide |
| 108. | (S)-4-(5-(3-((2-((S)-4-((N,N-dimethylsulfamoyl)amino)-3-methyl-4-oxobutanoyl)- |
| | 6-methoxybenzo[b]thiophen-5-yl)oxy)propoxy)-6-methoxyisoindolin-2-yl)-2-methyl-4- |
| | oxobutanoic acid |
| 109. | (S)-4-(6-methoxy-5-(3-((6-methoxy-2-((S)-3-methyl-4-(methylsulfonamido)-4- |
| | oxobutanoyl)isoindolin-5-yl)oxy)propoxy)benzo[b]thiophen-2-yl)-2-methyl-N-(methyl- |
| | sulfonyl)-4-oxobutanamide |
| 110. | (S)-4-(5-methoxy-6-(3-((6-methoxy-2-((S)-3-methyl-4-(methylsulfonamido)-4-oxobutanoyl |
| | )benzo[b]thiophen-5-yl)oxy)propoxy)isoindolin-2-yl)-2-methyl-4-oxobutanoic acid |
| 111. | (S)-4-(4-(3-((2-((S)-3-carboxybutanoyl)-6-methoxybenzo[b]thiophen-5-yl)oxy)propoxy)-6- |
| | methoxyisoindolin-2-yl)-2-methyl-4-oxobutanoic acid |
| 112. | (S)-4-(5-(3-((2-((S)-3-carboxybutanoyl)-6-methoxybenzo[b]thiophen-4-yl)oxy)propoxy)-6- |
| | methoxyisoindolin-2-yl)-2-methyl-4-oxobutanoic acid |
| 113. | (S)-4-(4-(4-((2-((S)-3-carboxybutanoyl)-6-methoxybenzo[b]thiophen-5-yl)oxy)butyl)-6- |
| | methoxyisoindolin-2-yl)-2-methyl-4-oxobutanoic acid |
| 114. | (S)-4-(4-(4-(2-((S)-3-carboxybutanoyl)-6-methoxybenzo[b]thiophen-5-yl)butoxy)-6- |
| | methoxyisoindolin-2-yl)-2-methyl-4-oxobutanoic acid |
| 115. | (S)-4-(5-(4-((2-((S)-3-carboxybutanoyl)-6-methoxybenzo[b]thiophen-4-yl)oxy)butyl)- |
| | 6-methoxyisoindolin-2-yl)-2-methyl-4-oxobutanoic acid |
| 116. | (S)-4-(5-(4-(2-((S)-3-carboxybutanoyl)-6-methoxybenzo[b]thiophen-4-yl)butoxy)-6- |
| | methoxyisoindolin-2-yl)-2-methyl-4-oxobutanoic acid |
| 117. | (S)-4-(5-(3-((2-((S)-3-carboxybutanoyl)-6-methoxyisoindolin-5-yl)oxy)propoxy)-6- |
| | methylbenzo[b]thiophen-2-yl)-2-methyl-4-oxobutanoic acid |
| 118. | (S)-4-(5-(3-((2-((S)-3-carboxybutanoyl)-6-ethylbenzo[b]thiophen-5-yl)oxy)propoxy)- |
| | 6-methoxyisoindolin-2-yl)-2-methyl-4-oxobutanoic acid |
| 119. | (S)-4-(5-(3-((2-((S)-3-carboxybutanoyl)-6-methoxyisoindolin-5-yl)oxy)propoxy)-6- |
| | vinylbenzo[b]thiophen-2-yl)-2-methyl-4-oxobutanoic acid |
| 120. | (S)-4-(5-(3-((2-((S)-3-carboxybutanoyl)-6-ethynylbenzo[b]thiophen-5-yl)oxy)propoxy)- |
| | 6-methoxyisoindolin-2-yl)-2-methyl-4-oxobutanoic acid |
| 121. | (S)-4-(5-(3-((6-amino-2-((S)-3-carboxybutanoyl)benzo[b]thiophen-5-yl)oxy)propoxy)- |
| | 6-methoxyisoindolin-2-yl)-2-methyl-4-oxobutanoic acid |
| 122. | (S)-4-(5-(3-((2-((S)-3-carboxybutanoyl)-6-(methylamino)benzo[b]thiophen-5-yl)oxy)pro- |
| | poxy)-6-methoxyisoindolin-2-yl)-2-methyl-4-oxobutanoic acid |
| 123. | (S)-4-(5-(3-((6-bromo-2-((S)-3-carboxybutanoyl)benzo[b]thiophen-5-yl)oxy)propoxy)- |
| | 6-methoxyisoindolin-2-yl)-2-methyl-4-oxobutanoic acid |
| 124. | (S)-4-(5-(3-((2-((S)-3-carboxybutanoyl)-6-methoxybenzo[b]thiophen-5-yl)oxy)propoxy)-6- |
| | methylisoindolin-2-yl)-2-methyl-4-oxobutanoic acid |
| 125. | (S)-4-(5-(3-((2-((S)-3-carboxybutanoyl)-6-ethylisoindolin-5-yl)oxy)propoxy)-6-methoxy- |
| | benzo[b]thiophen-2-yl)-2-methyl-4-oxobutanoic acid |
| 126. | (S)-4-(5-(3-((2-((S)-3-carboxybutanoyl)-6-methoxybenzo[b]thiophen-5-yl)oxy)propoxy)-6- |
| | vinylisoindolin-2-yl)-2-methyl-4-oxobutanoic acid |
| 127. | (S)-4-(5-(3-((2-((S)-3-carboxybutanoyl)-6-ethynylisoindolin-5-yl)oxy)propoxy)-6- |
| | methoxybenzo[b]thiophen-2-yl)-2-methyl-4-oxobutanoic acid |
| 128. | (S)-4-(5-(3-((6-amino-2-((S)-3-carboxybutanoyl)isoindolin-5-yl)oxy)propoxy)-6- |
| | methoxybenzo[b]thiophen-2-yl)-2-methyl-4-oxobutanoic acid |
| 129. | (S)-4-(5-(3-((2-((S)-3-carboxybutanoyl)-6-(methylamino)isoindolin-5-yl)oxy)propoxy)- |
| | 6-methoxybenzo[b]thiophen-2-yl)-2-methyl-4-oxobutanoic acid |
| 130. | (S)-4-(5-(3-((6-bromo-2-((S)-3-carboxybutanoyl)isoindolin-5-yl)oxy)propoxy)-6- |
| | methoxybenzo[b]thiophen-2-yl)-2-methyl-4-oxobutanoic acid |
| 131. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-methoxybenzo[b]thiophen-2-yl)-2-methyl-4-oxobutanoic acid |
| 132. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-4-fluoro-6-methoxybenzo[b]thiophen-2-yl)-2-methyl-4-oxobutanoic |
| | acid |
| 133. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-4-chloro-6-methoxybenzo[b]thiophen-2-yl)-2-methyl-4-oxobutanoic |
| | acid |
| 134. | (S)-4-(5-(3-((6-((S)-3-carboxybutanoyl)-3-hydroxy-6,7-dihydro-5H-pyrrolo[3,4-b]pyridin- |
| | 2-yl)oxy)propoxy)-6-methoxythieno[3,2-b]pyridin-2-yl)-2-methyl-4-oxobutanoic acid |
| 135. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-hydroxythieno[3,2-b]pyridin-2-yl)-2-methyl-4-oxobutanoic acid |
| 136. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-methoxythieno[3,2-b]pyridin-2-yl)-2-methyl-4-oxobutanoic acid |
| 137. | (S)-4-(5-(3-((6-((S)-3-carboxybutanoyl)-4-fluoro-3-methoxy-6,7-dihydro-5H-pyrrolo[3,4-b] |
| | pyridin-2-yl)oxy)propoxy)-6-methoxythieno[3,2-b]pyridin-2-yl)-2-methyl-4-oxobutanoic |
| | acid |
| 138. | (S)-4-(5-(3-((6-((S)-3-carboxybutanoyl)-4-chloro-3-methoxy-6,7-dihydro-5H-pyrrolo[3,4- |
| | b]pyridin-2-yl)oxy)propoxy)-6-methoxythieno[3,2-b]pyridin-2-yl)-2-methyl-4- |
| | oxobutanoic acid |
| 139. | (S)-4-(5-(3-((2-((S)-3-carboxybutanoyl)-6-methoxyisoindolin-5-yl)oxy)propoxy)-6- |
| | methoxythieno[3,2-b]pyridin-2-yl)-2-methyl-4-oxobutanoic acid |
| 140. | (S)-4-(5-(3-((2-((S)-3-carboxybutanoyl)-4-fluoro-6-methoxyisoindolin-5-yl)oxy)pro- |
| | poxy)-6-methoxythieno[3,2-b]pyridin-2-yl)-2-methyl-4-oxobutanoic acid |
| 141. | (S)-4-(5-(3-((2-((S)-3-carboxybutanoyl)-4-chloro-6-methoxyisoindolin-5-yl)oxy)propoxy)- |
| | 6-methoxythieno[3,2-b]pyridin-2-yl)-2-methyl-4-oxobutanoic acid |
| 142. | (S)-4-(5-(3-((2-((S)-3-carboxybutanoyl)-4,7-dichloro-6-methoxyisoindolin-5-yl)oxy)pro- |
| | poxy)-6-methoxythieno[3,2-b]pyridin-2-yl)-2-methyl-4-oxobutanoic acid |
| 143. | (S)-4-(5-(3-((2-((S)-3-carboxybutanoyl)-4-chloro-7-fluoro-6-methoxyisoindolin-5- |
| | yl)oxy)propoxy)-6-methoxythieno[3,2-b]pyridin-2-yl)-2-methyl-4-oxobutanoic acid |
| 144. | (S)-4-(5-(3-((2-((S)-3-carboxybutanoyl)-4,7-difluoro-6-methoxyisoindolin-5-yl)oxy)pro- |
| | poxy)-6-methoxythieno[3,2-b]pyridin-2-yl)-2-methyl-4-oxobutanoic acid |
| 145. | (S)-4-(5-(3-((2-((S)-3-carboxybutanoyl)-4,7-difluoro-6-hydroxyisoindolin-5-yl)oxy)pro- |
| | poxy)-6-methoxythieno[3,2-b]pyridin-2-yl)-2-methyl-4-oxobutanoic acid |
| 146. | (S)-4-(5-(3-((2-((S)-3-carboxybutanoyl)-4-fluoro-6-hydroxybenzo[b]thiophen-5- |
| | yl)oxy)propoxy)-4-fluoro-6-methoxyisoindolin-2-yl)-2-methyl-4-oxobutanoic acid |
| 147. | (S)-4-(5-(3-((2-((S)-3-carboxybutanoyl)-4-chloro-6-hydroxybenzo[b]thiophen-5- |
| | yl)oxy)propoxy)-4-fluoro-6-methoxyisoindolin-2-yl)-2-methyl-4-oxobutanoic acid |
| 148. | (S)-4-(5-(3-((2-((S)-3-carboxybutanoyl)-4-chloro-6-methoxyisoindolin-5-yl)oxy)propoxy)- |
| | 4-fluoro-6-hydroxybenzo[b]thiophen-2-yl)-2-methyl-4-oxobutanoic acid |
| 149. | (S)-4-(5-(3-((2-((S)-3-carboxybutanoyl)-4-chloro-6-hydroxybenzo[b]thiophen-5- |
| | yl)oxy)propoxy)-4-chloro-6-methoxyisoindolin-2-yl)-2-methyl-4-oxobutanoic acid |
| 150. | (S)-4-(6-(3-((2-((S)-3-carboxybutanoyl)-6-hydroxybenzo[b]thiophen-5-yl)oxy)propoxy)- |
| | 4-fluoro-5-methoxyisoindolin-2-yl)-2-methyl-4-oxobutanoic acid |
| 151. | (S)-4-(6-(3-((2-((S)-3-carboxybutanoyl)-7-fluoro-6-hydroxybenzo[b]thiophen-5- |
| | yl)oxy)propoxy)-4-fluoro-5-methoxyisoindolin-2-yl)-2-methyl-4-oxobutanoic acid |
| 152. | (S)-4-(6-(3-((2-((S)-3-carboxybutanoyl)-7-chloro-6-hydroxybenzo[b]thiophen-5- |
| | yl)oxy)propoxy)-4-fluoro-5-methoxyisoindolin-2-yl)-2-methyl-4-oxobutanoic acid |
| 153. | (S)-4-(5-(3-((2-((S)-3-carboxybutanoyl)-7-chloro-6-methoxyisoindolin-5-yl)oxy)propoxy)- |
| | 7-fluoro-6-hydroxybenzo[b]thiophen-2-yl)-2-methyl-4-oxobutanoic acid |
| 154. | (S)-4-(5-(3-((2-((S)-3-carboxybutanoyl)-4,7-difluoro-6-methoxyisoindolin-5-yl)oxy)pro- |
| | poxy)-7-chloro-6-hydroxybenzo[b]thiophen-2-yl)-2-methyl-4-oxobutanoic acid |
| 155. | (S)-4-(5-(3-((2-((S)-3-carboxybutanoyl)-4,7-difluoro-6-methoxyisoindolin-5-yl)oxy)pro- |
| | poxy)-7-fluoro-6-hydroxybenzo[b]thiophen-2-yl)-2-methyl-4-oxobutanoic acid |
| 156. | (S)-4-(5-(3-((2-((S)-3-carboxybutanoyl)-4-chloro-7-fluoro-6-methoxyisoindolin-5- |
| | yl)oxy)propoxy)-7-fluoro-6-hydroxybenzo[b]thiophen-2-yl)-2-methyl-4-oxobutanoic acid |
| 157. | (S)-4-(6-(3-((2-((S)-3-carboxybutanoyl)-4,7-difluoro-6-hydroxybenzo[b]thiophen-5-yl)oxy) |
| | propoxy)-4-fluoro-5-methoxyisoindolin-2-yl)-2-methyl-4-oxobutanoic acid |
| 158. | (S)-4-(6-(3-((2-((S)-3-carboxybutanoyl)-7-chloro-4-fluoro-6-hydroxybenzo[b]thiophen-5- |
| | yl)oxy)propoxy)-4-fluoro-5-methoxyisoindolin-2-yl)-2-methyl-4-oxobutanoic acid |
| 159. | (S)-4-(6-(3-((2-((S)-3-carboxybutanoyl)-4,7-dichloro-6-hydroxybenzo[b]thiophen-5-yl)oxy) |
| | propoxy)-4-fluoro-5-methoxyisoindolin-2-yl)-2-methyl-4-oxobutanoic acid |
| 160. | (S)-4-(5-(3-((2-((S)-3-carboxybutanoyl)-5-methoxybenzo[b]thiophen-6-yl)oxy)propoxy)-6- |
| | methoxyisoindolin-2-yl)-2-methyl-4-oxobutanoic acid |
| 161. | 4-(5-(3-((2-(3-carboxypropanoyl)-6-methoxyisoindolin-5-yl)oxy)propoxy)-6-methoxy- |
| | thieno[2,3-b]pyridin-2-yl)-4-oxobutanoic acid |
| 162. | 4-(5-(3-((2-(4-(((4R,5R)-5-hydroxy-1,2-dithian-4-yl)oxy)-4-oxobutanoyl)-6-methoxythieno |
| | [2,3-b]pyridin-5-yl)oxy)propoxy)-6-methoxyisoindolin-2-yl)-4-oxobutanoic acid |
| 163. | 4-(5-(3-((6-(3-carboxypropanoyl)-3-methoxy-6,7-dihydro-5H-pyrrolo[3,4-b]pyridin-2- |
| | yl)oxy)propoxy)-6-methoxybenzo[b]thiophen-2-yl)-4-oxobutanoic acid |
| 164. | 4-(2-(3-((2-(4-(((4R,5R)-5-hydroxy-1,2-dithian-4-yl)oxy)-4-oxobutanoyl)-6-methoxy- |
| | benzo[b]thiophen-5-yl)oxy)propoxy)-3-methoxy-5,7-dihydro-6H-pyrrolo[3,4-b]pyridin- |
| | 6-yl)-4-oxobutanoic acid |
| 165. | 4-(5-(3-((6-(3-carboxypropanoyl)-2-methoxy-6,7-dihydro-5H-pyrrolo[3,4-b]pyridin- |
| | 3-yl)oxy)propoxy)-6-methoxybenzo[b]thiophen-2-yl)-4-oxobutanoic acid |
| 166. | 4-(3-(3-((2-(4-(((4R,5R)-5-hydroxy-1,2-dithian-4-yl)oxy)-4-oxobutanoyl)-6-meth- |
| | oxybenzo[b]thiophen-5-yl)oxy)propoxy)-2-methoxy-5,7-dihydro-6H-pyrrolo[3,4-b]py- |
| | ridin-6-yl)-4-oxobutanoic acid |
| 167. | 4-(2-(3-((2-(4-(((4R,5R)-5-hydroxy-1,2-dithian-4-yl)oxy)-4-oxobutanoyl)-6-meth- |
| | oxythieno[3,2-b]pyridin-5-yl)oxy)propoxy)-3-methoxy-5,7-dihydro-6H-pyrrolo[3,4- |
| | b]pyridin-6-yl)-4-oxobutanoic acid |
| 168. | 4-(5-(3-((2-(3-carboxypropanoyl)-5-methoxythieno[2,3-b]pyridin-6-yl)oxy)propoxy)- |
| | 6-methoxyisoindolin-2-yl)-4-oxobutanoic acid |
| 169. | (S)-4-(5-(3-((2-(3-carboxypropanoyl)-5-methoxythieno[2,3-b]pyridin-6-yl)oxy)propoxy)-6- |
| | methoxyisoindolin-2-yl)-2-methyl-4-oxobutanoic acid |
| 170. | (S)-4-(6-(3-((2-(3-carboxypropanoyl)-6-methoxyisoindolin-5-yl)oxy)propoxy)-5- |
| | methoxythieno[2,3-b]pyridin-2-yl)-2-methyl-4-oxobutanoic acid |
| 171. | (S)-4-(5-(3-((2-((S)-3-carboxybutanoyl)-5-methoxythieno[2,3-b]pyridin-6-yl)oxy)propoxy)- |
| | 6-methoxyisoindolin-2-yl)-2-methyl-4-oxobutanoic acid |
| 172. | 4-(5-(3-((2-(4-(((4R,5R)-5-hydroxy-1,2-dithian-4-yl)oxy)-4-oxobutanoyl)-5-methoxythieno |
| | [2,3-b]pyridin-6-yl)oxy)propoxy)-6-methoxyisoindolin-2-yl)-4-oxobutanoic acid |

In some embodiments, the compound is an isotopic derivative (e.g., isotopically labeled compound) of any one of the compounds of the formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the isotopic derivative is a deuterium labeled compound.

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

### Methods of Preparation

The compounds of the present invention can be prepared in a number ways well known to one skilled in the art of organic synthesis using the methods described below or variations thereon as appreciated by those skilled in the art. The references cited herein are hereby incorporated by reference in their entirety.

The methods of synthesis described herein after are intended as an illustration of the invention, without restricting its subject matter and the scope of the compounds claimed to these examples. Where the preparation of starting compounds is not described, they are commercially obtainable or may be prepared analogously to known compounds or methods described herein. Compounds of any of the formulae described herein may be synthesized by reference to methods illustrated in the following schemes. As shown herein, the end compound is a product having the same structural formula depicted as any of formulas. It will be understood that any compound of the formulas may be prepared by the suitable selection of reagents with appropriate substitution. Solvents, temperature, pressures, and other reaction conditions may be readily selected by one of ordinary skill in the art. Protecting groups are manipulated according to standard methods of organic synthesis (T. W. Green and P. G. M. Wuts (1999) Protective Groups in Organic Synthesis, 3^{th} edition, John Wiley & Sons). These groups are removed at certain stage of the compound synthesis using the methods that are apparent to those skilled in the art.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate thereof; and/or (iv) forming a prodrug thereof.

For illustrative purposes, Schemes 1 and 2 show a general synthetic method for preparing the compounds described herein. For a more detailed description of the individual reaction steps, see the Examples section below. Those skilled in the art will appreciate that other synthetic routes may be used to synthesize the compounds. In addition, many of the compounds prepared by the methods described below can be further modified in light of this disclosure using conventional chemistry well known those skilled in the art.

General routes to compounds illustrated in the invention is described in Scheme 1 and 2, where the W, R₁, X₁, X₂, X₃, R₂, R₃, and R₄, etc. substituents are defined previously in the text or a functional group that can be converted to the desired final substituent.

In the following Methods and Schemes, LG represents a leaving group, which may be a halide or triflate group. The variables included in the Methods and Schemes have the meanings provided; exemplary catalysts are defined in the Abbreviations (below).

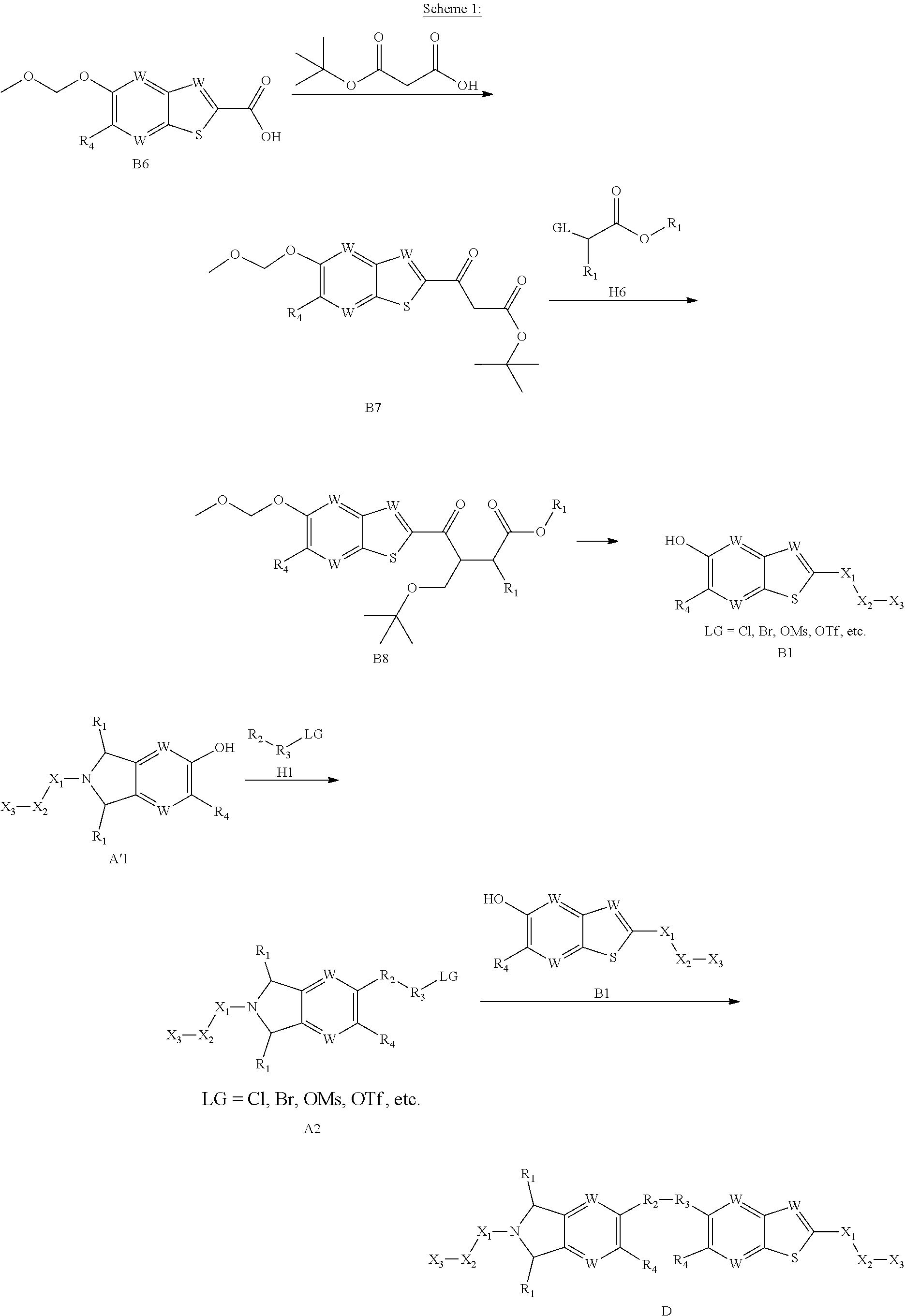

B6 reacts with 3-(tert-butoxy)-3-oxopropanoic acid in the presence of CDI, MgCl₂ and basic conditions to afford B7. B7 reacts with H6 in the presence of LDA or K₂CO₃ to afford B8. B8 reacts with TFA to obtain B1.

A′1 and H1 with an substitution reaction under basic conditions to afford A2.

A2 with B1 with an substitution reaction under basic conditions to afford D.

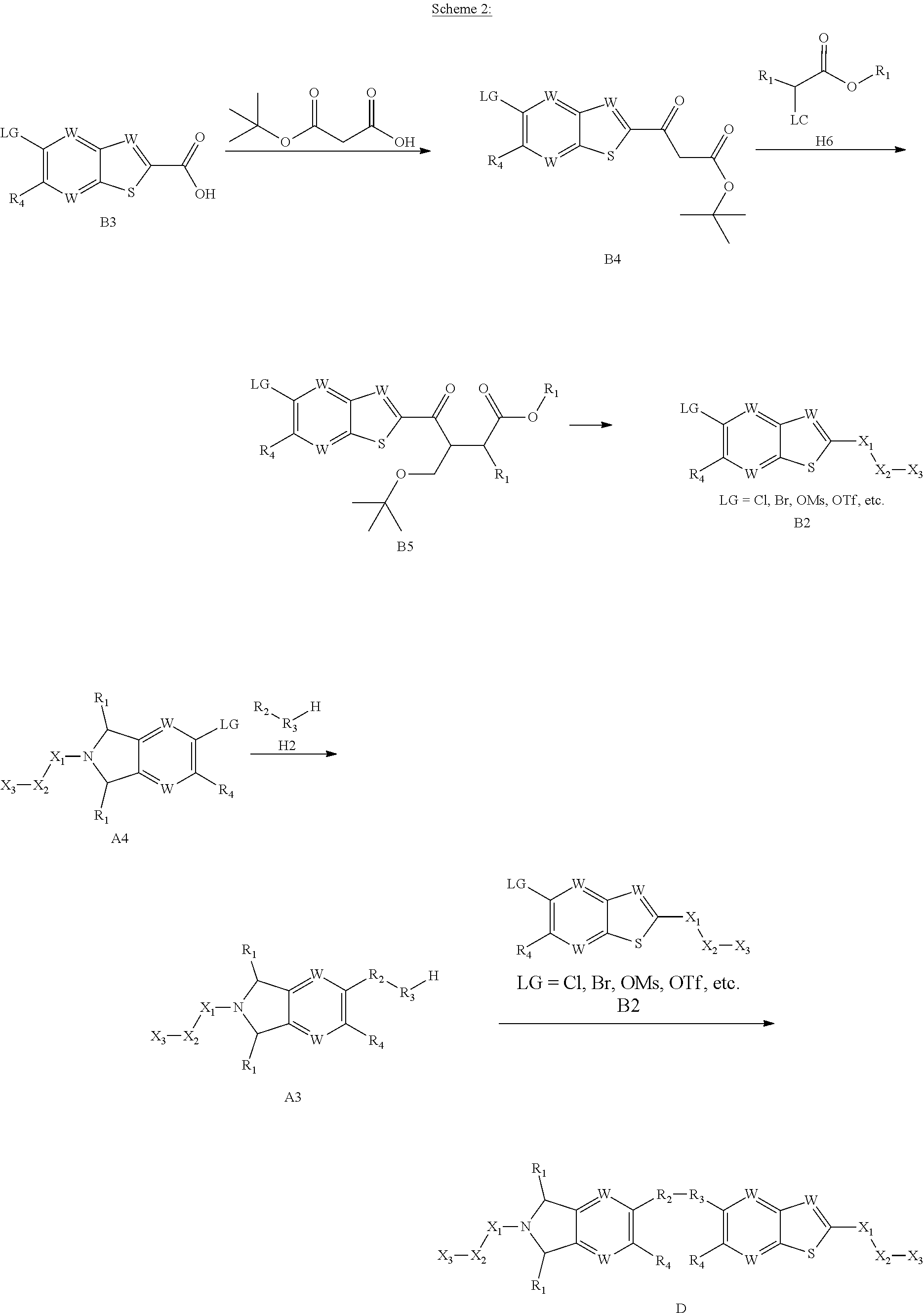

B3 reacts with 3-(tert-butoxy)-3-oxopropanoic acid in the presence of CDI, MgCl₂ and basic conditions to afford B4. B4 reacts with H6 in the presence of basic conditions to afford B5. B5 reacts with TFA to remove tert butyl to obtain B2.

A4 reacts with h2 in the presence of NaH to afford A3.

A3 reacts with B2 in the presence of a palladium catalyst to afford D.

The present invention also provides a pharmaceutical composition comprising at least one compound or pharmaceutically acceptable salt thereof of Formula I, I-1, II, III, IV or V and at least one pharmaceutically acceptable carrier. Furthermore, in the composition, the said compound or pharmaceutically acceptable salt thereof of Formula I, I-1, II, III, IV or V is in a weight ratio to the said carrier within the range from about 0.0001 to about 10.

The pharmaceutical composition of the this invention may further comprise at least one of additional active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents, etc.

The present invention additionally provided using at least one compound or pharmaceutically acceptable salt thereof of Formula I, I-1, II, III, IV or V, or pharmaceutical composition described above, which is for the the manufacture of a medicament.

In some embodiments, the medicament is used for inducing an immune response, inducing STING-dependent type I interferon production, inducing a STING-dependent cytokine production, or treating a cell proliferation disorder in a subject.

In some embodiments, the cell proliferation disorder is cancer, cancer metastasis, cardiovascular disease, an immunological disorder, fibrosis, or an ocular disorder.

At least one compound or pharmaceutically acceptable salt thereof of Formula I, I-1, II, III, IV or V, and/or a pharmaceutical composition described herein, which is for use in therapy.

At least one compound or pharmaceutically acceptable salt thereof of Formula I, I-1, II, III, IV or V, and/or a pharmaceutical composition described herein, which is for use in inducing an immune response, inducing STING-dependent type I interferon production, inducing a STING-dependent cytokine production, or treating a cell proliferation disorder in a subject.

In some embodiments, the cell proliferation disorder is cancer, cancer metastasis, cardiovascular disease, an immunological disorder, fibrosis, or an ocular disorder.

At least one compound or pharmaceutically acceptable salt thereof of Formula I, I-1, II, III, IV or V, and/or a pharmaceutical composition described herein, which is used as a STING agonists.

At least one compound or pharmaceutically acceptable salt thereof of Formula I, I-1, II, III, IV or V, and/or a pharmaceutical composition described herein, which is used as a medicament.

The present invention additionally provided a method of inducing an immune response in a subject, said method comprising administering to the patient a therapeutically effective amount of at least one compound of Formula I, I-1, II, III, IV or V or pharmaceutically acceptable salt thereof, or the pharmaceutical composition described above.

The present invention additionally provided a method of inducing a STING-dependent type I interferon production in a subject, said method comprising administering to the patient a therapeutically effective amount of at least one compound of Formula I, I-1, II, III, IV or V or pharmaceutically acceptable salt thereof, or the pharmaceutical composition described above.

The present invention additionally provided a method of inducing a STING-dependent cytokine production in a subject, said method comprising administering to the patient a therapeutically effective amount of at least one compound of Formula I, I-1, II, III, IV or V or pharmaceutically acceptable salt thereof, or the pharmaceutical composition described above.

The present invention additionally provided a method of treating a cell proliferation disorder in a subject, said method comprising administering to the patient a therapeutically effective amount of at least one compound of Formula I, I-1, II, III, IV or V or pharmaceutically acceptable salt thereof, or the pharmaceutical composition described above.

In some embodiments, the cell proliferation disorder is cancer, cancer metastasis, cardiovascular disease, an immunological disorder, fibrosis, or an ocular disorder.

The compounds disclosed herein may be STING agonists. These compounds are potentially useful in treating diseases or disorders including, but not limited to, cell proliferation disorders. Cell-proliferation disorders include, but are not limited to, cancers, benign papillomatosis, gestational trophoblastic diseases, and benign neoplastic diseases, such as skin papilloma (warts) and genital papilloma.

In specific embodiments, the disease or disorder to be treated is a cell proliferation disorder. In certain embodiments, the cell proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In specific embodiments, the cancer is selected from brain and spinal cancers. In particular embodiments, the cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and supratentorial primordial neuroectodermal tumors (sPET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas.

In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosarcoma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “administration of and or “administering” a compound should be understood to include providing a compound described herein, or a pharmaceutically acceptable salt thereof, and compositions of the foregoing to a subject.

The amount of a compound administered to a subject is an amount sufficient to induce an immune response and/or to induce STING-dependent type I interferon production in the subject. In an embodiment, the amount of a compound can be an “effective amount” or “therapeutically effective amount,” such that the subject compound is administered in an amount that will elicit, respectively, a biological or medical (i.e., intended to treat) response of a tissue, system, animal, or human that is being sought by a researcher, veterinarian, medical doctor, or other clinician. An effective amount does not necessarily include considerations of toxicity and safety related to the administration of a compound.

An effective amount of a compound will vary with the particular compound chosen (e.g., considering the potency, efficacy, and/or half-life of the compound); the route of administration chosen; the condition being treated; the severity of the condition being treated; the age, size, weight, and physical condition of the subject being treated; the medical history of the subject being treated; the duration of the treatment; the nature of a concurrent therapy; the desired therapeutic effect; and like factors and can be routinely determined by the skilled artisan.

The term “subject” (alternatively referred to herein as “patient”) as used herein refers to an animal, preferably a mammal, most preferably a human, who has been the object of treatment, observation or experiment.

As used herein, the term “immune response” relates to any one or more of the following: specific immune response, non-specific immune response, both specific and nonspecific response, innate response, primary immune response, adaptive immunity, secondary immune response, memory immune response, immune cell activation, immune cell proliferation, immune cell differentiation, and cytokine expression. In certain embodiments, a compound of general Formula I, I-1, II, III, IV or V, or a pharmaceutically acceptable salt of the foregoing, is administered in conjunction with one or more additional therapeutic agents including anti-viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc.

The term “halogen”, as used herein, unless otherwise indicated, means fluoro, chloro, bromo or iodo. The preferred halogen groups include F, Cl and Br. The terms “haloC₁₋₆alkyl”, “haloC₂₋₆alkenyl”, “haloC₂₋₆alkynyl” and “haloC₁₋₆alkoxy” mean a C₁₋₆alkyl, C₂₋₆alkenyl, C₂₋₆alkynyl or C₁₋₆alkoxy in which one or more (in particular, 1, 2 or 3) hydrogen atoms have been replaced by halogen atoms, especially fluorine or chlorine atoms. In some embodiment, preferred are fluoroC₁₋₆alkyl, fluoroC₂₋₆alkenyl, fluoroC₂₋₆alkynyl and fluoroC₁₋₆alkoxy groups, in particular fluoroC₁₋₃alkyl, for example, CF₃, CHF₂, CH₂F, CH₂CH₂F, CH₂CHF₂, CH₂CF₃ and fluoroC₁₋₃alkoxy groups, for example, OCF₃, OCHF₂, OCH₂F, OCH₂CH₂F, OCH₂CHF₂ or OCH₂CF₃, and most especially CF₃, OCF₃ and OCHF₂.

As used herein, unless otherwise indicated, alkyl includes saturated monovalent hydrocarbon radicals having straight, branched or cyclic moieties. For example, alkyl radicals include methyl, ethyl, propyl, isopropyl, cyclopropyl, n-butyl, isobutyl, sec-butyl, t-butyl, cyclobutyl, n-pentyl, 3-(2-methyl) butyl, 2-pentyl, 2-methylbutyl, neopentyl, cyclopentyl, n-hexyl, 2-hexyl, 2-methylpentyl and cyclohexyl. Similarly, C₁₋₈, as in C₁₋₈alkyl is defined to identify the group as having 1, 2, 3, 4, 5, 6, 7 or 8 carbon atoms in a linear or branched arrangement.

Alkylene means a difunctional group obtained by removal of a hydrogen atom from an alkyl group that is defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

As used herein, the term “alkenyl” refers to a monovalent straight or branched chain, unsaturated aliphatic hydrocarbon radical having a number of carbon atoms in the specified range and including one or more double bonds.

As used herein, the term “alkenylene” refers to a bivalent straight chain, unsaturated aliphatic hydrocarbon radical having a number of carbon atoms in the specified range and including one or more double bonds.

As used herein, the term “alkynyl” refers to a monovalent straight or branched chain, unsaturated aliphatic hydrocarbon radical having a number of carbon atoms in the specified range and including one or more triple bonds.

As used herein, the term “alkynylene” refers to a bivalent straight chain, unsaturated aliphatic hydrocarbon radical having a number of carbon atoms in the specified range and including one or more triple bonds.

As used herein, the term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxy ethane (also referred to as ‘ethyl methyl ether’).

The term “aryl”, as used herein, unless otherwise indicated, by itself or as part of another substituent refers to a monocyclic or polycyclic aromatic hydrocarbon. The preferred aryls are mono cyclic or bicyclic 6-10 membered aromatic ring systems. Phenyl and naphthyl are preferred aryls. The most preferred aryl is phenyl.

The term “heterocyclic”, “heterocyclyl”, or “heterocyclic”, as used herein, unless otherwise indicated, by itself or as part of another substituent refers to unsubstituted and substituted mono- or polycyclic non-aromatic, partially unsaturated or fully saturated ring system containing one or more heteroatoms. Preferred heteroatoms include N, O, and S, including N-oxides, sulfur oxides, and dioxides. Preferably the ring is three to eight membered and is either fully saturated or has one or more degrees of unsaturation. Multiple degrees of substitution, preferably one, two or three, are included within the present definition.

Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, by itself or as part of another substituent refers to an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “carbocyclic” refers to a substituted or unsubstituted monocyclic ring, bicyclic ring, bridged ring, fused ring, spiro ring non-aromatic ring system only containing carbon atoms. Exemplary “carbocyclic” groups include but not limited to, cyclopropyl, cyclobutyl, cyclopentyl, cyclohexyl and so on.

The term “cycloalkyl” as used herein, unless otherwise indicated, by itself or as part of another substituent refers to a substituted or unsubstituted monocyclic, bicyclic or polycyclic non-aromatic saturated or partially unsaturated hydrocarbon group, which optionally includes an alkylene linker through which the cycloalkyl may be attached. Exemplary “cycloalkyl” groups includes but not limited to, cyclopropyl, cyclobutyl, cyclopentyl, cyclohexyl and so on.

The term “carbonyl”, “—C═O”, “C═O”, “—CO”, “—C(O)”, and “CO” refer to the group

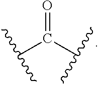 [SMILES: *C(*)=O]

The term “oxo” refers to the radical ═O.

Whenever the term “alkyl” or “aryl” or either of their prefix roots appear in a name of a substituent (e.g., aralkyl or dialkylamino), unless otherwise indicated, by itself or as part of another substituent, it shall be interpreted as including those limitations given above for “alkyl” and “aryl”. Designated numbers of carbon atoms (e.g., C₁₋₆) shall refer independently to the number of carbon atoms in an alkyl moiety or to the alkyl portion of a larger substituent in which alkyl appears as its prefix root.

As used herein, the term “fused ring” refers to a cyclic group formed by substituents on separate atoms in a straight or branched alkane, or to a cyclic group formed by substituents on separate atoms in another ring.

As used herein, the term “spirocycle” or “spirocyclic ring” refers to a pendant cyclic group formed by substituents on a single atom.

The term “ring systems” as used herein, unless otherwise indicated, include but not limited to a carbocyclic ring, a heterocyclic ring, a heteroaromatic ring, etc., may also include only a heterocyclic ring, and/or a heteroaromatic ring, and the like, specifically includes which rings need to be determined according to the context, but anyway the “ring systems” do not include the cycloalkyl based on a C₁₋₆ alkyl or C₁₋₃ alkyl group, and do not include the cycloalkoxy based on a C₁₋₆ alkoxy or C₁₋₃ alkoxy group.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents.

As used herein, the term “one or more” item includes a single item selected from the list as well as mixtures of two or more items selected from the list.

Wherein the term “substituted” refers to a group mentioned above in which one or more (preferably 1-6, more preferably 1-3) hydrogen atoms are each independently replaced with the same or different substituent(s). Typical substituents include, but are not limited to, X, C₁₋₆alkyl, C₁₋₆alkoxy, C₃₋₂₀ cycloalkyl, —OR₁₃, SR₁₃, ═O, ═S, —C(O)R₁₃, —C(S)R₁₃, ═NR₁₃, —C(O)OR₁₃, —C(S)OR₁₃, —NR₁₃R₁₄, —C(O)NR₁₃R₁₄, cyano, nitro, —S(O)₂R₁₃, —OS(O₂)OR₁₃, —OS(O)₂R₁₃, or —OP(O)(OR₁₃)(OR₁₄); wherein each X is independently a halogen (F, Cl, Br or I), and R₁₃ and R₁₄ is independently selected from —H, C₁₋₆ alkyl and C₁₋₆ haloalkyl. In some embodiments, the substituent(s) is independently selected from the group consisting of —F, —Cl, —Br, —I, —OH, trifluoromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl. Particularly preferred substituent(s) is —F, —Cl or —Br.

The substituents the “W” of Formula I, I-1, II, III, IV or V can be the same or different. Similar to “W”, and the “R₁” “X₁” “X₂” or “X₃” of Formula I, I-1, II, III, IV or V can be the same or different.

Compounds described herein can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention. In some embodiments, one or more hydrogen atoms of any of the compounds described herein can be substituted with deuterium to provide the corresponding deuterium-labeled or -enriched compounds.

Where the plural form (e.g. compounds, salts) is used, this includes the singular (e.g. a single compound, a single salt). “A compound” does not exclude that (e.g. in a pharmaceutical formulation) more than one compound of the Formula I, I-1, II, III, IV or V (or a salt thereof) is present, the “a” merely representing the indefinite article. “A” can thus preferably be read as “one or more”, less preferably alternatively as “one”.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985. It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

Compounds described herein, such as certain compounds of Formula I, I-1, II, III, IV or V may contain asymmetrically substituted carbon atoms (or chiral centers) in the R or S configuration. The present invention includes racemic mixtures, relative and absolute stereoisomers, and mixtures of relative and absolute stereoisomers.

The compounds described herein, when specifically designated as the R- or S-isomer, either in a chemical name or in a drawing, should be understood as an enriched R-isomer or S-isomer, respectively. For example, in any of the embodiments described herein, such enriched R- or S-designated isomer can be substantially free (e.g., with less than 5%, less than 1%, or non-detectable, as determined by chiral HPLC) of the other isomer for the respective chiral center. The enriched R- or S-isomers can be prepared by methods exemplified in this application, such as by using a chiral auxiliary such as R- or S-tert-butylsulfinamide in the synthetic process. Other methods for preparing the enriched R- or S-isomers herein include, but are not limited to, chiral HPLC purifications of a stereoisomeric mixture, such as a racemic mixture. General methods for separating stereoisomers (such as enantiomers and/or diastereomers) using HPLC are known in the art.

Compounds of Formula I, I-1, II, III, IV or V may have different isomeric forms. For example, any asymmetric carbon atom may be present in the (R)-, (S)- or (R,S)-configuration, preferably in the (R)- or (S)-configuration. Substituents at a double bond or especially a ring may be present cis-(=Z-) or trans (=E-) form. The compounds may thus be present as mixtures of isomers or preferably as pure isomers, preferably as pure diastereomers or pure enantiomers.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I, I-1, II, III, IV or V is shown without a definitive stereochemistry at certain positions.

The present invention includes all stereoisomers of Formula I, I-1, II, III, IV or V and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I, I-1, II, III, IV or V exists, the present invention includes any possible tautomers and pharmaceutically acceptable salts thereof, and mixtures thereof, except where specifically stated otherwise.

When the compound of Formula I, I-1, II, III, IV or V and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds of Formula I, I-1, II, III, IV or V are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I, I-1, II, III, IV or V (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, I-1, II, III, IV or V, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, I-1, II, III, IV or V, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier, and a compound or a pharmaceutically acceptable salt of Formula I, I-1, II, III, IV or V. The compounds of Formula I, I-1, II, III, IV or V, or pharmaceutically acceptable salts thereof, can also be included in pharmaceutical compositions in combination with one or more additional therapeutically active agents.

The additional active agent(s) may be one or more agents selected from the group consisting of STING agonist compounds, anti-viral compounds, antigens, adjuvants, anti-cancer agents, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines. It will be understood that such additional active agent(s) may be provided as a pharmaceutically acceptable salt. It will be understood the descriptions of the above additional active agents may be overlapping. It will also be understood that the treatment combinations are subject to optimization, and it is understood that the best combination to use of the compounds of general Formula I, I-1, II, III, IV or V or pharmaceutically acceptable salts of the foregoing, and one or more additional active agents will be determined based on the individual patient needs.

A compound disclosed herein may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I, I-1, II, III, IV or V, II, III or IV of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration and the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, I-1, II, III, IV or V, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

### EXAMPLES

The following Examples are provided to better illustrate the present invention. All parts and percentages are by weight and all temperatures are degrees Celsius, unless explicitly stated otherwise.

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only. While particular embodiments of the present disclosure are described, the skilled artisan will appreciate that various changes and modifications can be made without departing from the spirit and scope of the disclosure.

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.

| | |
|---|---|
| NBS | N-Bromosuccinimide |
| EA | Ethyl acetate |
| ACN | Acetonitrile |
| THF | Tetrahydrofuran |
| DCM | Dichloromethane |
| DMF | Dimethylformamide |
| TFA | Trifluoroacetic acid |
| TEA | Triethylamine |
| LDA | Lithium diisopropylamide |
| OXONE | PotassiumPeroxomonosulphate |
| FA | Formic acid |
| DIEA | N,N-Diisopropylethylamine |
| Pd(dppf)Cl₂ | [1,1′-Bis(diphenylphosphino)ferrocene]di- |
| | chloropalladium(II) |
| CDI | N,N-Carbonyldiimidazole |
| RockPhos | Methanesulfonato(2-(di-t-butylphosphino)- |
| Palladacycle Gen.3 | 3-methoxy-6-methyl-2′,4′,6′-tri-i-propyl-1,1′- |
| | biphenyl)(2′-amino-1,1′-biphenyl-2-yl)pal- |
| | ladium(II) |
| MeOH | Methanol |
| EtOH | Ethanol |
| INT | Intermediate |
| h | hour(s) |
| aq | aqueous |
| sat | saturated |
| TLC | Thin layer chromatography |
| Pre-TLC | Preparative thin layer chromatography |
| Prep - HPLC | Preparation high performance liquid |
| | chromatography |

### Intermediate

### INT A1: Ethyl 4-(5-hydroxy-6-methoxy-benzothiophen-2-yl)-4-oxo-butanoate

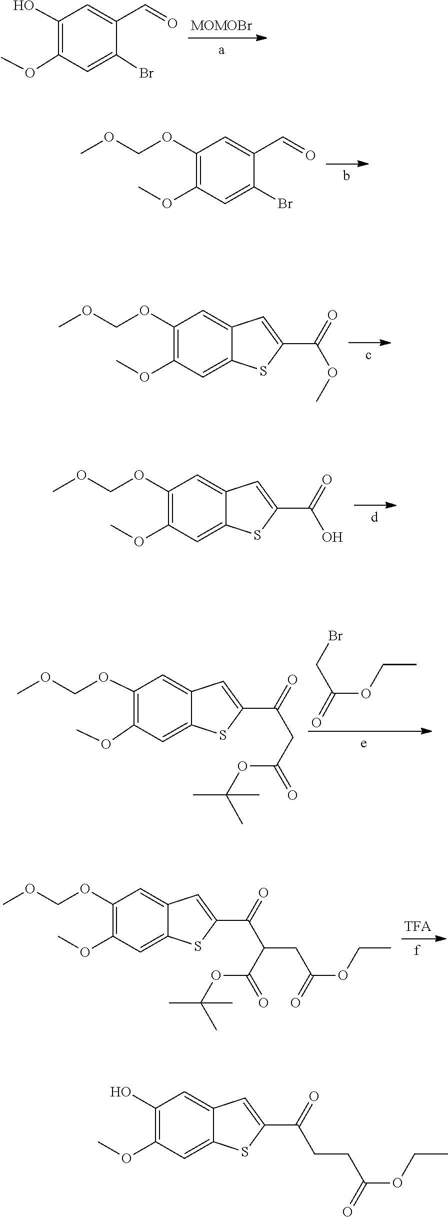

Step a: Bromomethyl methyl ether, (3.15 g, 25.2073 mmol) was added to a solution of DIEA (4.10 g, 46.5111 mmol) and 2-Bromoisovanillin (4.98 g, 21.5544 mmol) in Dichloromethane (200 mL) at 0° C. The reaction mixture was stirred at 20° C. for 2 h. The resulting solution was quenched by water (10 mL) and washed with water (100 mL) and brine (100 mL). The organics was dried over Na₂SO₄, filtered and evaporated under reduced pressure. There was 2-bromo-4-methoxy-5-(methoxymethoxy)benzaldehyde (5.83 g, 21.1926 mmol, 98.3216% yield) obtained as a white solid. ¹H NMR (400 MHz, DMSO-d₆) δ 10.06 (s, 1H), 7.51 (s, 1H), 7.39 (s, 1H), 5.25 (s, 2H), 3.93 (s, 3H), 3.38 (d, J=9.3 Hz, 3H).

Step b: Methyl thioglycolate (2.30 g, 21.6688 mmol) was added to a solution of 2-bromo-4-methoxy-5-(methoxymethoxy)benzaldehyde (5.83 g, 21.1926 mmol) and Cs₂CO₃ (13.99 g, 42.9380 mmol) in N,N-Dimethylformamide (100 mL) at 20° C. The reaction mixture was heated to 50° C. and stirred for 16 h. The reaction mixture was cooled to 20° C., and Methyl Iodide (1.81 g, 12.7520 mmol) was added to the reaction solution, and stirred for 2 h at 20° C. The reaction mixture was diluted with EA (500 mL), washed with water (2×100 mL) and brine (100 mL). The organics was evaporated under reduced pressure. The residue was purified on silica gel column EA/hept (0-50%). The pure fractions was concentrated and dried under vacuo. There was methyl 6-methoxy-5-(methoxymethoxy)benzothiophene-2-carboxylate (2.98 g, 10.5557 mmol, 49.8084% yield) obtained as a yellow solid. ¹H NMR (400 MHz, DMSO-d₆) δ 8.05 (s, 1H), 7.66 (s, 1H), 7.63 (s, 1H), 5.23 (s, 2H), 3.87 (d, J=9.2 Hz, 6H), 3.42 (s, 3H).

Step c: LiOH (0.37 g, 15.4499 mmol) was added to a solution of methyl 6-methoxy-5-(methoxymethoxy)benzothiophene-2-carboxylate (2.98 g, 10.5557 mmol) in Tetrahydrofuran (100 mL) and Water (25 mL) at 20° C. The reaction mixture was heated to 50° C. and stirred for 2 h. The reaction mixture was evaporated under reduced pressure and diluted with water and adjusted pH=3 with HCl (1 M). The precipitate was filtered. Filter cake was washed with water (20 mL). There was 6-methoxy-5-(methoxymethoxy) benzothiophene-2-carboxylic acid (2.41 g, 8.9830 mmol, 85.1008% yield) obtained as a white solid. ¹H NMR (400 MHz, DMSO-d₆) δ 13.22 (s, 1H), 7.93 (s, 1H), 7.62 (d, J=10.5 Hz, 2H), 5.24 (d, J=14.6 Hz, 2H), 3.87 (s, 3H), 3.52-3.35 (m, 3H).

Step d: To a stirred solution of 6-methoxy-5-(methoxymethoxy)benzothiophene-2-carboxylic acid (2.41 g, 8.9830 mmol) in N,N-Dimethylformamide (20 mL) was added 1,1′-Carbonyldiimidazole (2.84 g, 17.5148 mmol) at 20° C. The resulting mixture was stirred for 1 h at 20° C. under nitrogen atmosphere. To the mixture above was added 3-tert-Butoxy-3-oxopropanoic acid and Magnesium chloride (1.31 g, 13.7589 mmol). The reaction mixture was stirred at 20° C. for 16 h. The reaction mixture was concentrated and diluted with EA (500 mL), washed with NaHCO₃ aq. (2×300 mL) and brine (200 mL). The organics was dried over Na₂SO₄, filtered and evaporated under reduced pressure. The residue was purified on silica gel column EA/PE (0-80%). The pure fractions was concentrated and dried under vacuo. There was tert-butyl 3-[6-methoxy-5-(methoxymethoxy) benzothiophen-2-yl]-3-oxo-propanoate (2.63 g, 7.1774 mmol, 79.9000% yield) obtained as a yellow oil. ¹H NMR (400 MHz, DMSO-d₆) δ 8.20 (s, 1H), 7.67 (s, 1H), 7.62 (s, 1H), 5.24 (s, 2H), 4.03 (d, J=9.8 Hz, 2H), 3.89 (s, 3H), 3.42 (s, 3H), 1.41 (s, 9H).

Step e: To a stirred solution of tert-butyl 3-[6-methoxy-5-(methoxymethoxy)benzothiophen-2-yl]-3-oxo-propanoate (2.62 g, 7.1501 mmol) in N,N-Dimethylformamide (20 mL) was added Potassium carbonate (2.0621 g, 14.9207 mmol) and Ethyl bromoacetate (1.3648 g, 8.1724 mmol) at 20° C. The resulting mixture was stirred for 3 h at room temperature under nitrogen atmosphere. The reaction mixture was concentrated and diluted with EA (200 mL), washed water with (100 mL) and brine (50 mL). The organics was dried over Na₂SO₄, filtered and evaporated under reduced pressure. The residue was purified on silica gel column EA/PE (0-80%). The pure fractions was concentrated and dried under vacuo. There was 01-tert-butyl 04-ethyl 2-[6-methoxy-5-(methoxymethoxy) benzothiophene-2-carbonyl]butanedioate (2.82 g, 6.2318 mmol, 87.1569% yield) obtained as a yellow oil. ¹H NMR (400 MHz, DMSO-d₆) δ 8.35 (s, 1H), 7.67 (s, 2H), 5.31-5.20 (m, 2H), 4.79 (t, J=7.3 Hz, 1H), 4.15-3.96 (m, 2H), 3.89 (d, J=6.7 Hz, 3H), 3.42 (d, J=4.2 Hz, 3H), 3.32 (s, 3H), 2.98-2.81 (m, 2H), 1.31 (s, 9H).

Step f: TFA (5 mL) was added to 01-tert-butyl 04-ethyl 2-[6-methoxy-5-(methoxymethoxy)benzothiophene-2-carbonyl]butanedioate (2.82 g, 6.2318 mmol) in Toluene (25 mL) at 20° C. The reaction mixture was heated to 50° C. and stirred for 30 min. The reaction mixture was evaporated under reduced pressure. The reaction mixture was concentrated and diluted with EA (200 mL), washed with water (100 mL) and brine (50 mL). The organics was dried over Na₂SO₄, filtered and evaporated under reduced pressure. The residue was purified on silica gel column EA/Heptane (0-100%). The pure fractions was concentrated and dried under vacuo to afford crude product. The residue was purified on silica gel column MeCN/water (0-100%). The pure fractions was concentrated and dried under vacuo. There was ethyl 4-(5-hydroxy-6-methoxy-benzothiophen-2-yl)-4-oxo-butanoate (0.79 g, 2.5620 mmol, 41.1122% yield) obtained as a white solid. ¹H NMR (400 MHz, DMSO-d₆) δ 9.39 (s, 1H), 8.17 (s, 1H), 7.54 (s, 1H), 7.32 (s, 1H), 4.06 (q, J=7.1 Hz, 2H), 3.87 (s, 3H), 3.31-3.26 (m, 2H), 2.65 (t, J=6.3 Hz, 2H), 1.27-1.05 (m, 3H).

### INT A2: Methyl 4-(5-chloro-6-methoxythieno[3,2-b]pyridin-2-yl)-4-oxobutanoate

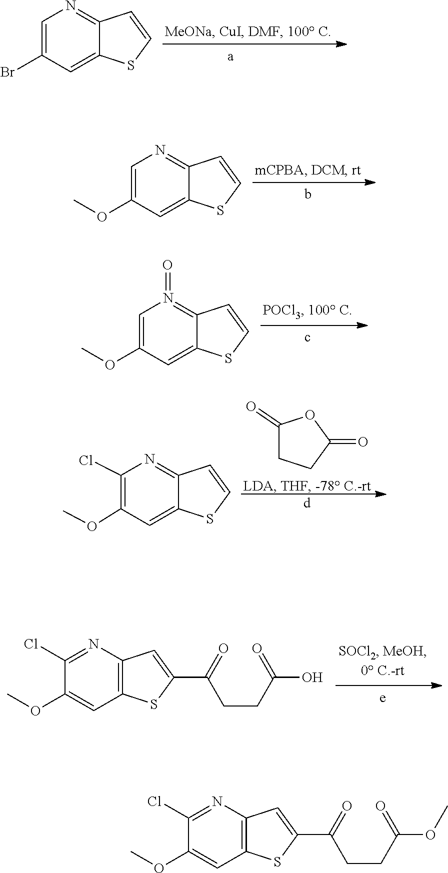

Step a: To a solution of 6-bromothieno[3,2-b]pyridine (30.0 g, 140.1 mmol) in DMF (300 mL) was added a solution of 25% MeONa in MeOH (605.6 g, 2802.6 mmol) and CuI (26.7 g, 140.1 mmol). The reaction mixture was heated to 100° C. and stirred for 2 h. Then the reaction mixture was cooled to room temperature, poured into ice water (900 mL) and filtered. The filtrate was diluted with NH₃·H₂O (300 mL), extracted with EtOAc (1.0 L×2) and washed with brine (1.0 L). The organic layer was dried over Na₂SO₄, filtered and the filtrate was concentrated to afford 6-methoxythieno[3,2-b]pyridine (19.0 g, 82.3%) as a yellow solid which was used for next step without further purification. ¹H NMR (300 MHz, CDCl₃): δ 8.43 (d, J=2.7 Hz, 1H), 7.62 (d, J=2.4 Hz, 1H), 7.53-7.51 (m, 1H), 7.47-7.45 (m, 1H), 3.91 (s, 3H). LCMS: (ESI, m/z): [M+H]⁺=166.2.

Step b: To a solution of 6-methoxythieno[3,2-b]pyridine (19.0 g, 115.0 mmol) in DCM (200 mL) was added m-CPBA (19.8 g, 115.0 mmol). The reaction mixture was stirred at room temperature for 16 h. Then the reaction mixture was diluted with DCM/MeOH=10:1 (300 mL) and washed with saturated sodium bicarbonate solution (300 mL×2) and brine (300 mL). The organic layer was dried over Na₂SO₄, filtered and the filtrate was concentrated to afford 6-methoxythieno[3,2-b]pyridine 4-oxide (13.0 g, 57.7%) as a yellow solid which was used for next step without further purification. ¹H NMR (300 MHz, CDCl₃): δ 8.14 (d, J=2.1 Hz, 1H), 7.74 (dd, J=5.7 Hz, 0.6 Hz, 1H), 7.48-7.46 (m, 1H), 7.31-7.30 (m, 1H), 3.91 (s, 3H). LCMS: (ESI, m/z): [M+H]⁺=182.0.

Step c: Add 6-methoxythieno[3,2-b]pyridine 4-oxide (15.5 g, 85.5 mmol) portion wise to POCl₃ (200 mL) at room temperature. The reaction mixture was heated to 100° C. and stirred for 16 h. Then the reaction mixture was cooled to room temperature and concentrated. The residue was diluted with water (200 mL), adjusted to pH=8 with saturated sodium bicarbonate solution and extracted with DCM (300 mL×2). The organic layers were washed with brine (200 mL), dried over Na₂SO₄, filtered and the filtrate was concentrated to give the residue which was purified by column chromatography on silica gel (eluted with petroleum ether/EtOAc=10:1) to afford 5-chloro-6-methoxythieno[3,2-b]pyridine (9.0 g, 52.6%) as a white solid. ¹H NMR (300 MHz, CDCl₃): δ 7.65 (s, 1H), 7.58-7.56 (m, 1H), 7.42 (dd, J=5.7 Hz, 0.6 Hz, 1H), 3.99 (s, 3H). LCMS: (ESI, m/z): [M+H]⁺=200.0.

Step d: To a solution of 5-chloro-6-methoxythieno[3,2-b]pyridine (8.0 g, 40.1 mmol) in THF (80 mL) was added LDA (2.0 M, 80 mL) dropwise at −78° C. under nitrogen. After 15 min, a solution of dihydrofuran-2,5-dione (22.0 g, 220.4 mmol) in THF (240 mL) was added to the mixture dropwise at −40° C. under nitrogen. Then the reaction mixture was warmed to room temperature and stirred for 2 h. After that the reaction was quenched with 2N HCl (100 mL) and extracted with EtOAc (300 mL×2). The organic layers were washed with brine (200 mL), dried over Na₂SO₄, filtered and the filtrate was concentrated. The residue was triturated with petroleum ether/EtOAc=1:1 and filtered to afford 4-(5-chloro-6-methoxythieno[3,2-b]pyridin-2-yl)-4-oxobutanoic acid (6.0 g, crude) as a white solid which was used for next step without further purification. LCMS: (ESI, m/z): [M−H]⁻=298.8.

Step e: To a solution of 4-(5-chloro-6-methoxythieno[3,2-b]pyridin-2-yl)-4-oxobutanoic acid (6.0 g crude, 20.0 mmol) in MeOH (120 mL) was added SOCl₂ (12.0 g, 100.1 mmol) dropwise at 0° C. Then the reaction mixture was warmed to room temperature and stirred for 2 h. After that the reaction was concentrated, diluted with water (100 mL), adjusted to pH=8 with saturated sodium bicarbonate solution and extracted with DCM/MeOH (200 mL×2). The organic layers were washed with brine (100 mL), dried over Na₂SO₄, filtered.

The filtrate was concentrated, white solid precipitated and filtered to afford methyl 4-(5-chloro-6-methoxythieno[3,2-b]pyridin-2-yl)-4-oxobutanoate (1.1 g, 8.5% in total of d and e two steps) as a white solid ¹H NMR (400 MHz, CDCl₃): δ 8.01 (s, 1H), 7.59 (s, 1H), 4.02 (s, 3H), 3.71 (s, 3H), 3.35 (t, J=6.8 Hz, 2H), 2.80 (t, J=6.8 Hz, 2H), LCMS: (ESI, m/z): [M+H]⁺=569.20.

### INT A3: Methyl trans-2-(5-bromo-6-methoxybenzo[b]thiophene-2-carbonyl)cyclopropane-1-carboxylate

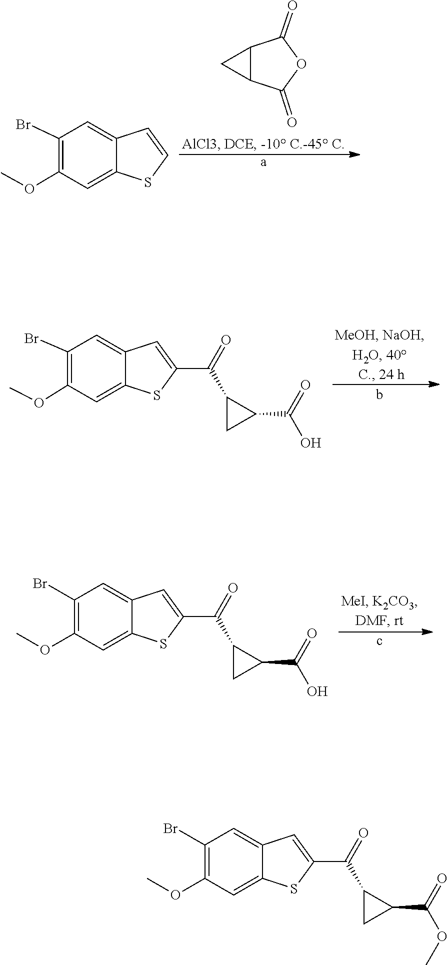 [SMILES: COc1cc2sccc2cc1Br ; O=C1OC(=O)C2CC12 ; COc1cc2sc(C(=O)[C@H]3C[C@H]3C(=O)O)cc2cc1Br ; COc1cc2sc(C(=O)[C@H]3C[C@@H]3C(=O)O)cc2cc1Br ; COC(=O)[C@H]1C[C@@H]1C(=O)c1cc2cc(Br)c(OC)cc2s1]

Step a: To a solution of 3-oxabicyclo [3.1.0]hexane-2,4-dione (6.3 g, 56.1 mmol) and AlCl₃ (12.3 g, 92.6 mmol) in DCE (150 mL) was added a solution of 5-bromo-6-methoxybenzo[b]thiophene (15.0 g, 61.7 mmol) in DCE (50 mL) at −10° C. After that the reaction mixture was heated to 45° C. and stirred for 16 h. Then the reaction mixture was poured into ice-water (200 mL) and extracted with DCM/MeOH=10:1 (100 mL×2). The organic layer washed with brine (100 mL), dried over Na₂SO₄, filtered and the filtrate was concentrated to dryness. The residue was triturated with Petroleum ether/EtOAc=1:1 (50 mL) and filtered to afford cis-2-(5-bromo-6-methoxybenzo[b]thiophene-2-carbonyl)cyclopropane-1-carboxylic acid (3.0 g, yield 15.1%) as a yellow solid. ¹H NMR (400 MHz, DMSO-d₆): δ 8.32 (s, 1H), 8.26 (s, 1H), 7.80 (s, 1H), 4.05 (s, 3H), 3.01-3.09 (m, 1H), 2.25-2.31 (m, 1H), 1.65-1.68 (m, 1H), 1.58-1.52 (m, 1H).

Step b: To a solution of cis-2-(5-bromo-6-methoxybenzo[b]thiophene-2-carbonyl)cyclopropane-1-carboxylic acid (3.0 g, 8.4 mmol) in MeOH (30 mL) was added 50% aq. NaOH (45 mL) at room temperature. Then the reaction mixture was heated to 40° C. and stirred for 24 h. After the reaction completed analysis by HPLC, the solvent was removed by concentrated. The residue was diluted with water (30 mL) and was adjusted pH˜2 with 6 N HCl. The white solid was collected by filtration. The solid was dryness under vacuum at 50° C. to afford trans-2-(5-bromo-6-methoxybenzo[b]thiophene-2-carbonyl)cyclopropane-1-carboxylic acid (2.5 g, yield 83.3%) as a yellow solid. ¹H NMR (400 MHz, DMSO-d₆): δ 8.52 (s, 1H), 8.27 (s, 1H), 7.80 (s, 1H), 3.96 (s, 3H), 3.27-3.23 (m, 1H), 2.16-2.11 (m, 1H), 1.54-1.49 (m, 2H).

Step c: To a solution of trans-2-(5-bromo-6-methoxybenzo[b]thiophene-2-carbonyl)cyclopropane-1-carboxylic acid (2.5 g, 7.0 mmol) in DMF (25 mL) was added K₂CO₃ (2.4 g, 17.5 mmol) and Mel (2.0 g, 14.1 mmol) at room temperature. The reaction mixture was stirred for 16 h. Then the mixture was poured into water (50 mL) and extracted with EtOAc (50 mL×2). The organic layer was washed with water (50 mL×2) and brine (50 mL×2). The organic layer was dried with anhydrous Na₂SO₄, filtered and the filtrate was concentrated to dryness. The residue was triturated with Petroleum ether/EtOAc=5:1 (50 mL) and filtered to afford methyl trans-2-(5-bromo-6-methoxybenzo[b]thiophene-2-carbonyl)cyclopropane-1-carboxylate (2 g, yield 77.5%) as a yellow solid. LCMS: (ESI, m/z): [M+H]⁺=368.8, 370.8. ¹H NMR (400 MHz, CDCl₃): δ 8.07 (s, 1H), 7.96 (s, 1H), 7.30 (s, 1H), 3.99 (s, 3H), 3.75 (s, 3H), 3.15-3.11 (m, 1H), 2.45-2.40 (m, 1H), 1.70-1.64 (m, 2H).

### INT A4: Ethyl 4-(4-chloro-5-hydroxy-6-methoxybenzo[b]thiophen-2-yl)-4-oxobutanoate

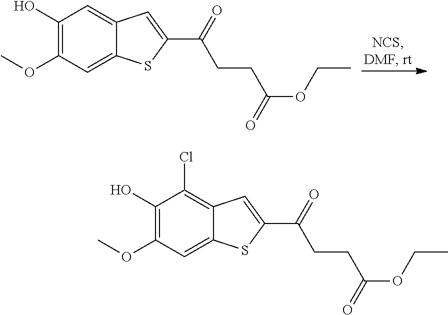

To a solution of ethyl 4-(5-hydroxy-6-methoxybenzo[b]thiophen-2-yl)-4-oxobutanoate (300 mg, 0.97 mmol) in DMF (6 mL) was added NCS (156 mg, 1.14 mmol) portion wise at room temperature. After that the reaction mixture was stirred for 5 h. Then the reaction mixture was poured into ice-water (18 mL). The mixture was filtered and the filter cake was dissolved with EtOAc (20 mL). The organic layer was washed with sat. aq. NaHCO₃ (15 mL) and brine (10 mL), dried over Na₂SO₄ and concentrated to afford a crude product which was purified by HPLC to give ethyl 4-(4-chloro-5-hydroxy-6-methoxybenzo[b]thiophen-2-yl)-4-oxobutanoate (240 mg, yield 72%) as a white solid. ¹H NMR (300 MHz, CDCl₃): δ 8.02 (s, 1H), 7.17 (s, 1H), 6.04 (brs, 1H), 4.20-4.12 (m, 2H), 4.02 (s, 3H), 3.36 (t, J=8.8 Hz, 2H), 2.79 (t, J=8.8 Hz, 2H), 1.30-1.20 (m, 3H). LCMS: (ESI, m/z): [M+H]⁺=342.9.

### INT A5: Ethyl 4-(5-methoxyisoindolin-2-yl)-4-oxo-butanoate

### INT A6: Ethyl 4-(5-bromo-6-methoxy-isoindolin-2-yl)-4-oxo-butanoate

### INT A7: Ethyl 4-(5-hydroxy-6-methoxyisoindolin-2-yl)-4-oxobutanoate and INT A8: Ethyl 4-(5-(3-bromopropoxy)-6-methoxyisoindolin-2-yl)-4-oxobutanoate

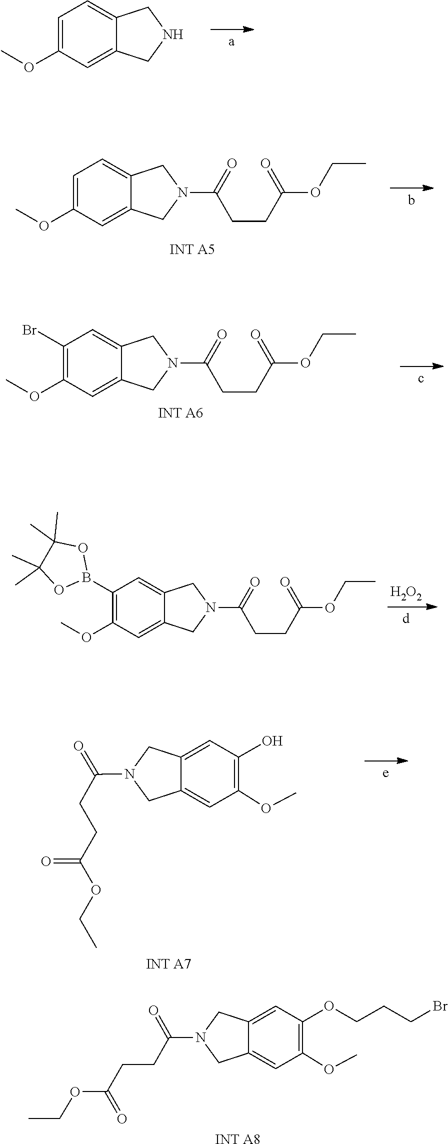

Step a: Succinic anhydride (4.40 g, 43.9681 mmol) was added to a solution of 5-Methoxyisoindoline hydrochloride (4.89 g, 26.3399 mmol) and Triethylamine (4.73 g, 46.7440 mmol) in Ethanol (200 mL) at 20° C. The reaction mixture was stirred for 1 h at 20° C. SOCl₂ (20 mL) was added to the solution above at 0° C. The reaction mixture was stirred for 3 h at 20° C. The reaction mixture was evaporated under reduced pressure. The reaction mixture was diluted with EA (200 mL), washed with water (100 mL) and brine (50 mL). The organics was dried over Na₂SO₄, filtered and evaporated under reduced pressure. There was ethyl 4-(5-methoxyisoindolin-2-yl)-4-oxo-butanoate (17.5 g, 31.5526 mmol, 119.7901% yield) obtained as a yellow solid.

¹H NMR (400 MHz, DMSO-d₆) δ 7.28-7.21 (m, 1H), 6.94 (s, 0.5H), 6.92 (s, 0.5H), 6.88 (s, 0.5H), 6.86 (s, 0.5H), 4.81 (s, 1H), 4.76 (s, 1H), 4.58 (s, 1H), 4.54 (s, 1H), 4.02-3.95 (m, 2H), 3.75 (s, 3H), 2.65-2.58 (m, 2H), 2.58-2.54 (m, 2H), 1.17 (t, J=3.5 Hz, 3H).

Step b: NBS (10.51 g, 59.0503 mmol) was added to ethyl 4-(5-methoxyisoindolin-2-yl)-4-oxo-butanoate in Tetrahydrofuran (100 mL) and Acetonitrile (100 mL) at 20° C. The reaction mixture was stirred overnight at 20° C. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fractions was concentrated and dried under vacuo. There was ethyl 4-(5-bromo-6-methoxy-isoindolin-2-yl)-4-oxo-butanoate (7.26 g, 20.3812 mmol, 64.5943% yield) obtained as a yellow solid.

¹H NMR (400 MHz, DMSO-d₆) δ 7.58 (s, 0.5H), 7.57 (s, 0.5H), 7.15 (s, 0.5H), 7.12 (s, 0.5H), 4.80 (s, 1H), 4.77 (s, 1H), 4.57 (s, 1H), 4.54 (s, 1H), 4.09-4.01 (m, 2H), 3.84 (s, 3H), 2.65-2.59 (m, 2H), 2.56-2.53 (m, 2H), 1.18 (t, J=7.1 Hz, 3H).

Step c: Potassium Acetate (3.74 g, 38.1080 mmol) was added to Pd(dppf)Cl₂ (0.72 g, 984.0050 mol), ethyl 4-(5-bromo-6-methoxy-isoindolin-2-yl)-4-oxo-butanoate (3.64 g, 10.2187 mmol) and 4,4,4′,4′,5,5,5′,5′-Octamethyl-2,2′-bi(1,3,2-dioxaborolane) (3.84 g, 15.1218 mmol) in 1,4-Dioxane (100 mL) at 20° C. The reaction mixture was stirred at 100° C. for 16 h. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column EA/Hept (0-100%). The pure fractions was concentrated and dried under vacuo. There was ethyl 4-(5-methoxy-6-(4,4,5,5-tetramethyl-1,3,2-dioxaborolan-2-yl)isoindolin-2-yl)-4-oxobutanoate (2.45 g, 6.0752 mmol, 59.7809% yield) obtained as a yellow solid.

¹H NMR (400 MHz, DMSO-d₆) δ 7.49 (s, 0.5H), 7.48 (s, 0.5H), 6.98 (s, 0.5H), 6.95 (s, 0.5H), 4.83 (s, 1H), 4.74 (s, 1H), 4.60 (s, 1H), 4.53 (s, 1H), 4.09-4.01 (m, 2H), 3.74 (t, J=6.2 Hz, 3H), 2.65-2.58 (m, 2H), 2.58-2.52 (m, 2H), 1.27 (s, 12H), 1.21-1.13 (m, 3H).

Step d: Sodium perborate tetrahydrate (1 g, 6.4994 mmol) was added to ethyl 4-(5-methoxy-6-(4,4,5,5-tetramethyl-1,3,2-dioxaborolan-2-yl)isoindolin-2-yl)-4-oxobutanoate (2.45 g, 6.0752 mmol) in Tetrahydrofuran (20 mL) and Water (20 mL) at 20° C. The reaction mixture was stirred for 1 h at 20° C. The reaction mixture was evaporated under reduced pressure. The reaction mixture was concentrated and diluted with DCM (500 mL), washed with NaHCO₃ aq. (2×300 mL) and brine (200 mL). The organics was dried over Na₂SO₄, filtered and evaporated under reduced pressure. The residue was purified on silica gel column MeOH/DCM (0-10%). The pure fractions was concentrated and dried under vacuo. There was ethyl 4-(5-hydroxy-6-methoxyisoindolin-2-yl)-4-oxobutanoate (1.37 g, 4.6708 mmol, 76.8818% yield) obtained as a yellow solid.

¹H NMR (400 MHz, DMSO-d₆) δ 8.97 (s, 0.5H), 8.96 (s, 0.5H), 6.91 (s, 0.5H), 6.89 (s, 0.5H), 6.73 (s, 0.5H), 6.73 (s, 0.5H), 4.71 (s, 1H), 4.69 (s, 1H), 4.50 (s, 1H), 4.47 (s, 1H), 4.08-4.00 (m, 2H), 3.75 (s, 3H), 2.64-2.58 (m, 2H), 2.56-2.53 (m, 2H), 1.18 (t, J=7.1 Hz, 3H).

Step e: To a solution of ethyl 4-(5-hydroxy-6-methoxy-isoindolin-2-yl)-4-oxo-butanoate (0.72 g, 2.4547 mmol) in N,N-Dimethylformamide (10 mL) was added 1,3-Dibromopropane (0.73 g, 3.6159 mmol) and potassium carbonate (0.86 g, 6.2226 mmol)2. This mixture was stirred for 16 hours at 50° C. The reaction mixture was diluted with Ethyl acetate (100 mL), and washed sequentially with water (2×100 mL) and saturated brine (1×100 mL). The organic layer was dried over Na₂SO₄, filtered and evaporated to afford crude product. The crude product was purified by flash silica chromatography, elution gradient 0 to 100% Ethyl acetate in heptane. There was ethyl 4-(5-(3-bromopropoxy)-6-methoxyisoindolin-2-yl)-4-oxobutanoate (0.43 g, 1.0379 mmol, 42.2829% yield) obtained as a white solid.

¹H NMR (400 MHz, DMSO-d₆) δ 7.01-6.93 (m, 2H), 4.75 (s, 2H), 4.53 (s, 2H), 4.10-4.00 (m, 4H), 3.76 (s, 3H), 3.71-3.62 (m, 2H), 2.65-2.58 (m, 2H), 2.58-2.53 (m, 2H), 2.28-2.18 (m, 2H), 1.18 (t, J=7.1 Hz, 3H).

### INT A9: Tert-butyl 3-(5-bromo-4-fluoro-6-methoxy-benzothiophen-2-yl)-3-oxo-propanoate

### Step a: 3-bromo-2-fluoro-4-methoxy-benzaldehyde

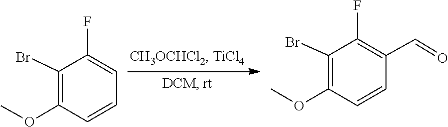

Titanium tetrachloride (38.06 g, 200.6548 mmol) was added dropwise to a solution of 2-Bromo-3-fluoroanisole (10.17 g, 49.6039 mmol) in Dichloromethane (250 mL) at 0° C. for 1 h. The reaction mixture was stirred for 3 h at 20° C. The reaction mixture was quenched with adding of ice-water (200 mL) at 0° C. The reaction mixture was extracted with DCM (2×100 mL), washed with NaHCO₃ (1×200 mL) and brine (100 mL). The organics dried over Na₂SO₄, filtered and evaporated under reduced pressure. The residue was dried under vacuo at 60° C. There was 3-bromo-2-fluoro-4-methoxy-benzaldehyde (11.42 g, 49.0057 mmol, 98.7940% yield) obtained as a white solid. LCMS: (ESI, m/z): [M+H]⁺=232.954. ¹H NMR (400 MHz, CDCl₃-d₆) δ 10.24 (s, 1H), 7.87 (t, J=8.3 Hz, 1H), 6.84 (d, J=8.8 Hz, 1H), 4.05-3.99 (m, 3H).

### Step b: (5E)-5-[(3-bromo-2-fluoro-4-methoxy-phenyl)methylene]-2-thioxo-thiazolidin-4-one

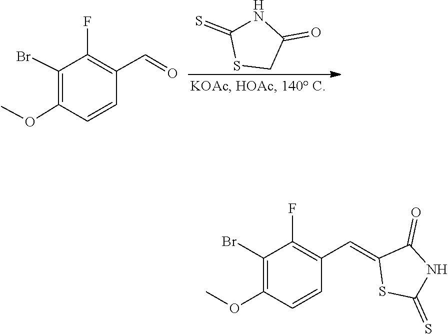 [SMILES: COc1ccc(C=O)c(F)c1Br ; O=C1CSC(=S)N1 ; COc1ccc(/C=C2\SC(=S)NC2=O)c(F)c1Br]

Potassium Acetate (12.67 g, 129.0984 mmol) was added to a mixture of 3-bromo-2-fluoro-4-methoxy-benzaldehyde (10.01 g, 42.9551 mmol) and Rhodanine (5.72 g, 42.9456 mmol) in Acetic acid (100 mL) at 20° C. The reaction mixture was heated to 140° C. and stirred overnight. The reaction mixture was added to water (1000 mL) and stirred for 10 min. The precipitate was collected by filtration, washed with water (200 mL). The filtrate cake was dried under vacuo at 60° C. There was (5E)-5-[(3-bromo-2-fluoro-4-methoxy-phenyl)methylene]-2-thioxo-thiazolidin-4-one (9.21 g, 26.4495 mmol, 61.5748% yield) obtained as a yellow solid. LCMS: (ESI, m/z): [M+H]⁺=347.909. ¹H NMR (400 MHz, DMSO-d₆) δ 13.87 (s, 1H), 7.68-7.32 (m, 2H), 7.16 (d, J=8.7 Hz, 1H), 3.97 (s, 3H).

### Step c: (Z)-3-(3-bromo-2-fluoro-4-methoxy-phenyl)-2-sulfanyl-prop-2-enoic Acid

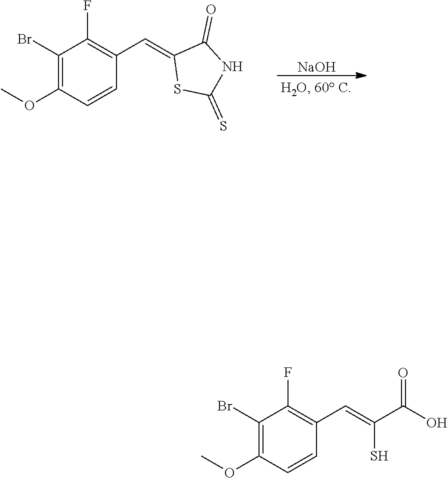 [SMILES: COc1ccc(/C=C2\SC(=S)NC2=O)c(F)c1Br ; "NaOH" ; COc1ccc(/C=C(\S)C(=O)O)c(F)c1Br]

To a stirred mixture of (5E)-5-[(3-bromo-2-fluoro-4-methoxy-phenyl)methylene]-2-thioxo-thiazolidin-4-one (8.03 g, 23.0607 mmol) in Water (200 mL) was added NaOH (9.15 g, 228.7666 mmol) in water (80 mL) at 20° C. The reaction mixture was stirred at 60° C. for 1 h. After the reaction completed, adjusted to pH=3 with HCl. The precipitate was collected by filtration, washed with water (1×100 mL). The filtrate cake was dried under vacuo at 60° C. There was (Z)-3-(3-bromo-2-fluoro-4-methoxy-phenyl)-2-sulfanyl-prop-2-enoic acid (7.37 g, 23.9959 mmol, 104.0552% yield) obtained as a yellow solid. LCMS: (ESI, m/z): [M+H]⁺=306.936.

### Step d: 5-bromo-4-fluoro-6-methoxy-benzothiophene-2-carboxylic Acid

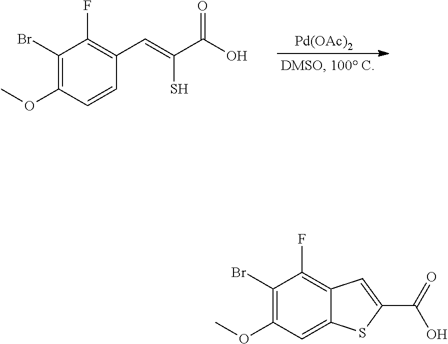 [SMILES: COc1ccc(/C=C(\S)C(=O)O)c(F)c1Br ; COc1cc2sc(C(=O)O)cc2c(F)c1Br ; "Pd(OAc)2"]

Palladium (II) acetate (4.0934 g, 18.2330 mmol) was added to a solution of (Z)-3-(3-bromo-2-fluoro-4-methoxy-phenyl)-2-sulfanyl-prop-2-enoic acid (7 g, 22.7912 mmol) in Methyl sulfoxide (50 mL) at 20° C. under N₂ atmosphere. The reaction mixture was heated to 100° C. and stirred for 1 h. The reaction mixture was purified on C-18 column MeCN/water (0-100%). The pure fractions was concentrated and dried under vacuo. There was 5-bromo-4-fluoro-6-methoxy-benzothiophene-2-carboxylic acid (3.9 g, 12.7819 mmol, 56.0824% yield) obtained as a yellow solid. LCMS: (ESI, m/z): [M+H]⁺=304.921. ¹H NMR (400 MHz, DMSO-d₆) δ 7.96 (s, 1H), 7.70 (s, 1H), 3.97 (s, 3H).

### Step e: Tert-butyl 3-(5-bromo-4-fluoro-6-methoxy-benzothiophen-2-yl)-3-oxo-propanoate

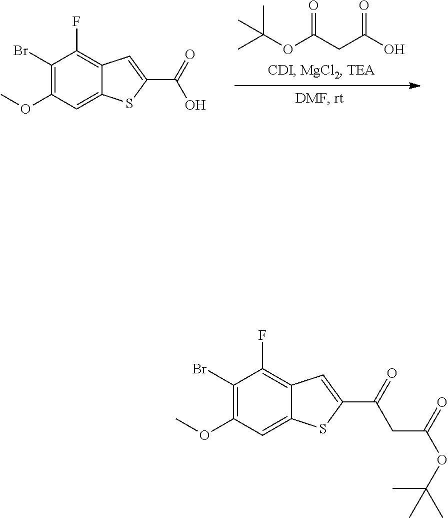 [SMILES: COc1cc2sc(C(=O)O)cc2c(F)c1Br ; CC(C)(C)OC(=O)CC(=O)O ; COc1cc2sc(C(=O)CC(=O)OC(C)(C)C)cc2c(F)c1Br]

To a stirred solution of 5-bromo-4-fluoro-6-methoxy-benzothiophene-2-carboxylic acid (3.88 g, 12.7163 mmol) in DMF (50 mL) was added Carbonyl diimidazole (4.12 g, 25.4088 mmol) at 20° C. The resulting mixture was stirred for 1 h at 20° C. under nitrogen atmosphere. To the mixture above was added Magnesium chloride (1.81 g, 19.0104 mmol), 3-tert-Butoxy-3-oxopropanoic acid (3.13 g, 19.5421 mmol) and Triethylamine (3.91 g, 38.6404 mmol). The reaction mixture was stirred overnight at 20° C. The reaction mixture was concentrated and diluted with EA (500 mL), washed with NaHCO₃ aq (2×500 mL) and brine (100 mL). The organics dried over Na₂SO₄, filtered and evaporated under reduced pressure. The residue was purified by flash silica chromatography, elution gradient 0 to 100% Ethyl acetate in heptane. The pure fractions was concentrated and dried under vacuo. There was tert-butyl 3-(5-bromo-4-fluoro-6-methoxy-benzothiophen-2-yl)-3-oxo-propanoate (3.29 g, 8.1585 mmol, 64.1574% yield) obtained as a yellow solid. LCMS: (ESI, m/z): [M+H]⁺=402.994. ¹H NMR (400 MHz, DMSO-d₆) δ 8.38 (d, J=2.2 Hz, 1H), 7.74 (d, J=8.5 Hz, 1H), 4.16 (s, 2H), 3.97 (d, J=8.2 Hz, 3H), 1.41 (d, J=1.6 Hz, 10H).

### INT A10: Tert-butyl 4-fluoro-5-hydroxy-6-methoxy-isoindoline-2-carboxylate

### Step a: 4-fluoro-5-methoxy-isoindoline

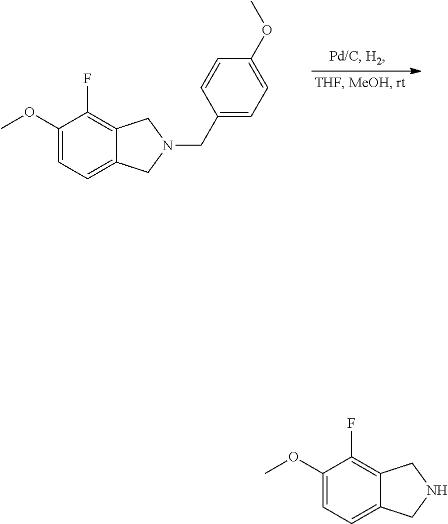 [SMILES: COc1ccc(CN2Cc3ccc(OC)c(F)c3C2)cc1 ; COc1ccc2c(c1F)CNC2]

10% Pd/C (7.68 g, contain 55% H₂O) was added to a solution of 4-fluoro-5-methoxy-2-[(4-methoxyphenyl)methyl]isoindoline (7.18 g, 24.9889 mmol) in THF (70 mL) and MeOH (70 mL) at 20° C. The reaction mixture was stirred overnight at 20° C. The reaction mixture was filtered and the filter cake was washed with MeOH (70 mL). The filtrate was evaporated under reduced pressure. There was 4-fluoro-5-methoxy-isoindoline (4.88 g, 29.1901 mmol, 100% yield) obtained as a brown oil, which was used directly in the next step. LCMS: (ESI, m/z): [M+H]⁺=168.200.

### Step b: 4-fluoroisoindolin-5-ol Hydrobromate

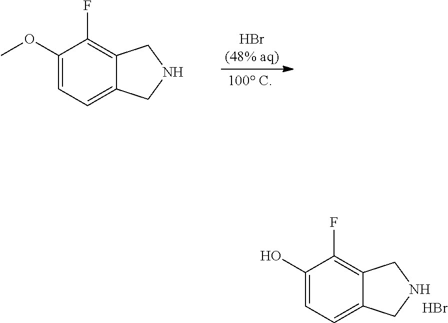

4-fluoro-5-methoxy-isoindoline (4.5 g, 26.9171 mmol) was dissolved in Hydrobromic acid (120 mL, 48% aq) at 20° C. The reaction mixture was heated to 100° C. and stirred for 6 h. The resulting reaction mixture was evaporated under reduced pressure. The residue was treated with n-hexane/EA (1:2). The solid was collected by filtration, and dried under reduced pressure. There was 4-fluoroisoindolin-5-ol hydrobromate (4.70 g, 20.0799 mmol, 74.60% yield) obtained as a brown solid. LCMS: (ESI, m/z): [M+H]⁺=154.200. ¹H NMR (400 MHz, DMSO-d₆) δ 10.09 (s, 1H), 9.58 (s, 2H), 7.10-6.93 (m, 2H), 4.57 (s, 2H), 4.44 (s, 2H).

### Step c: Tert-butyl 4-fluoro-5-hydroxy-isoindoline-2-carboxylate

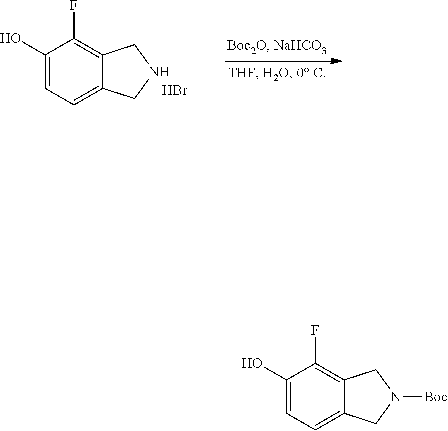 [SMILES: Oc1ccc2c(c1F)CNC2 ; Br ; CC(C)(C)OC(=O)N1Cc2ccc(O)c(F)c2C1]

NaHCO₃ (3.43 g, 40.8301 mmol) was added to a solution of 4-fluoroisoindolin-5-ol hydrobromate (4.70 g, 20.0799 mmol) in Water (80 mL) at 0° C. After the reaction mixture was stirred for 20 min, THF (60 mL) was added to the reaction mixture, and Di-tert-butyl dicarbonate (4.45 g, 20.3898 mmol) in THF (20 mL) was added dropwise to the mixture at 0° C. After stirring for 20 min, the reaction mixture was warmed to 20° C. and stirred for 2 h. The resulting reaction mixture was diluted with EA (150 mL) and washed with brine (150 mL). The organic layer was dried over Na₂SO₄, filtered and evaporated under reduced pressure. The residue was purified by flash chromatography, eluting with ethyl acetate in n-hexane (0-40%). The pure fractions was concentrated and dried under reduced pressure. There was tert-butyl 4-fluoro-5-hydroxy-isoindoline-2-carboxylate (3.14 g, 12.3979 mmol, 60.78% yield) obtained as a reddish brown solid. LCMS: (ESI, m/z): [M−H]⁻=252.000. ¹H NMR (400 MHz, CDCl₃-d) δ 7.00-6.93 (m, 1H), 6.93-6.83 (m, 1H), 4.72 (s, 2H), 4.64 (s, 2H), 1.54 (s, 9H).

### Step d: Tert-butyl 6-bromo-4-fluoro-5-hydroxy-isoindoline-2-carboxylate

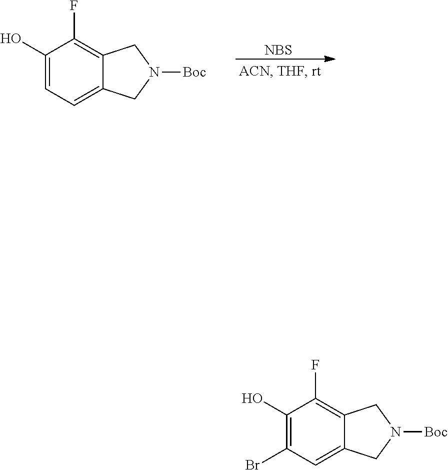 [SMILES: CC(C)(C)OC(=O)N1Cc2ccc(O)c(F)c2C1 ; "NBS" ; CC(C)(C)OC(=O)N1Cc2cc(Br)c(O)c(F)c2C1]

NBS (2.20 g, 12.3607 mmol) was added to a solution of tert-butyl 4-fluoro-5-hydroxy-isoindoline-2-carboxylate (3.10 g, 12.2400 mmol) in ACN (40 mL) and THF (20 mL) at 0° C. The reaction mixture was stirred for 2.5 h at 20° C. The reaction mixture was concentrated under reduced pressure. The residue was dissolved with EA/MeOH (200 mL/10 mL), and washed with H₂O (200 mL). The organic layer was dried over Na₂SO₄, filtered and evaporated in vacuo. The residue was purified by flash chromatography, eluting with MeOH in DCM (0-5%). The pure fractions was concentrated and dried under reduced pressure. There was tert-butyl 6-bromo-4-fluoro-5-hydroxy-isoindoline-2-carboxylate (2.70 g, 8.1285 mmol, 66.34% yield) obtained as a white solid. LCMS: (ESI, m/z): [M−H]=331.950. ¹H NMR (400 MHz, DMSO-d₆) δ 10.36 (s, 1H), 7.33 (s, 0.5H), 7.32 (s, 0.5H), 4.58 (s, 1H), 4.55 (s, 1H), 4.52 (s, 1H), 4.50 (s, 1H), 1.45 (s, 9H).

### Step e: Tert-butyl 5-benzyloxy-6-bromo-4-fluoro-isoindoline-2-carboxylate

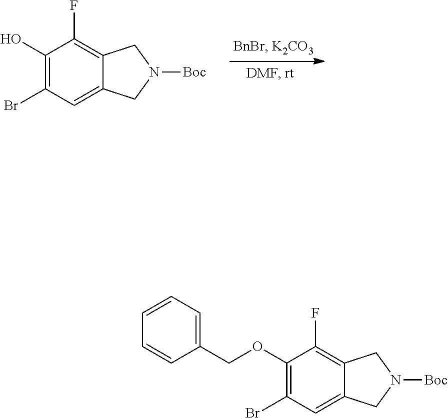 [SMILES: CC(C)(C)OC(=O)N1Cc2cc(Br)c(O)c(F)c2C1 ; CC(C)(C)OC(=O)N1Cc2cc(Br)c(OCc3ccccc3)c(F)c2C1]

K₂CO₃ (2.20 g, 15.9183 mmol) was added to a solution of tert-butyl 6-bromo-4-fluoro-5-hydroxy-isoindoline-2-carboxylate (2.70 g, 8.1285 mmol) in DMF (50 mL) at 0° C. After the reaction mixture was stirred for 0.5 h at 0° C., BnBr (1.90 g, 11.1089 mmol) was added to the reaction mixture. The reaction mixture was stirred for 9 h at 20° C. The reaction mixture was diluted with EA (150 mL) and washed with water (150 mL) and brine (2×150 mL). The organics dried over Na₂SO₄, filtered and evaporated under reduced pressure. The residue was purified by flash chromatography, eluting with ethyl acetate in n-hexane (0-10%). The pure fractions was concentrated and dried under reduced pressure. There was tert-butyl 5-benzyloxy-6-bromo-4-fluoro-isoindoline-2-carboxylate (3.43 g, 8.1224 mmol, 99.93% yield) obtained as a white semi-solid. LCMS: (ESI, m/z): [M-tBu+ACN]⁺=407.050. H NMR (400 MHz, CDCl₃-d) δ 7.54 (d, J=7.3 Hz, 2H), 7.45-7.35 (m, 3H), 7.27 (s, 0.5H), 7.20 (s, 0.5H), 5.13 (s, 2H), 4.70 (s, 1H), 4.65 (s, 2H), 4.62 (s, 1H), 1.54 (s, 9H).

### Step f: Tert-butyl 5-benzyloxy-4-fluoro-6-hydroxy-isoindoline-2-carboxylate

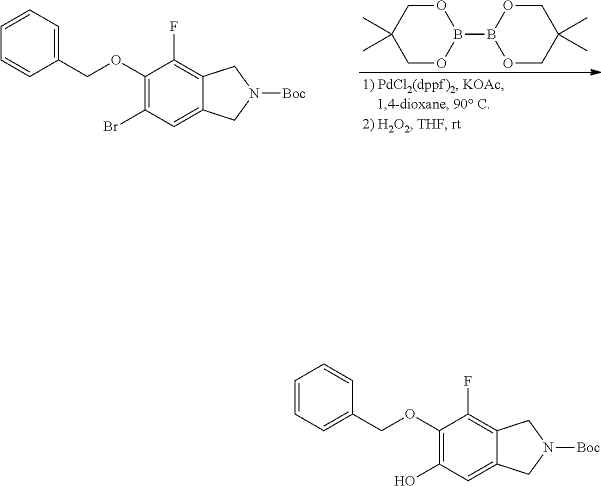

Pd(dppf)Cl₂ (1.12 g, 1.5307 mmol) was added to a solution of tert-butyl 5-benzyloxy-6-bromo-4-fluoro-isoindoline-2-carboxylate (3.30 g, 7.8146 mmol), bis(neopentylglycolato)diboron (5.46 g, 24.1716 mmol) and KOAc (2.32 g, 23.6392 mmol) in 1,4-Dioxane (80 mL) at 20° C. The reaction mixture was heated to 90° C. and stirred overnight under nitrogen atmosphere. The reaction mixture was diluted with EA (100 mL) and filtered through a celite. The filtrate was evaporated under reduced pressure. H₂O₂ (88.1974 mmol, 10 mL, 30% aqueous solution) was added to a mixture of the residue and NaHCO₃ (11.9038 mmol, 20 mL, 5% aqueous solution) in THF (80 mL) at 0° C. The reaction mixture was stirred for 2 h at 20° C. The resulting reaction mixture was diluted with brine (150 mL) and saturated NaHSO₃ (100 mL), and extracted with EA (200 mL). The organic layer was collected and dried over Na₂SO₄, filtered and evaporated under reduced pressure. The residue was purified on silica gel column chromatography, eluting with ethyl acetate/n-hexane (0-10%). There was tert-butyl 5-benzyloxy-4-fluoro-6-hydroxy-isoindoline-2-carboxylate (1.93 g, 5.3702 mmol, 68.93% yield) obtained as an off-white solid. LCMS: (ESI, m/z): [M−H]⁻=358.100. ¹H NMR (400 MHz, CDCl₃-d) δ 7.46-7.36 (m, 5H), 6.62 (s, 0.5H), 6.58 (s, 0.5H), 5.15 (s, 2H), 4.68 (s, 1H), 4.65 (s, 1H), 4.61 (s, 1H), 4.58 (s, 1H), 1.54 (s, 9H).

### Step g: Tert-butyl 5-benzyloxy-4-fluoro-6-methoxy-isoindoline-2-carboxylate

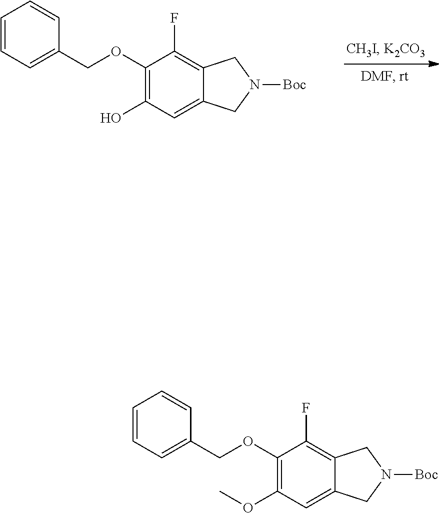 [SMILES: CC(C)(C)OC(=O)N1Cc2cc(O)c(OCc3ccccc3)c(F)c2C1 ; COc1cc2c(c(F)c1OCc1ccccc1)CN(C(=O)OC(C)(C)C)C2]

K₂CO₃ (1.18 g, 8.5380 mmol) was added to a solution of tert-butyl 5-benzyloxy-4-fluoro-6-hydroxy-isoindoline-2-carboxylate (1.9 g, 5.2867 mmol) in DMF (25 mL) at 0° C. After the mixture was stirred for 30 min at 0° C., CH₃I (1.12 g, 7.8908 mmol) was added. The reaction mixture was stirred for 3 h at 20° C. The resulting reaction mixture was diluted with water (200 mL) and extracted with EA (200 mL). The organic layer was separated and washed with brine (2×150 mL). The organic layer was collected and dried over Na₂SO₄, filtered and evaporated under reduced pressure. There was tert-butyl 5-benzyloxy-4-fluoro-6-methoxy-isoindoline-2-carboxylate (1.98 g, 5.3024 mmol, 100% yield) obtained as a colorless oil, which was directly used in the next step without purification. LCMS: (ESI, m/z): [M-tBu+ACN]⁺=359.150. ¹H NMR (400 MHz, CDCl₃-d) δ 7.39 (d, J=7.4 Hz, 2H), 7.31-7.21 (m, 3H), 6.51 (s, 1H), 4.99 (s, 2H), 4.55 (s, 4H), 3.77 (s, 3H), 1.44 (s, 9H).

### Step h: Tert-butyl 4-fluoro-5-hydroxy-6-methoxy-isoindoline-2-carboxylate

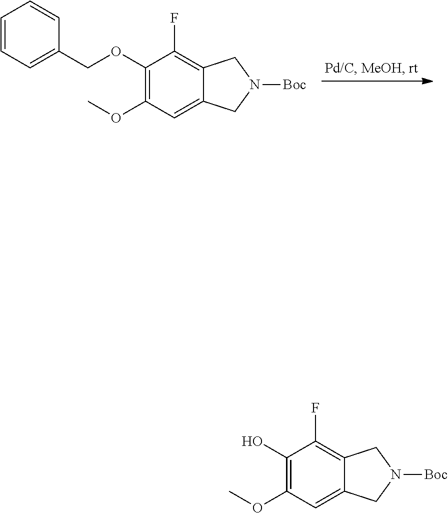 [SMILES: COc1cc2c(c(F)c1OCc1ccccc1)CN(C(=O)OC(C)(C)C)C2 ; COc1cc2c(c(F)c1O)CN(C(=O)OC(C)(C)C)C2]

10% Pd/C (1.98 g, contain 55% H₂O) was added to a solution of tert-butyl 5-benzyloxy-4-fluoro-6-methoxy-isoindoline-2-carboxylate (1.96 g, 5.2488 mmol) in MeOH (40 mL) and THF (10 mL) at 20° C. The reaction mixture was stirred for 5 h at 20° C. under H₂ atmosphere. The reaction mixture was diluted with EA (20 mL) and filtered through a celite. The filtrate was evaporated under reduced pressure. The residue was purified on silica gel column chromatography, eluting with ethyl acetate/n-hexane (0-40%). The pure fractions was concentrated and dried under reduced pressure. There was tert-butyl 4-fluoro-5-hydroxy-6-methoxy-isoindoline-2-carboxylate (1.21 g, 4.2712 mmol, 80.67% yield) obtained as a brown semi-solid. LCMS: (ESI, m/z): [M−H]⁻=282.100. ¹H NMR (400 MHz, CDCl₃-d) δ 6.58 (s, 1H), 4.67 (s, 2H), 4.63 (s, 2H), 3.92 (s, 3H), 1.53 (s, 9H).

### INT A11: Ethyl 4-(4-fluoro-5-hydroxy-6-methoxy-isoindolin-2-yl)-4-oxo-butanoate

### Step a: 7-fluoro-6-methoxy-2-[(4methoxyphenyl) methyl] isoindolin-1-one

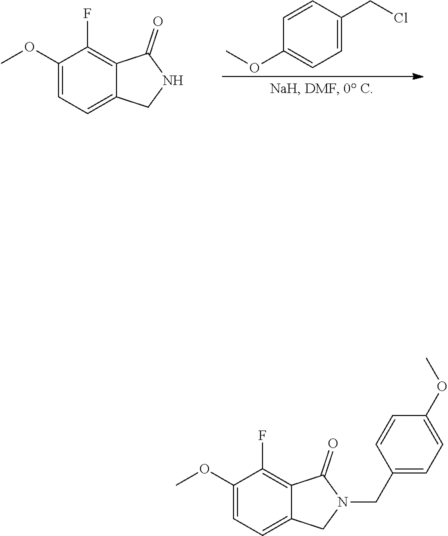

4-Methoxybenzylchloride (4.141 g, 26.4416 mmol) was added to a solution of NaH (1.000 g, 25.0024 mmol) and 7-fluoro-6-methoxyisoindolin-1-one (4.336 g, 23.9342 mmol) in DMF (100 mL) at 0° C. under N₂ atmosphere. The reaction mixture was stirred for 3 h and warmed up to 20° C. naturally. The reaction mixture was quenched with adding to water (300 mL) at 20° C., extracted with EA (3×200 mL) and washed with brine (150 mL). The organics dried over Na₂SO₄, filtered and evaporated under reduced pressure. The residue was purified on silica gel column EA/n-Hex (0-100%). The pure fractions was concentrated and dried under vacuo. There was 7-fluoro-6-methoxy-2-[(4methoxyphenyl) methyl] isoindolin-1-one (6.51 g, 21.6055 mmol, 90.2706% yield) obtained as a yellow solid. LCMS: (ESI, m/z): [M+H]⁺=302.110. ¹H NMR (400 MHz, DMSO-d₆) δ 7.39 (t, J=7.9 Hz, 1H), 7.27 (d, J=8.2 Hz, 1H), 7.22 (d, J=8.2 Hz, 2H), 6.91 (d, J=8.3 Hz, 2H), 4.60 (s, 2H), 4.25 (s, 2H), 3.87 (s, 3H), 3.73 (s, 3H).

### Step b: 4-fluoro-5-methoxy-2[(4methoxyphenyl)methyl]isoindoline

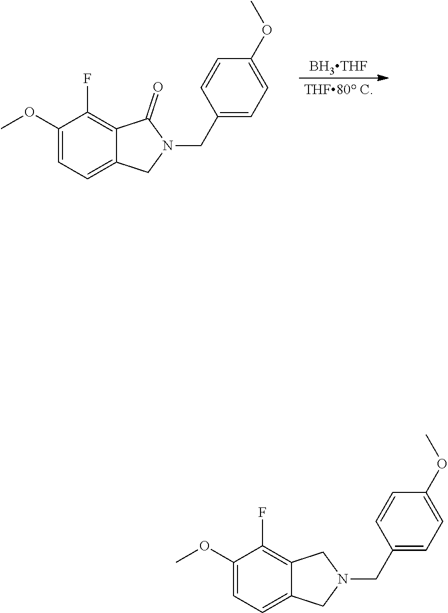 [SMILES: COc1ccc(CN2Cc3ccc(OC)c(F)c3C2=O)cc1 ; COc1ccc(CN2Cc3ccc(OC)c(F)c3C2)cc1 ; "BH3·THF"]

Borane-tetrahydrofuran complex (120 mL, 120 mmol) was added to a solution of 7-fluoro-6-methoxy-2-(4-methoxybenzyl)isoindolin-1-one (6.17 g, 20.4771 mmol) in THF (60 mL) at 20° C. under N₂ atmosphere. The reaction mixture was heated to 80° C. and stirred for 5 h. The reaction mixture was quenched with pouring into MeOH (300 mL) at 20° C. and evaporated under reduced pressure. The residue was diluted with MeOH (100 mL). The precipitate was collected by filtration, washed with MeOH (50 mL). The filter cake was dried under vacuo. There was 4-fluoro-5-methoxy-2[(4methoxyphenyl)methyl]isoindoline (3.854 g, 13.4132 mmol, 65.5036% yield) obtained as a white solid. LCMS: (ESI, m/z): [M+H]⁺=288.130. ¹H NMR (400 MHz, DMSO-d₆) δ 7.41 (d, J=8.0 Hz, 2H), 7.06-6.93 (m, 2H), 6.88 (d, J=8.0 Hz, 2H), 4.53-4.37 (m, 2H), 4.22-4.08 (m, 4H), 3.79 (s, 3H), 3.74 (s, 3H).

### Step c: Ethyl 4-(4-fluoro-5-methoxy-isoindolin-2-yl)-4-oxo-butanoate

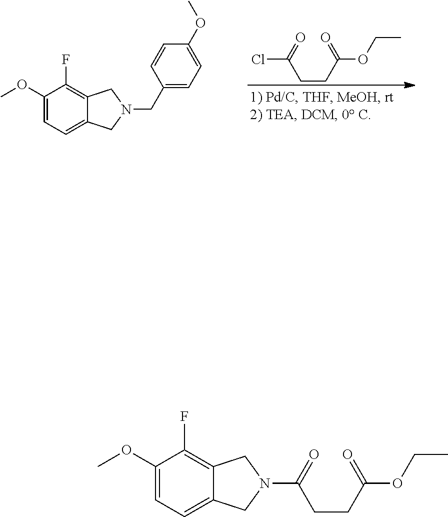

Pd/C (2.17 g, 2.0391 mmol) was added to a solution of 4-fluoro-5-methoxy-2-(4-methoxybenzyl)isoindoline (3.36 g, 11.6940 mmol) in THF (20 mL) and MeOH (20 mL) at 20° C. The reaction mixture was stirred overnight at 20° C. under H₂ atmosphere. The precipitate was collected by filtration, washed with MeOH (50 mL). The filtrate was evaporated under reduced pressure. Ethyl Succinyl Chloride (5.05 g, 30.6830 mmol) was added to a solution of the residue and TEA (3.91 g, 38.6404 mmol) in DCM (50 mL) at 0° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on silica gel column EA/n-Hex (0-45%). The pure fractions was concentrated and dried under vacuo. There was ethyl 4-(4-fluoro-5-methoxy-isoindolin-2-yl)-4-oxo-butanoate (2.619 g, 8.8688 mmol, 75.8406% yield) obtained as a yellow solid. LCMS: (ESI, m/z): [M+H]⁺=296.120. ¹H NMR (400 MHz, DMSO-d₆) δ 7.16-7.09 (m, 2H), 4.91 (s, 1H), 4.81 (s, 1H), 4.64 (s, 1H), 4.58 (s, 1H), 4.05 (q, J=7.2 Hz, 2H), 3.84 (s, 3H), 2.73-2.53 (m, 4H), 1.18 (t, J=7.2 Hz, 3H).

### Step d: Ethyl 4-(4-fluoro-5-hydroxy-isoindolin-2-yl)-4-oxo-butanoate

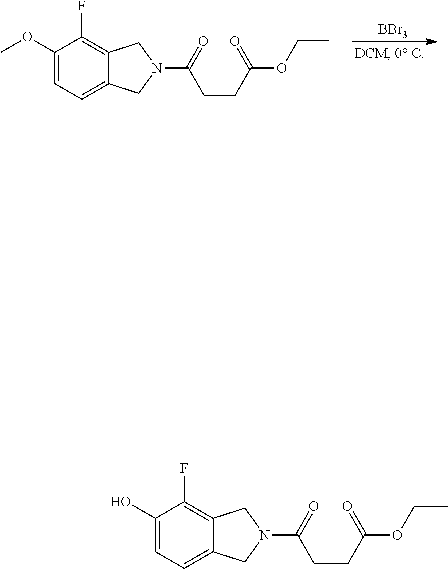 [SMILES: CCOC(=O)CCC(=O)N1Cc2ccc(OC)c(F)c2C1 ; "BBr3" ; CCOC(=O)CCC(=O)N1Cc2ccc(O)c(F)c2C1]

Tribromoboron (20 mL, 20 mmol) was added to a solution of ethyl 4-(4-fluoro-5-methoxyisoindolin-2-yl)-4-oxobutanoate (2.597 g, 8.7943 mmol) in DCM (40 mL) at 0° C. under N₂ atmosphere. The reaction mixture was stirred for 2 h at 20° C. The reaction mixture was quenched with adding to EtOH (100 mL) at 20° C., The reaction mixture was evaporated under reduced pressure and diluted with H₂O (200 mL), extracted with EA (2×100 mL) and washed with brine (100 mL). The organics dried over Na₂SO₄, filtered and evaporated under reduced pressure. There was ethyl 4-(4-fluoro-5-hydroxy-isoindolin-2-yl)-4-oxo-butanoate (2.408 g, 8.5609 mmol, 97.3461% yield) obtained as a yellow solid. LCMS: (ESI, m/z): [M+H]⁺=282.110. ¹H NMR (400 MHz, DMSO-d₆) δ 9.83 (s, 1H), 6.99-6.85 (m, 2H), 4.88 (s, 1H), 4.77 (s, 1H), 4.62 (s, 1H), 4.54 (s, 1H), 4.05 (q, J=7.2 Hz, 2H), 2.66-2.53 (m, 4H), 1.18 (t, J=7.2 Hz, 3H).

### Step e: Ethyl 4-(6-bromo-4-fluoro-5-hydroxy-isoindolin-2-yl)-4-oxo-butanoate

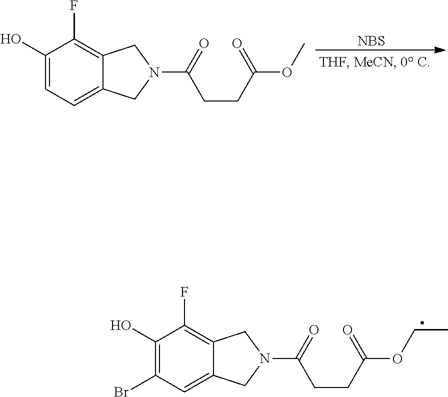

NBS (1.194 g, 6.7085 mmol) was added to a solution of ethyl 4-(4-fluoro-5-hydroxy-isoindolin-2-yl)-4-oxo-butanoate (2.106 g, 7.4872 mmol) in MeCN (20 mL) and THF (20 mL) at 0° C. under N₂ atmosphere. The reaction mixture was stirred for 2 h and warmed up to 20° C. naturally. The precipitate was collected by filtration. The filter cake was dried under vacuo. There was ethyl 4-(6-bromo-4-fluoro-5-hydroxy-isoindolin-2-yl)-4-oxo-butanoate (2.84 g, 7.8851 mmol, 105.3134% yield) obtained as a white solid. LCMS: (ESI, m/z): [M+H]⁺=359.010. ¹H NMR (400 MHz, DMSO-d₆) δ 10.41 (s, 1H), 7.36 (s, 0.5H), 7.35 (s, 0.5H), 4.87 (s, 1H), 4.78 (s, 1H), 4.60 (s, 1H), 4.55 (s, 1H), 4.14-3.97 (q, J=7.2 Hz, 2H), 2.70-2.52 (m, 4H), 1.18 (t, J=7.2 Hz, 3H).

### Step f: Ethyl 4-(6-bromo-4-fluoro-5-(methoxymethoxy)isoindolin-2-yl)-4-oxobutanoate

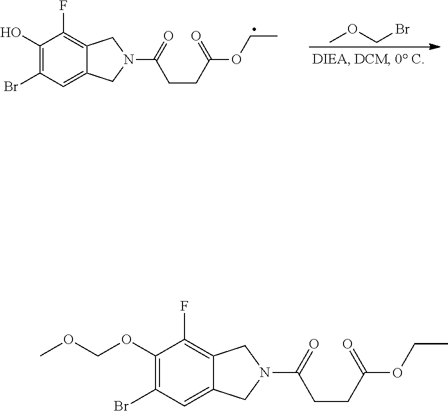

Bromomethyl methyl ether (1.20 g, 9.6028 mmol) was added to a solution of ethyl 4-(6-bromo-4-fluoro-5-hydroxy-isoindolin-2-yl)-4-oxo-butanoate (2.23 g, 6.1914 mmol) and DIEA (2.55 g, 19.7304 mmol) in DCM (50 mL) at 0° C. under N₂ atmosphere. The reaction mixture was stirred for 2 h and warmed up to 20° C. naturally. The reaction mixture was quenched with adding of water (50 mL) at 20° C., extracted with DCM (3×50 mL) and washed with brine (50 mL). The organics dried over Na₂SO₄, filtered and evaporated under reduced pressure. The residue was purified on C18 column ACN/H₂O (0.1% FA)(0-50%). The pure fractions was concentrated and dried under vacuo. There was ethyl 4-(6-bromo-4-fluoro-5-(methoxymethoxy)isoindolin-2-yl)-4-oxobutanoate (1.809 g, 4.4752 mmol, 72.2806% yield) obtained as a yellow solid. LCMS: (ESI, m/z): [M+H]⁺=403.140. ¹H NMR (400 MHz, DMSO-d₆) δ 7.52 (s, 0.5H), 7.51 (s, 0.5H), 5.16 (s, 2H), 4.90 (s, 1H), 4.85 (s, 1H), 4.63 (s, 1H), 4.61 (s, 1H), 4.05 (q, J=7.2 Hz, 2H), 3.54 (s, 3H), 2.72-2.53 (m, 4H), 1.24-1.12 (t, J=7.2 Hz, 3H).

### Step g: Ethyl 4-(4-fluoro-6-hydroxy-5-(methoxymethoxy)isoindolin-2-yl)-4-oxobutanoate

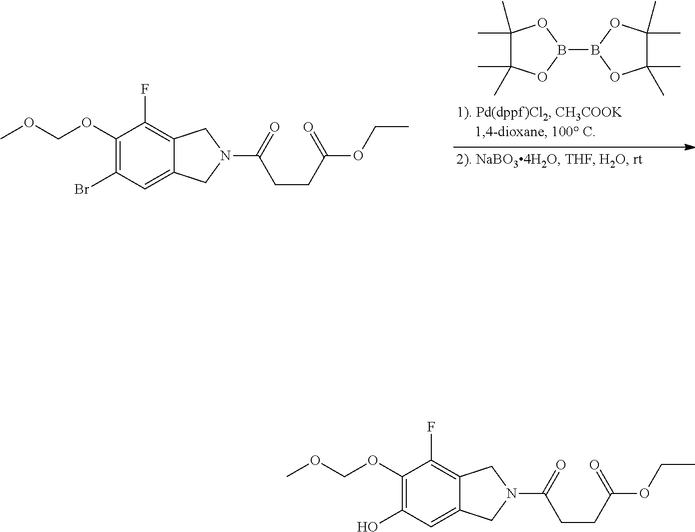

Pd(dppf)Cl₂ (0.228 g, 311.6016 μmol), 4,4,4′,4′,5,5,5′,5′-Octamethyl-2,2′-bi(1,3,2-dioxaborolane) (1.170 g, 4.6074 mmol) and Potassium Acetate (0.743 g, 7.5706 mmol) was added to a solution of ethyl 4-(6-bromo-4-fluoro-5-(methoxymethoxy)isoindolin-2-yl)-4-oxobutanoate (0.735 g, 1.8183 mmol) in 1,4-Dioxane (20 mL) at 20° C. under N₂ atmosphere. The reaction mixture was heated to 100° C. and stirred for 12 h. The reaction mixture was quenched with adding of water (150 mL) at 20° C., extracted with EA (3×150 mL) and washed with brine (50 mL). The organics dried over Na₂SO₄, filtered and evaporated under reduced pressure. Sodium perborate (0.91 g, 5.9145 mmol) was added to a solution of the residue in THF (10 mL) and H₂O (10 mL) at 20° C. under N₂ atmosphere. The reaction mixture was stirred for 1 h at 20° C. The reaction mixture was evaporated under reduced pressure. The residue was purified on C18 column ACN/H₂O (0.1% FA)(0-38%). The pure fractions was concentrated and dried under vacuo. There was ethyl 4-(4-fluoro-6-hydroxy-5-(methoxymethoxy)isoindolin-2-yl)-4-oxobutanoate (0.365 g, 1.0693 mmol, 58.8% yield) obtained as a white solid. LCMS: (ESI, m/z): [M+H]⁺=342.130. ¹H NMR (400 MHz, DMSO-d₆) δ 9.97 (s, 1H), 6.68 (s, 1H), 5.04 (s, 2H), 4.80 (s, 1H), 4.76 (s, 1H), 4.55 (s, 1H), 4.53 (s, 1H), 4.05 (q, J=7.2 Hz, 2H), 3.47 (s, 3H), 2.67-2.51 (m, 4H), 1.18 (t, J=7.2 Hz, 3H).

### Step h: Ethyl 4-(4-fluoro-5-hydroxy-6-methoxy-isoindolin-2-yl)-4-oxo-butanoate

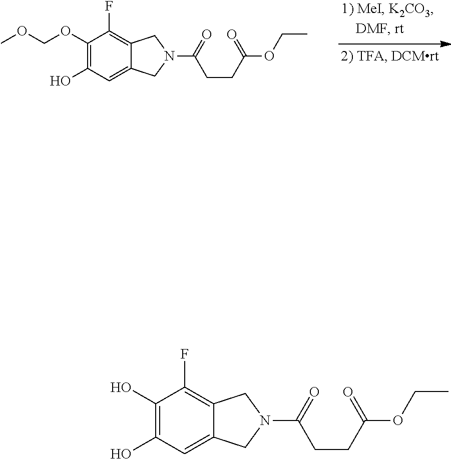 [SMILES: CCOC(=O)CCC(=O)N1Cc2cc(O)c(OCOC)c(F)c2C1 ; CCOC(=O)CCC(=O)N1Cc2cc(O)c(O)c(F)c2C1]

Methyl Iodide (0.345 g, 2.4306 mmol) was added to a mixture of Potassium carbonate (0.448 g, 3.2415 mmol) and ethyl 4-(4-fluoro-6-hydroxy-5-(methoxymethoxy)isoindolin-2-yl)-4-oxobutanoate (0.328 g, 960.9441 μmol) in DMF (10 mL) at 20° C. under N₂ atmosphere. The reaction mixture was stirred for 1 h at 20° C. The reaction mixture was quenched with adding of water (50 mL) at 20° C., extracted with DCM (3×50 mL) and washed with brine (50 mL). The organics dried over Na₂SO₄, filtered and evaporated under reduced pressure. Trifluoroacetic acid (2 mL) was added to a solution of the residue (0.446 g, 1.2551 mmol) in DCM (5 mL) at 20° C. under N₂ atmosphere. The reaction mixture was stirred for 2 h at 20° C. The reaction mixture was evaporated under reduced pressure. The residue was purified on C18 column ACN/H₂O (0-80%). The pure fractions was concentrated and dried under vacuo. There was ethyl 4-(4-fluoro-5-hydroxy-6-methoxy-isoindolin-2-yl)-4-oxo-butanoate (0.274 g, 880.1663 μmol, 91.6% yield) obtained as a white solid. LCMS: (ESI, m/z): [M+H]⁺=312.12. ¹H NMR (400 MHz, DMSO-d₆) δ 9.17 (s, 1H), 6.83 (s, 0.5H), 6.80 (s, 0.5H), 4.82 (s, 1H), 4.77 (s, 1H), 4.57 (s, 1H), 4.54 (s, 1H), 4.05 (q, J=7.2 Hz, 2H), 3.80 (d, J=2.0 Hz, 3H), 2.68-2.51 (m, 4H), 1.18 (t, J=7.2 Hz, 3H).

### INT A12: Ethyl 4-(6-hydroxy-5-methoxythieno[3,2-b]pyridin-2-yl)-4-oxobutanoate

### Step a: 6-bromo-5-methoxythieno[3,2-b]pyridine

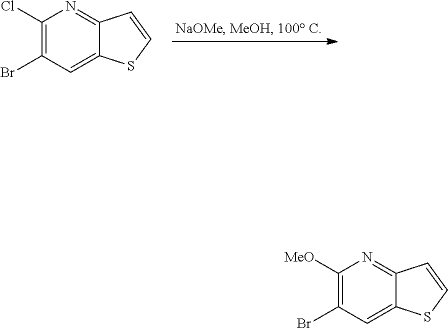 [SMILES: Clc1nc2ccsc2cc1Br ; COc1nc2ccsc2cc1Br]

A solution of 6-bromo-5-chlorothieno[3,2-b]pyridine (1.0 g, 4.0 mmol) and NaOMe (9.3 ml, 40 mmol) in MeOH (6 mL) was stirred at 100° C. for 1 h under N₂ atmosphere in microwave irradiation reactor. 1 N citric acid (80 mL) was added, then extracted with EtOAc (70 mL×3). The combined organic solution was dried over Na₂SO₄, concentrated to give 6-bromo-5-methoxythieno[3,2-b]pyridine (1.0 g, 4.0 mmol, 99% yield) as a brown solid. ¹H NMR (300 MHz, DMSO-d₆): δ 8.75 (d, J=0.5 Hz, 1H), 8.08 (d, J=5.5 Hz, 1H), 7.42 (dd, J=5.5, 0.5 Hz, 1H), 3.98 (s, 3H). LCMS: (ESI, m/z): [M+H]⁺=244.0.

### Step b: 6-bromo-5-methoxythieno[3,2-b]pyridine-2-carboxylic Acid

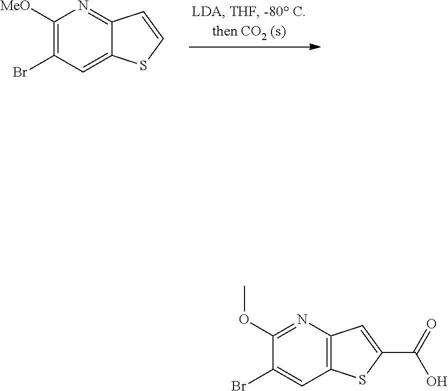

To a solution of 6-bromo-5-methoxythieno[3,2-b]pyridine (5.0 g, 20.0 mmol) in THF (200 mL) was added LDA (20 ml, 40 mmol) at −80° C. The reaction mixture was stirred at −80° C. for 30 min under N₂ atmosphere. Then the solution was poured onto CO₂ solid. The reaction mixture was stirred for about 1 h. 1 N HCl (50 mL) and water (200 ml) was added, then extracted with EtOAc (70 mL×3). The combined organic solution was dried over Na₂SO₄, concentrated to give 6-bromo-5-methoxythieno[3,2-b]pyridine-2-carboxylic acid (4.9 g, 17.0 mmol, 85% yield) as a brown solid. ¹H NMR (300 MHz, DMSO-d₆): δ 8.84 (s, 1H), 7.92 (s, 1H), 4.00 (s, 3H). LCMS: (ESI, m/z): [M+H]⁺=287.9.

### Step c: Tert-butyl 3-(6-bromo-5-methoxythieno[3,2-b]pyridin-2-yl)-3-oxopropanoate

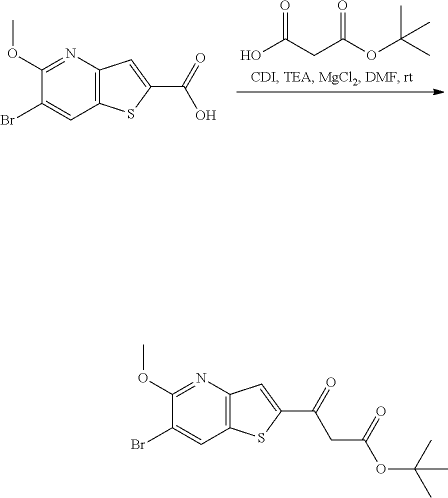 [SMILES: COc1nc2cc(C(=O)O)sc2cc1Br ; CC(C)(C)OC(=O)CC(=O)O ; COc1nc2cc(C(=O)CC(=O)OC(C)(C)C)sc2cc1Br]

To a solution of 6-bromo-5-methoxythieno[3,2-b]pyridine-2-carboxylic acid (4.9 g, 17.0 mmol) in DMF (147 mL) was added CDI (5.5 g, 34.0 mmol). After stirring at RT for 2 h under N₂ atmosphere, 3-tert-Butoxy-3-oxopropanoic acid (5.5 g, 34.0 mmol), TEA (5.2 g, 51.0 mmol, 3.0 eq) and MgCl₂ (3.3 g, 34.0 mmol) was added, then stirred at RT for overnight. Upon completion, sat. NaHCO₃ solution (200 mL) was added, then extracted with EtOAc (200 mL×3). The combined organic solution was dried over Na₂SO₄, concentrated to give a residue, purified by column chromatography (Petroleum ether/Ethyl acetate=20:1) to give tert-butyl 3-(6-bromo-5-methoxythieno[3,2-b]pyridin-2-yl)-3-oxopropanoate (5.0 g, 12.9 mmol, 62% yield) as a yellow solid.

¹H NMR (300 MHz, DMSO-d₆): δ 8.87 (s, 1H), 8.33 (s, 1H), 4.17 (s, 2H), 4.01 (s, 3H), 1.40 (s, 9H).

LCMS: (ESI, m/z): [M+H]⁺=386.0.

### Step d: 1-(tert-butyl) 4-ethyl 2-(6-bromo-5-methoxythieno[3,2-b]pyridine-2-carbonyl)succinate

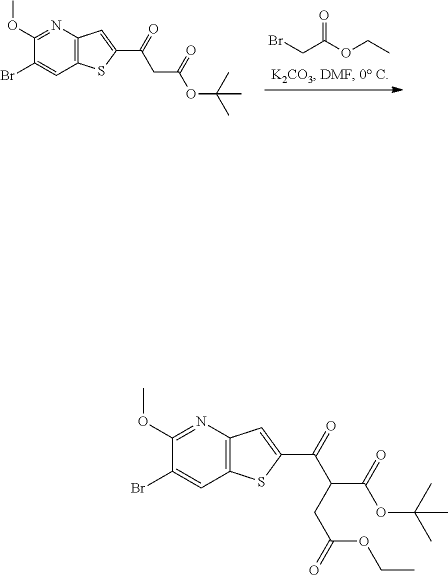

To a solution of tert-butyl 3-(6-bromo-5-methoxythieno[3,2-b]pyridin-2-yl)-3-oxopropanoate (3.0 g, 7.8 mmol) in DMF (60 mL) was added K₂CO₃ (1.8 g, 12.5 mmol) and Ethyl bromoacetate (1.3 g, 78 mmol) at 0° C.

The reaction mixture was stirred at 0° C. for 5 h under N₂ atmosphere. NaHCO₃ solution (100 mL) was added, then extracted with EtOAc (100 mL×3). The combined organic solution was dried over Na₂SO₄, concentrated to give a residue, purified by column chromatography (Petroleum ether/Ethyl acetate=20:1) to give 1-(tert-butyl) 4-ethyl 2-(6-bromo-5-methoxythieno[3,2-b]pyridine-2-carbonyl)succinate (3.3 g, 7.0 mmol, 89% yield) as a white solid.

¹H NMR (300 MHz, DMSO-d₆): δ 8.89 (s, 1H), 8.47 (s, 1H), 5.00 (t, J=7.3 Hz, 1H), 4.12-3.93 (m, 5H), 2.94 (t, J=7.3 Hz, 2H), 1.31 (d, J=4.5 Hz, 9H), 1.13 (t, J=7.1 Hz, 3H).

LCMS: (ESI, m/z): [M+H]⁺=472.0.

### Step e: Ethyl 4-(6-bromo-5-methoxythieno[3,2-b]pyridin-2-yl)-4-oxobutanoate

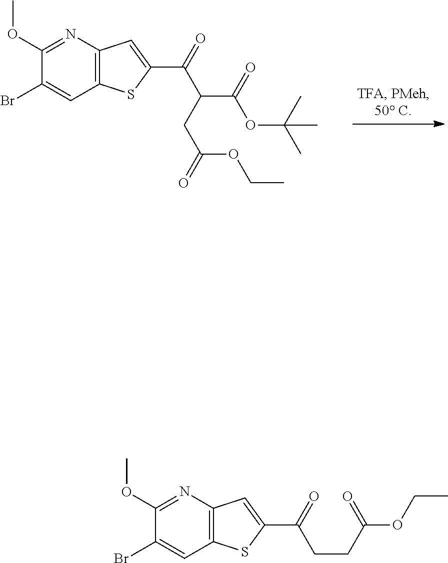 [SMILES: CCOC(=O)CC(C(=O)OC(C)(C)C)C(=O)c1cc2nc(OC)c(Br)cc2s1 ; CCOC(=O)CCC(=O)c1cc2nc(OC)c(Br)cc2s1]

To a solution of 1-(tert-butyl) 4-ethyl 2-(6-bromo-5-methoxythieno[3,2-b]pyridine-2-carbonyl)succinate (3.3 g, 7.0 mmol) in PhMe (66 mL) was added TFA (33 ml). After stirring at 50° C. for 1.5 h under N₂ atmosphere, the solution was concentrated to give a residue, and the obtained solid was triturated with Petroleum ether/ethyl acetate (10/1) to obtain crude compound ethyl 4-(6-bromo-5-methoxythieno[3,2-b]pyridin-2-yl)-4-oxobutanoate (2.2 g, 5.9 mmol, 84% yield) as a brown solid. ¹H NMR (300 MHz, DMSO-d₆): δ 8.87 (s, 1H), 8.37 (s, 1H), 4.11-3.98 (m, 5H), 3.45-3.36 (m, 2H), 2.71-2.63 (m, 2H), 1.17 (t, J=7.1 Hz, 3H).

LCMS: (ESI, m/z): [M+H]⁺=372.0.

### Step f: (2-(4-ethoxy-4-oxobutanoyl)-5-methoxythieno[3,2-b]pyridin-6-yl)boronic Acid

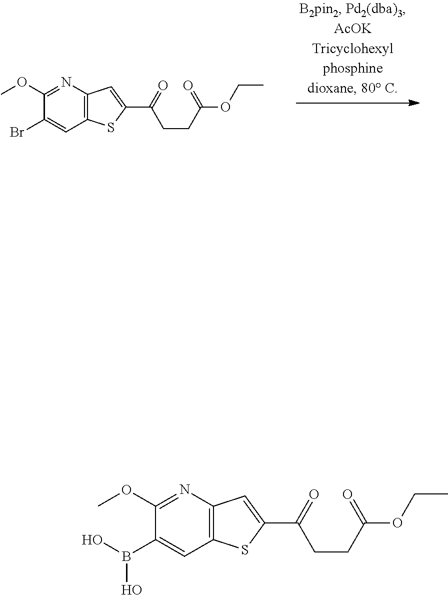 [SMILES: CCOC(=O)CCC(=O)c1cc2nc(OC)c(Br)cc2s1 ; CCOC(=O)CCC(=O)c1cc2nc(OC)c(B(O)O)cc2s1]

Ethyl 4-(6-bromo-5-methoxythieno[3,2-b]pyridin-2-yl)-4-oxobutanoate (1.6 g, 4.3 mmol), B₂Pin₂ (1.4 g, 5.3 mmol), AcOK (688 mg, 6.9 mmol), Pd2(dba)3 (112 mg, 0.1 mmol) and Tricyclohexyl phosphine (128 mg, 0.43 mmol) were placed in the reaction bottle. The bottle was evacuated and filled with argon for three times. Then dioxane (32 mL) was added at room temperature. The mixture was stirred at 80° C. for 1 h. Water (50 mL) was added to the reaction mixture, extracted with EtOAc (3×50 mL), and the combined organic layers were washed with saturated brine and dried over anhydrous sodium sulfate. The solution was concentrated to give (2-(4-ethoxy-4-oxobutanoyl)-5-methoxythieno[3,2-b]pyridin-6-yl)boronic acid (3.2 g) a crude residue. It was used for next step without any further purification. LCMS: (ESI, m/z): [M+H]⁺=338.1.

### Step g: Ethyl 4-(6-hydroxy-5-methoxythieno[3,2-b]pyridin-2-yl)-4-oxobutanoate

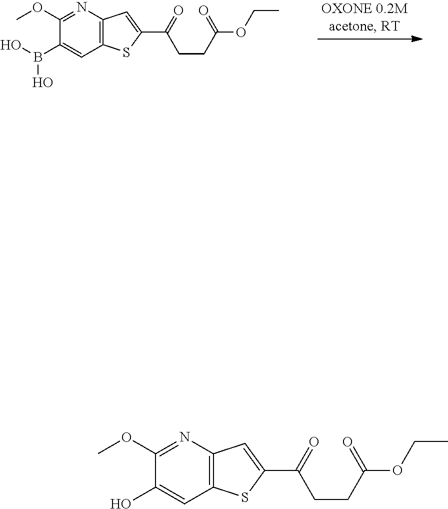

To a solution of (2-(4-ethoxy-4-oxobutanoyl)-5-methoxythieno[3,2-b]pyridin-6-yl)boronic acid (3.2 g, 4.3 mmol, crude) in acetone (48 mL) was added OXONE (36 ml, 6.5 mmol). After stirring at RT for 2 h. Sat. NaHSO₃ solution (120 mL) was added, after stirring at RT for 1 h, the mixture was extracted with EtOAc (3×30 mL), and the combined organic layers were washed with saturated brine and dried over anhydrous sodium sulfate. The solution was concentrated to give a crude residue, purified by column chromatography (Petroleum ether/Ethyl acetate=10:1 to 5:1) to give ethyl 4-(6-hydroxy-5-methoxythieno[3,2-b]pyridin-2-yl)-4-oxobutanoate (340 mg, 1.1 mmol, 25% yield) as a yellow solid. ¹H NMR (400 MHz, DMSO-d₆): δ 10.44 (s, 1H), 8.22 (s, 1H), 7.65 (s, 1H), 4.05 (q, J=7.1 Hz, 2H), 3.97 (s, 3H), ˜3.30-3.31 (br, 2 h), 2.65 (t, J=6.1 Hz, 2H), 1.17 (t, J=7.1 Hz, 3H).

LCMS: (ESI, m/z): [M+H]⁺=310.1.

The following intermediates were synthesized with the above steps or improved procedure with the corresponding starting materials and intermediates.

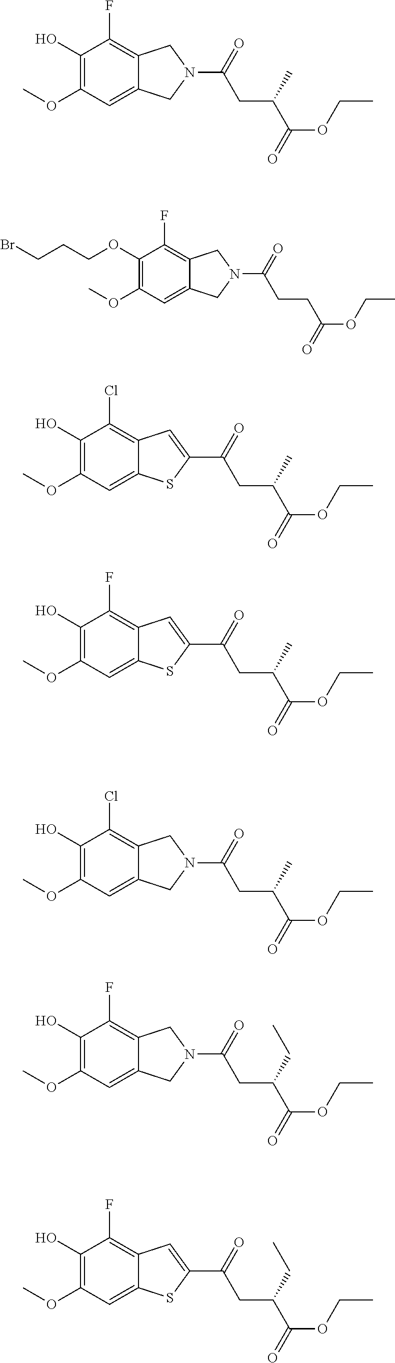

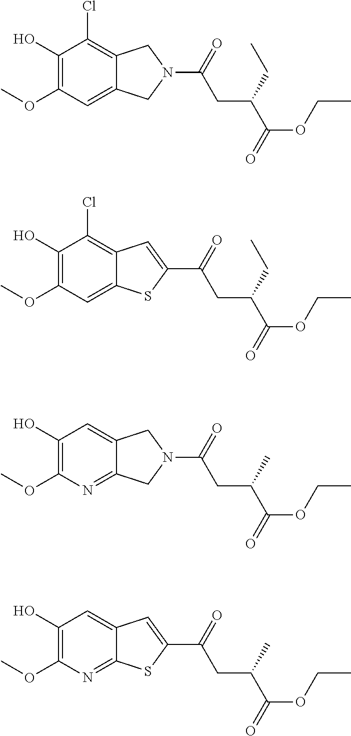

### Example 1

### 4-(5-(3-((2-(3-carboxypropanoyl)-6-methoxybenzo[b]thiophen-5-yl)oxy)propoxy)-6-methoxyisoindolin-2-yl)-4-oxobutanoic Acid

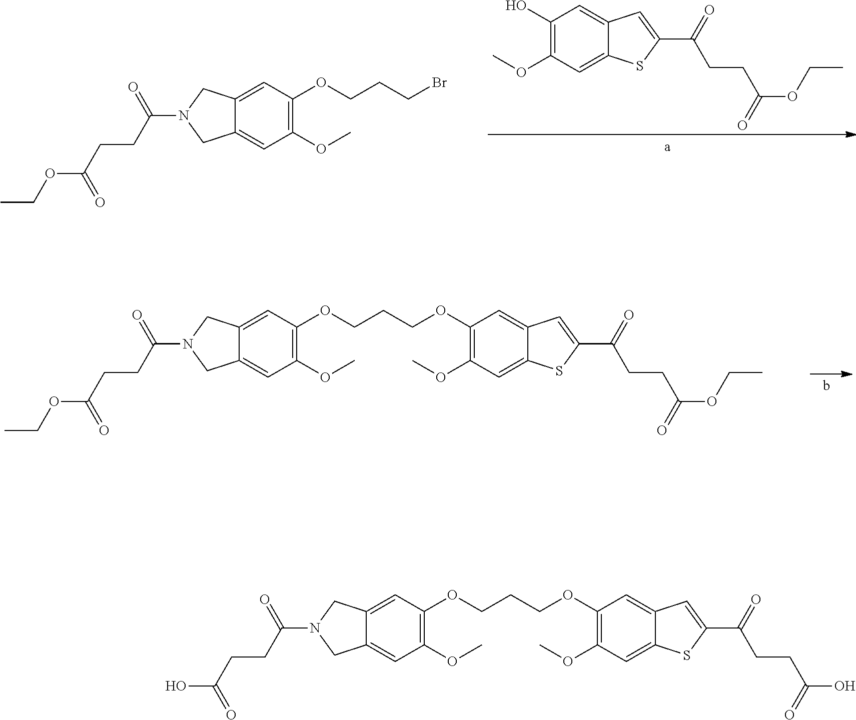 [SMILES: CCOC(=O)CCC(=O)N1Cc2cc(OC)c(OCCCBr)cc2C1 ; CCOC(=O)CCC(=O)c1cc2cc(O)c(OC)cc2s1 ; CCOC(=O)CCC(=O)c1cc2cc(OCCCOc3cc4c(cc3OC)CN(C(=O)CCC(=O)OCC)C4)c(OC)cc2s1 ; COc1cc2c(cc1OCCCOc1cc3cc(C(=O)CCC(=O)O)sc3cc1OC)CN(C(=O)CCC(=O)O)C2]

Step a: Potassium carbonate (0.240 g, 1.7365 mmol) was added to a solution of ethyl 4-(5-(3-bromopropoxy)-6-methoxyisoindolin-2-yl)-4-oxobutanoate (0.240 g, 579.3045 μmol) and ethyl 4-(5-hydroxy-6-methoxybenzo[b]thiophen-2-yl)-4-oxobutanoate (0.169 g, 576.1737 μmol) in N,N-Dimethylformamide (10 mL) at 20° C. The reaction mixture was heated to 50° C. and stirred for 3 h. The reaction mixture was evaporated under reduced pressure. The reaction mixture was concentrated and diluted with EA (500 mL), washed with NaHCO₃ aq. (2×300 mL) and brine (200 mL). The organics was dried over Na₂SO₄, filtered and evaporated under reduced pressure. The residue was purified on silica gel column EA/Hept (0-100%). The pure fractions was concentrated and dried under vacuo. There was ethyl 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoate (0.147 g, 229.0694 μmol, 62.8484% yield) obtained as a yellow solid.

¹H NMR (400 MHz, DMSO-d₆) δ 8.18 (s, 1H), 7.61 (d, J=1.9 Hz, 1H), 7.53 (s, 1H), 6.98 (dd, J=15.7, 11.9 Hz, 2H), 4.73 (d, J=9.4 Hz, 2H), 4.52 (s, 2H), 4.17 (dd, J=30.4, 7.5 Hz, 4H), 4.05 (qd, J=7.1, 3.4 Hz, 4H), 3.86 (d, J=1.6 Hz, 3H), 3.75 (d, J=2.0 Hz, 3H), 2.70-2.53 (m, 6H), 2.30-2.15 (m, 2H), 1.24-1.11 (m, 6H).

Step b: LiOH (0.022 g, 918.6456 μmol) was added to a solution of ethyl 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoate in Tetrahydrofuran (10 mL) and Water (2 mL) at 20° C. The reaction mixture was stirred for 2 h. The reaction mixture was evaporated under reduced pressure and diluted with water and adjusted pH=3 with HCl (1 M). The precipitate was filtered. Filter cake was washed with water (20 mL). The filtrate cake was dried under vacuo at 60° C. There was 4-(5-(3-((2-(3-carboxypropanoyl)-6-methoxybenzo[b]thiophen-5-yl)oxy)propoxy)-6-methoxyisoindolin-2-yl)-4-oxobutanoic acid (0.119 g, 203.2031 μmol, 90.5562% yield) obtained as a yellow solid.

¹H NMR (400 MHz, DMSO-d₆) δ 12.12 (s, 2H), 8.17 (s, 1H), 7.67-7.55 (m, 1H), 7.53 (s, 1H), 6.98 (dd, J=16.6, 12.5 Hz, 2H), 4.73 (d, J=8.2 Hz, 2H), 4.53 (s, 2H), 4.18 (ddd, J=23.1, 13.6, 6.3 Hz, 4H), 3.86 (d, J=1.0 Hz, 3H), 3.75 (d, J=1.9 Hz, 3H), 3.25 (d, J=6.6 Hz, 2H), 2.67-2.53 (m, 4H), 2.30-2.11 (m, 2H).

### Example 2

### 4-(5-(3-((2-(3-carboxypropanoyl)-4-chloro-6-methoxyisoindolin-5-yl)oxy)propoxy)-4-fluoro-6-methoxy benzo[b]thiophen-2-yl)-4-oxobutanoic Acid

### Step a: O1-tert-butyl O4-ethyl 2-(5-bromo-4-fluoro-6-methoxy-benzothiophene-2-carbonyl)butanedioate

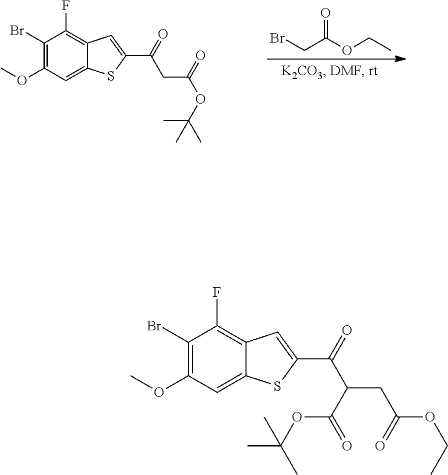

To a stirred solution of tert-butyl 3-(5-bromo-4-fluoro-6-methoxy-benzothiophen-2-yl)-3-oxo-propanoate (1.58 g, 3.9180 mmol) in N,N-Dimethylformamide (10 mL) was added K₂CO₃ (1.10 g, 7.9592 mmol) and ethyl bromoacetate (0.72 g, 4.3114 mmol) at 20° C. The resulting mixture was stirred for 2 h at 20° C. under nitrogen atmosphere. The reaction mixture was concentrated and diluted with EA (200 mL), washed with water (100 mL) and brine (50 mL). The organics dried over Na₂SO₄, filtered and evaporated under reduced pressure. The residue was purified by flash silica chromatography, elution gradient 0 to 100% Ethyl acetate in heptane. The pure fractions was concentrated and dried under vacuo. There was 01-tert-butyl 04-ethyl 2-(5-bromo-4-fluoro-6-methoxy-benzothiophene-2-carbonyl)butanedioate (1.925 g, 3.9338 mmol, 100.4016% yield) obtained as a yellow oil. LCMS: (ESI, m/z): [M+H]⁺=489.030.

### Step b: Ethyl 4-(5-bromo-4-fluoro-6-methoxy-benzothiophen-2-yl)-4-oxo-butanoate

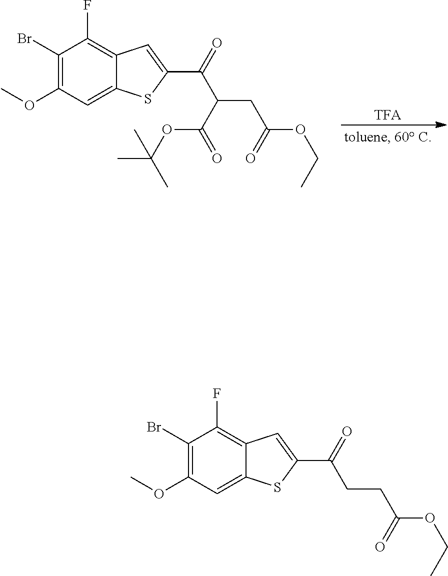 [SMILES: CCOC(=O)CC(C(=O)OC(C)(C)C)C(=O)c1cc2c(F)c(Br)c(OC)cc2s1 ; CCOC(=O)CCC(=O)c1cc2c(F)c(Br)c(OC)cc2s1]

Trifluoroacetic acid (2 mL) was added to a solution of 01-tert-butyl 04-ethyl 2-(5-bromo-4-fluoro-6-methoxy-benzothiophene-2-carbonyl)butanedioate (1.85 g, 3.7805 mmol) in Toluene (10 mL) at 20° C. The reaction mixture was stirred at 60° C. for 2 h. The reaction mixture was evaporated under reduced pressure. The residue was concentrated and diluted with DCM (200 mL), washed with NaHCO₃ aq (2×100 mL) and brine (100 mL). The organics dried over Na₂SO₄, filtered and evaporated under reduced pressure. The residue was purified by flash silica chromatography, elution gradient 0 to 100% Ethyl acetate in heptane. The pure fractions was concentrated and dried under vacuo. There was ethyl 4-(5-bromo-4-fluoro-6-methoxy-benzothiophen-2-yl)-4-oxo-butanoate (0.727 g, 1.8678 mmol, 49.4050% yield) obtained as a yellow solid. LCMS: (ESI, m/z): [M+H]⁺=388.978. ¹H NMR (400 MHz, DMSO-d₆) δ 8.39 (s, 1H), 7.71 (s, 1H), 4.06 (q, J=6.9 Hz, 2H), 3.98 (s, 3H), 3.39 (d, J=6.2 Hz, 2H), 2.73-2.62 (m, 2H), 1.18 (t, J=7.1 Hz, 3H).

### Step c: Ethyl 4-(4-fluoro-6-methoxy-5-(4,4,5,5-tetramethyl-1,3,2-dioxaborolan-2-yl)benzo[b]thiophen-2-yl)-4-oxobutanoate

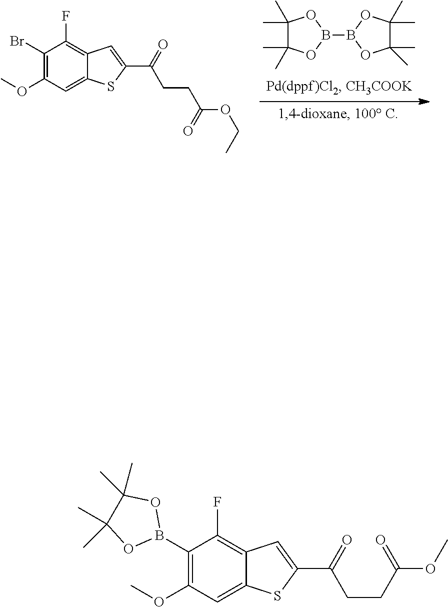

Pd(dppf)Cl₂ (0.15 g, 205.0010 μmol) was added to a mixture of Potassium Acetate (0.60 g, 6.1136 mmol), 4,4,4′,4′,5,5,5′,5′-Octamethyl-2,2′-bi(1,3,2-dioxaborolane) (0.97 g, 3.8198 mmol) and ethyl 4-(5-bromo-4-fluoro-6-methoxy-benzothiophen-2-yl)-4-oxo-butanoate (0.72 g, 1.8498 mmol) in 1,4-Dioxane (30 mL) at 20° C. The reaction mixture was stirred at 100° C. for 20 h under N₂ atmosphere. The reaction mixture was evaporated under reduced pressure. The residue was concentrated and diluted with EA (200 mL), washed with NaHCO₃ aq (2×100 mL) and brine (100 mL). The organics dried over Na₂SO₄, filtered and evaporated under reduced pressure. The residue was purified by flash silica chromatography, elution gradient 0 to 100% Ethyl acetate in heptane. The pure fractions was concentrated and dried under vacuo. There was ethyl 4-(4-fluoro-6-methoxy-5-(4,4,5,5-tetramethyl-1,3,2-dioxaborolan-2-yl)benzo[b]thiophen-2-yl)-4-oxobutanoate (0.38 g, 870.9586 μmol, 47.0845% yield) obtained as a white solid. LCMS: (ESI, m/z): [M+H]⁺=437.153.

### Step d: Ethyl 4-(4-fluoro-5-hydroxy-6-methoxy-benzothiophen-2-yl)-4-oxo-butanoate

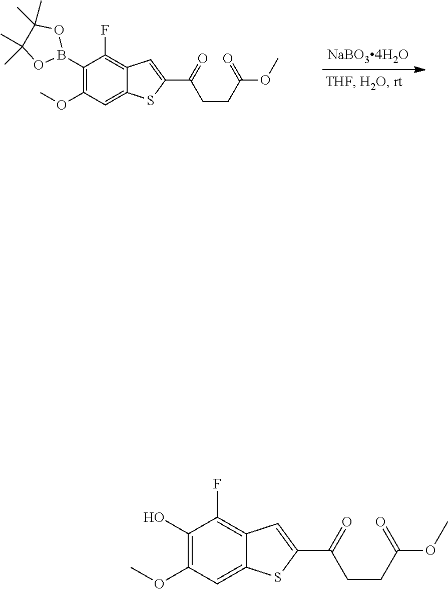

Sodium perborate tetrahydrate (0.21 g, 1.3649 mmol) was added to a solution of ethyl 4-(4-fluoro-6-methoxy-5-(4,4,5,5-tetramethyl-1,3,2-dioxaborolan-2-yl)benzo[b]thiophen-2-yl)-4-oxobutanoate in Tetrahydrofuran (10 mL) and water (10 mL) at 20° C. The reaction mixture was stirred at 20° C. for 1 h. The reaction mixture was concentrated and diluted with DCM (100 mL), washed with NaHCO₃ aq (2×500 mL) and brine (50 mL). The organics dried over Na₂SO₄, filtered and evaporated under reduced pressure. The residue was purified on silica gel column MeOH/DCM (0-5%). The pure fractions was concentrated and dried under vacuo. There was ethyl 4-(4-fluoro-5-hydroxy-6-methoxy-benzothiophen-2-yl)-4-oxo-butanoate (0.263 g, 805.9094 μmol, 92.5313% yield) obtained as a yellow solid. LCMS: (ESI, m/z): [M+H]⁺=327.062. ¹H NMR (400 MHz, DMSO-d₆) δ 9.53 (s, 1H), 8.25 (s, 1H), 7.47 (s, 1H), 4.06 (q, J=7.1 Hz, 2H), 3.88 (s, 3H), 3.39-3.34 (m, 2H), 2.69-2.60 (m, 2H), 1.17 (t, J=7.1 Hz, 3H).

### Step e: Ethyl 4-(4-chloro-5-hydroxy-6-methoxy-isoindolin-2-yl)-4-oxo-butanoate

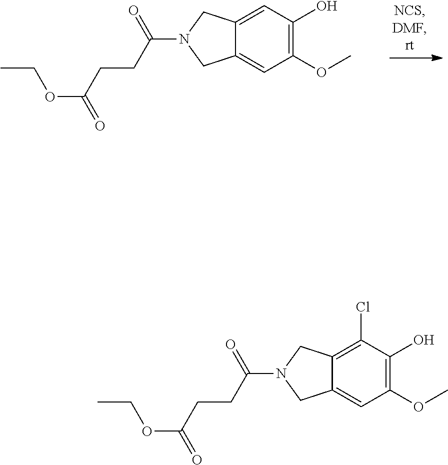

NCS (0.105 g, 786.3231 μmol) was added to a solution of ethyl 4-(5-hydroxy-6-methoxyisoindolin-2-yl)-4-oxobutanoate (0.205 g, 698.9090 μmol) in DMF (10 mL) at 0° C. under N₂ atmosphere. The reaction mixture was stirred for 12 h and warmed up to 20° C. naturally. The reaction mixture was purified on C18 column ACN/H₂O (0-25%). The pure fractions was concentrated and dried under vacuo. There was ethyl 4-(4-chloro-5-hydroxy-6-methoxy-isoindolin-2-yl)-4-oxo-butanoate (0.148 g, 451.5507 μmol, 64.6079% yield) obtained as a white solid. LCMS: (ESI, m/z): [M+H]⁺=328.100. ¹H NMR (400 MHz, DMSO-d₆) δ 9.41 (s, 1H), 6.97 (s, 0.5H), 6.95 (s, 0.5H), 4.81 (s, 1H), 4.76 (s, 1H), 4.59 (s, 1H), 4.51 (s, 1H), 4.05 (q, J=7.2 Hz, 2H), 3.82 (s, 3H), 2.69-2.53 (m, 4H), 1.18 (t, J=7.2 Hz, 3H).

### Step f: Ethyl 4-[5-(3-bromopropoxy)-4-chloro-6-methoxy-isoindolin-2-yl]-4-oxo-butanoate

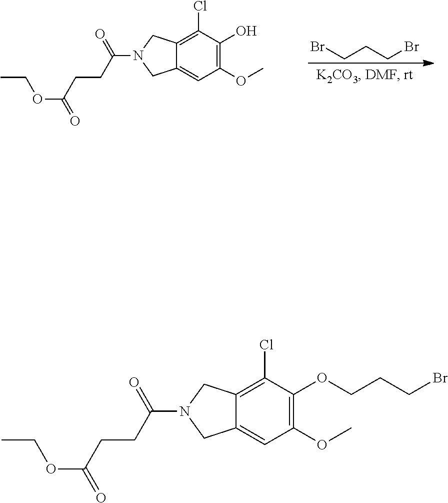

1,3-Dibromopropane (0.495 g, 2.4519 mmol) was added to a mixture of K₂CO₃ (0.199 g, 1.4399 mmol), ethyl 4-(4-chloro-5-hydroxy-6-methoxyisoindolin-2-yl)-4-oxobutanoate (0.149 g, 454.6023 μmol) in DMF (5 mL) at 20° C. under N₂ atmosphere. The reaction mixture was stirred for 4 h at 20° C. The reaction mixture was purified on C18 column ACN/H₂O (0.1% FA)(0-50%). The pure fractions was concentrated and dried under vacuo. There was ethyl 4-(5-(3-bromopropoxy)-4-chloro-6-methoxyisoindolin-2-yl)-4-oxobutanoate (0.163 g, 363.2433 μmol, 79.9035% yield) obtained as a white solid. LCMS: (ESI, m/z): [M+H]⁺=448.040. ¹H NMR (400 MHz, DMSO-d₆) δ 7.10 (s, 0.5H), 7.08 (s, 0.5H), 4.87 (s, 1H), 4.79 (s, 1H), 4.64 (s, 1H), 4.53 (s, 1H), 4.06-4.02 (m, 4H), 3.83 (d, J=2.4 Hz, 3H), 3.78-3.70 (m, 2H), 2.71-2.53 (m, 4H), 2.23-2.20 (m, 2H), 1.18 (t, J=7.2 Hz, 3H).

### Step g: Ethyl 4-(5-(3-((4-chloro-2-(4-ethoxy-4-oxobutanoyl)-6-methoxyisoindolin-5-yl)oxy)propoxy)-4-fluoro-6-methoxybenzo[b]thiophen-2-yl)-4-oxobutanoate

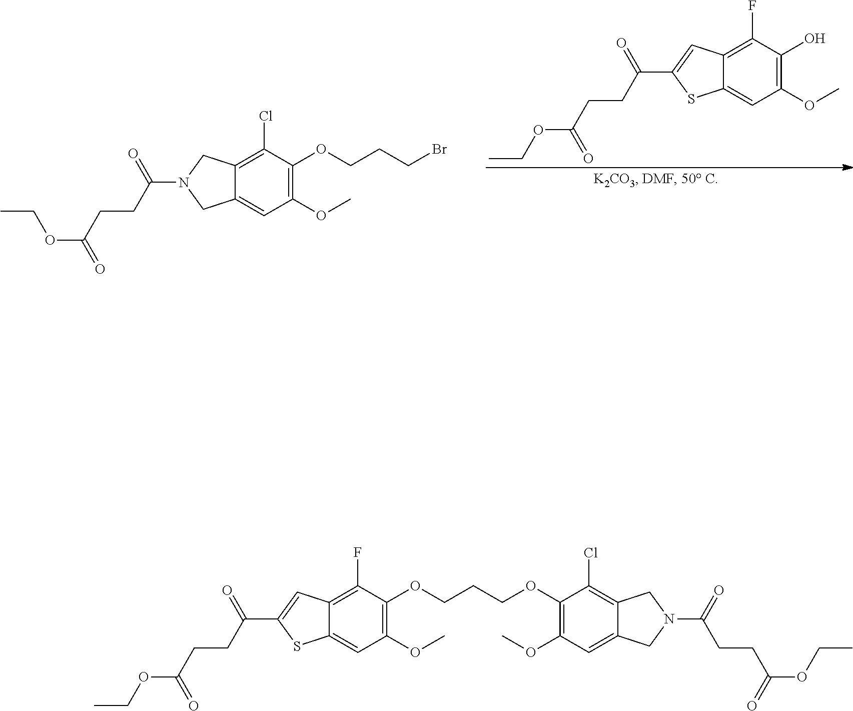 [SMILES: CCOC(=O)CCC(=O)N1Cc2cc(OC)c(OCCCBr)c(Cl)c2C1 ; CCOC(=O)CCC(=O)c1cc2c(F)c(O)c(OC)cc2s1 ; CCOC(=O)CCC(=O)c1cc2c(F)c(OCCCOc3c(OC)cc4c(c3Cl)CN(C(=O)CCC(=O)OCC)C4)c(OC)cc2s1]

K₂CO₃ (0.115 g, 832.0942 μmol) was added to a solution of ethyl 4-(5-(3-bromopropoxy)-4-chloro-6-methoxyisoindolin-2-yl)-4-oxobutanoate (0.068 g, 151.5371 μmol), ethyl 4-(4-fluoro-5-hydroxy-6-methoxybenzo[b]thiophen-2-yl)-4-oxobutanoate (0.049 g, 150.1504 μmol) in DMF (3 mL) at 20° C. under N₂ atmosphere. The reaction mixture was stirred for 4 h at 50° C. The reaction mixture was purified on C18 column ACN/H₂O (0.1% FA) (0-60%). The pure fractions was concentrated and dried by lyophilization. There was ethyl 4-(5-(3-((4-chloro-2-(4-ethoxy-4-oxobutanoyl)-6-methoxyisoindolin-5-yl)oxy)propoxy)-4-fluoro-6-methoxy benzo[b]thiophen-2-yl)-4-oxobutanoate (0.040 g, 57.6234 μmol, 38.0259% yield) obtained as a white solid. LCMS: (ESI, m/z): [M+H]⁺=694.180. ¹H NMR (400 MHz, DMSO-d₆) δ 8.31 (s, 1H), 7.57 (s, 1H), 7.07 (s, 0.5H), 7.04 (s, 0.5H), 4.84 (s, 1H), 4.73 (s, 1H), 4.62 (s, 1H), 4.49 (s, 1H), 4.29 (t, J=6.0 Hz, 2H), 4.17 (t, J=6.0 Hz, 2H), 4.10-4.01 (m, 4H), 3.89 (s, 3H), 3.80 (d, J=2.0 Hz, 3H), 3.40-3.34 (m, 2H), 2.72-2.53 (m, 6H), 2.09-2.06 (m, 2H), 1.22-1.15 (m, 6H).

### Step h: 4-(5-(3-((2-(3-carboxypropanoyl)-4-chloro-6-methoxyisoindolin-5-yl)oxy)propoxy)-4-fluoro-6-methoxybenzo[b]thiophen-2-yl)-4-oxobutanoic Acid

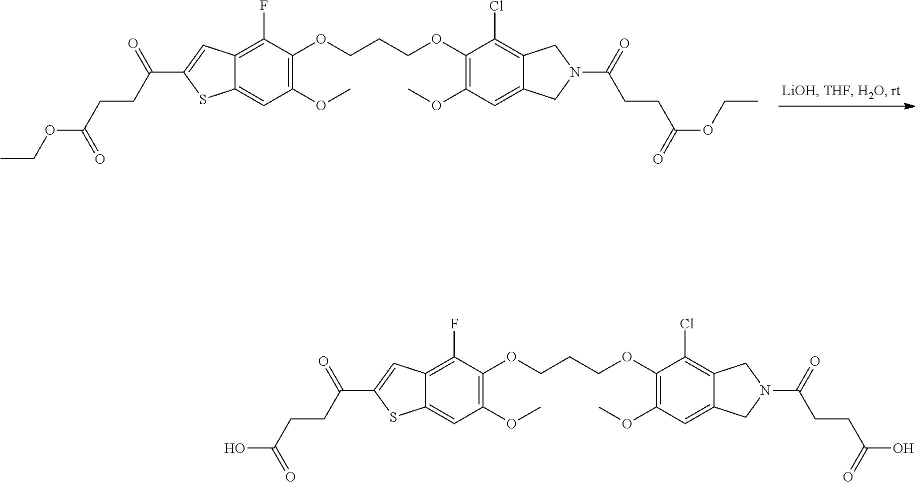 [SMILES: CCOC(=O)CCC(=O)c1cc2c(F)c(OCCCOc3c(OC)cc4c(c3Cl)CN(C(=O)CCC(=O)OCC)C4)c(OC)cc2s1 ; COc1cc2c(c(Cl)c1OCCCOc1c(OC)cc3sc(C(=O)CCC(=O)O)cc3c1F)CN(C(=O)CCC(=O)O)C2]

LiOH (0.048 g, 2.0043 mmol) was added to a solution of ethyl 4-(5-(3-((4-chloro-2-(4-ethoxy-4-oxobutanoyl)-6-methoxyisoindolin-5-yl)oxy)propoxy)-4-fluoro-6-methoxy benzo[b]thiophen-2-yl)-4-oxobutanoate (0.027 g, 38.8958 μmol) in THF (1 mL) and Water (1 mL) at 20° C. The reaction mixture was stirred for 1 h at 20° C. The reaction mixture was adjusted to pH=3 with HCl (1 mol/L), The reaction mixture was purified on C18 column ACN/H₂O (0.1% FA)(0-52%). The pure fractions was concentrated and dried by lyophilization. There was 4-(5-(3-((2-(3-carboxypropanoyl)-4-chloro-6-methoxyisoindolin-5-yl)oxy)propoxy)-4-fluoro-6-methoxybenzo[b]thiophen-2-yl)-4-oxobutanoic acid (0.017 g, 26.6434 μmol, 69.6846% yield) obtained as a white solid. LCMS: (ESI, m/z): [M+H]⁺=638.150. ¹H NMR (400 MHz, DMSO-d₆) δ 12.2 (s, 2H), 8.29 (s, 1H), 7.56 (s, 1H), 7.07 (s, 0.5H), 7.03 (s, 0.5H), 4.83 (s, 1H), 4.72 (s, 1H), 4.62 (s, 1H), 4.49 (s, 1H), 4.32-4.25 (m, 2H), 4.17 (t, J=6.1 Hz, 2H), 3.89 (s, 3H), 3.80 (d, J=1.7 Hz, 3H), 3.30-3.27 (m, 2H), 2.61-2.55 (m, 4H), 2.48-2.46 (m, 2H), 2.09-2.06 (m, 2H).

### Example 3

### 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic Acid

### Step a: Tert-butyl 5-(3-bromopropoxy)-4-fluoro-6-methoxy-isoindoline-2-carboxylate

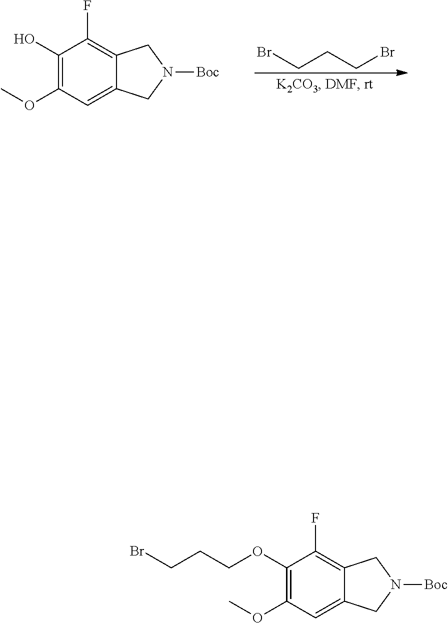 [SMILES: COc1cc2c(c(F)c1O)CN(C(=O)OC(C)(C)C)C2 ; BrCCCBr ; COc1cc2c(c(F)c1OCCCBr)CN(C(=O)OC(C)(C)C)C2]

To a solution of tert-butyl 4-fluoro-5-hydroxy-6-methoxy-isoindoline-2-carboxylate (0.50 g, 1.7649 mmol) in DMF (10 mL), was added 1,3-Dibromopropane (2.48 g, 12.2841 mmol) and K₂CO₃ (0.71 g, 5.1373 mmol). The reaction mixture was stirred for 3 h at 20° C. The reaction mixture was diluted with Ethyl acetate (200 mL), and washed sequentially with water (2×100 mL) and saturated brine (100 mL). The organic layer was dried over Na₂SO₄, filtered and evaporated to afford crude product. The crude product was purified on flash silica chromatography, elution gradient 0% to 100% Ethyl acetate in heptane. There was tert-butyl 5-(3-bromopropoxy)-4-fluoro-6-methoxy-isoindoline-2-carboxylate (0.637 g, 1.5757 mmol, 89.2763% yield) obtained as a white solid. LCMS: (ESI, m/z): [M+H]⁺=331.100.

### Step b: Tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate

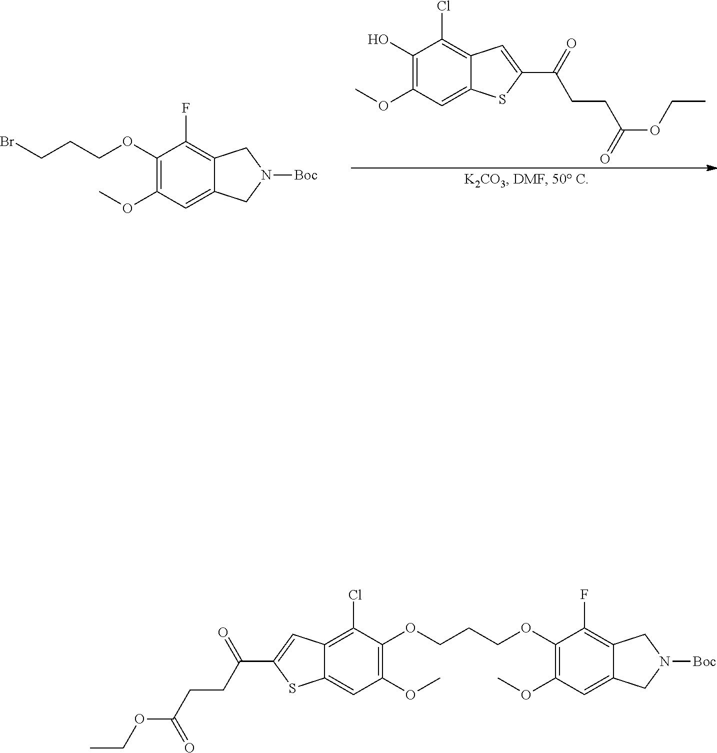 [SMILES: COc1cc2c(c(F)c1OCCCBr)CN(C(=O)OC(C)(C)C)C2 ; CCOC(=O)CCC(=O)c1cc2c(Cl)c(O)c(OC)cc2s1 ; CCOC(=O)CCC(=O)c1cc2c(Cl)c(OCCCOc3c(OC)cc4c(c3F)CN(C(=O)OC(C)(C)C)C4)c(OC)cc2s1]

K₂CO₃ (0.166 g, 1.2011 mmol) was added to a solution of ethyl 4-(4-chloro-5-hydroxy-6-methoxybenzo[b]thiophen-2-yl)-4-oxobutanoate (0.147 g, 428.8290 μmol) and tert-butyl 5-(3-bromopropoxy)-4-fluoro-6-methoxy-isoindoline-2-carboxylate (0.173 g, 427.9318 μmol) in DMF (5 mL) at 20° C. under N₂ atmosphere. The reaction mixture was stirred for 3 h at 50° C. The reaction mixture was purified on C18 column ACN/H₂O (0.1% FA)(0-75%). The pure fractions was concentrated and dried under vacuo. There was tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate (0.138 g, 207.1598 μmol, 48.3083% yield) obtained as a yellow solid. LCMS: (ESI, m/z): [M+H]⁺=666.190. ¹H NMR (400 MHz, DMSO-d₆) δ 8.21 (s, 1H), 7.74 (s, 1H), 6.89 (s, 0.5H), 6.87 (s, 0.5H), 4.53 (s, 4H), 4.21 (d, J=6.2, 4H), 4.06 (q, J=7.1 Hz, 2H), 3.90 (s, 3H), 3.77 (d, J=3.7 Hz, 3H), 3.39 (t, J=6.4 Hz, 2H), 2.66 (t, J=6.3 Hz, 2H), 2.11-2.09 (m, 2H), 1.45 (s, 9H), 1.18 (t, J=7.1 Hz, 3H).

### Step c: Ethyl 4-(4-chloro-5-(3-((4-fluoro-6-methoxyisoindolin-5-yl)oxy)propoxy)-6-methoxybenzo[b]thiophen-2-yl)-4-oxobutanoate

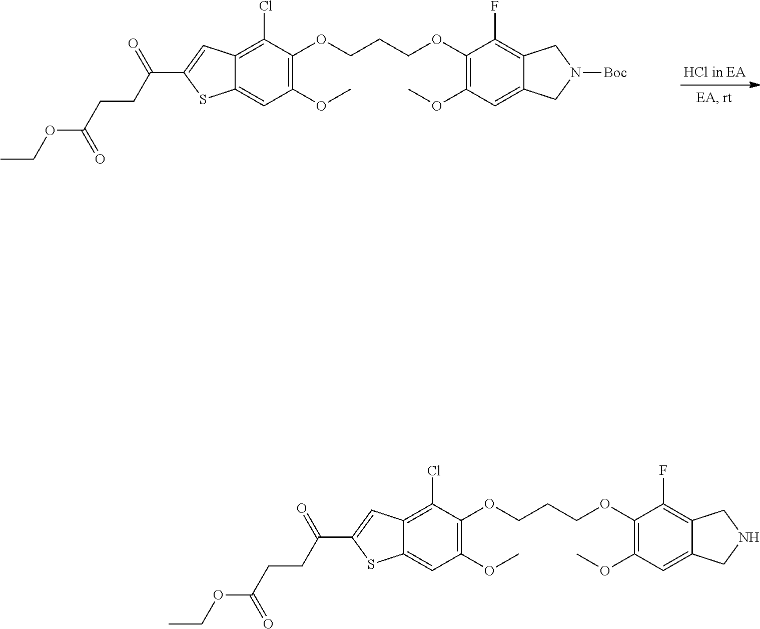 [SMILES: CCOC(=O)CCC(=O)c1cc2c(Cl)c(OCCCOc3c(OC)cc4c(c3F)CN(C(=O)OC(C)(C)C)C4)c(OC)cc2s1 ; CCOC(=O)CCC(=O)c1cc2c(Cl)c(OCCCOc3c(OC)cc4c(c3F)CNC4)c(OC)cc2s1]

HCl (5 mL, 20 mmol, 4 M in EA) was added to a solution of tert-butyl 5-(3-((4-chloro-2-(4-ethoxy-4-oxobutanoyl)-6-methoxybenzo[b]thiophen-5-yl)oxy)propoxy)-4-fluoro-6-methoxyisoindoline-2-carboxylate (0.117 g, 175.6356 μmol) in EA (5 mL) at 20° C. The reaction mixture was stirred for 2 h at 20° C. The reaction mixture was evaporated under reduced pressure. There was ethyl 4-(4-chloro-5-(3-((4-fluoro-6-methoxyisoindolin-5-yl)oxy)propoxy)-6-methoxybenzo[b]thiophen-2-yl)-4-oxobutanoate (0.182 g, 321.5338 μmol, 183.0687% yield) obtained as a brown solid. LCMS: (ESI, m/z): [M+H]⁺=566.130. ¹H NMR (400 MHz, DMSO-d₆) δ 10.34 (s, 2H), 8.22 (s, 1H), 7.75 (s, 1H), 6.97 (s, 1H), 4.49 (s, 2H), 4.46 (s, 2H), 4.23-4.20 (m, 4H), 4.06 (q, J=7.1 Hz, 2H), 3.90 (s, 3H), 3.79 (s, 3H), 2.67 (t, J=6.3 Hz, 2H), 2.09-2.11 (m, 2H), 1.18 (t, J=7.1 Hz, 3H).

### Step d: Ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate

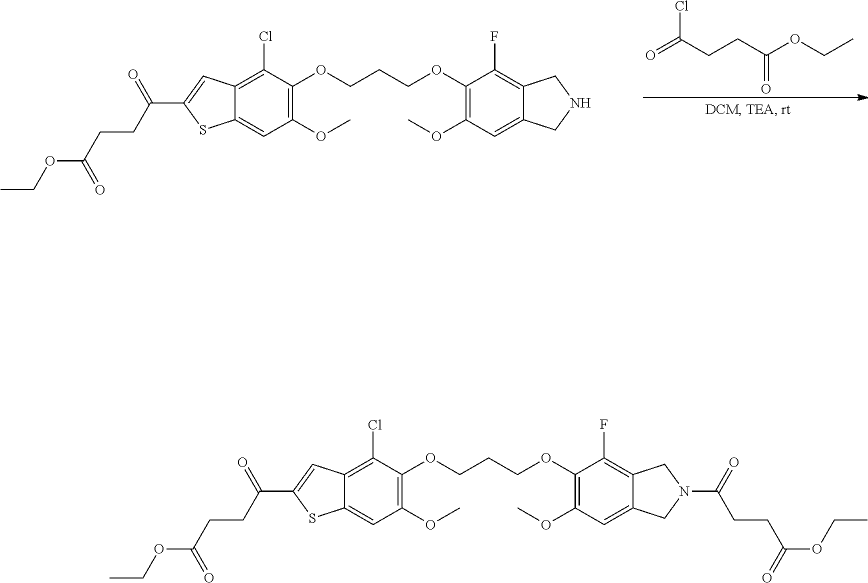

Ethyl Succinyl Chloride (0.097 g, 589.3557 μmol) was added to a solution of ethyl 4-(4-chloro-5-(3-((4-fluoro-6-methoxyisoindolin-5-yl)oxy)propoxy)-6-methoxybenzo[b]thiophen-2-yl)-4-oxobutanoate (0.167 g, 295.0337 μmol) and TEA (0.164 g, 1.6207 mmol) in DCM (5 mL) at 20° C. under N₂ atmosphere. The reaction mixture was stirred for 1 h at 20° C. The reaction mixture was evaporated under reduced pressure. The residue was purified on C18 column ACN/H₂O (0.1% FA)(0-60%). The pure fractions was concentrated and dried by lyophilization. There was ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate (0.081 g, 116.6874 μmol, 39.5505% yield) obtained as a white solid. LCMS: (ESI, m/z): [M+H]⁺=694.180. ¹H NMR (400 MHz, DMSO-d₆) δ 8.22 (s, 1H), 7.74 (d, J=2.2 Hz, 1H), 6.91 (s, 0.5H), 6.89 (s, 0.5H), 4.81 (s, 1H), 4.80 (s, 1H), 4.57 (s, 2H), 4.22 (q, J=5.9 Hz, 4H), 4.09-4.03 (m, 4H), 3.90 (d, J=1.1 Hz, 3H), 3.78 (d, J=2.5 Hz, 3H), 3.40 (s, 2H), 2.70-2.58 (m, 4H), 2.57-2.53 (m, 2H), 2.15-2.04 (m, 2H), 1.26-1.14 (m, 6H).

### Step e: 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic Acid

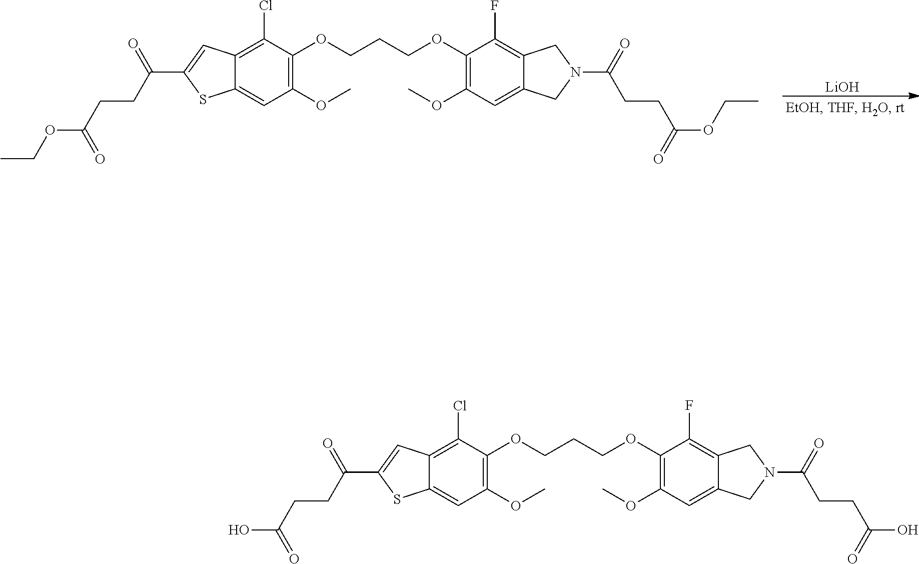 [SMILES: CCOC(=O)CCC(=O)c1cc2c(Cl)c(OCCCOc3c(OC)cc4c(c3F)CN(C(=O)CCC(=O)OCC)C4)c(OC)cc2s1 ; "LiOH" ; COc1cc2c(c(F)c1OCCCOc1c(OC)cc3sc(C(=O)CCC(=O)O)cc3c1Cl)CN(C(=O)CCC(=O)O)C2]

LiOH (0.045 g, 1.8790 mmol) was added to a solution of ethyl 4-(5-(3-((4-chloro-2-(4-ethoxy-4-oxobutanoyl)-6-methoxybenzo[b]thiophen-5-yl)oxy)propoxy)-4-fluoro-6-methoxyisoindolin-2-yl)-4-oxobutanoate (0.044 g, 63.3858 μmol) in THF (2 mL), H₂O (2 mL) and Ethanol (1 mL) at 20° C. The reaction mixture was stirred for 1 h at 20° C. The reaction mixture was adjusted pH=3 with HCl (1 mol/L). The reaction mixture was evaporated under reduced pressure. The residue was purified on C18 column ACN/H₂O (0.1% FA)(0-52%). The pure fractions was concentrated and dried by lyophilization. There was 4-(5-(3-((2-(3-carboxypropanoyl)-4-chloro-6-methoxybenzo[b]thiophen-5-yl)oxy)propoxy)-4-fluoro-6-methoxyisoindolin-2-yl)-4-oxobutanoic acid (0.032 g, 50.1523 μmol, 79.1223% yield) obtained as a white solid. LCMS: (ESI, m/z): [M+H]⁺=638.120. ¹H NMR (400 MHz, DMSO-d₆) δ 8.19 (s, 1H), 7.73 (s, 1H), 6.91 (s, 0.5H), 6.88 (s, 0.5H), 4.79 (s, 2H), 4.57 (s, 2H), 4.25-4.19 (m, 4H), 3.90 (s, 3H), 3.78 (d, J=1.7 Hz, 3H), 3.33 (t, J=12.0 Hz, 2H), 2.63-2.54 (m, 4H), 2.49-2.45 (m, 2H), 2.15-2.04 (m, 2H).

### Example 4

### 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic Acid

### Step a: Ethyl 4-(5-(3-bromopropoxy)-4-fluoro-6-methoxyisoindolin-2-yl)-4-oxobutanoate

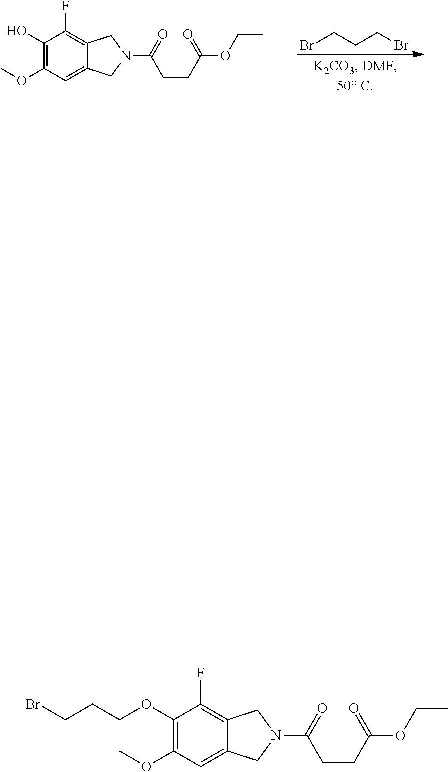 [SMILES: CCOC(=O)CCC(=O)N1Cc2cc(OC)c(O)c(F)c2C1 ; BrCCCBr ; CCOC(=O)CCC(=O)N1Cc2cc(OC)c(OCCCBr)c(F)c2C1]

1,3-Dibromopropane (0.708 g, 3.5069 mmol) was added to a mixture of ethyl 4-(4-fluoro-5-hydroxy-6-methoxy-isoindolin-2-yl)-4-oxo-butanoate (0.217 g, 697.0656 μmol) and Potassium carbonate (0.346 g, 2.5035 mmol) in DMF (5 mL) at 20° C. The reaction mixture was stirred for 3 h at 20° C. The reaction mixture was purified on C18 column ACN/H₂O (0.1% FA)(0-45%). The pure fractions was concentrated and dried under vacuo. There was ethyl 4-(5-(3-bromopropoxy)-4-fluoro-6-methoxyisoindolin-2-yl)-4-oxobutanoate (0.211 g, 488.1091 μmol, 70.0234% yield) obtained as a white solid. LCMS: (ESI, m/z): [M+H]⁺=432.070.

### Step b: Ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate

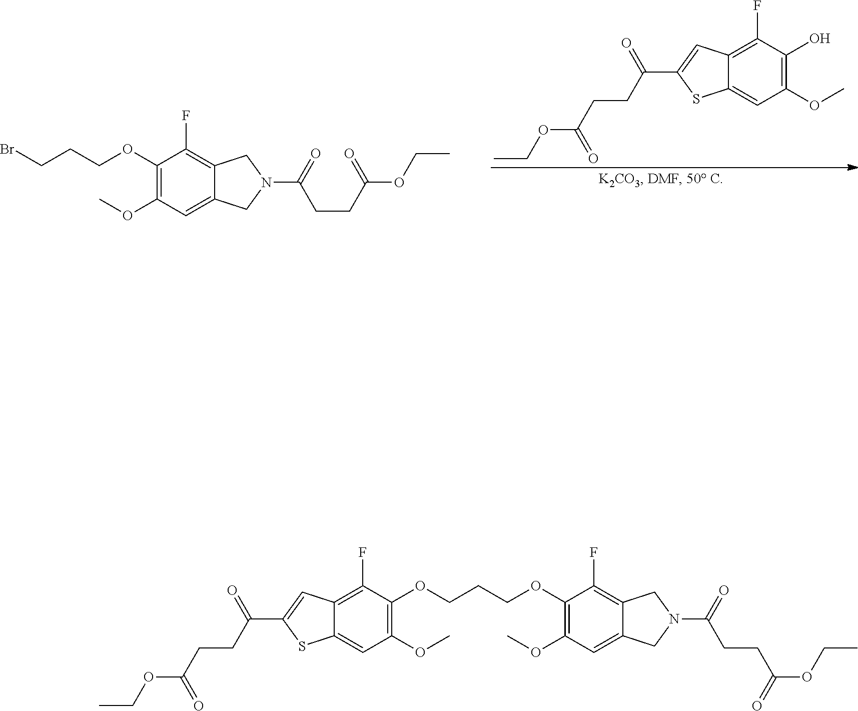

Potassium carbonate (0.079 g, 571.6126 μmol) was added to a solution of ethyl 4-(5-(3-bromopropoxy)-4-fluoro-6-methoxyisoindolin-2-yl)-4-oxobutanoate (0.070 g, 161.9319 μmol) and ethyl 4-(4-fluoro-5-hydroxy-6-methoxybenzo[b]thiophen-2-yl)-4-oxobutanoate (0.065 g, 199.1791 μmol) in N,N-Dimethylformamide (3 mL) at 20° C. under N₂ atmosphere. The reaction mixture was stirred overnight at 50° C. The reaction mixture was purified on C18 column ACN/H₂O (0.1% FA)(0-70%). The pure fractions was concentrated and dried by lyophilization. There was ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate (0.056 g, 82.6315 μmol, 51.0285% yield) obtained as a yellow solid. LCMS: (ESI, m/z): [M+H]⁺=678.210. ¹H NMR (400 MHz, DMSO-d₆) δ 8.31 (s, 1H), 7.56 (s, 1H), 6.89 (s, 0.5H), 6.86 (s, 0.5H), 4.78 (s, 2H), 4.56 (s, 1H), 4.54 (s, 1H), 4.25 (t, J=6.0 Hz, 2H), 4.19 (t, J=6.0 Hz, 2H), 4.09-4.02 (m, 4H), 3.88 (s, 3H), 3.77 (d, J=2.0 Hz, 3H), 3.36 (t, J=6.4 Hz, 2H), 2.70-2.51 (m, 6H), 2.10-1.99 (m, 2H), 1.22-1.14 (m, 6H).

### Step c: 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic Acid

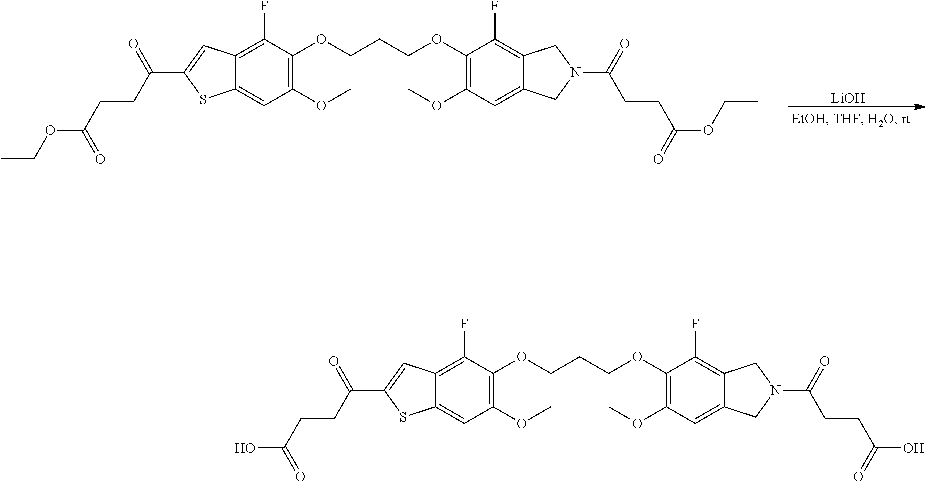 [SMILES: CCOC(=O)CCC(=O)c1cc2c(F)c(OCCCOc3c(OC)cc4c(c3F)CN(C(=O)CCC(=O)OCC)C4)c(OC)cc2s1 ; "LiOH" ; COc1cc2c(c(F)c1OCCCOc1c(OC)cc3sc(C(=O)CCC(=O)O)cc3c1F)CN(C(=O)CCC(=O)O)C2]

LiOH (0.043 g, 1.7955 mmol) was added to a solution of ethyl 4-(5-(3-((2-(4-ethoxy-4-oxobutanoyl)-4-fluoro-6-methoxybenzo[b]thiophen-5-yl)oxy)propoxy)-4-fluoro-6-methoxyisoindolin-2-yl)-4-oxobutanoate (0.039 g, 57.5469 μmol) in THF (2 mL), H₂O (2 mL) and Ethanol (1 mL) at 20° C. The reaction mixture was stirred overnight at 20° C. The reaction mixture was adjusted to pH=3 with HCl (aq. 1 M), The reaction mixture was evaporated under reduced pressure. The residue was purified on C18 column ACN/H₂O (0.1% FA)(0-50%). The pure fractions was concentrated and dried under vacuo. There was 4-(5-(3-((2-(3-carboxypropanoyl)-4-fluoro-6-methoxybenzo[b]thiophen-5-yl)oxy)propoxy)-4-fluoro-6-methoxyisoindolin-2-yl)-4-oxobutanoic acid (0.012 g, 19.3050 μmol, 33.5465% yield) obtained as a white solid. LCMS: (ESI, m/z): [M+H]⁺=622.150. ¹H NMR (400 MHz, DMSO-d₆) δ 12.16 (s, 2H), 8.29 (s, 1H), 7.56 (s, 1H), 6.90 (s, 0.5H), 6.86 (s, 0.5H), 4.77 (s, 2H), 4.56 (s, 1H), 4.55 (s, 1H), 4.29-4.15 (m, 4H), 3.88 (s, 3H), 3.77 (s, 3H), 3.31-3.29 (m, 2H), 2.63-2.53 (m, 4H), 2.50-2.46 (m, 2H), 2.10-1.99 (m, 2H).

### Example 5

### Sodium (S)-4-(5-(3-((2-((S)-3-carboxylatobutanoyl)-6-methoxybenzo[b]thiophen-5-yl)oxy)propoxy)-6-methoxyisoindolin-2-yl)-2-methyl-4-oxobutanoate

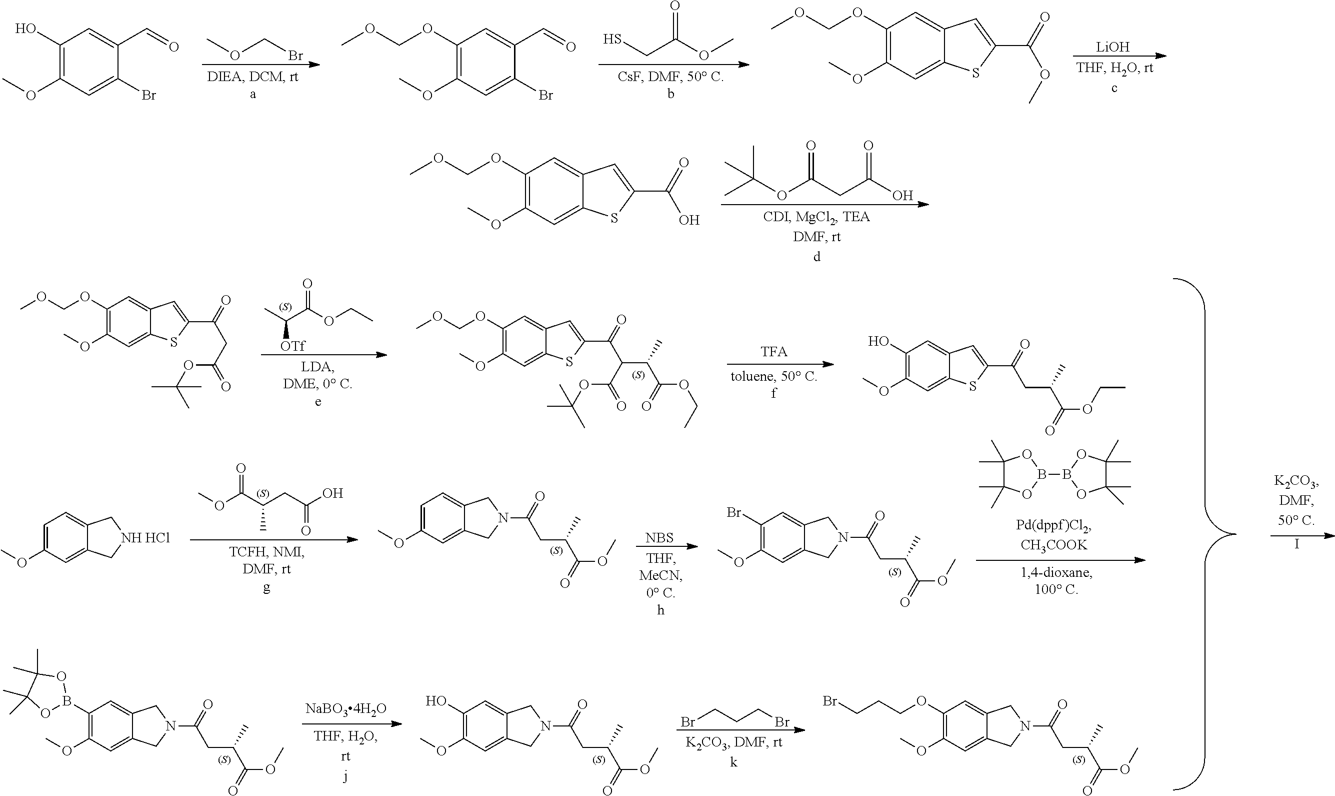

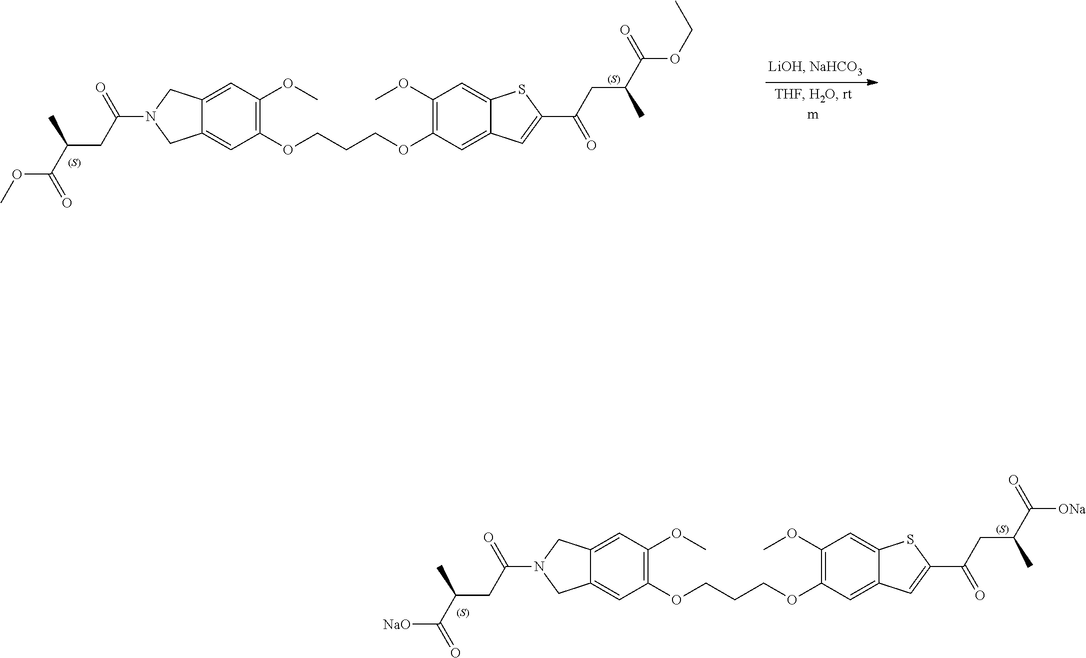

Step a: Bromomethyl methyl ether (60.56 g, 484.6196 mmol) was added to a solution of N,N-Diisopropylethylamine (97.64 g, 755.4796 mmol) and 2-bromo-5-hydroxy-4-methoxybenzaldehyde (102.80 g, 444.9386 mmol) in DCM (1500 mL) at 0° C. under N₂ atmosphere. The reaction mixture was stirred at 25° C. for 2 h. The resulting solution was quenched with adding of water (1000 mL) at 25° C. The resulting mixture was extracted with DCM (2×1000 mL), washed with water (500 mL) and brine (500 mL). The organics dried over Na₂SO₄, filtered and evaporated under reduced pressure. There was 2-bromo-4-methoxy-5-(methoxymethoxy)benzaldehyde (115.25 g, 418.9454 mmol, 94.1580% yield) obtained as a light yellow solid. LCMS: (ESI, m/z): [M+H]⁺=274.984. ¹H NMR (400 MHz, DMSO-d₆) δ 10.06 (s, 1H), 7.51 (s, 1H), 7.39 (s, 1H), 5.25 (s, 2H), 3.93 (s, 3H), 3.39 (s, 3H).

Step b: Methyl thioglycolate (53.415 g, 503.2348 mmol) was added to a solution of 2-bromo-4-methoxy-5-(methoxymethoxy)benzaldehyde (115.11 g, 418.4365 mmol) and Caesium fluoride (130.32 g, 857.9136 mmol) in N,N-Dimethylformamide (1200 mL) at 25° C. under N₂ atmosphere. The reaction mixture was heated to 50° C. and stirred overnight. The reaction mixture was pour into the water (10 L) and stirred for 1 h. The precipitate was collected by filtration, washed with water (3×200 mL). The filter cake was dried under vacuo at 60° C. to obtain a crude product. The crude product was diluted with DCM (200 mL). Pour the mixture into n-Hexane (2000 mL) and stirred for 1 h. The precipitate was collected by filtration, washed with n-Hexane (3×100 mL). The filter cake was dried under vacuo at 60° C. There was methyl 6-methoxy-5-(methoxymethoxy)benzothiophene-2-carboxylate (65.50 g, 232.0131 mmol, 55.4476% yield) obtained as a yellow solid. LCMS: (ESI, m/z): [M+H]⁺=284.056. ¹H NMR (400 MHz, DMSO-d₆) δ 8.07 (s, 1H), 7.69 (s, 1H), 7.65 (s, 1H), 5.25 (s, 2H), 3.90 (s, 3H), 3.88 (s, 3H), 3.44 (s, 3H).

Step c: LiOH (17.58 g, 734.0813 mmol) was added to a solution of methyl 6-methoxy-5-(methoxymethoxy)benzothiophene-2-carboxylate (51.16 g, 181.2181 mmol) in THF (600 mL) and Water (200 mL) at 25° C. The reaction mixture was stirred for 4 h at 25° C. The reaction mixture was evaporated under reduced pressure. The residue was diluted with water and adjusted to pH=4 with HCl (1 M). The precipitate was collected by filtration, washed with water (2×50 mL). The filtrate cake was dried under vacuo. There was 6-methoxy-5-(methoxymethoxy)benzothiophene-2-carboxylic acid (44.17 g, 164.6382 mmol, 90.8509% yield) obtained as a yellow solid. LCMS: (ESI, m/z): [M−H]⁻=267.041. ¹H NMR (400 MHz, DMSO-d₆) δ 13.17 (s, 1H), 7.95 (s, 1H), 7.64 (s, 1H), 7.62 (s, 1H), 5.23 (s, 2H), 3.88 (s, 3H), 3.43 (s, 3H).

Step d: To a stirred solution of 6-methoxy-5-(methoxymethoxy)benzothiophene-2-carboxylic acid (53.21 g, 198.3337 mmol) in DMF (1000 mL) was added CDI (64.55 g, 398.0919 mmol) at 25° C. The reaction mixture was stirred for 2 h at 25° C. To the mixture above was added 3-(tert-butoxy)-3-oxopropanoic acid (49.03 g, 306.1174 mmol), TEA (62.33 g, 615.9736 mmol) and Magnesium chloride (29.11 g, 305.7420 mmol). The reaction mixture was stirred overnight at 25° C. The reaction mixture was concentrated and diluted with EA (3000 mL), washed with water (2×3000 mL) and brine (3000 mL). The organics dried over Na₂SO₄, filtered and evaporated under reduced pressure. The residue was purified on silica gel column EA/n-Hexane (0-50%). The pure fractions was concentrated and dried under vacuo. There was tert-butyl 3-(6-methoxy-5-(methoxymethoxy)benzo[b]thiophen-2-yl)-3-oxopropanoate (36.19 g, 98.7643 mmol, 49.7971% yield) obtained as a yellow oil. LCMS: (ESI, m/z): [M+H]⁺=367.114. ¹H NMR (400 MHz, DMSO-d₆) δ 8.20 (s, 1H), 7.67 (s, 1H), 7.62 (s, 1H), 5.24 (s, 2H), 4.05 (s, 2H), 3.89 (s, 3H), 3.42 (s, 3H), 1.41 (s, 9H).

Step e: LDA (7.9020 g, 73.7662 mmol) was added to a solution of tert-butyl 3-(6-methoxy-5-(methoxymethoxy)benzo[b]thiophen-2-yl)-3-oxopropanoate (18.02 g, 49.1775 mmol) in 2,5-Dioxahexane (200 mL) at 0° C. under N₂ atmosphere. The reaction mixture was stirred for 40 min at 0° C. under N₂ atmosphere. Ethyl (S)-2-(((trifluoromethyl)sulfonyl)oxy)propanoate (18.06 g, 72.1843 mmol) was added to the mixture at 0° C. under N₂ atmosphere. The reaction mixture was stirred for 3 h at 0° C. under N₂ atmosphere. The reaction mixture was quenched with adding of KHCO₃ (100 mL) at 0° C. The reaction mixture was concentrated and diluted with EA (500 mL), washed with water (200 mL) and brine (100 mL). The organics dried over Na₂SO₄, filtered and evaporated under reduced pressure. The residue was purified on silica gel column EA/n-Hexane (0-100%). The pure fractions was concentrated and dried under vacuo. There was 1-(tert-butyl) 4-ethyl(3S)-2-(6-methoxy-5-(methoxymethoxy)benzo[b]thiophene-2-carbonyl)-3-methylsuccinate (21.55 g, 46.1908 mmol, 93.9267% yield) obtained as a yellow oil. LCMS: (ESI, m/z): [M+H]⁺=467.166.

Step f: TFA (101.31 g, 888.5026 mmol) was added to a solution of 1-(tert-butyl) 4-ethyl(3S)-2-(6-methoxy-5-(methoxymethoxy)benzo[b]thiophene-2-carbonyl)-3-methylsuccinate (21.52 g, 46.1265 mmol) in Toluene (198 mL) at 25° C. The reaction mixture was heated to 50° C. and stirred for 2 h. The reaction mixture was evaporated under reduced pressure. The residue was purified on silica gel column EA/n-Hexane (0-50%). The pure fractions was concentrated and dried under vacuo. There was ethyl (2S)-4-(5-hydroxy-6-methoxy-benzothiophen-2-yl)-2-methyl-4-oxo-butanoate (6.78 g, 21.0314 mmol, 45.5950% yield) obtained as a yellow solid. LCMS: (ESI, m/z): [M+H]⁺=323.087. ¹H NMR (400 MHz, DMSO-d₆) δ 9.39 (s, 1H), 8.17 (s, 1H), 7.54 (s, 1H), 7.31 (s, 1H), 4.10-3.98 (m, 2H), 3.87 (s, 3H), 3.44-3.35 (m, 1H), 3.19-3.08 (m, 1H), 2.98-2.88 (m, 1H), 1.21-1.10 (m, 6H).

Step g: NMI (7.96 g, 96.9507 mmol) was added to a solution of TCFH (8.11 g, 28.9045 mmol) and (S)-4-methoxy-3-methyl-4-oxobutanoic acid (2.81 g, 19.2280 mmol) in DMF (100 mL) at 25° C. The reaction mixture was stirred for 10 min at 20° C. 5-methoxyisoindoline hydrochloride (4.99 g, 26.8785 mmol) was added to the solution at 25° C. The reaction mixture was stirred 2 h at 25° C. The reaction mixture was concentrated and diluted with H₂O (200 mL), extracted with EA (2×200 mL) and washed with brine (100 mL). The organics dried over Na₂SO₄, filtered and evaporated under reduced pressure. The residue was purified on silica gel column EA/n-Hexane (0-55%). The pure fractions was concentrated and dried under vacuo. There was methyl (2S)-4-(5-methoxyisoindolin-2-yl)-2-methyl-4-oxo-butanoate (3.49 g, 12.5850 mmol, 65.4512% yield) obtained as a yellow solid. LCMS: (ESI, m/z): [M+H]⁺=278.130. ¹H NMR (400 MHz, DMSO-d₆) δ 7.23 (t, J=8.0 Hz, 1H), 6.95-6.84 (m, 2H), 4.78 (s, 1H), 4.73 (s, 1H), 4.56 (s, 1H), 4.52 (s, 1H), 3.75 (d, J=1.8 Hz, 3H), 3.60 (s, 3H), 2.91-2.81 (m, 1H), 2.76-2.66 (m, 1H), 2.54-2.51 (m, 0.5H), 2.49-2.45 (m, 0.5H), 1.18-1.14 (m, 3H).

Step h: NBS (3.717 g, 20.8839 mmol) was added to a solution of methyl (2S)-4-(5-methoxyisoindolin-2-yl)-2-methyl-4-oxo-butanoate (3.426 g, 12.3542 mmol) in THF (30 mL) and MeCN (30 mL) at 0° C. The reaction mixture was stirred for 3 h at 25° C. The reaction mixture was concentrated and diluted with DCM (200 mL), washed with H₂O (100 mL), KHCO₃ (aq)(2×100 mL) and brine (100 mL). The organics dried over Na₂SO₄, filtered and evaporated under reduced pressure. The residue was purified on silica gel column EA/n-Hexane (0-60%). The pure fractions was concentrated and dried under vacuo. There was methyl (2S)-4-(5-bromo-6-methoxy-isoindolin-2-yl)-2-methyl-4-oxo-butanoate (3.02 g, 8.4781 mmol, 68.6255% yield) obtained as a yellow solid. LCMS: (ESI, m/z): [M+H]⁺=356.040. ¹H NMR (400 MHz, DMSO-d₆) δ 7.57 (s, 0.5H), 7.55 (s, 0.5H), 7.14 (s, 0.5H), 7.09 (s, 0.5H), 4.78 (s, 1H), 4.75 (s, 1H), 4.56 (s, 1H), 4.53 (s, 1H), 3.86 (s, 3H), 3.59 (s, 3H), 2.93-2.79 (m, 1H), 2.78-2.64 (m, 1H), 2.49-2.46 (m, 1H), 1.15 (d, J=7.1 Hz, 3H).

Step i: Potassium Acetate (2.81 g, 28.6319 mmol) was added to a mixture of methyl (2S)-4-(5-bromo-6-methoxy-isoindolin-2-yl)-2-methyl-4-oxo-butanoate (3.00 g, 8.4220 mmol), [1,1′-Bis(diphenylphosphino)ferrocene]dichloropalladium(II) (0.74 g, 1.0113 mmol) and 4,4,4′,4′,5,5,5′,5′-Octamethyl-2,2′-bi(1,3,2-dioxaborolane) (3.18 g, 12.5228 mmol) in 1,4-Dioxane (100 mL) at 25° C. The reaction mixture was heated to 100° C. and stirred overnight. The reaction mixture was diluted with EA (200 mL), The precipitate was collected by filtration, washed with EA (1×100 mL). The filtrate was evaporated under reduced pressure. The residue was purified on silica gel column EA/n-Hexane (0-50%). The pure fractions was concentrated and dried under vacuo. There was methyl (2S)-4-[5-methoxy-6-(4,4,5,5-tetramethyl-1,3,2-dioxaborolan-2-yl)isoindolin-2-yl]-2-methyl-4-oxo-butanoate (2.38 g, 5.9017 mmol, 70.0746% yield) obtained as a brown solid. LCMS: (ESI, m/z): [M+H]⁺=404.222. ¹H NMR (400 MHz, DMSO-d₆) δ 7.49 (s, 0.5H), 7.46 (s, 0.5H), 6.97 (s, 0.5H), 6.93 (s, 0.5H), 4.81 (s, 1H), 4.73 (s, 1H), 4.59 (s, 1H), 4.52 (s, 1H), 3.73 (s, 3H), 3.59 (s, 3H), 2.90-2.81 (m, 1H), 2.76-2.65 (m, 1H), 2.54-2.51 (m, 1H), 1.27 (s, 12H), 1.15 (d, J=6.6 Hz, 3H).

Step j: Sodium perborate tetrahydrate (0.80 g, 5.1995 mmol) was added to a solution of methyl (2S)-4-[5-methoxy-6-(4,4,5,5-tetramethyl-1,3,2-dioxaborolan-2-yl)isoindolin-2-yl]-2-methyl-4-oxo-butanoate (1.99 g, 4.9346 mmol) in THF (25 mL) and H₂O (25 mL) at 25° C. The reaction mixture was stirred for 1 h at 25° C. The reaction mixture was concentrated and diluted with H₂O (200 mL), extracted with DCM (3×100 mL) and washed with brine (100 mL). The organics dried over Na₂SO₄, filtered and evaporated under reduced pressure. The residue was purified on C18 column ACN/H₂O (0-40%). The pure fractions was concentrated and dried under vacuo. There was methyl (2S)-4-(5-hydroxy-6-methoxy-isoindolin-2-yl)-2-methyl-4-oxo-butanoate (1.23 g, 4.1935 mmol, 84.9809% yield) obtained as a white solid. LCMS: (ESI, m/z): [M+H]⁺=294.130.

Step k: Potassium carbonate (0.462 g, 3.3428 mmol) was added to a solution of methyl (2S)-4-(5-hydroxy-6-methoxy-isoindolin-2-yl)-2-methyl-4-oxo-butanoate (0.311 g, 1.0603 mmol) and 1,3-Dibromopropane (1.158 g, 5.7359 mmol) in DMF (20 mL) at 25° C. The reaction mixture was stirred for 3 h at 25° C. The reaction mixture was purified on C18 column ACN/H₂O (0-45%). The pure fractions was concentrated and dried under vacuo. There was methyl (2R)-4-[5-(3-bromopropoxy)-6-methoxy-isoindolin-2-yl]-2-methyl-4-oxo-butanoate (0.314 g, 757.9234 μmol, 71.4822% yield) obtained as a colorless oil. LCMS: (ESI, m/z): [M+H]⁺=414.080.

Step l: Potassium carbonate (0.214 g, 1.5484 mmol) was added to a solution of methyl (S)-4-(5-(3-bromopropoxy)-6-methoxyisoindolin-2-yl)-2-methyl-4-oxobutanoate (0.206 g, 497.2362 μmol) and ethyl (2S)-4-(5-hydroxy-6-methoxy-benzothiophen-2-yl)-2-methyl-4-oxo-butanoate (0.243 g, 753.7805 mol) in DMF (5 mL) at 25° C. The reaction mixture was heated to 50° C. and stirred overnight. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-100%). The pure fractions was concentrated and dried under vacuo. There was ethyl (S)-4-(6-methoxy-5-(3-((6-methoxy-2-((S)-4-methoxy-3-methyl-4-oxobutanoyl)isoindolin-5-yl)oxy)propoxy)benzo[b]thiophen-2-yl)-2-methyl-4-oxobutanoate (0.130 g, 198.2454 μmol, 39.8695% yield) obtained as a yellow solid. LCMS: (ESI, m/z): [M+H]⁺=656.245.

Step m: LiOH (0.017 g, 709.8625 μmol) was added to a solution of ethyl (S)-4-(6-methoxy-5-(3-((6-methoxy-2-((S)-4-methoxy-3-methyl-4-oxobutanoyl)isoindolin-5-yl)oxy)propoxy)benzo[b]thiophen-2-yl)-2-methyl-4-oxobutanoate (0.120 g, 182.9957 μmol) in THF (4 mL) and Water (1 mL) at 25° C. The reaction mixture was stirred overnight at 25° C. The reaction mixture was adjusted to pH=7 with HCl (1 M). The reaction mixture was evaporated under reduced pressure to obtain a crude product. The crude product was purified by Prep-HPLC with the following conditions: (CXTH LC6000, HPLC-P1): Column, Agela Durashell C18, 30 mm*250 mm, 10 um; mobile phase, Water (0.1% NH3·H2O) and MeCN-(5-20-20% B (2-32-60 min)); Detector, uv 210 nm). The solvent was removed by lyophilization. NaHCO₃ (0.019 g, 226.1727 μmol) was added to the lyophilized product in water, and stirred for 30 min at 25° C. The solvent was removed by lyophilization. There was sodium (S)-4-(5-(3-((2-((S)-3-carboxylatobutanoyl)-6-methoxybenzo[b]thiophen-5-yl)oxy)propoxy)-6-methoxyisoindolin-2-yl)-2-methyl-4-oxobutanoate (0.064 g, 97.3180 μmol, 53.1805% yield) obtained as a white solid. LCMS: (ESI, m/z): [M+H]⁺=614.198. ¹H NMR (400 MHz, DMSO-d₆) δ 8.08 (s, 0.5H), 8.05 (s, 0.5H), 7.56 (s, 1H), 7.51 (s, 0.5H), 7.50 (s, 0.5H), 7.02-6.89 (m, 2H), 4.90-4.77 (m, 1H), 4.73-4.62 (m, 1H), 4.50 (s, 2H), 4.24-4.16 (m, 2H), 4.15-4.05 (m, 2H), 3.85 (s, 3H), 3.74 (s, 3H), 3.45-3.38 (m, 2H), 2.75-2.57 (m, 2H), 2.47-2.39 (m, 1H), 2.27-2.14 (m, 2H), 2.11-1.97 (m, 1H), 1.02 (t, J=6.5 Hz, 6H).

### Example 6

### Sodium (S)-4-(5-(3-((2-((S)-3-carboxylatobutanoyl)-4-fluoro-6-methoxybenzo[b]thiophen-5-yl)oxy)propoxy)-6-methoxyisoindolin-2-yl)-2-methyl-4-oxobutanoate

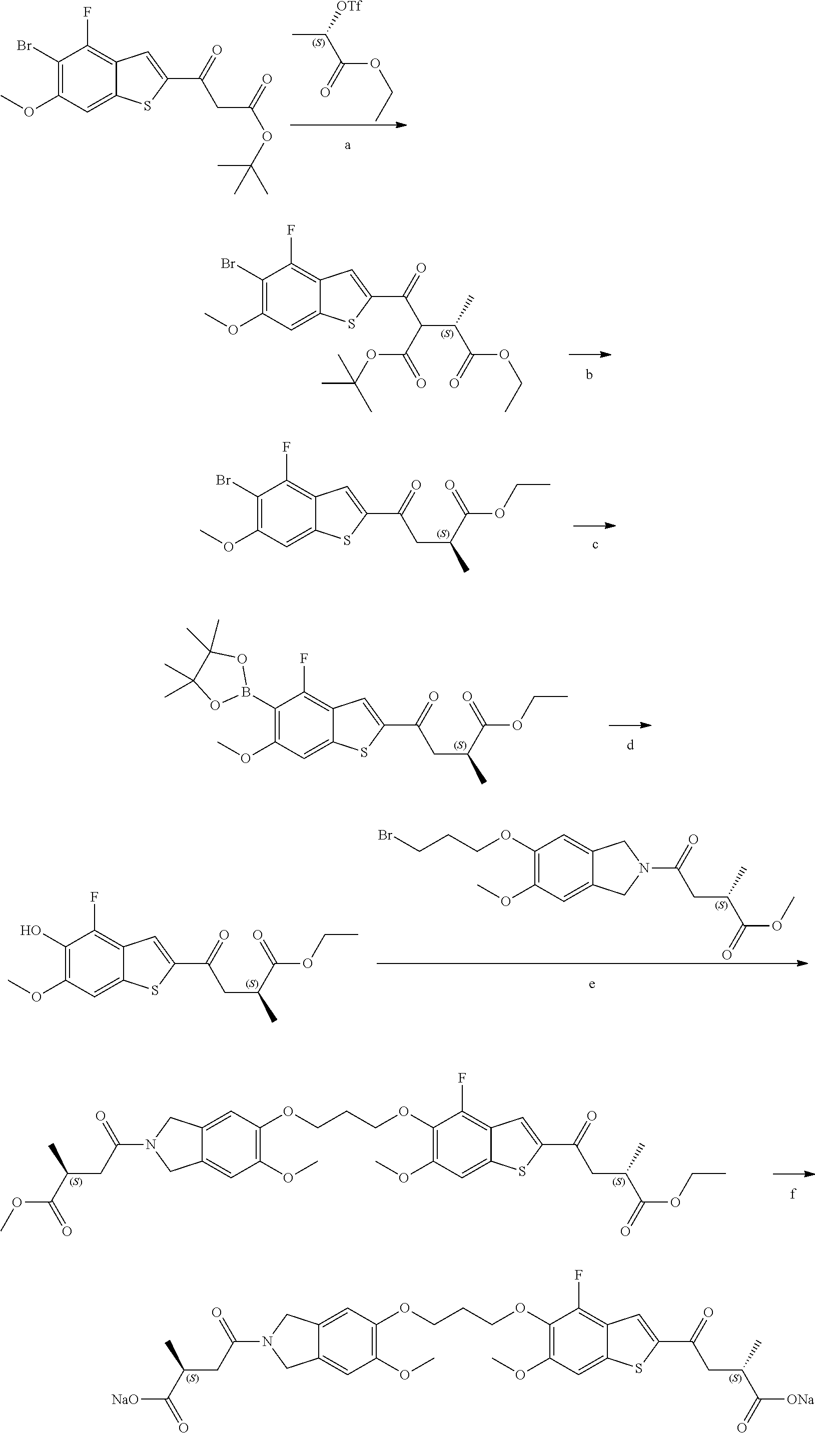

Step a: Lithium diisopropylamide, (552.5297 mg, 5.1579 mmol) was added to a solution of tert-butyl 3-(5-bromo-4-fluoro-6-methoxybenzo[b]thiophen-2-yl)-3-oxopropanoate (1.6 g, 3.9676 mmol) in 2,5-Dioxahexane (50 mL) at 0° C. under N₂ atmosphere. The reaction mixture was stirred for 40 min at 0° C. under N₂ atmosphere. Ethyl (S)-2-(((trifluoromethyl)sulfonyl)oxy)propanoate (2.9780 g, 11.9029 mmol) was added to the mixture at 0° C. under N₂ atmosphere. The reaction mixture was stirred overnight at 25° C. under N₂ atmosphere. The reaction mixture was concentrated and diluted with EA (100 mL), washed with NaHCO₃ (aq) (2×500 mL) and brine (50 mL). The organics dried over Na₂SO₄, filtered and evaporated under reduced pressure. The residue was purified on silica gel column EA/n-Hexane (0-20%). The pure fractions was concentrated and dried under vacuo. There was 1-(tert-butyl) 4-ethyl (3S)-2-(5-bromo-4-fluoro-6-methoxybenzo[b]thiophene-2-carbonyl)-3-methylsuccinate (1.15 g, 2.2846 mmol, 57.5800% yield) obtained as a colorless oil. LCMS: (ESI, m/z): [M+H]⁺=503.046. ¹H NMR (400 MHz, DMSO-d₆) δ 8.61 (s, 0.5H), 8.46 (s, 0.5H), 7.73 (s, 1H), 4.93-4.77 (m, 1H), 4.13-4.02 (m, 2H), 4.00-3.94 (m, 3H), 3.24-3.11 (m, 1H), 1.31 (d, J=11.5 Hz, 9H), 1.25-1.16 (m, 3H), 1.10 (t, J=7.8 Hz, 3H).

Step b: Trifluoroacetic acid (4 mL) was added to a solution of 1-(tert-butyl) 4-ethyl (3S)-2-(5-bromo-4-fluoro-6-methoxybenzo[b]thiophene-2-carbonyl)-3-methylsuccinate (1.02 g, 2.0263 mmol) in Toluene (20 mL) at 25° C. The reaction mixture was stirred at 50° C. for 2 h. The reaction mixture was concentrated and purified on C-18 column MeCN/water (0-100%). The pure fractions was concentrated and dried under vacuo. There was ethyl (S)-4-(5-bromo-4-fluoro-6-methoxybenzo[b]thiophen-2-yl)-2-methyl-4-oxobutanoate (0.76 g, 1.8846 mmol, 93.0079% yield) obtained as a yellow solid. LCMS: (ESI, m/z): [M+H]⁺=402.994. ¹H NMR (400 MHz, MeOD-d₄) δ 8.14 (s, 1H), 7.40 (s, 1H), 4.19-4.03 (m, 2H), 3.98 (s, 3H), 3.60-3.39 (m, 1H), 3.25-2.97 (m, 2H), 1.28 (d, J=7.1 Hz, 3H), 1.23 (t, J=7.1 Hz, 3H).

Step c: [1,1′-Bis(diphenylphosphino)ferrocene]dichloropalladium(II) (0.13 g, 177.6676 μmol) was added to a mixture of Potassium Acetate (0.53 g, 5.4003 mmol) ethyl (2S)-4-(5-bromo-4-fluoro-6-methoxy-benzothiophen-2-yl)-2-methyl-4-oxo-butanoate (0.70 g, 1.7358 mmol) and 4,4,4′,4′,5,5,5′,5′-Octamethyl-2,2′-bi(1,3,2-dioxaborolane) (0.87 g, 3.4260 mmol) in 1,4-Dioxane (30 mL) at 25° C. The reaction mixture was stirred overnight at 100° C. under N₂ atmosphere. The reaction mixture was evaporated under reduced pressure. The residue was concentrated and diluted with EA (200 mL), washed with NaHCO₃ (aq) (2×100 mL) and brine (100 mL). The organics dried over Na₂SO₄, filtered and evaporated under reduced pressure. The residue was purified on silica gel column EA/Hept (0-50%). The pure fractions was concentrated and dried under vacuo. There was ethyl (S)-4-(4-fluoro-6-methoxy-5-(4,4,5,5-tetramethyl-1,3,2-dioxaborolan-2-yl)benzo[b]thiophen-2-yl)-2-methyl-4-oxobutanoate (452 mg, 1.0037 mmol, 57.8229% yield) obtained as a white solid. LCMS: (ESI, m/z): [M+H]⁺=451.168. ¹H NMR (400 MHz, DMSO-d₆) δ 8.29 (s, 1H), 7.49 (s, 1H), 4.15-3.99 (m, 2H), 3.86 (s, 3H), 3.57-3.40 (m, 2H), 3.18 (s, 1H), 1.41-1.23 (m, 12H), 1.19-1.14 (m, 6H).

Step d: Sodium perborate tetrahydrate (0.23 g, 1.4949 mmol) was added to a solution of ethyl (S)-4-(4-fluoro-6-methoxy-5-(4,4,5,5-tetramethyl-1,3,2-dioxaborolan-2-yl)benzo[b]thiophen-2-yl)-2-methyl-4-oxobutanoate (0.46 g, 1.0215 mmol) in Tetrahydrofuran (20 mL) and Water (20 mL) at 25° C. The reaction mixture was stirred at 25° C. for 1 h. The reaction mixture was evaporated under reduced pressure. The residue was concentrated and diluted with DCM (200 mL), washed with water (2×100 mL) and brine (200 mL). The organics dried over Na₂SO₄, filtered and evaporated under reduced pressure. There was ethyl (2S)-4-(4-fluoro-5-hydroxy-6-methoxy-benzothiophen-2-yl)-2-methyl-4-oxo-butanoate (0.28 g, 822.6441 μmol, 80.5345% yield) obtained as a yellow solid. LCMS: (ESI, m/z): [M+H]⁺=341.078. ¹H NMR (400 MHz, DMSO-d₆) δ 9.51 (s, 1H), 8.25 (s, 1H), 7.46 (s, 1H), 4.09-4.00 (m, 2H), 3.96-3.88 (m, 3H), 3.52-3.41 (m, 1H), 3.24-3.16 (m, 1H), 2.94 (s, 1H), 1.22-1.10 (m, 6H).

Step e: Ethyl (2S)-4-(4-fluoro-5-hydroxy-6-methoxy-benzothiophen-2-yl)-2-methyl-4-oxo-butanoate (0.16 g, 470.0822 μmol) was added to a mixture of methyl (S)-4-(5-(3-bromopropoxy)-6-methoxyisoindolin-2-yl)-2-methyl-4-oxobutanoate (0.20 g, 482.7536 μmol) and Potassium carbonate (0.15 g, 1.0853 mmol) in N,N-Dimethylformamide (5 mL) at 25° C. The reaction mixture was stirred overnight at 25° C. The reaction mixture was purified on C18 column ACN/H₂O (0-45%). The pure fractions was concentrated and dried under vacuo. There was ethyl (S)-4-(4-fluoro-6-methoxy-5-(3-((6-methoxy-2-((S)-4-methoxy-3-methyl-4-oxobutanoyl)isoindolin-5-yl)oxy)propoxy)benzo[b]thiophen-2-yl)-2-methyl-4-oxobutanoate (0.268 g, 397.7772 μmol, 82.3976% yield) obtained as a colorless oil. LCMS: (ESI, m/z): [M+H]⁺=674.236. ¹H NMR (400 MHz, DMSO-d₆) δ 8.31 (s, 1H), 7.57 (s, 0.5H), 7.54 (s, 0.5H), 7.03-6.83 (m, 2H), 4.72 (s, 2H), 4.52 (s, 2H), 4.31-4.09 (m, 4H), 4.09-3.96 (m, 2H), 3.87 (s, 3H), 3.72 (s, 3H), 3.59 (s, 3H), 3.51-3.41 (m, 1H), 3.24-3.15 (m, 1H), 3.02-2.65 (m, 4H), 2.12 (s, 2H), 1.32-0.96 (m, 9H).

Step f: LiOH (0.030 g, 1.2527 mmol) was added to a solution of ethyl (2S)-4-[4-fluoro-6-methoxy-5-[3-[6-methoxy-2-[(3S)-4-methoxy-3-methyl-4-oxo-butanoyl]isoindolin-5-yl]oxypropoxy]benzothiophen-2-yl]-2-methyl-4-oxo-butanoate (0.258 g, 382.9348 μmol) in Tetrahydrofuran (10 mL) and Water (2 mL) at 25° C. The reaction mixture was stirred overnight at 25° C. The reaction mixture was purified by HPLC. The pure fractions was concentrated and dried under vacuo to obtain the free acid. NaHCO₃ (0.016 g, 190.4612 μmol) was added to the free acid in Acetonitrile (10 mL) and Water (10 mL) at 25° C. The mixture was stirred at 25° C. for 1 h. The solvent was removed by lyophilization. There was sodium (S)-4-(5-(3-((2-((S)-3-carboxylatobutanoyl)-4-fluoro-6-methoxybenzo[b]thiophen-5-yl)oxy)propoxy)-6-methoxyisoindolin-2-yl)-2-methyl-4-oxobutanoate (0.063 g, 93.2466 μmol, 24.3505% yield) obtained as a white solid. LCMS: (ESI, m/z): [M+H]⁺=632.289. ¹H NMR (400 MHz, DMSO-d₆) δ 8.17-8.05 (m, 1H), 7.51 (s, 1H), 6.96-6.80 (m, 2H), 4.92-4.72 (m, 2H), 4.74-4.58 (m, 2H), 4.48 (s, 2H), 4.29-4.08 (m, 5H), 3.87 (s, 3H), 3.74-3.65 (m, 3H), 2.80-2.57 (m, 4H), 2.10 (s, 3H), 1.03 (d, J=6.9 Hz, 6H).

### Example 7

### Sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate

### Step a: 5-bromo-2-fluoro-4-(methoxymethoxy)benzaldehyde

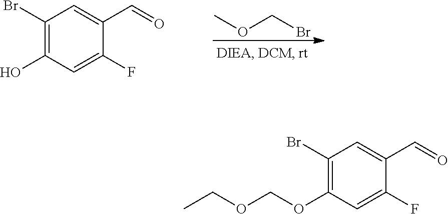

Bromomethyl methyl ether (0.71 g, 5.6816 mmol) was added to a solution of DIEA (0.83 g, 6.4220 mmol) and 5-bromo-2-fluoro-4-hydroxybenzaldehyde (0.94 g, 4.2921 mmol) in DCM (20 mL) at 0° C. The reaction mixture was stirred for 1 h at 25° C. The reaction mixture was quenched with adding of water (10 mL) at 20° C. The resulted mixture was washed with water (20 mL) and brine (20 mL). The organics dried over Na₂SO₄, filtered and evaporated under reduced pressure. There was a crude 5-bromo-2-fluoro-4-(methoxymethoxy)benzaldehyde (1.16 g, 4.4096 mmol, 102.7388% yield) obtained as a white solid. LCMS: (ESI, m/z): [M+H]⁺=262.950. ¹H NMR (400 MHz, DMSO-d₆) δ 10.03 (s, 1H), 8.02 (d, J=7.5 Hz, 1H), 7.28 (d, J=12.6 Hz, 1H), 5.45 (s, 2H), 3.43 (s, 3H).

### Step b: Methyl 5-bromo-6-(methoxymethoxy)benzothiophene-2-carboxylate

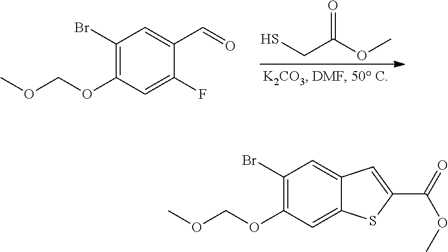

Methyl thioglycolate (0.4 mL, 4.4732 mmol) was added to a solution of 5-bromo-2-fluoro-4-(methoxymethoxy)benzaldehyde (1.06 g, 4.0295 mmol) and Potassium carbonate (0.15 g, 1.0853 mmol) in DMF (10 mL) at 25° C. The reaction mixture was heated to 50° C. and stirred for 2 h. The reaction mixture was quenched with adding of water (100 mL) at 20° C. The precipitate was collected by filtration, washed with water (3×20 mL). The filtrate cake was dried under vacuo at 60° C. There was methyl 5-bromo-6-(methoxymethoxy)benzothiophene-2-carboxylate (1.12 g, 3.3818 mmol, 83.9267% yield) obtained as a white solid. LCMS: (ESI, m/z): [M+H]⁺=331.100.

### Step c: 5-bromo-6-(methoxymethoxy) benzothiophene-2-carboxylic Acid

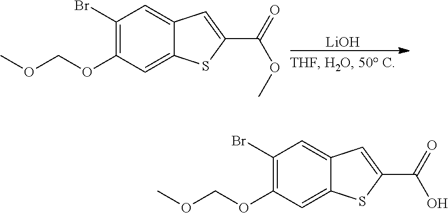

LiOH (0.18 g, 7.5162 mmol) was added to a solution of methyl 5-bromo-6-(methoxymethoxy) benzothiophene-2-carboxylate (1.10 g, 3.3214 mmol) in THF (40 mL) and Water (10 mL) at 25° C. The reaction mixture was stirred for 2 h at 50° C. The reaction mixture was evaporated under reduced pressure. The residue was diluted with water and acidified pH=3 with HCl (aq., 1 M). The precipitate was filtered and filtrate cake was washed with water (20 mL). The filtrate cake was dried under vacuo at 60° C. There was 5-bromo-6-(methoxymethoxy) benzothiophene-2-carboxylic acid (1.04 g, 3.2791 mmol, 98.7268% yield) obtained as a white solid. LCMS: (ESI, m/z): [M−H]⁻=314.900. ¹H NMR (400 MHz, DMSO-d₆) δ 13.49 (s, 1H), 8.30 (s, 1H), 7.99 (s, 1H), 7.86 (s, 1H), 5.39 (s, 2H), 3.44 (s, 3H).

### Step d: Tert-butyl 3-(5-bromo-6-(methoxymethoxy)benzo[b]thiophen-2-yl)-3-oxopropanoate

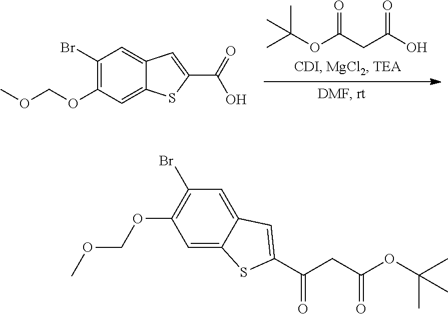

CDI (1.13 g, 6.9689 mmol) was added to a solution of 5-bromo-6-(methoxymethoxy) benzothiophene-2-carboxylic acid (1.01 g, 3.1846 mmol) in DMF (20 mL) at 25° C. The reaction mixture was stirred for 2 h at 25° C. under nitrogen atmosphere. Magnesium chloride (0.64 g, 6.7219 mmol), 3-tert-Butoxy-3-oxopropanoic acid (0.81 g, 5.0572 mmol) and TEA (1.06 g, 10.4754 mmol) were added to the reaction mixture. The reaction mixture was stirred overnight at 25° C. The reaction mixture was concentrated and diluted with EA (100 mL), washed with saturated aqueous NaHCO₃ solution (2×50 mL) and brine (50 mL). The organics dried over Na₂SO₄, filtered and evaporated under reduced pressure. The residue was purified on silica gel column EA/n-Hexane (0-20%). The pure fractions was concentrated and dried under vacuo. There was tert-butyl 3-(5-bromo-6-(methoxymethoxy)benzo[b]thiophen-2-yl)-3-oxopropanoate (1.12 g, 2.6969 mmol, 84.6855% yield) obtained as a yellow solid. LCMS: (ESI, m/z): [M−H]⁻=412.950. ¹H NMR (400 MHz, DMSO-d₆) δ 8.33 (s, 1H), 8.22 (s, 1H), 7.87 (s, 1H), 5.39 (d, J=10.0 Hz, 2H), 4.06 (d, J=8.4 Hz, 2H), 3.45 (s, 3H), 1.40 (s, 9H).

### Step e: 1-(tert-butyl) 4-ethyl 2-(5-bromo-6-(methoxymethoxy)benzo[b]thiophene-2-carbonyl)succinate

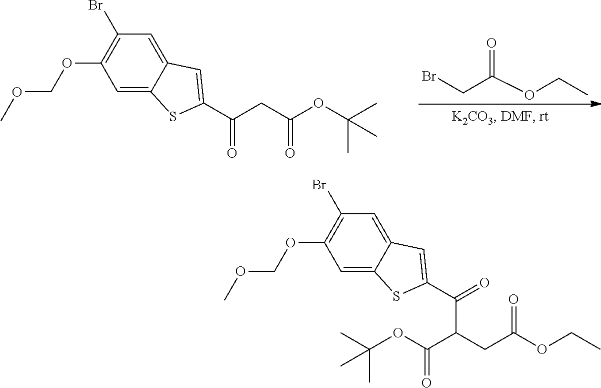

Ethyl 2-bromoacetate (0.356 g, 2.1317 mmol) was added to a solution of tert-butyl 3-(5-bromo-6-(methoxymethoxy)benzo[b]thiophen-2-yl)-3-oxopropanoate (0.891 g, 2.1454 mmol) and Potassium carbonate (0.587 g, 4.2473 mmol) in DMF (10 mL) at 25° C. The reaction mixture was stirred overnight at 25° C. under nitrogen atmosphere. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-50%). The pure fractions was concentrated and dried under vacuo. There was 1-(tert-butyl) 4-ethyl 2-(5-bromo-6-(methoxymethoxy)benzo[b]thiophene-2-carbonyl)succinate (0.771 g, 1.5377 mmol, 71.6743% yield) obtained as a yellow oil. LCMS: (ESI, m/z): [M+H]⁺=501.050.

### Step f: Ethyl 4-(5-bromo-6-hydroxy-benzothiophen-2-yl)-4-oxo-butanoate

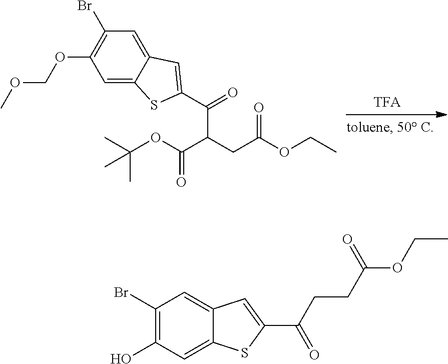 [SMILES: CCOC(=O)CC(C(=O)OC(C)(C)C)C(=O)c1cc2cc(Br)c(OCOC)cc2s1 ; CCOC(=O)CCC(=O)c1cc2cc(Br)c(O)cc2s1]

Trifluoroacetic acid (3 mL) was added to a solution of 1-(tert-butyl) 4-ethyl 2-(5-bromo-6-(methoxymethoxy)benzo[b]thiophene-2-carbonyl)succinate (0.750 g, 1.4959 mmol) in toluene (9 mL) at 25° C. The reaction mixture was heated to 50° C. and stirred for 2 h. The reaction mixture was evaporated under reduced pressure. The residue was purified on C18 column MeCN/water (0-50%). The pure fractions was concentrated and dried under vacuo. There was ethyl 4-(5-bromo-6-hydroxy-benzothiophen-2-yl)-4-oxo-butanoate (0.431 g, 1.2065 mmol, 80.6593% yield) obtained as a white solid. LCMS: (ESI, m/z): [M+H]⁺=357.000.

### Step g: Ethyl 4-(5-bromo-6-(methoxymethoxy)benzo[b]thiophen-2-yl)-4-oxobutanoate

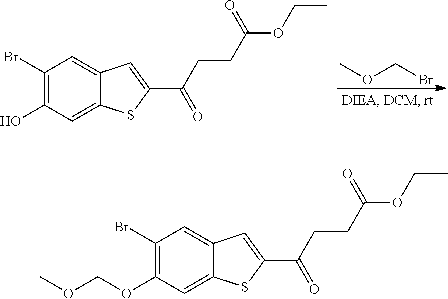

Bromomethyl methyl ether (0.198 g, 1.5845 mmol) was added to a solution of ethyl 4-(5-bromo-6-hydroxy-benzothiophen-2-yl)-4-oxo-butanoate (0.427 g, 1.1953 mmol) and DIEA (0.235 g, 1.8183 mmol) in DCM (10 mL) at 25° C. The reaction mixture was stirred for 1 h at 25° C. The reaction mixture was concentrated and diluted with DCM (100 mL), washed with water (50 mL) and brine (50 mL). The organics dried over Na₂SO₄, filtered and evaporated under reduced pressure. There was ethyl 4-(5-bromo-6-(methoxymethoxy)benzo[b]thiophen-2-yl)-4-oxobutanoate (0.503 g, 1.1282 mmol, 94.3798% yield) obtained as a yellow solid. LCMS: (ESI, m/z): [M+H]⁺=401.100.

### Step h: Ethyl 4-(6-(methoxymethoxy)-5-(4,4,5,5-tetramethyl-1,3,2-dioxaborolan-2-yl)benzo[b]thiophen-2-yl)-4-oxobutanoate

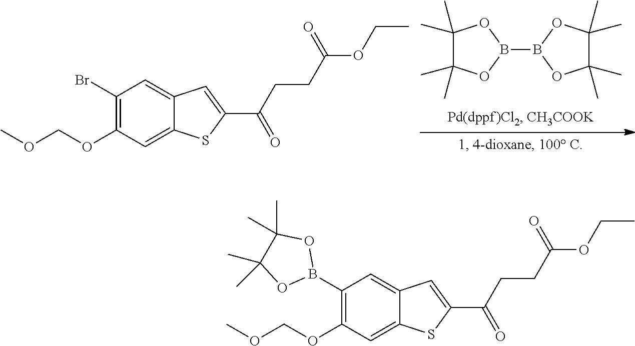

A mixture of [1,1′-Bis(diphenylphosphino)ferrocene]dichloropalladium(II) (0.110 g, 150.3341 μmol), ethyl 4-(5-bromo-6-(methoxymethoxy)benzo[b]thiophen-2-yl)-4-oxobutanoate (0.478 g, 1.1912 mmol), 4,4,4′,4′,5,5,5′,5′-Octamethyl-2,2′-bi(1,3,2-dioxaborolane) (0.491 g, 1.9335 mmol) and Potassium Acetate (0.389 g, 3.9636 mmol) dissolve in 1,4-Dioxane (10 mL) at 25° C. The reaction mixture was heated to 100° C. and stirred for 4.5 h under N₂ atmosphere. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-50%). The pure fractions was concentrated and dried under vacuo. There was ethyl 4-(6-(methoxymethoxy)-5-(4,4,5,5-tetramethyl-1,3,2-dioxaborolan-2-yl)benzo[b]thiophen-2-yl)-4-oxobutanoate (0.420 g, 936.7968 μmol, 78.6423% yield) obtained as a yellow oil. LCMS: (ESI, m/z): [M+H]⁺=449.200.

### Step i: Ethyl 4-(5-hydroxy-6-(methoxymethoxy)benzo[b]thiophen-2-yl)-4-oxobutanoate

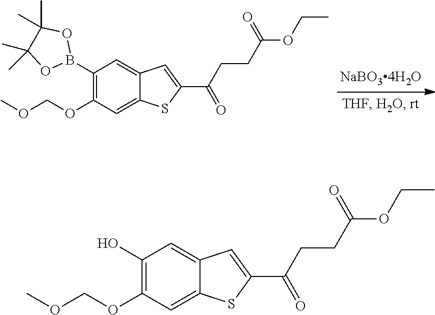

Sodium perborate tetrahydrate (0.200 g, 1.2999 mmol) was added to a solution of ethyl 4-(6-(methoxymethoxy)-5-(4,4,5,5-tetramethyl-1,3,2-dioxaborolan-2-yl)benzo[b]thiophen-2-yl)-4-oxobutanoate (0.407 g, 907.8013 μmol) in THF (5 mL) and Water (5 mL) at 25° C. The reaction mixture was stirred for 2 h at 25° C. The reaction mixture was evaporated under reduced pressure. The residue was purified on C18 column MeCN/water (0-50%). The pure fractions was concentrated and dried under vacuo. There was ethyl 4-(5-hydroxy-6-(methoxymethoxy)benzo[b]thiophen-2-yl)-4-oxobutanoate (0.250 g, 738.8257 μmol, 81.3863% yield) obtained as a yellow solid. LCMS: (ESI, m/z): [M+H]⁺=339.100.

### Step j: Ethyl 4-(5-methoxyisoindolin-2-yl)-4-oxo-butanoate

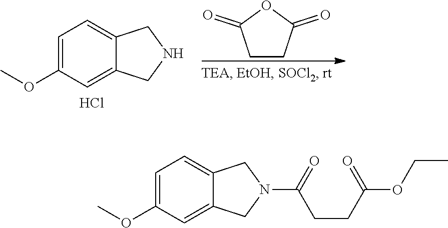 [SMILES: COc1ccc2c(c1)CNC2 ; O=C1CCC(=O)O1 ; Cl ; CCOC(=O)CCC(=O)N1Cc2ccc(OC)cc2C1]

Succinic anhydride (4.40 g, 43.9681 mmol) was added to a solution of 5-Methoxyisoindoline hydrochloride (4.89 g, 26.3399 mmol) and TEA (4.73 g, 46.7440 mmol) in Ethanol (200 mL) at 25° C. The reaction mixture was stirred for 1 h at 25° C. Thionyl chloride (20 mL) was added to the solution above at 0° C. The reaction mixture was stirred for 3 h at 25° C. The reaction mixture was evaporated under reduced pressure. The residue was diluted with EA (200 mL), washed with water (100 mL) and brine (50 mL). The organics dried over Na₂SO₄, filtered and evaporated under reduced pressure. There was ethyl 4-(5-methoxyisoindolin-2-yl)-4-oxo-butanoate (17.50 g, 31.5526 mmol, 119.7901% yield) obtained as a yellow solid. LCMS: (ESI, m/z): [M+H]⁺=278.150. ¹H NMR (400 MHz, DMSO-d₆) δ 7.28-7.21 (m, 1H), 6.94 (s, 0.5H), 6.92 (s, 0.5H), 6.88 (s, 0.5H), 6.86 (s, 0.5H), 4.81 (s, 1H), 4.76 (s, 1H), 4.58 (s, 1H), 4.54 (s, 1H), 4.02-3.95 (m, 2H), 3.75 (s, 3H), 2.65-2.58 (m, 2H), 2.58-2.54 (m, 2H), 1.17 (t, J=3.5 Hz, 3H).

### Step k: Ethyl 4-(5-bromo-6-methoxy-isoindolin-2-yl)-4-oxo-butanoate

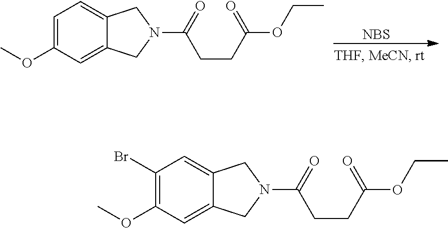

NBS (10.51 g, 59.0503 mmol) was added to a solution of ethyl 4-(5-methoxyisoindolin-2-yl)-4-oxo-butanoate (17.50 g, 31.5526 mmol) in THF (100 mL) and MeCN (100 mL) at 25° C. The reaction mixture was stirred overnight at 25° C. The reaction mixture was evaporated under reduced pressure. The residue was diluted with DCM (500 mL), washed with saturated aqueous NaHCO₃ solution (2×300 mL) and brine (200 mL). The organics dried over Na₂SO₄, filtered and evaporated under reduced pressure. The residue was purified on silica gel column MeOH/DCM (0-10%). The pure fractions was concentrated and dried under vacuo. There was ethyl 4-(5-bromo-6-methoxy-isoindolin-2-yl)-4-oxo-butanoate (7.26 g, 20.3812 mmol, 64.5943% yield) obtained as a yellow solid. LCMS: (ESI, m/z): [M+H]⁺=356.000. ¹H NMR (400 MHz, DMSO-d₆) δ 7.58 (s, 0.5H), 7.57 (s, 0.5H), 7.15 (s, 0.5H), 7.12 (s, 0.5H), 4.80 (s, 1H), 4.77 (s, 1H), 4.57 (s, 1H), 4.54 (s, 1H), 4.09-4.01 (m, 2H), 3.84 (s, 3H), 2.65-2.59 (m, 2H), 2.56-2.53 (m, 2H), 1.18 (t, J=7.1 Hz, 3H).

### Step l: Ethyl 4-(5-methoxy-6-(4,4,5,5-tetramethyl-1,3,2-dioxaborolan-2-yl)isoindolin-2-yl)-4-oxobutanoate

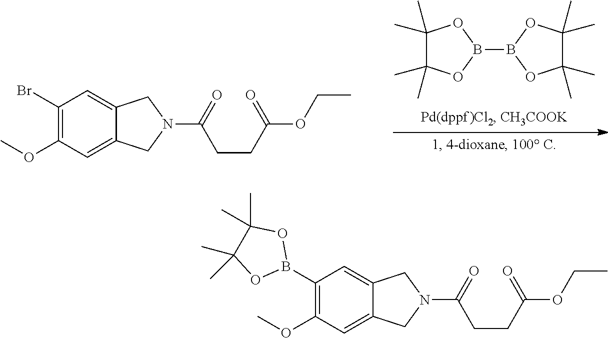

A mixture of Potassium Acetate (3.74 g, 38.1080 mmol), [1,1′-Bis(diphenylphosphino)ferrocene]dichloropalladium(II) (0.72 g, 984.0050 μmol), ethyl 4-(5-bromo-6-methoxy-isoindolin-2-yl)-4-oxo-butanoate (3.64 g, 10.2187 mmol) and 4,4,4′,4′,5,5,5′,5′-Octamethyl-2,2′-bi(1,3,2-dioxaborolane) (3.84 g, 15.1218 mmol) dissolve in 1,4-Dioxane (100 mL) at 25° C. The reaction mixture was heated to 100° C. and stirred overnight under N₂ atmosphere. The reaction mixture was diluted with EA (400 mL). The precipitate was filtrated by celite, the filtrate cake was washed with EA (1×100 mL). The combined filtrate was evaporated under reduced pressure. The residue was purified on silica gel column EA/n-Hexane (0-100%). The pure fractions was concentrated and dried under vacuo. There was ethyl 4-(5-methoxy-6-(4,4,5,5-tetramethyl-1,3,2-dioxaborolan-2-yl)isoindolin-2-yl)-4-oxobutanoate (2.70 g, 6.6952 mmol, 65.5190% yield) obtained as a yellow solid. LCMS: (ESI, m/z): [M+H]⁺=404.200. ¹H NMR (400 MHz, DMSO-d₆) δ 7.49 (s, 0.5H), 7.48 (s, 0.5H), 6.98 (s, 0.5H), 6.95 (s, 0.5H), 4.83 (s, 1H), 4.74 (s, 1H), 4.60 (s, 1H), 4.53 (s, 1H), 4.09-4.01 (m, 2H), 3.74 (t, J=6.2 Hz, 3H), 2.65-2.58 (m, 2H), 2.58-2.52 (m, 2H), 1.27 (s, 12H), 1.21-1.13 (m, 3H).

### Step m: Ethyl 4-(5-hydroxy-6-methoxy-isoindolin-2-yl)-4-oxo-butanoate

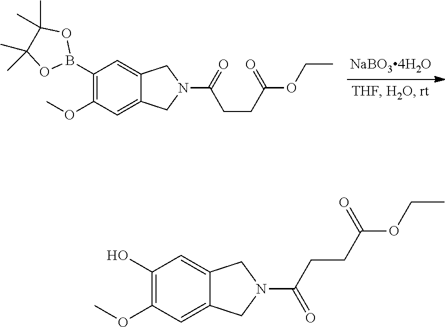

Sodium perborate tetrahydrate (1.00 g, 6.4994 mmol) was added to a solution of ethyl 4-(5-methoxy-6-(4,4,5,5-tetramethyl-1,3,2-dioxaborolan-2-yl)isoindolin-2-yl)-4-oxobutanoate (2.45 g, 6.0752 mmol) in THF (20 mL) and Water (20 mL) at 25° C. The reaction mixture was stirred for 1 h at 20° C. The reaction mixture was evaporated under reduced pressure. The reaction mixture was concentrated and diluted with DCM (500 mL), washed with saturated aqueous NaHCO₃ solution (2×300 mL) and brine (200 mL). The organics dried over Na₂SO₄, filtered and evaporated under reduced pressure. The residue was purified on silica gel column MeOH/DCM (0-10%). The pure fractions was concentrated and dried under vacuo. There was ethyl 4-(5-hydroxy-6-methoxy-isoindolin-2-yl)-4-oxo-butanoate (1.37 g, 4.6708 mmol, 76.8818% yield) obtained as a yellow solid. LCMS: (ESI, m/z): [M+H]⁺=294.100. ¹H NMR (400 MHz, DMSO-d₆) δ 8.97 (s, 0.5H), 8.96 (s, 0.5H), 6.91 (s, 0.5H), 6.89 (s, 0.5H), 6.73 (s, 0.5H), 6.73 (s, 0.5H), 4.71 (s, 1H), 4.69 (s, 1H), 4.50 (s, 1H), 4.47 (s, 1H), 4.08-4.00 (m, 2H), 3.75 (s, 3H), 2.64-2.58 (m, 2H), 2.56-2.53 (m, 2H), 1.18 (t, J=7.1 Hz, 3H).

### Step n: Ethyl 4-(5-(3-bromopropoxy)-6-methoxyisoindolin-2-yl)-4-oxobutanoate

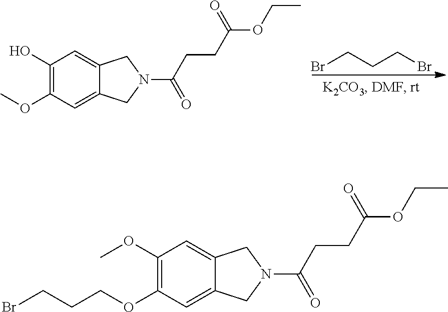 [SMILES: CCOC(=O)CCC(=O)N1Cc2cc(O)c(OC)cc2C1 ; BrCCCBr ; CCOC(=O)CCC(=O)N1Cc2cc(OC)c(OCCCBr)cc2C1]

1,3-Dibromopropane (0.73 g, 3.6159 mmol) was added to a solution of ethyl 4-(5-hydroxy-6-methoxy-isoindolin-2-yl)-4-oxo-butanoate (0.72 g, 2.4547 mmol) and Potassium carbonate (0.86 g, 6.2226 mmol) in DMF (10 mL). The reaction mixture was stirred overnight at 50° C. The reaction mixture was diluted with EA (100 mL), and washed sequentially with water (2×100 mL) and saturated brine (100 mL). The organic layer was dried over Na₂SO₄, filtered and evaporated to afford crude product. The crude product was purified by flash silica chromatography, elution gradient 0 to 100% Ethyl acetate in heptane. There was ethyl 4-(5-(3-bromopropoxy)-6-methoxyisoindolin-2-yl)-4-oxobutanoate (0.43 g, 1.0379 mmol, 42.2829% yield) obtained as a white solid. LCMS: (ESI, m/z): [M+H]⁺=414.050. ¹H NMR (400 MHz, DMSO-d₆) δ 7.01-6.93 (m, 2H), 4.75 (s, 2H), 4.53 (s, 2H), 4.10-4.00 (m, 4H), 3.76 (s, 3H), 3.71-3.62 (m, 2H), 2.65-2.58 (m, 2H), 2.58-2.53 (m, 2H), 2.28-2.18 (m, 2H), 1.18 (t, J=7.1 Hz, 3H).

### Step o: Ethyl 4-(5-(3-((2-(4-ethoxy-4-oxobutanoyl)-6-(methoxymethoxy)benzo[b]thiophen-5-yl)oxy)propoxy)-6-methoxy isoindolin-2-yl)-4-oxobutanoate

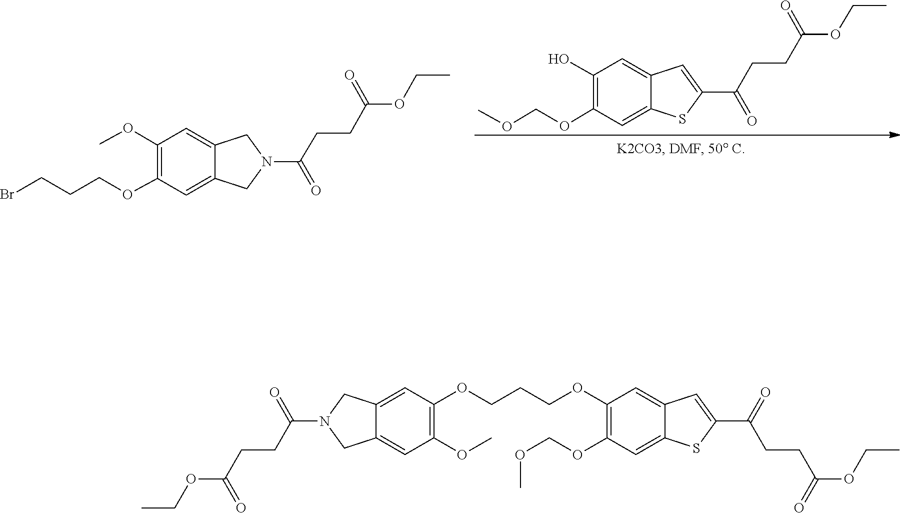

Ethyl 4-[5-hydroxy-6-(methoxymethoxy) benzothiophen-2-yl]-4-oxo-butanoate (0.101 g, 298.4856 μmol) was added to a solution of ethyl 4-(5-(3-bromopropoxy)-6-methoxyisoindolin-2-yl)-4-oxobutanoate (0.127 g, 306.5486 μmol) and Potassium carbonate (0.142 g, 1.0275 mmol) in DMF (5 mL) at 25° C. The reaction mixture was heated to 50° C. and stirred overnight. The reaction mixture was concentrated and diluted with EA (200 mL), washed with water (100 mL) and brine (100 mL). The organics dried over Na₂SO₄, filtered and evaporated under reduced pressure. The residue was purified on silica gel column EA/n-Hexane (0-100%). The pure fractions was concentrated and dried under vacuo. There was ethyl 4-(5-(3-((2-(4-ethoxy-4-oxobutanoyl)-6-(methoxymethoxy)benzo[b]thiophen-5-yl)oxy)propoxy)-6-methoxyisoindolin-2-yl)-4-oxobutanoate (0.055 g, 81.8754 μmol, 27.4303% yield) obtained as a yellow solid. LCMS: (ESI, m/z): [M+H]⁺=672.250.

### Step p: Ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate

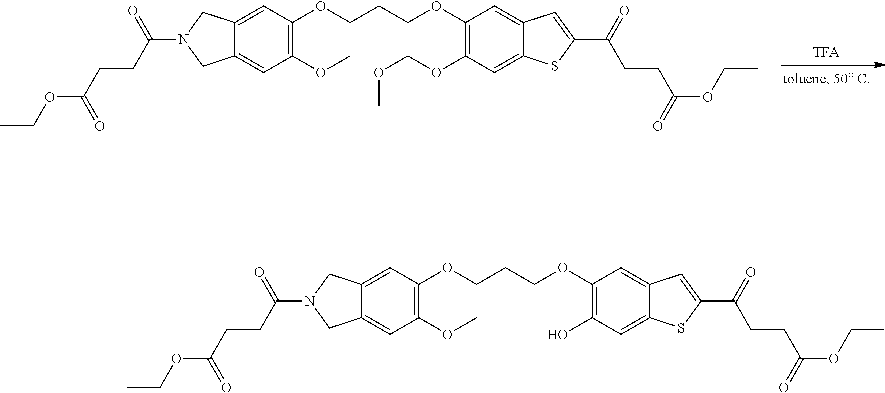

Trifluoroacetic acid (1.0 mL, 13.4622 mmol) was added to a solution of ethyl 4-(5-(3-((2-(4-ethoxy-4-oxobutanoyl)-6-(methoxymethoxy)benzo[b]thiophen-5-yl)oxy)propoxy)-6-methoxyisoindolin-2-yl)-4-oxobutanoate (0.054 g, 80.3867 μmol) in toluene (2 mL) at 25° C. The reaction mixture was heated to 50° C. and stirred for 30 min. The reaction mixture was evaporated under reduced pressure. The residue was diluted with water and neutralized pH=8 with saturated aqueous NaHCO₃ solution. The resulting mixture was extracted with EA (100 mL), washed with water (50 mL) and brine (50 mL). The organics dried over Na₂SO₄, filtered and evaporated under reduced pressure. There was ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate (0.057 g, 77.1865 μmol, 96.0190% yield) obtained as a yellow solid. LCMS: (ESI, m/z): [M+H]⁺=628.200.

### Step q: Sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate

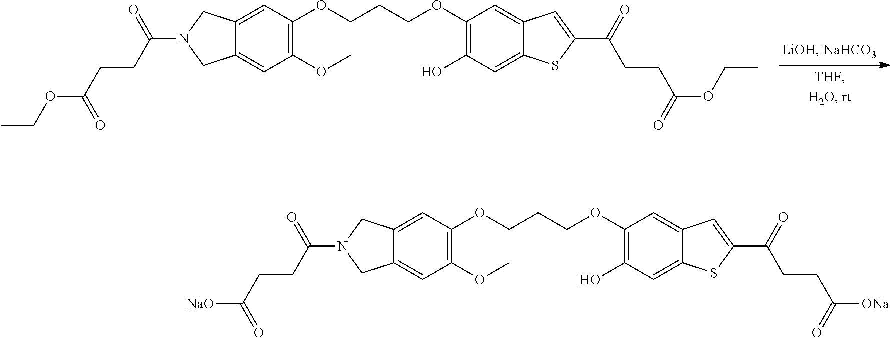

LiOH (0.023 g, 960.4022 μmol) was added to a solution of ethyl 4-(5-(3-((2-(4-ethoxy-4-oxobutanoyl)-6-hydroxybenzo[b]thiophen-5-yl)oxy)propoxy)-6-methoxyisoindolin-2-yl)-4-oxobutanoate (0.055 g, 87.6215 μmol) in THF (4 mL) and Water (1 mL) at 25° C. The reaction mixture was stirred overnight at 25° C. The reaction mixture was evaporated under reduced pressure. The residue was acidified pH=4 with HCl (1 M). The reaction mixture was evaporated under reduced pressure to obtain a crude product. The crude product was purified by Prep-HPLC with the following conditions: (CXTH LC6000, HPLC-P1): Column, Daisogel-C18, 50 mm*250 mm, 10 um; mobile phase, Water (0.1% TFA) and MeCN-(15-40-60% B (2-30-60 min); Detector, uv 216 nm). The solvent was removed by lyophilization. NaHCO₃ (0.004 g, 47.6153 mol) was added to the lyophilized product in water, and stirred for 30 min at 25° C. The solvent was removed by lyophilization. There was sodium 4-(5-(3-((2-(3-carboxylatopropanoyl)-6-hydroxybenzo[b]thiophen-5-yl)oxy)propoxy)-6-methoxyisoindolin-2-yl)-4-oxobutanoate (0.012 g, 19.4945 μmol, 22.2485% yield) obtained as a light yellow solid. LCMS: (ESI, m/z): [M+H]⁺=572.150. ¹H NMR (400 MHz, DMSO-d₆) δ 8.00 (s, 0.5H), 7.97 (s, 0.5H), 7.39 (s, 0.5H), 7.37 (s, 0.5H), 7.30 (s, 0.5H), 7.28 (s, 0.5H), 6.99-6.87 (m, 2H), 4.74 (s, 1H), 4.70 (s, 1H), 4.49 (s, 1H), 4.47 (s, 1H), 4.20-4.08 (m, 4H), 3.73 (s, 3H), 3.07 (t, J=6.8 Hz, 2H), 2.47-2.40 (m, 2H), 2.36-2.16 (m, 6H).

### Example 8

### Sodium 4-(5-(3-((2-(3-carboxylatopropanoyl)-6-methoxyisoindolin-5-yl)oxy)propoxy)-6-methoxythieno[3,2-b]pyridin-2-yl)-4-oxobutanoate

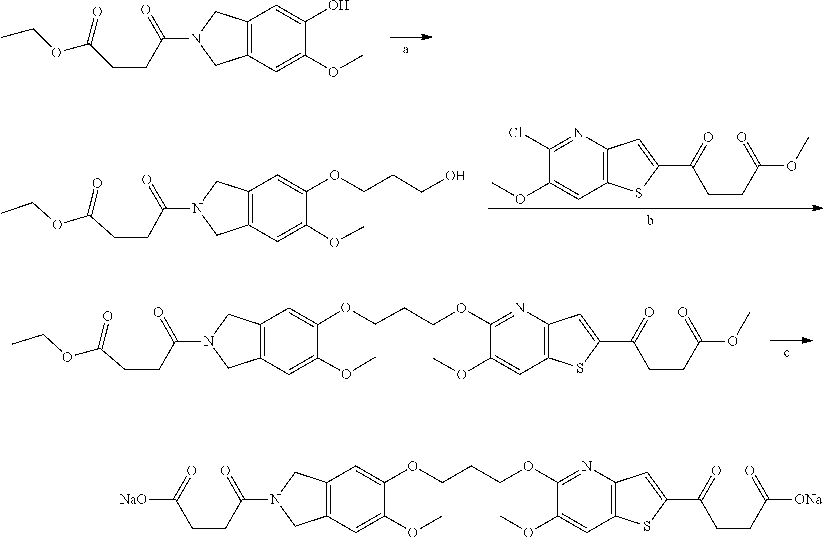

Step a: (3-Bromopropoxy)-tert-butyldimethylsilane (518 mg, 2.0456 mmol) and K₂CO₃ (320 mg, 2.3154 mmol) was added to a solution of ethyl 4-(5-hydroxy-6-methoxyisoindolin-2-yl)-4-oxobutanoate (200 mg, 681.8624 μmol) in N,N-Dimethylformamide (8 mL) at 25° C. The reaction mixture was stirred for 2 h at 25° C. Tetrabutylammonium fluoride (178 mg, 681.8624 μmol) was added to the mixture. The reaction mixture was stirred for 2 h at 25° C. The reaction mixture was concentrated and diluted with EA (100 mL), washed with NaHCO₃ (aq)(100 mL) and brine (50 mL). The organics dried over Na₂SO₄, filtered and evaporated under reduced pressure. The residue was purified on silica gel column MeOH/DCM (0-10%). The pure fractions was concentrated and dried under vacuo. There was ethyl 4-(5-(3-hydroxypropoxy)-6-methoxyisoindolin-2-yl)-4-oxobutanoate (106 mg, 301.6564 μmol, 44.2401% yield) obtained as a white solid. LCMS: (ESI, m/z): [M+H]⁺=352.168. ¹H NMR (400 MHz, DMSO-d₆) δ 8.31 (s, 1H), 6.95 (d, J=9.4 Hz, 2H), 4.74 (s, 2H), 4.53 (s, 2H), 4.00 (t, J=6.1 Hz, 2H), 3.75 (s, 3H), 3.20-3.13 (m, 2H), 2.63-2.51 (m, 4H), 1.57 (s, 2H), 1.38-1.21 (m, 2H), 0.94 (t, J=7.1 Hz, 3H).

Step b: K₃PO₄ (151 mg, 711.3706 μmol) and RockPhos Palladacycle Gen.3 (11 mg, 13.1041 μmol) was added to a solution of ethyl 4-(5-(3-hydroxypropoxy)-6-methoxyisoindolin-2-yl)-4-oxobutanoate (50 mg, 142.2907 μmol) and methyl 4-(5-chloro-6-methoxy-thieno[3,2-b]pyridin-2-yl)-4-oxo-butanoate (66 mg, 210.3544 μmol) in Toluene (10 mL) at 25° C. The reaction mixture was stirred at 130° C. for 16 h under N₂ atmosphere. The reaction mixture was purified on C-18 column MeCN/water (0-100%). The pure fractions was concentrated and dried under vacuo. There was ethyl 4-(5-methoxy-6-(3-((6-methoxy-2-(4-methoxy-4-oxobutanoyl)thieno[3,2-b]pyridin-5-yl)oxy)propoxy)isoindolin-2-yl)-4-oxobutanoate (4.1 mg, 6.5215 μmol, 4.5832% yield) obtained as a yellow oil. LCMS: (ESI, m/z): [M+H]⁺=629.209.

Step c: LiOH (0.006 g, 250.5397 μmol) was added to a solution of ethyl 4-(5-methoxy-6-(3-((6-methoxy-2-(4-methoxy-4-oxobutanoyl)thieno[3,2-b]pyridin-5-yl)oxy)propoxy)isoindolin-2-yl)-4-oxobutanoate (0.013 g, 20.6780 μmol) in Tetrahydrofuran (4 mL) and Water (1 mL) at 25° C. The reaction mixture was stirred for 2 h at 25° C. The reaction mixture was evaporated under reduced pressure. The residue was concentrated and diluted with water (2 mL). The resulting mixture was adjusted to pH=7 with HCl (1 M) at 25° C. and evaporated under reduced pressure. The residue was purified by HPLC. The pure fractions was concentrated and dried by lyophilization to obtain the free acid. NaHCO₃ (0.001 g, 11.9038 μmol) was added to a mixture of the free acid in Acetonitrile (2 mL) and Water (1 mL) at 25° C. The mixture was stirred at 25° C. for 1 h and dried by lyophilization. There was sodium 4-(5-(3-((2-(3-carboxylatopropanoyl)-6-methoxyisoindolin-5-yl)oxy)propoxy)-6-methoxythieno[3,2-b]pyridin-2-yl)-4-oxobutanoate (0.0043 g, 6.8192 μmol, 32.9781% yield) obtained as a white solid. LCMS: (ESI, m/z): [M+H]⁺=587.162. ¹H NMR (400 MHz, DMSO-d₆) δ 8.07 (s, 0.5H), 8.03 (s, 0.5H), 7.96 (s, 1H), 7.02-6.93 (m, 2H), 4.80-4.73 (m, 2H), 4.50 (s, 4H), 4.13 (s, 2H), 3.88 (s, 3H), 3.75-3.73 (m, 3H), 3.08 (s, 2H), 2.43 (s, 2H), 2.24 (s, 4H), 2.15 (s, 2H).

### Example 9

### 4-(5-(3-((6-bromo-2-(3-carboxypropanoyl)benzo[b]thiophen-5-yl)oxy)propoxy)-6-methoxyisoindolin-2-yl)-4-oxobutanoic Acid

### Step a: Ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate

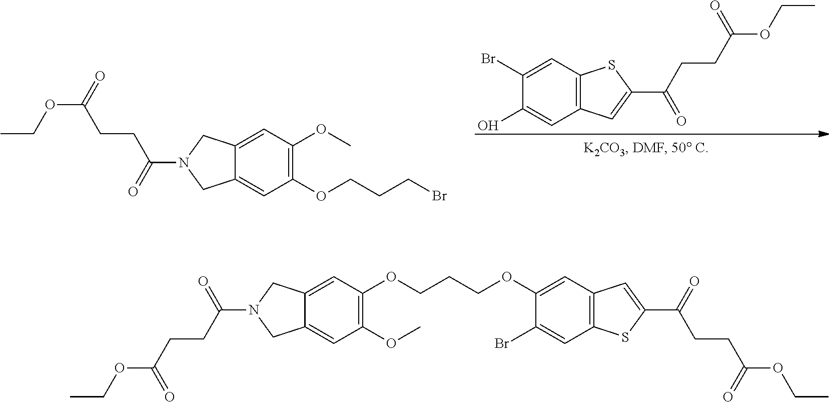 [SMILES: CCOC(=O)CCC(=O)N1Cc2cc(OC)c(OCCCBr)cc2C1 ; CCOC(=O)CCC(=O)c1cc2cc(O)c(Br)cc2s1 ; CCOC(=O)CCC(=O)c1cc2cc(OCCCOc3cc4c(cc3OC)CN(C(=O)CCC(=O)OCC)C4)c(Br)cc2s1]

Potassium carbonate (0.080 g, 578.8482 μmol) was added to a solution of ethyl 4-(5-(3-bromopropoxy)-6-methoxyisoindolin-2-yl)-4-oxobutanoate (0.075 g, 181.0327 μmol) and ethyl 4-(6-bromo-5-hydroxybenzo[b]thiophen-2-yl)-4-oxobutanoate (0.071 g, 198.7576 μmol) in DMF (2 mL) at 25° C. under N₂ atmosphere. The reaction mixture was heated to 50° C. and stirred overnight. The reaction mixture was purified on C18 column ACN/H₂O (0.1% FA)(0-60%). The pure fractions was concentrated and dried by lyophilization. There was ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate (0.080 g, 115.8418 μmol, 63.9894% yield) obtained as a white solid. LCMS: (ESI, m/z): [M+H]⁺=690.13. ¹H NMR (400 MHz, DMSO-d₆) δ 8.39 (s, 1H), 8.26 (s, 1H), 7.69 (s, 1H), 7.05-6.92 (m, 2H), 4.73 (s, 1H), 4.72 (s, 1H), 4.51 (d, J=3.6 Hz, 2H), 4.29 (s, 2H), 4.19 (t, J=6.4 Hz, 2H), 4.11-4.00 (m, 4H), 3.73 (d, J=2.0 Hz, 3H), 3.40-3.34 (m, 2H), 2.68 (t, J=6.3 Hz, 2H), 2.63-2.53 (m, 4H), 2.26 (t, J=6.3 Hz, 2H), 1.18 (t, J=7.1 Hz, 6H).

### Step b: 4-(5-(3-((6-bromo-2-(3-carboxypropanoyl)benzo[b]thiophen-5-yl)oxy)propoxy)-6-methoxyisoindolin-2-yl)-4-oxobutanoic Acid

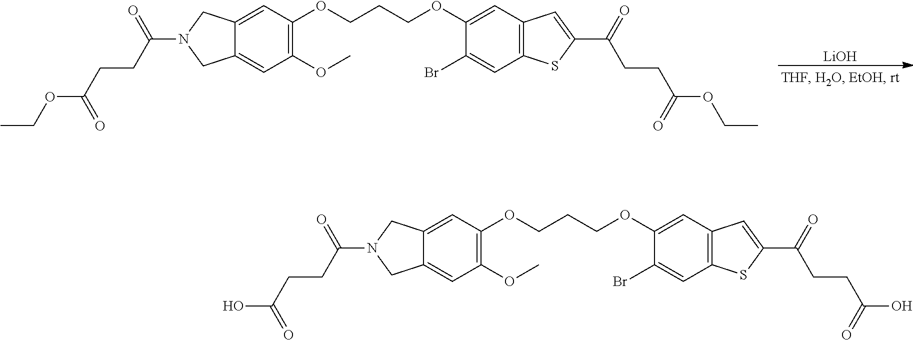 [SMILES: CCOC(=O)CCC(=O)c1cc2cc(OCCCOc3cc4c(cc3OC)CN(C(=O)CCC(=O)OCC)C4)c(Br)cc2s1 ; "LiOH" ; COc1cc2c(cc1OCCCOc1cc3cc(C(=O)CCC(=O)O)sc3cc1Br)CN(C(=O)CCC(=O)O)C2]

LiOH (0.050 g, 2.0878 mmol) was added to a solution of ethyl 4-(5-(3-((6-bromo-2-(4-ethoxy-4-oxobutanoyl)benzo[b]thiophen-5-yl)oxy)propoxy)-6-methoxyisoindolin-2-yl)-4-oxobutanoate (0.044 g, 63.7130 μmol) in THF (2 mL), EtOH (1 mL) and Water (2 mL) at 25° C. The reaction mixture was stirred for 2 h at 25° C. The reaction mixture was adjusted to pH=3 with HCl (1 mol/L). The reaction mixture was purified on C18 column ACN/H₂O (0.1% FA)(0-55%). The pure fractions was concentrated and dried by lyophilization. There was 4-(5-(3-((6-bromo-2-(3-carboxypropanoyl)benzo[b]thiophen-5-yl)oxy)propoxy)-6-methoxyisoindolin-2-yl)-4-oxobutanoic acid (0.012 g, 18.9128 μmol, 29.6844% yield) obtained as a white solid. LCMS: (ESI, m/z): [M+H]⁺=634.07. ¹H NMR (400 MHz, DMSO-d₆) δ 8.37 (s, 1H), 8.24 (d, J=3.1 Hz, 1H), 7.68 (d, J=3.8 Hz, 1H), 7.04-6.92 (m, 2H), 4.73 (s, 1H), 4.71 (s, 1H), 4.51 (d, J=4.7 Hz, 2H), 4.29 (s, 2H), 4.23-4.15 (m, 2H), 3.73 (s, 3H), 3.33-3.25 (m, 2H), 2.63-2.54 (m, 4H), 2.53-2.50 (m, 2H), 2.31-2.20 (m, 2H).

### Example 10

### 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic Acid

### Step a: Ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate

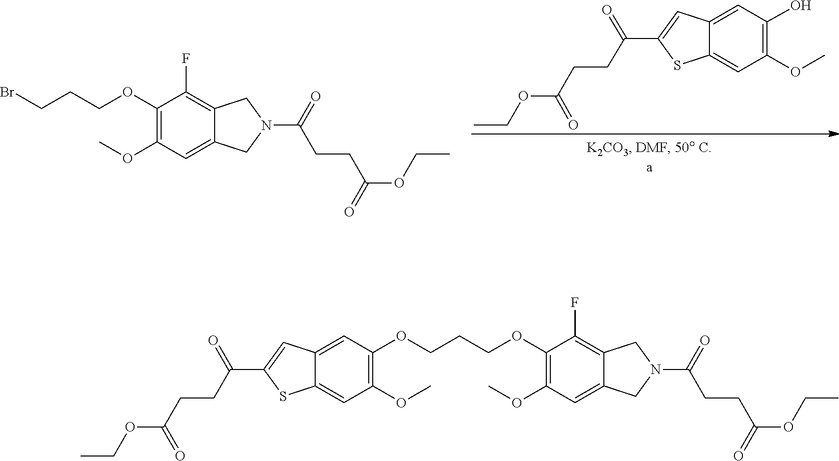

Potassium carbonate (0.099 g, 716.3246 μmol) was added to a solution of ethyl 4-(5-hydroxy-6-methoxybenzo[b]thiophen-2-yl)-4-oxobutanoate (0.062 g, 201.0709 μmol) and ethyl 4-(5-(3-bromopropoxy)-4-fluoro-6-methoxyisoindolin-2-yl)-4-oxobutanoate (0.069 g, 159.6186 μmol) in DMF (3 mL) at 25° C. under N₂ atmosphere. The reaction mixture was stirred overnight at 50° C. The mixture was purified on C18 column ACN/H₂O (0.1% FA)(0-50%). The pure fractions was concentrated and dried further in lyophilization. There was ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate (0.071 g, 107.6218 μmol, 67.4244% yield) obtained as a white solid. LCMS: (ESI, m/z): [M+H]⁺=660.220. ¹H NMR (400 MHz, DMSO-d₆) δ 8.22 (s, 1H), 7.59 (s, 1H), 7.50 (d, J=3.4 Hz, 1H), 6.92 (s, 0.5H), 6.88 (s, 0.5H), 4.80 (s, 1H), 4.78 (s, 1H), 4.57 (s, 2H), 4.27-4.20 (m, 2H), 4.17 (t, J=6.0 Hz, 2H), 4.10-4.02 (m, 4H), 3.85 (s, 3H), 3.76 (s, 3H), 3.31-3.28 (m, 2H), 2.69-2.53 (m, 6H), 2.20-2.09 (m, 2H), 1.18 (t, J=7.1 Hz, 6H).

### Step b: 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic Acid

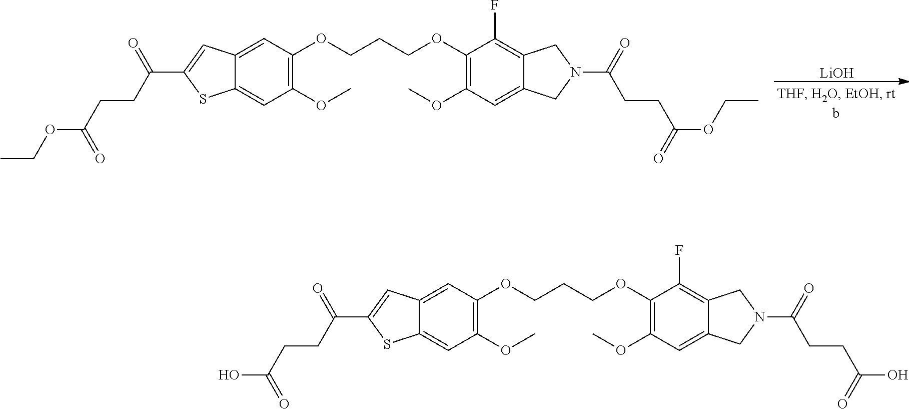 [SMILES: CCOC(=O)CCC(=O)c1cc2cc(OCCCOc3c(OC)cc4c(c3F)CN(C(=O)CCC(=O)OCC)C4)c(OC)cc2s1 ; "LiOH" ; COc1cc2sc(C(=O)CCC(=O)O)cc2cc1OCCCOc1c(OC)cc2c(c1F)CN(C(=O)CCC(=O)O)C2]

LiOH (0.044 g, 1.8373 mmol) was added to a mixture of ethyl 4-(5-(3-((2-(4-ethoxy-4-oxobutanoyl)-4-fluoro-6-methoxyisoindolin-5-yl)oxy)propoxy)-6-methoxybenzo[b]thiophen-2-yl)-4-oxobutanoate (0.100 g, 155.1150 μmol) in THF (2 mL), EtOH (2 mL) and Water (2 mL) at 25° C. The reaction mixture was stirred for 2 h at 25° C. The reaction mixture was adjusted to pH=3 with HCl (1 mol/L), The mixture was evaporated under reduced pressure. The residue was purified on C18 column ACN/H₂O (0.1% FA)(0-40%). The pure fractions was concentrated and dried under vacuo. There was 4-(5-(3-((2-(3-carboxypropanoyl)-4-fluoro-6-methoxyisoindolin-5-yl)oxy)propoxy)-6-methoxybenzo[b]thiophen-2-yl)-4-oxobutanoic acid (0.026 g, 43.0741 μmol, 66.0854% yield) obtained as a white solid. LCMS: (ESI, m/z): [M+H]⁺=604.160. ¹H NMR (400 MHz, DMSO-d₆) δ 8.21 (s, 1H), 7.59 (s, 1H), 7.50 (d, J=3.0 Hz, 1H), 6.91 (s, 0.5H), 6.88 (s, 0.5H), 4.79 (s, 1H), 4.77 (s, 1H), 4.56 (s, 2H), 4.27-4.20 (m, 2H), 4.17 (t, J=6.0 Hz, 2H), 3.85 (s, 3H), 3.76 (s, 3H), 3.26-3.24 (m, 2H), 2.63-2.54 (m, 4H), 2.48-2.44 (m, 2H), 2.19-2.10 (m, 2H).

### Example 11

### 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic Acid

### Step a: 3-methoxy-2-(3-tetrahydropyran-2-yloxypropoxy)-6,7-dihydro-5H-pyrrolo[3,4-b]pyridine

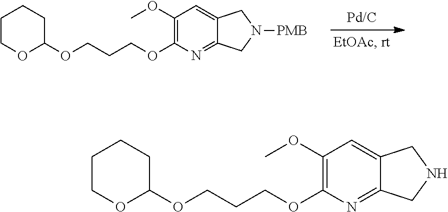

Pd/C (2.553 g, 2.3990 mmol) was added to a solution of 3-methoxy-6-(4-methoxybenzyl)-2-(3-((tetrahydro-2H-pyran-2-yl)oxy)propoxy)-6,7-dihydro-5H-pyrrolo[3,4-b]pyridine (2.464 g, 5.7500 mmol) in Ethyl acetate (60 mL) at 25° C. The reaction mixture was stirred overnight at 25° C. under H₂ atmosphere. The resulting mixture was filtered. The filter cake was washed with EA (3×100 mL). The filtrate was concentrated under reduced pressure. There was 3-methoxy-2-(3-tetrahydropyran-2-yloxypropoxy)-6,7-dihydro-5H-pyrrolo[3,4-b]pyridine (1.936 g, 4.3947 mmol, 76.4291% yield) obtained as a yellow oil. LCMS: (ESI, m/z): [M+H]⁺=309.174.

### Step b: 3-((3-methoxy-6,7-dihydro-5H-pyrrolo[3,4-b]pyridin-2-yl)oxy)propan-1-ol

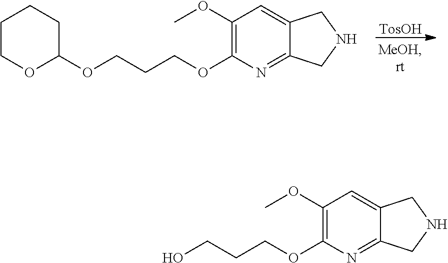 [SMILES: COc1cc2c(nc1OCCCOC1CCCCO1)CNC2 ; "TosOH" ; COc1cc2c(nc1OCCCO)CNC2]

4-methylbenzenesulfonic acid (0.69 g, 2.5430 mmol) was added to a solution of 3-methoxy-2-(3-tetrahydropyran-2-yloxypropoxy)-6,7-dihydro-5H-pyrrolo[3,4-b]pyridine (1.91 g, 4.3357 mmol) in MeOH (40 mL) at 25° C. The reaction mixture was stirred for 2 h at 25° C. The reaction mixture was evaporated under reduced pressure. The residue was purified on C18 column MeCN/water (0-50%). The pure fractions was concentrated and dried under vacuo. There was 3-((3-methoxy-6,7-dihydro-5H-pyrrolo[3,4-b]pyridin-2-yl)oxy)propan-1-ol (0.814 g, 3.6298 mmol, 83.7191% yield) obtained as a yellow oil. LCMS: (ESI, m/z): [M+H]⁺=225.116.

### Step c: Ethyl 4-(2-(3-hydroxypropoxy)-3-methoxy-5,7-dihydro-6H-pyrrolo[3,4-b]pyridin-6-yl)-4-oxobutanoate

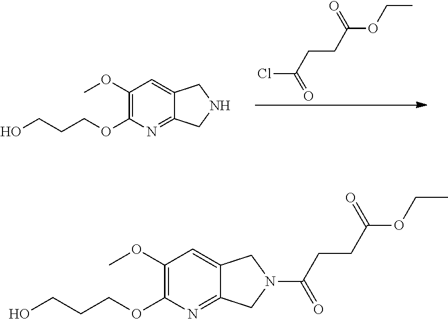

Ethyl Succinyl Chloride (0.232 g, 1.4096 mmol) was added to a solution of 3-((3-methoxy-6,7-dihydro-5H-pyrrolo[3,4-b]pyridin-2-yl)oxy)propan-1-ol (0.366 g, 1.6321 mmol) and Triethylamine (0.363 g, 3.5873 mmol) in Tetrahydrofuran (20 mL) at 0° C. under N₂ atmosphere. The reaction mixture was stirred at 25° C. for 1 h. The reaction mixture was purified on C-18 column MeCN/water (0-100%). The pure fractions was concentrated and dried under vacuo. There was ethyl 4-(2-(3-hydroxypropoxy)-3-methoxy-5,7-dihydro-6H-pyrrolo[3,4-b]pyridin-6-yl)-4-oxobutanoate (224 mg, 635.6750 μmol, 38.9491% yield) obtained as a white solid. LCMS: (ESI, m/z): [M+H]⁺=353.163. ¹H NMR (400 MHz, DMSO-d₆) δ 7.38-7.26 (m, 1H), 4.81-4.73 (m, 1H), 4.68 (s, 1H), 4.54 (s, 2H), 4.42 (s, 1H), 4.30 (t, J=6.3 Hz, 2H), 4.11-3.99 (m, 2H), 3.78 (s, 3H), 3.57-3.50 (m, 2H), 2.66-2.59 (m, 2H), 2.58-2.52 (m, 2H), 1.91-1.80 (m, 2H), 1.18 (t, J=7.1 Hz, 3H).

### Step d: Ethyl 4-(3-methoxy-2-(3-((6-methoxy-2-(4-methoxy-4-oxobutanoyl)thieno[3,2-b]pyridin-5-yl)oxy)propoxy)-5,7-dihydro-6H-pyrrolo[3,4-b]pyridin-6-yl)-4-oxobutanoate

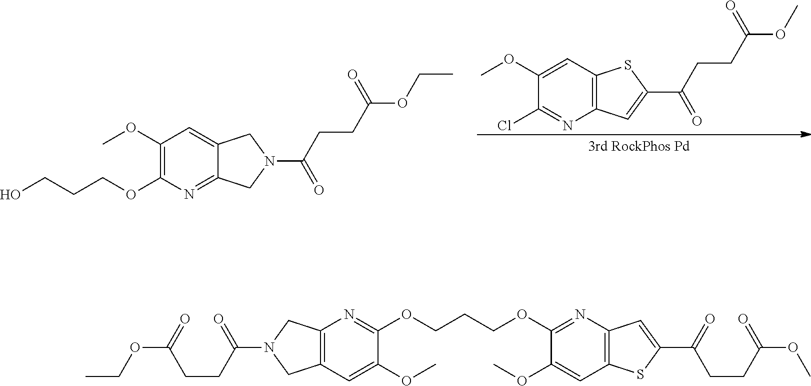

Potassium phosphate tribasic (0.212 g, 998.7454 μmol) and RockPhos Palladacycle Gen.3 (0.022 g, 26.2082 μmol) was added to a solution of ethyl 4-(2-(3-hydroxypropoxy)-3-methoxy-5,7-dihydro-6H-pyrrolo[3,4-b]pyridin-6-yl)-4-oxobutanoate (0.087 g, 246.8916 μmol) and methyl 4-(5-chloro-6-methoxy-thieno[3,2-b]pyridin-2-yl)-4-oxo-butanoate (0.120 g, 382.4626 μmol) in Toluene (20 mL) at 25° C. The reaction mixture was stirred overnight at 25° C. under N₂ atmosphere. The reaction mixture was evaporated under reduced pressure. The residue was purified on C-18 column MeCN/water (0-100%). The pure fractions was concentrated and dried under vacuo. The residue was purified by HPLC. The pure fractions was concentrated and lyophilization. There was ethyl 4-(3-methoxy-2-(3-((6-methoxy-2-(4-methoxy-4-oxobutanoyl)thieno[3,2-b]pyridin-5-yl)oxy)propoxy)-5,7-dihydro-6H-pyrrolo[3,4-b]pyridin-6-yl)-4-oxobutanoate (20 mg, 31.7623 μmol, 12.8649% yield) obtained as a white solid. LCMS: (ESI, m/z): [M+H]⁺=630.204. ¹H NMR (400 MHz, DMSO-d₆) δ 8.22 (d, J=3.6 Hz, 1H), 7.97 (d, J=4.8 Hz, 1H), 7.37-7.28 (m, 1H), 4.73 (s, 1H), 4.61 (s, 1H), 4.55-4.48 (m, 3H), 4.47-4.36 (m, 3H), 4.09-4.01 (m, 2H), 3.89 (s, 3H), 3.78 (s, 3H), 3.61 (d, J=5.6 Hz, 3H), 2.68 (d, J=6.5 Hz, 2H), 2.62-2.54 (m, 3H), 2.25 (d, J=4.2 Hz, 2H), 1.18 (t, J=7.1 Hz, 3H).

### Step e: 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic Acid

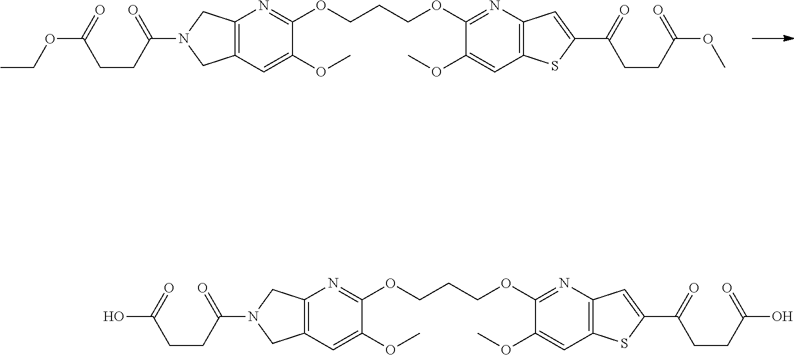 [SMILES: CCOC(=O)CCC(=O)N1Cc2cc(OC)c(OCCCOc3nc4cc(C(=O)CCC(=O)OC)sc4cc3OC)nc2C1 ; COc1cc2c(nc1OCCCOc1nc3cc(C(=O)CCC(=O)O)sc3cc1OC)CN(C(=O)CCC(=O)O)C2]

LiOH (0.004 g, 167.0265 μmol) was added to a solution of ethyl 4-(3-methoxy-2-(3-((6-methoxy-2-(4-methoxy-4-oxobutanoyl)thieno[3,2-b]pyridin-5-yl)oxy)propoxy)-5,7-dihydro-6H-pyrrolo[3,4-b]pyridin-6-yl)-4-oxobutanoate (0.010 g, 15.8812 μmol) in Tetrahydrofuran (4 mL) and Water (1 mL) at 25° C. The reaction mixture was stirred at 25° C. for 2 h. The reaction mixture was evaporated under reduced pressure. The residue was concentrated and diluted with water (2 mL). The resulting mixture was adjusted to pH=7 with HCl (1 M) at 25° C. and evaporated under reduced pressure. The residue was purified by HPLC. The pure fractions was concentrated and lyophilization. There was 4-(5-(3-((6-(3-carboxypropanoyl)-3-methoxy-6,7-dihydro-5H-pyrrolo[3,4-b]pyridin-2-yl)oxy)propoxy)-6-methoxythieno[3,2-b]pyridin-2-yl)-4-oxobutanoic acid (2.6 mg, 4.4248 μmol, 27.8619% yield) obtained as a white solid. LCMS: (ESI, m/z): [M+H]⁺=588.157. ¹H NMR (400 MHz, DMSO-d₆) δ 8.20 (s, 1H), 7.97 (d, J=3.4 Hz, 1H), 7.35-7.28 (m, 1H), 4.73 (s, 2H), 4.61 (s, 2H), 4.51 (s, 4H), 4.47-4.33 (m, 4H), 3.88 (s, 3H), 3.78 (s, 3H), 2.71-2.61 (m, 4H), 2.26 (s, 2H).

### Example 12

### 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoic Acid

### Step a: Tert-butyl 5-methoxyisoindoline-2-carboxylate

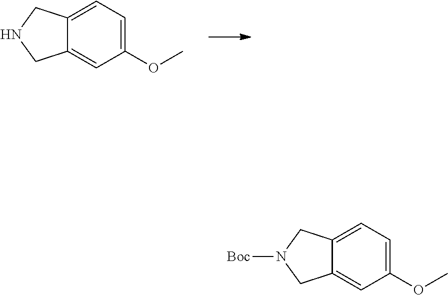 [SMILES: COc1ccc2c(c1)CNC2 ; COc1ccc2c(c1)CN(C(=O)OC(C)(C)C)C2]

TEA (33.12 g, 327.3070 mmol) was added to a solution of 5-Methoxyisoindoline hydrochloride (20.01 g, 107.7833 mmol) and Di-tert-butyl dicarbonate (35.28 g, 161.6523 mmol) in DCM (500 mL) at 20° C. The reaction mixture was stirred overnight at 20° C. The reaction mixture was evaporated under reduced pressure. The residue was purified on silica gel column EA/n-Hexane (0-50%). The pure fractions was concentrated and dried under vacuo. There was tert-butyl 5-methoxyisoindoline-2-carboxylate (30.66 g, 104.5347 mmol, 96.9859% yield) obtained as a white solid. LCMS: (ESI, m/z): [M+H-tBu+ACN]⁺=235.136. ¹H NMR (400 MHz, DMSO-d₆) δ 7.24-7.18 (m, 1H), 6.90 (d, J=4.5 Hz, 1H), 6.85 (s, 0.5H), 6.83 (s, 0.5H), 4.58-4.44 (m, 4H), 3.74 (s, 3H), 1.45 (s, 9H).

### Step b: Tert-butyl 5-bromo-6-methoxy-isoindoline-2-carboxylate

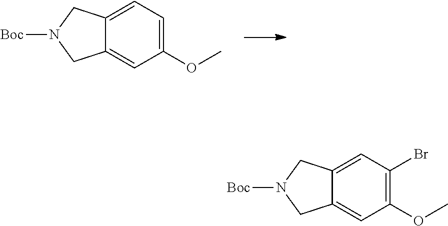 [SMILES: COc1ccc2c(c1)CN(C(=O)OC(C)(C)C)C2 ; COc1cc2c(cc1Br)CN(C(=O)OC(C)(C)C)C2]

NBS (43.64 g, 245.1906 mmol) was added to a solution of tert-butyl 5-methoxyisoindoline-2-carboxylate (30.40 g, 121.9390 mmol) in Tetrahydrofuran (300 mL) and Acetonitrile (300 mL) at 20° C. The reaction mixture was stirred overnight at 20° C. The reaction mixture was evaporated under reduced pressure. The reaction mixture was concentrated and diluted with EA (1000 mL), washed with NaHCO₃ (aq)(3×200 mL) and brine (300 mL). The organics dried over Na₂SO₄, filtered and evaporated under reduced pressure. The residue was purified on silica gel column EA/n-Hexane (0-50%). The pure fractions was concentrated and dried under vacuo. There was tert-butyl 5-bromo-6-methoxy-isoindoline-2-carboxylate (17.61 g, 53.6562 mmol, 44.0025% yield) obtained as a yellow solid. LCMS: (ESI, m/z): [M+H-tBu+ACN]⁺=313.047. ¹H NMR (400 MHz, DMSO-d₆) δ 7.54 (d, J=5.3 Hz, 1H), 7.11 (d, J=4.2 Hz, 1H), 4.51 (t, J=9.8 Hz, 4H), 3.82 (d, J=3.7 Hz, 3H), 1.45 (s, 9H).

### Step c: Tert-butyl 5-methoxy-6-(4,4,5,5-tetramethyl-1,3,2-dioxaborolan-2-yl)isoindoline-2-carboxylate

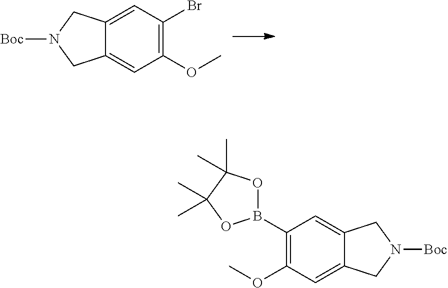 [SMILES: COc1cc2c(cc1Br)CN(C(=O)OC(C)(C)C)C2 ; COc1cc2c(cc1B1OC(C)(C)C(C)(C)O1)CN(C(=O)OC(C)(C)C)C2]

[1,1′-Bis(diphenylphosphino)ferrocene]dichloropalladium(II) (2.10 g, 2.8700 mmol), 4,4,4′,4′,5,5,5′,5′-Octamethyl-2,2′-bi(1,3,2-dioxaborolane) (11.71 g, 46.1137 mmol) and Potassium Acetate (9.76 g, 99.4475 mmol) was added to a solution of tert-butyl 5-bromo-6-methoxy-isoindoline-2-carboxylate (10.04 g, 30.5910 mmol) in 1,4-Dioxane (200 mL) at 20° C. The reaction mixture was heated to 100° C. and stirred overnight under N₂ atmosphere. The reaction mixture was concentrated and diluted with EA (500 mL), washed with water (200 mL) and brine (200 mL). The organics dried over Na₂SO₄, filtered and evaporated under reduced pressure. The residue was purified on silica gel column EA/n-Hexane (0-50%). The pure fractions was concentrated and dried under vacuo. There was tert-butyl 5-methoxy-6-(4,4,5,5-tetramethyl-1,3,2-dioxaborolan-2-yl)isoindoline-2-carboxylate (11.00 g, 29.3126 mmol, 95.8208% yield) obtained as a oil. LCMS: (ESI, m/z): [M+H-tBu+ACN]⁺=361.222.

### Step d: Tert-butyl 5-hydroxy-6-methoxy-isoindoline-2-carboxylate

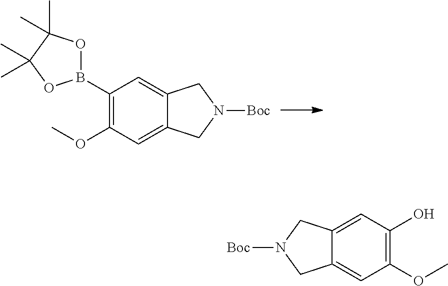 [SMILES: COc1cc2c(cc1B1OC(C)(C)C(C)(C)O1)CN(C(=O)OC(C)(C)C)C2 ; COc1cc2c(cc1O)CN(C(=O)OC(C)(C)C)C2]

Sodium perborate tetrahydrate (6.28 g, 40.8164 mmol) was added to a solution of tert-butyl 5-methoxy-6-(4,4,5,5-tetramethyl-1,3,2-dioxaborolan-2-yl)isoindoline-2-carboxylate (9.75 g, 25.9816 mmol) in Tetrahydrofuran (150 mL) and Water (150 mL) at 20° C. The reaction mixture was stirred for 2 h at 20° C. The reaction mixture was concentrated and diluted with EA (600 mL), washed with water (300 mL) and brine (300 mL). The organics dried over Na₂SO₄, filtered and evaporated under reduced pressure. The residue was purified on silica gel column EA/DCM (0-100%). The pure fractions was concentrated and dried under vacuo. There was tert-butyl 5-hydroxy-6-methoxy-isoindoline-2-carboxylate (6.58 g, 24.8017 mmol, 95.4589% yield) obtained as a yellow oil. LCMS: (ESI, m/z): [M+H-tBu+ACN]⁺=251.131. ¹H NMR (400 MHz, DMSO-d₆) δ 8.92 (s, 1H), 6.88 (d, J=5.8 Hz, 1H), 6.70 (d, J=2.3 Hz, 1H), 4.44 (t, J=9.6 Hz, 4H), 3.74 (d, J=3.4 Hz, 3H), 1.44 (s, 9H).

### Step e: Tert-butyl 5-(3-bromopropoxy)-6-methoxy-isoindoline-2-carboxylate

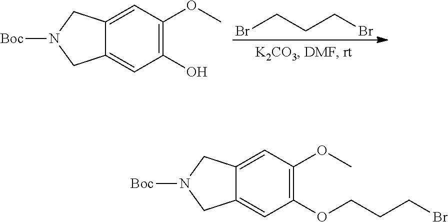

1,3-Dibromopropane (0.564 g, 2.7936 mmol) was added to a mixture of Potassium carbonate (0.286 g, 2.0694 mmol) and tert-butyl 5-hydroxy-6-methoxyisoindoline-2-carboxylate (0.179 g, 674.6977 μmol) in DMF (3 mL) at 20° C. under N₂ atmosphere. The reaction mixture was stirred for 5 h at 20° C. The mixture was purified on C18 column ACN/H₂O (0.1% FA)(0-50%). The pure fractions was concentrated and dried under vacuo. There was tert-butyl 5-(3-bromopropoxy)-6-methoxy-isoindoline-2-carboxylate (0.154 g, 398.6747 μmol, 59.0894% yield) obtained as a white oil. LCMS: (ESI, m/z): [M+H]⁺=386.090. ¹H NMR (400 MHz, DMSO-d₆) δ 6.96 (t, J=5.8 Hz, 2H), 4.50 (s, 2H), 4.48 (s, 2H), 4.03 (q, J=5.5 Hz, 2H), 3.74 (d, J=3.4 Hz, 3H), 3.66 (t, J=6.5 Hz, 2H), 2.27-2.19 (m, 2H), 1.45 (s, 9H).

### Step f: Tert-butyl 5-(3-((2-(4-ethoxy-4-oxobutanoyl)-6-methoxybenzo[b]thiophen-5-yl)oxy)propoxy)-6-methoxyisoindoline-2-carboxylate

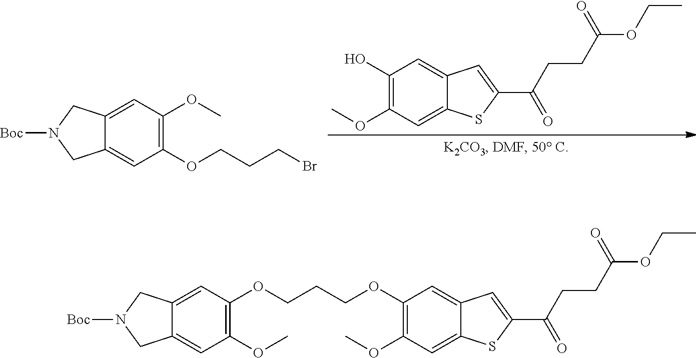 [SMILES: COc1cc2c(cc1OCCCBr)CN(C(=O)OC(C)(C)C)C2 ; CCOC(=O)CCC(=O)c1cc2cc(O)c(OC)cc2s1 ; CCOC(=O)CCC(=O)c1cc2cc(OCCCOc3cc4c(cc3OC)CN(C(=O)OC(C)(C)C)C4)c(OC)cc2s1]

Potassium carbonate (0.079 g, 571.6126 μmol) was added to a solution of tert-butyl 5-(3-bromopropoxy)-6-methoxyisoindoline-2-carboxylate (0.079 g, 204.5150 μmol) and ethyl 4-(5-hydroxy-6-methoxybenzo[b]thiophen-2-yl)-4-oxobutanoate (0.064 g, 207.5571 μmol) in DMF (3 mL) at 20° C. under N₂ atmosphere. The reaction mixture was stirred overnight at 50° C. The reaction mixture was quenched with adding of water (50 mL) at 20° C., extracted with EA (3×50 mL) and washed with brine (50 mL). The organics dried over Na₂SO₄, filtered and evaporated under reduced pressure. There was a crude tert-butyl 5-(3-((2-(4-ethoxy-4-oxobutanoyl)-6-methoxybenzo[b]thiophen-5-yl)oxy)propoxy)-6-methoxyisoindoline-2-carboxylate (0.088 g, 143.3886 μmol, 70.1115% yield) obtained as a yellow oil which was used directly in the next step. LCMS: (ESI, m/z): [M+H]⁺=614.230.

### Step g: 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoic Acid

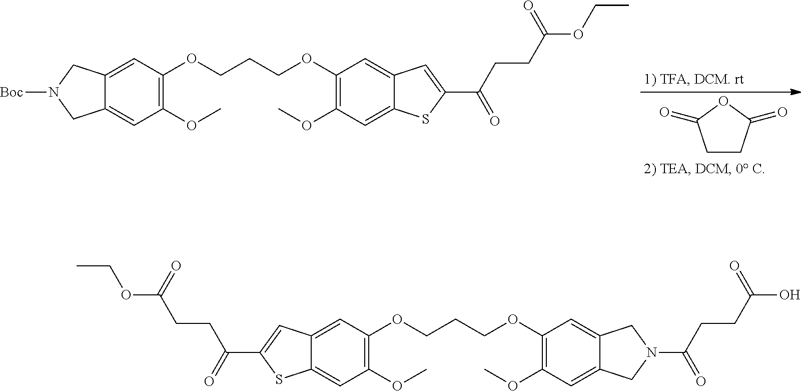

Trifluoroacetic acid (1 mL) was added to a solution of tert-butyl 5-(3-((2-(4-ethoxy-4-oxobutanoyl)-6-methoxybenzo[b]thiophen-5-yl)oxy)propoxy)-6-methoxyisoindoline-2-carboxylate (0.084 g, 136.8709 μmol) in DCM (3 mL) at 20° C. The reaction mixture was stirred for 1 h at 20° C. The reaction mixture was evaporated under reduced pressure. Dihydrofuran-2,5-dione (0.050 g, 499.6373 μmol) and TEA (0.195 g, 1.9271 mmol) was added to a solution of the residue in DCM (5 mL) at 0° C. under N₂ atmosphere. The reaction mixture was stirred and warmed up to 20° C. naturally. The reaction mixture was quenched with adding of water (50 mL) at 20° C. and adjusted to pH=3 with HCl (1 mol/L), extracted with EA (3×50 mL) and washed with brine (50 mL). The organics dried over Na₂SO₄, filtered and evaporated under reduced pressure. The residue was purified on C18 column ACN/H₂O (0.1% FA)(0-40%). The pure fractions was concentrated and dried under vacuo. There was 4-(5-(3-((2-(4-ethoxy-4-oxobutanoyl)-6-methoxybenzo[b]thiophen-5-yl)oxy)propoxy)-6-methoxyisoindolin-2-yl)-4-oxobutanoic acid (0.038 g, 61.9221 μmol, 45.2413% yield) obtained as a white solid. LCMS: (ESI, m/z): [M+H]⁺=614.200. ¹H NMR (400 MHz, DMSO-d₆) δ 8.18 (s, 1H), 7.60 (s, 1H), 7.53 (s, 1H), 7.02 (s, 0.5H), 6.98 (s, 0.5H), 6.97 (s, 0.5H), 6.95 (s, 0.5H), 4.74 (s, 1H), 4.72 (s, 1H), 4.52 (s, 2H), 4.20 (t, J=6.3 Hz, 2H), 4.14 (t, J=6.3 Hz, 2H), 4.06 (q, J=7.1 Hz, 2H), 3.86 (s, 3H), 3.75 (s, 3H), 3.30-3.28 (m, 2H), 2.70-2.64 (m, 2H), 2.58-2.52 (m, 4H), 2.27-2.18 (m, 2H), 1.17 (t, J=7.1 Hz, 3H).

### Example 13

### Sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate

### Step a: 2,3-dimethoxy-5-vinyl-pyridine

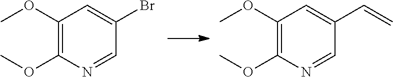

2-ethenyl-4,4,5,5-tetramethyl-1,3,2-dioxaborolane (9.25 g, 60.0595 mmol), Potassium carbonate (18.91 g, 136.8252 mmol) and Pd(dppf)Cl₂ (1.97 g, 2.6923 mmol) was added to a solution of 5-Bromo-2,3-dimethoxypyridine (9.88 g, 45.3112 mmol) in 1,4-Dioxane (100 mL) and Water (20 mL) at 20° C. The reaction mixture was stirred at 80° C. for 2 h under nitrogen atmosphere. The reaction mixture was concentrated and diluted with EA (100 mL), washed with NaHCO₃ (aq)(100 mL) and brine (50 mL). The organics dried over Na₂SO₄, filtered and evaporated under reduced pressure. The residue was purified on silica gel column Ethyl acetate/Heptane (0-20%). The pure fractions was concentrated and dried under vacuo. There was 2,3-dimethoxy-5-vinyl-pyridine (5.22 g, 31.6002 mmol, 69.7404% yield) obtained as a colorless oil. LCMS: (ESI, m/z): [M+H]⁺=166.079. ¹H NMR (400 MHz, DMSO-d₆) δ 7.70 (d, J=1.9 Hz, 1H), 7.46 (t, J=5.8 Hz, 1H), 6.73-6.63 (m, 1H), 5.83 (dd, J=17.6, 0.8 Hz, 1H), 5.24 (dd, J=11.0, 0.8 Hz, 1H), 3.86 (s, 3H), 3.82 (s, 3H).

### Step b: 5,6-dimethoxypyridine-3-carbaldehyde

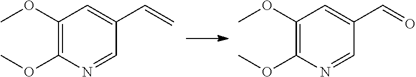

Potassium osmate(VI) dehydrate (0.55 g, 1.4927 mmol) in Water (25 mL) was added to a solution of 2,3-dimethoxy-5-vinyl-pyridine (5.17 g, 31.2975 mmol) and 4-Methylmorpholine N-oxide (7.44 g, 63.5105 mmol) in Tetrahydrofuran (100 mL) at 20° C. The reaction mixture was stirred at 20° C. for 30 min. Sodium periodate (6.82 g, 31.8854 mmol) was added to the mixture at 20° C. The reaction mixture was stirred overnight at 20° C. The reaction mixture was concentrated and diluted with EA (500 mL), washed with water (200 mL) and brine (50 mL). The organics dried over Na₂SO₄, filtered and evaporated under reduced pressure. The residue was purified on silica gel column EA/Hept (0-20%). The pure fractions was concentrated and dried under vacuo. There was 5,6-dimethoxypyridine-3-carbaldehyde (3.51 g, 20.9976 mmol, 67.0904% yield) obtained as a white solid. LCMS: (ESI, m/z): [M+H]⁺=168.058. ¹H NMR (400 MHz, DMSO-d₆) δ 9.95 (s, 1H), 8.34 (d, J=1.9 Hz, 1H), 7.53 (d, J=1.8 Hz, 1H), 3.99 (s, 3H), 3.87 (s, 3H).

### Step c: (5,6-dimethoxy-3-pyridyl)methanol

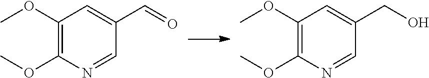

NaBH4 (0.4 g, 10.5729 mmol) was added to a solution of 5,6-dimethoxypyridine-3-carbaldehyde (3.51 g, 20.9976 mmol) in methanol (100 mL) at 20° C. The reaction mixture was stirred at 20° C. for 1 h. The reaction mixture was concentrated and diluted with EA (500 mL), washed with water (200 mL) and brine (200 mL). The organics dried over Na₂SO₄, filtered and evaporated under reduced pressure. The pure fractions was concentrated and dried under vacuo. There was (5,6-dimethoxy-3-pyridyl)methanol (3.33 g, 19.6835 mmol, 93.7418% yield) obtained as a yellow oil. LCMS: (ESI, m/z): [M+H]⁺=170.074. ¹H NMR (400 MHz, DMSO-d₆) δ 7.61 (d, J=1.6 Hz, 1H), 7.23 (t, J=6.4 Hz, 1H), 5.13 (t, J=5.7 Hz, 1H), 4.43 (d, J=5.7 Hz, 2H), 3.84 (s, 3H), 3.77 (s, 3H).

### Step d: (2-bromo-5,6-dimethoxy-3-pyridyl)methanol

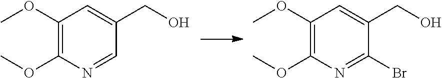

NBS (4.26 g, 23.9347 mmol) and Acetic acid (0.5 mL) was added to a solution of (5,6-dimethoxy-3-pyridyl)methanol (3.30 g, 19.5062 mmol) in Acetonitrile (100 mL) at 20° C. The reaction mixture was stirred at 20° C. for 2 h. The reaction mixture was evaporated under reduced pressure. The residue was diluted with EA (200 mL), washed with NaHCO₃ (aq)(2×100 mL) and brine (100 mL). The organics dried over Na₂SO₄, filtered and evaporated under reduced pressure. The residue was purified on silica gel column EA/Hept (0-100%). The pure fractions was concentrated and dried under vacuo. There was (2-bromo-5,6-dimethoxy-3-pyridyl)methanol (2.91 g, 11.7304 mmol, 60.1369% yield) obtained as a white solid. LCMS: (ESI, m/z): [M+H]⁺=247.984. ¹H NMR (400 MHz, DMSO-d₆) δ 7.40 (s, 1H), 5.45 (t, J=5.4 Hz, 1H), 4.42 (d, J=5.5 Hz, 2H), 3.85 (s, 3H), 3.80 (s, 3H).

### Step e: 2-bromo-5,6-dimethoxy-pyridine-3-carbaldehyde

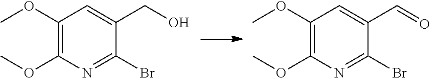

Dess-Martin periodinane (7.3 g, 17.2113 mmol) was added to a solution of (2-bromo-5,6-dimethoxy-3-pyridyl)methanol (2.91 g, 11.7304 mmol) in DCM (100 mL) at 20° C. The reaction mixture was stirred at 20° C. for 1 h. The reaction mixture was evaporated under reduced pressure. The reaction mixture was concentrated and diluted with EA (200 mL), washed with NaHCO₃ (aq)(2×100 mL) and brine (100 mL). The organics dried over Na₂SO₄, filtered and evaporated under reduced pressure. The residue was purified on silica gel column EA/Hept (0-100%). The pure fractions was concentrated and dried under vacuo. There was 2-bromo-5,6-dimethoxy-pyridine-3-carbaldehyde (2.7 g, 10.9730 mmol, 93.5437% yield) obtained as a white solid. LCMS: (ESI, m/z): [M+H]⁺=245.969. ¹H NMR (400 MHz, DMSO-d₆) δ 10.04 (s, 1H), 7.52 (s, 1H), 3.99 (s, 3H), 3.87 (s, 3H).

### Step f: 5,6-dimethoxythieno[2,3-b]pyridine-2-carboxylic Acid

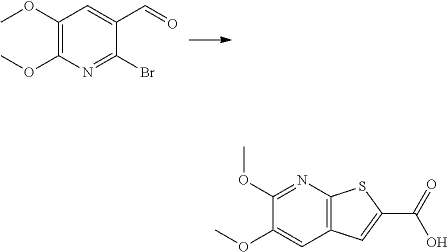 [SMILES: COc1cc(C=O)c(Br)nc1OC ; COc1cc2cc(C(=O)O)sc2nc1OC]

Methyl thioglycolate (1.1870 g, 11.1830 mmol) was added to a mixture of 2-bromo-5,6-dimethoxy-pyridine-3-carbaldehyde (2.68 g, 10.8918 mmol) and Potassium carbonate (4.65 g, 33.6455 mmol) in N,N-Dimethylformamide (50 mL) at 20° C. The reaction mixture was stirred overnight at 60° C. under N₂ atmosphere. The reaction mixture was concentrated and diluted with water (500 mL). The precipitate was collected by filtration, washed with water (100 mL). LiOH (0.40 g, 16.7026 mmol) was added to a solution of the filtrate cake in Tetrahydrofuran (50 mL) and Water (10 mL) at 20° C. The reaction mixture was stirred at 20° C. for 2 h. The reaction mixture was concentrated and diluted with water (200 mL). The mixture was adjusted to pH=3 with HCl (1 M). The precipitate was collected by filtration, washed with water (100 mL). The pure fractions was dried under vacuo. There was 5,6-dimethoxythieno[2,3-b]pyridine-2-carboxylic acid (2.32 g, 9.6971 mmol, 89.0313% yield) obtained as a white solid. LCMS: (ESI, m/z): [M+H]⁺=240.065. ¹H NMR (400 MHz, DMSO-d₆) δ 7.91 (s, 1H), 7.80 (s, 1H), 3.98 (s, 3H), 3.85 (s, 3H).

### Step g: Tert-butyl 3-(5,6-dimethoxythieno[2,3-b]pyridin-2-yl)-3-oxo-propanoate

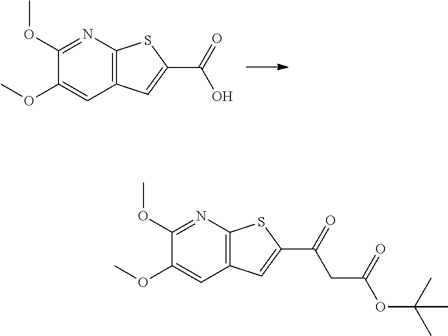 [SMILES: COc1cc2cc(C(=O)O)sc2nc1OC ; COc1cc2cc(C(=O)CC(=O)OC(C)(C)C)sc2nc1OC]

Carbonyl diimidazole (3.11 g, 19.1799 mmol) was added to a solution of 5,6-dimethoxythieno[2,3-b]pyridine-2-carboxylic acid (2.29 g, 9.5717 mmol) in N,N-Dimethylformamide (50 mL) at 20° C. The reaction mixture was stirred for 30 min at 20° C. under nitrogen atmosphere. 3-tert-Butoxy-3-oxopropanoic acid (3.16 g, 19.7294 mmol), Magnesium chloride (1.87 g, 19.6406 mmol) and Triethylamine (2.96 g, 29.2521 mmol) were added to the mixture at 20° C. The reaction mixture was stirred overnight at 20° C. The reaction mixture was concentrated and diluted with EA (500 mL), washed with NaHCO₃ (aq)(2×300 mL) and brine (200 mL). The organics dried over Na₂SO₄, filtered and evaporated under reduced pressure. The residue was purified on silica gel column EA/n-Hexane (0-50%). The pure fractions was concentrated and dried under vacuo. There was tert-butyl 3-(5,6-dimethoxythieno[2,3-b]pyridin-2-yl)-3-oxo-propanoate (2.37 g, 7.0245 mmol, 73.3885% yield) obtained as a white solid. LCMS: (ESI, m/z): [M+H]⁺=338.098. ¹H NMR (400 MHz, DMSO-d₆) δ 8.15 (d, J=9.8 Hz, 1H), 7.83 (d, J=7.7 Hz, 1H), 4.06 (d, J=8.5 Hz, 2H), 3.98 (s, 3H), 3.88 (s, 3H), 1.41 (s, 9H).

### Step h: O1-tert-butyl O4-ethyl 2-(5,6-dimethoxythieno[2,3-b]pyridine-2-carbonyl)butanedioate

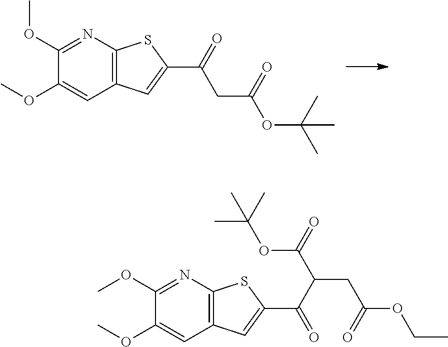

Ethyl bromoacetate (0.63 g, 3.7724 mmol) was added to a mixture of tert-butyl 3-(5,6-dimethoxythieno[2,3-b]pyridin-2-yl)-3-oxo-propanoate (1.2 g, 3.5567 mmol) and Potassium carbonate (0.80 g, 5.7885 mmol) in N,N-Dimethylformamide (20 mL) at 20° C. The reaction mixture was stirred for 6 h at 20° C. The reaction mixture was concentrated and diluted with EA (200 mL), washed with NaHCO₃ (aq)(200 mL) and brine (200 mL). The organics dried over Na₂SO₄, filtered and evaporated under reduced pressure. The residue was purified on silica gel column EA/n-Hexane (0-50%). The pure fractions was concentrated and dried under vacuo. There was 01-tert-butyl 04-ethyl 2-(5,6-dimethoxythieno[2,3-b]pyridine-2-carbonyl)butanedioate (1.72 g, 4.0616 mmol, 114.1951% yield) obtained as a yellow oil. LCMS: (ESI, m/z): [M+H]⁺=424.135. ¹H NMR (400 MHz, DMSO-d₆) δ 8.31 (s, 1H), 7.86 (s, 1H), 4.82 (t, J=7.3 Hz, 1H), 4.05 (m, 2H), 4.00 (s, 3H), 3.87 (s, 3H), 2.92 (d, J=7.2 Hz, 2H), 1.32 (s, 9H), 1.18-1.06 (m, 3H).

### Step i: Ethyl 4-(5,6-dimethoxythieno[2,3-b]pyridin-2-yl)-4-oxo-butanoate

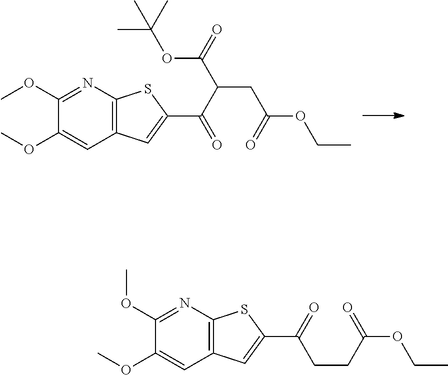

Trifluoroacetic acid (2 mL) was added to a solution of 01-tert-butyl 04-ethyl 2-(5,6-dimethoxythieno[2,3-b]pyridine-2-carbonyl)butanedioate (1.51 g, 3.5657 mmol) in Toluene (10 mL) at 20° C. The reaction mixture was stirred for 1 h at 50° C. The reaction mixture was evaporated under reduced pressure. The residue was purified on C-18 column MeCN/water (0-100%). The pure fractions was concentrated and dried under vacuo. There was ethyl 4-(5,6-dimethoxythieno[2,3-b]pyridin-2-yl)-4-oxo-butanoate (0.94 g, 2.9069 mmol, 81.5252% yield) obtained as a white solid. LCMS: (ESI, m/z): [M+H]⁺=324.083. ¹H NMR (400 MHz, DMSO-d₆) δ 8.20 (s, 1H), 7.79 (s, 1H), 4.00-4.08 (m, 2H), 3.99 (s, 3H), 3.87 (s, 3H), 3.30-3.33 (m, 2H), 2.67 (t, J=6.1 Hz, 2H), 1.18 (t, J=7.0 Hz, 3H).

### Step j: Ethyl 4-(6-chloro-5-methoxy-thieno[2,3-b]pyridin-2-yl)-4-oxo-butanoate

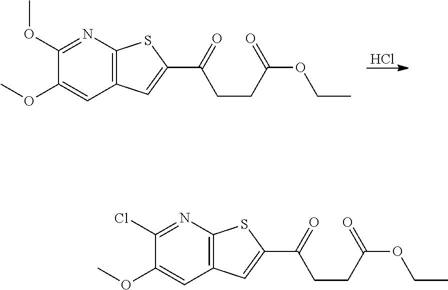

Ethyl 4-(5,6-dimethoxythieno[2,3-b]pyridin-2-yl)-4-oxo-butanoate (0.308 g, 952.4899 μmol) was added to HCl (10 mL) at 20° C. The reaction mixture was stirred at 100° C. for 1 h. Cooled the reaction mixture to 20° C., POCl3 (1 mL) was added to the reaction mixture. The reaction mixture was stirred at 100° C. for 2 h under N₂ atmosphere. The reaction mixture was evaporated under reduced pressure. The residue was purified on C-18 column MeCN/water (0-100%). The pure fractions was concentrated and dried under vacuo. There was ethyl 4-(6-chloro-5-methoxy-thieno[2,3-b]pyridin-2-yl)-4-oxo-butanoate (0.25 g, 762.7004 μmol, 80.0744% yield) obtained as a white solid. LCMS: (ESI, m/z): [M+H]⁺=328.033. ¹H NMR (400 MHz, DMSO-d₆) δ 8.31 (s, 1H), 8.14 (s, 1H), 4.00-4.08 (m 2H), 3.99 (s, 3H), 3.44-3.36 (m, 2H), 2.70 (t, J=6.2 Hz, 2H), 1.18 (t, J=7.1 Hz, 3H).

### Step k: Ethyl 4-(5-(3-((2-(4-ethoxy-4-oxobutanoyl)-5-methoxythieno[2,3-b]pyridin-6-yl)oxy)propoxy)-6-methoxyisoindolin-2-yl)-4-oxobutanoate

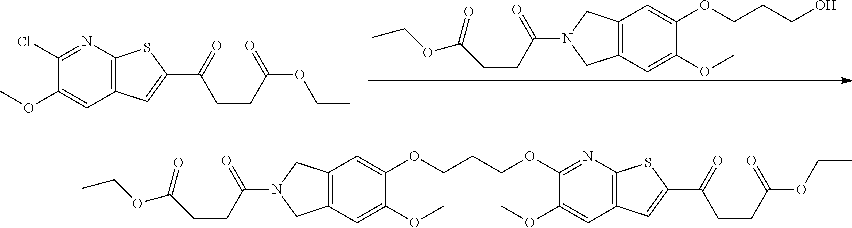 [SMILES: CCOC(=O)CCC(=O)c1cc2cc(OC)c(Cl)nc2s1 ; CCOC(=O)CCC(=O)N1Cc2cc(OC)c(OCCCO)cc2C1 ; CCOC(=O)CCC(=O)c1cc2cc(OC)c(OCCCOc3cc4c(cc3OC)CN(C(=O)CCC(=O)OCC)C4)nc2s1]

RockPhos Palladacycle Gen.3 (0.03 g, 35.7385 μmol) was added to a mixture of ethyl 4-(6-chloro-5-methoxy-thieno[2,3-b]pyridin-2-yl)-4-oxo-butanoate (0.10 g, 305.0799 μmol), ethyl 4-(5-(3-hydroxypropoxy)-6-methoxyisoindolin-2-yl)-4-oxobutanoate (0.18 g, 512.2470 μmol) and Potassium phosphate (0.20 g, 942.2127 μmol) in Toluene (10 mL) at 20° C. The reaction mixture was stirred overnight at 130° C. under N₂ atmosphere. The reaction mixture was evaporated under reduced pressure. The residue was purified on C-18 column MeCN/water (0-100%). The pure fractions was concentrated and dried under vacuo. There was ethyl 4-(5-(3-((2-(4-ethoxy-4-oxobutanoyl)-5-methoxythieno[2,3-b]pyridin-6-yl)oxy)propoxy)-6-methoxyisoindolin-2-yl)-4-oxobutanoate (0.013 g, 20.2267 μmol, 6.6300% yield) obtained as a yellow oil. LCMS: (ESI, m/z): [M+H]⁺=643.225.

### Step l: Sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate

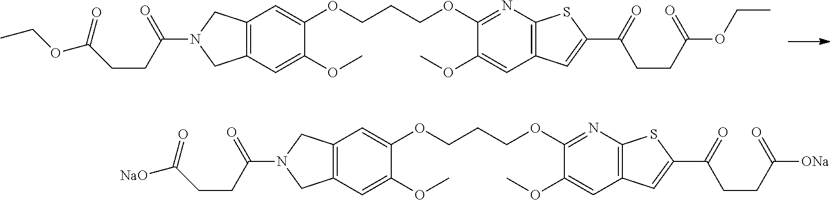

LiOH (3 mg, 125.2699 μmol) was added to a solution of ethyl 4-(5-(3-((2-(4-ethoxy-4-oxobutanoyl)-5-methoxythieno[2,3-b]pyridin-6-yl)oxy)propoxy)-6-methoxyisoindolin-2-yl)-4-oxobutanoate (0.018 g, 28.0062 μmol) in Tetrahydrofuran (4 mL) and Water (1 mL) at 20° C. The reaction mixture was stirred at 20° C. for 1 h. The reaction mixture was purified by Prep-HPLC. The pure fractions was concentrated and dried by lyophilization. NaHCO₃ (3.4 mg, 40.4730 μmol) was added to the lyophilized product in Water (5 mL), and stirred at 20° C. for 1 h. The solvent was removed by lyophilization. There was sodium 4-(5-(3-((2-(3-carboxylatopropanoyl)-5-methoxythieno[2,3-b]pyridin-6-yl)oxy)propoxy)-6-methoxyisoindolin-2-yl)-4-oxobutanoate (13 mg, 20.6162 μmol, 73.6129% yield) obtained as a white solid. LCMS: (ESI, m/z): [M+H]⁺=587.162.

### Example 14

### 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic Acid

### Step a: Ethyl 4-[5-(3-bromopropoxy)-6-methoxy-benzothiophen-2-yl]-4-oxo-butanoate

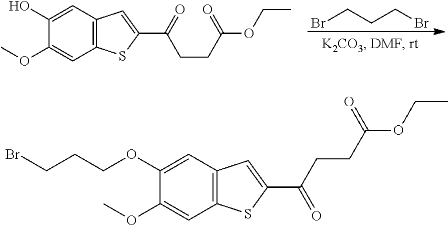

Potassium carbonate (0.113 g, 817.6230 μmol) was added to a solution of ethyl 4-(5-hydroxy-6-methoxybenzo[b]thiophen-2-yl)-4-oxobutanoate (0.081 g, 262.6894 μmol) and 1,3-dibromopropane (0.263 g, 1.3027 mmol) in DMF (5 mL) at 20° C. under N₂ atmosphere. The reaction mixture was stirred overnight at 20° C. The mixture was purified on C18 column ACN/H₂O (0-60%). The pure fractions was concentrated and dried under vacuo. There was ethyl 4-[5-(3-bromopropoxy)-6-methoxy-benzothiophen-2-yl]-4-oxo-butanoate (0.074 g, 172.3638 μmol, 65.6150% yield) obtained as a white solid. LCMS: (ESI, m/z): [M+H]⁺=309.070. ¹H NMR (400 MHz, DMSO-d₆) δ 8.21 (s, 1H), 7.62 (s, 1H), 7.53 (s, 1H), 4.14 (t, J=6.0 Hz, 2H), 4.06 (q, J=7.1 Hz, 2H), 3.87 (s, 3H), 3.70 (t, J=6.5 Hz, 2H), 3.30 (t, J=6.5 Hz, 2H), 2.66 (t, J=6.4 Hz, 2H), 2.35-2.26 (m, 2H), 1.18 (t, J=7.1 Hz, 3H).

### Step b: Ethyl 4-(5-(3-((4-chloro-2-(4-ethoxy-4-oxobutanoyl)-6-methoxyisoindolin-5-yl)oxy)propoxy)-6-methoxybenzo[b]thiophen-2-yl)-4-oxobutanoate

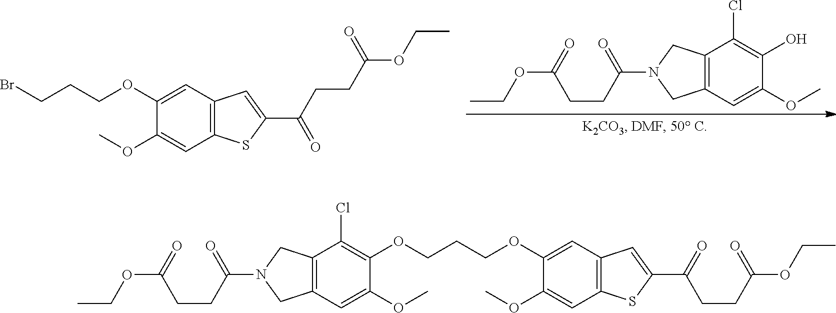 [SMILES: CCOC(=O)CCC(=O)c1cc2cc(OCCCBr)c(OC)cc2s1 ; CCOC(=O)CCC(=O)N1Cc2cc(OC)c(O)c(Cl)c2C1 ; CCOC(=O)CCC(=O)c1cc2cc(OCCCOc3c(OC)cc4c(c3Cl)CN(C(=O)CCC(=O)OCC)C4)c(OC)cc2s1]

Potassium carbonate (0.094 g, 680.1466 μmol) was added to a solution of ethyl 4-[5-(3-bromopropoxy)-6-methoxy-benzothiophen-2-yl]-4-oxo-butanoate (0.092 g, 214.2904 μmol) and ethyl 4-(4-chloro-5-hydroxy-6-methoxyisoindolin-2-yl)-4-oxobutanoate (0.072 g, 219.6763 μmol) in DMF (3 mL) at 20° C. under N₂ atmosphere. The reaction mixture was heated to 50° C. and stirred overnight. The mixture was purified on C18 column ACN/H₂O (0.1% FA)(0-60%). The pure fractions was concentrated and dried under vacuo. There was ethyl 4-(5-(3-((4-chloro-2-(4-ethoxy-4-oxobutanoyl)-6-methoxyisoindolin-5-yl)oxy)propoxy)-6-methoxybenzo[b]thiophen-2-yl)-4-oxobutanoate (0.089 g, 131.6233 μmol, 61.4229% yield) obtained as a white solid. LCMS: (ESI, m/z): [M+H]⁺=676.190. ¹H NMR (400 MHz, DMSO-d₆) δ 8.23 (s, 1H), 7.60 (s, 1H), 7.51 (d, J=4.3 Hz, 1H), 7.07 (s, 0.5H), 7.04 (s, 0.5H), 4.83 (s, 1H), 4.75 (s, 1H), 4.61 (s, 1H), 4.50 (s, 1H), 4.26 (s, 2H), 4.15 (t, J=6.0 Hz, 2H), 4.10-4.01 (m, 4H), 3.86 (s, 3H), 3.77 (s, 3H), 3.31-3.27 (m, 2H), 2.70-2.52 (m, 6H), 2.24-2.14 (m, 2H), 1.18 (t, J=7.1 Hz, 6H).

### Step c: 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic Acid

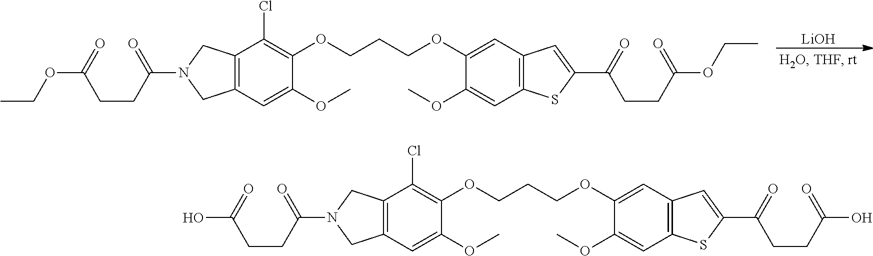

LiOH (0.051 g, 2.1296 mmol) was added to a solution of ethyl 4-(5-(3-((4-chloro-2-(4-ethoxy-4-oxobutanoyl)-6-methoxyisoindolin-5-yl)oxy)propoxy)-6-methoxybenzo[b]thiophen-2-yl)-4-oxobutanoate (0.040 g, 59.1565 μmol) in THF (2 mL) and H₂O (2 mL) at 20° C. The reaction mixture was stirred for 2 h at 20° C. The reaction mixture was adjusted to pH=3 with HCl (1 mol/L). The mixture was purified on C18 column ACN/H₂O (0.1% FA)(0-45%). The pure fractions was concentrated and dried by lyophilization. There was 4-(5-(3-((2-(3-carboxypropanoyl)-4-chloro-6-methoxyisoindolin-5-yl)oxy)propoxy)-6-methoxybenzo[b]thiophen-2-yl)-4-oxobutanoic acid (0.025 g, 40.3183 μmol, 68.1552% yield) obtained as a white solid. LCMS: (ESI, m/z): [M+H]⁺=620.130. ¹H NMR (400 MHz, DMSO-d₆) δ 8.21 (s, 1H), 7.59 (s, 1H), 7.50 (d, J=3.3 Hz, 1H), 7.06 (s, 0.5H), 7.03 (s, 0.5H), 4.82 (s, 1H), 4.74 (s, 1H), 4.61 (s, 1H), 4.50 (s, 1H), 4.31-4.22 (m, 2H), 4.15 (t, J=6.1 Hz, 2H), 3.86 (s, 3H), 3.77 (s, 3H), 3.26-3.24 (m, 2H), 2.61-2.55 (m, 4H), 2.54-2.44 (m, 2H), 2.19 (t, J=6.2 Hz, 2H).

### Example 15

### 4-(5-(3-((2-(3-carboxypropanoyl)-5-methoxythieno[3,2-b]pyridin-6-yl)oxy)propoxy)-6-methoxyisoindolin-2-yl)-4-oxobutanoic Acid

### Step a: Ethyl 4-(5-(3-((2-(4-ethoxy-4-oxobutanoyl)-5-methoxythieno[3,2-b]pyridin-6-yl)oxy)propoxy)-6-methoxyisoindolin-2-yl)-4-oxobutanoate

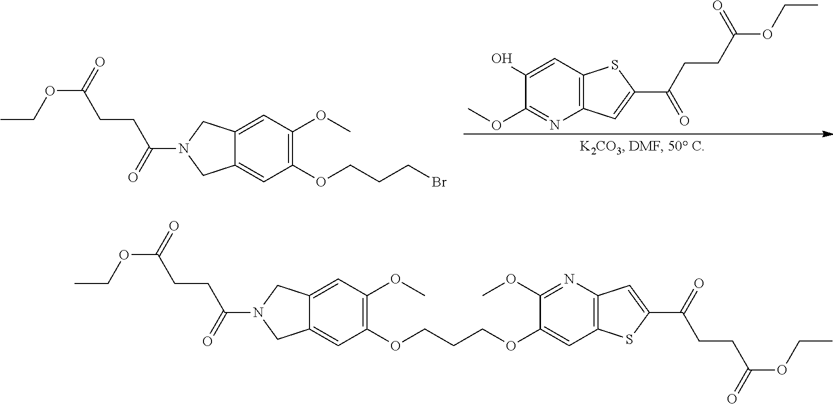 [SMILES: CCOC(=O)CCC(=O)N1Cc2cc(OC)c(OCCCBr)cc2C1 ; CCOC(=O)CCC(=O)c1cc2nc(OC)c(O)cc2s1 ; CCOC(=O)CCC(=O)c1cc2nc(OC)c(OCCCOc3cc4c(cc3OC)CN(C(=O)CCC(=O)OCC)C4)cc2s1]

Potassium carbonate (0.089 g, 643.9686 μmol) was added to a solution of ethyl 4-(6-hydroxy-5-methoxythieno[3,2-b]pyridin-2-yl)-4-oxobutanoate (0.069 g, 223.0577 μmol) and ethyl 4-(5-(3-bromopropoxy)-6-methoxyisoindolin-2-yl)-4-oxobutanoate (0.079 g, 190.6877 μmol) in DMF (2 mL) at 20° C. under N₂ atmosphere. The reaction mixture was heated to 50° C. and stirred overnight. The reaction mixture was purified on C18 column ACN/H₂O (0.1% FA)(0-55%). The pure fractions was concentrated and dried by lyophilization. There was ethyl 4-(5-(3-((2-(4-ethoxy-4-oxobutanoyl)-5-methoxythieno[3,2-b]pyridin-6-yl)oxy)propoxy)-6-methoxyisoindolin-2-yl)-4-oxobutanoate (0.075 g, 116.6925 μmol, 61.1956% yield) obtained as a white solid. LCMS: (ESI, m/z): [M+H]⁺=690.130. ¹H NMR (400 MHz, DMSO-d₆) δ 8.27 (s, 1H), 8.03 (s, 1H), 7.06-6.89 (m, 2H), 4.74 (s, 1H), 4.72 (s, 1H), 4.51 (s, 2H), 4.27 (t, J=6.2 Hz, 2H), 4.12 (t, J=6.7 Hz, 2H), 4.09-4.01 (m, 4H), 3.96 (s, 3H), 3.74 (d, J=2.1 Hz, 3H), 3.40-3.34 (m, 2H), 2.68-2.54 (m, 6H), 2.29-2.19 (m, 2H), 1.18 (t, J=7.1 Hz, 6H).

### Step b: 4-(5-(3-((2-(3-carboxypropanoyl)-5-methoxythieno[3,2-b]pyridin-6-yl)oxy)propoxy)-6-methoxyisoindolin-2-yl)-4-oxobutanoic Acid

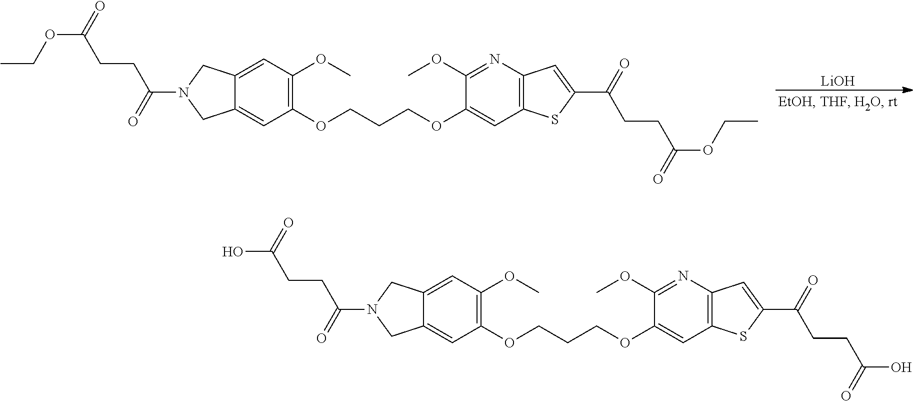 [SMILES: CCOC(=O)CCC(=O)c1cc2nc(OC)c(OCCCOc3cc4c(cc3OC)CN(C(=O)CCC(=O)OCC)C4)cc2s1 ; "LiOH" ; COc1cc2c(cc1OCCCOc1cc3sc(C(=O)CCC(=O)O)cc3nc1OC)CN(C(=O)CCC(=O)O)C2]

LiOH (0.050 g, 2.0878 mmol) was added to a solution of ethyl 4-(5-(3-((2-(4-ethoxy-4-oxobutanoyl)-5-methoxythieno[3,2-b]pyridin-6-yl)oxy)propoxy)-6-methoxyisoindolin-2-yl)-4-oxobutanoate (0.051 g, 79.3509 μmol) in THF (2 mL), H₂O (2 mL) and Ethanol (1 mL) at 20° C. The reaction mixture was stirred for 2 h at 20° C. The reaction mixture was adjusted to pH=3 with HCl (1 mol/L), The reaction mixture was purified on C18 column ACN/H₂O (0.1% FA)(0-45%). The pure fractions was concentrated and dried by lyophilization. There was 4-(5-(3-((2-(3-carboxypropanoyl)-5-methoxythieno[3,2-b]pyridin-6-yl)oxy)propoxy)-6-methoxyisoindolin-2-yl)-4-oxobutanoic acid (0.015 g, 25.5707 μmol, 32.2248% yield) obtained as a white solid. LCMS: (ESI, m/z): [M+H]⁺=586.160. ¹H NMR (400 MHz, DMSO-d₆) δ 8.25 (s, 1H), 8.02 (s, 1H), 7.02-6.92 (m, 2H), 4.73 (s, 1H), 4.71 (s, 1H), 4.51 (s, 2H), 4.27 (t, J=6.2 Hz, 2H), 4.12 (s, J=4.0 Hz, 2H), 3.96 (s, 3H), 3.74 (s, 3H), 3.40-3.34 (m, 2H), 2.63-2.46 (m, 6H), 2.24 (t, J=6.2 Hz, 2H).

### Example 16

### 4-(5-((2-(((2-(3-carboxypropanoyl)-4-chloro-6-methoxyisoindolin-5-yl)oxy)methyl)allyl)oxy)-4-fluoro-6-methoxybenzo[b]thiophen-2-yl)-4-oxobutanoic Acid

### Step a: Ethyl 4-[4-chloro-5-[2-(chloromethyl)allyloxy]-6-methoxy-isoindolin-2-yl]-4-oxo-butanoate

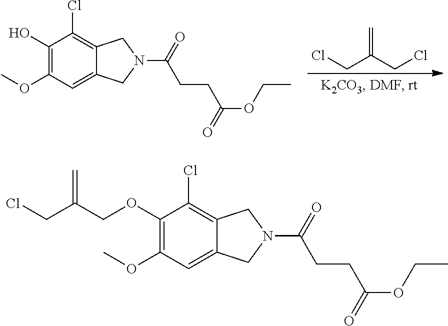

3-Chloro-2-(chloromethyl)prop-1-ene (0.507 g, 2.2635 mmol) was added to a mixture of Potassium carbonate (0.202 g, 1.4616 mmol) and ethyl 4-(4-chloro-5-hydroxy-6-methoxyisoindolin-2-yl)-4-oxobutanoate (0.154 g, 469.8574 μmol) in DMF (5 mL) at 20° C. under N₂ atmosphere. The reaction mixture was stirred for 3 h at 20° C. The reaction mixture was purified on C18 column ACN/H₂O (0.1% FA)(0-50%). The pure fractions was concentrated and dried under vacuo. There was ethyl 4-[4-chloro-5-[2-(chloromethyl)allyloxy]-6-methoxy-isoindolin-2-yl]-4-oxo-butanoate (0.151 g, 362.7238 mol, 77.1987% yield) obtained as a white solid. LCMS: (ESI, m/z): [M+H]⁺=416.100.

### Step b: Ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo[b]thiophen-2-yl)-4-oxobutanoate

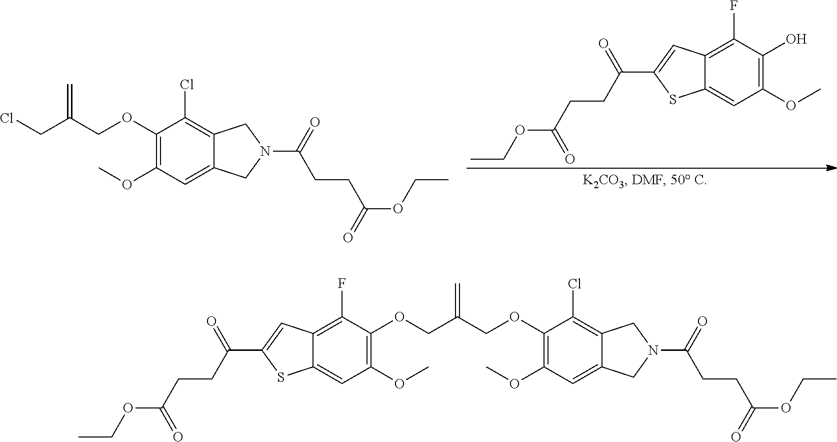

K₂CO₃ (0.092 g, 665.6754 μmol) was added to a mixture of ethyl 4-(4-chloro-5-((2-(chloromethyl)allyl)oxy)-6-methoxyisoindolin-2-yl)-4-oxobutanoate (0.068 g, 163.3458 mol) and ethyl 4-(4-fluoro-5-hydroxy-6-methoxybenzo[b]thiophen-2-yl)-4-oxobutanoate (0.049 g, 150.1504 mol) in DMF (3 mL) at 20° C. under N₂ atmosphere. The reaction mixture was stirred overnight at 50° C. The reaction mixture was purified on C18 column ACN/H₂O (0.1% FA)(0-60%). The pure fractions was concentrated and dried by lyophilization. There was ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo[b]thiophen-2-yl)-4-oxobutanoate (0.022 g, 31.1538 μmol, 19.0723% yield) obtained as a white solid. LCMS: (ESI, m/z): [M+H]⁺=706.180. ¹H NMR (400 MHz, DMSO-d₆) δ 8.30 (s, 1H), 7.56 (s, 1H), 7.07 (s, 0.5H), 7.04 (s, 0.5H), 5.34 (d, J=10.9 Hz, 2H), 4.83 (s, 1H), 4.77 (s, 2H), 4.70 (s, 1H), 4.66 (s, 2H), 4.61 (s, 1H), 4.47 (s, 1H), 4.06 (q, J=7.1 Hz, 4H), 3.89 (s, 3H), 3.81 (s, 3H), 3.40-3.34 (m, 2H), 2.69-2.54 (m, 6H), 1.21-1.14 (m, 6H).

### Step c: 4-(5-((2-(((2-(3-carboxypropanoyl)-4-chloro-6-methoxyisoindolin-5-yl)oxy)methyl)allyl)oxy)-4-fluoro-6-methoxybenzo[b]thiophen-2-yl)-4-oxobutanoic Acid

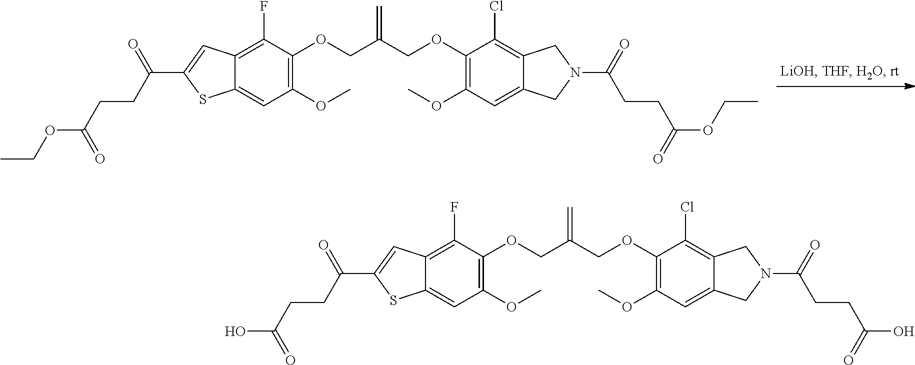 [SMILES: C=C(COc1c(OC)cc2c(c1Cl)CN(C(=O)CCC(=O)OCC)C2)COc1c(OC)cc2sc(C(=O)CCC(=O)OCC)cc2c1F ; C=C(COc1c(OC)cc2c(c1Cl)CN(C(=O)CCC(=O)O)C2)COc1c(OC)cc2sc(C(=O)CCC(=O)O)cc2c1F]

LiOH (0.037 g, 1.5450 mmol) was added to a solution of ethyl 4-(5-((2-(((4-chloro-2-(4-ethoxy-4-oxobutanoyl)-6-methoxyisoindolin-5-yl)oxy)methyl)allyl)oxy)-4-fluoro-6-methoxybenzo[b]thiophen-2-yl)-4-oxobutanoate (0.012 g, 16.9930 μmol) in THF (1 mL) and H₂O (1 mL) at 20° C. The reaction mixture was stirred for 1 h at 20° C. The reaction mixture was adjusted to pH=3 with HCl (1 mol/L). The reaction mixture was evaporated under reduced pressure. The residue was purified on C18 column ACN/H₂O (0.1% FA)(0-50%). The pure fractions was concentrated and dried by lyophilization. There was 4-(5-((2-(((2-(3-carboxypropanoyl)-4-chloro-6-methoxyisoindolin-5-yl)oxy)methyl) allyl)oxy)-4-fluoro-6-methoxybenzo[b]thiophen-2-yl)-4-oxobutanoic acid (0.011 g, 16.9213 μmol, 99.5782% yield) obtained as a white solid. LCMS: (ESI, m/z): [M−H]⁻=648.120. ¹H NMR (400 MHz, DMSO-d₆) δ 8.27 (d, J=3.0 Hz, 1H), 7.56 (s, 1H), 7.06 (s, 0.5H), 7.03 (s, 0.5H), 5.34 (d, J=10.8 Hz, 2H), 4.82 (s, 1H), 4.77 (d, J=3.3 Hz, 2H), 4.68 (s, 1H), 4.66 (s, 2H), 4.61 (s, 1H), 4.46 (s, 1H), 3.89 (s, 3H), 3.81 (s, 3H), 3.32-3.26 (m, 2H), 2.61-2.53 (m, 4H), 2.48-2.44 (m, 2H).

### Example 17

### (S)-4-(5-(3-((2-((S)-3-carboxybutanoyl)-4-fluoro-6-methoxybenzo[b]thiophen-5-yl)oxy)propoxy)-4-fluoro-6-methoxyisoindolin-2-yl)-2-methyl-4-oxobutanoic Acid

### Step a: 4-fluoro-6-methoxyisoindolin-5-ol Hydrochloride

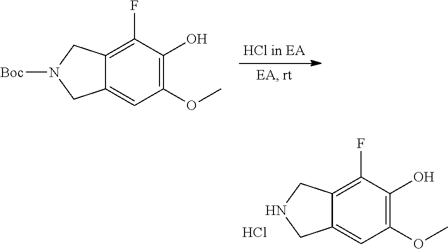 [SMILES: COc1cc2c(c(F)c1O)CN(C(=O)OC(C)(C)C)C2 ; Cl ; COc1cc2c(c(F)c1O)CNC2]

HCl in EA (3 mL, 12 mmol) was added to a solution of tert-butyl 4-fluoro-5-hydroxy-6-methoxyisoindoline-2-carboxylate (0.103 g, 363.5790 μmol) in EA (1 mL) at 20° C. The reaction mixture was stirred for 2 h at 20° C. The reaction mixture was evaporated under reduced pressure. There was 4-fluoro-6-methoxyisoindolin-5-ol hydrochloride (0.12 g, 655.0962 μmol, 180.1799% yield) obtained as a white solid, which was used directly to the next step. LCMS: (ESI, m/z): [M+H]⁺=184.070.

### Step b: Methyl (2S)-4-(4-fluoro-5-hydroxy-6-methoxy-isoindolin-2-yl)-2-methyl-4-oxo-butanoate

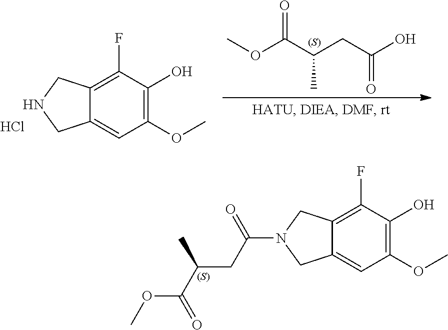

4-fluoro-6-methoxyisoindolin-5-ol hydrochloride (0.112 g, 611.4238 μmol) was added to a solution of (S)-4-methoxy-3-methyl-4-oxobutanoic acid (0.108 g, 739.0133 μmol), HATU (0.197 g, 518.1084 μmol) and DIEA (0.450 g, 3.4818 mmol) in DMF (10 mL) at 20° C. under N₂ atmosphere. The reaction mixture was stirred for 2 h at 20° C. The reaction mixture was purified on C18 column ACN/H₂O (0.1% FA)(0-55%). The pure fractions was concentrated and dried under vacuo. There was methyl (2S)-4-(4-fluoro-5-hydroxy-6-methoxy-isoindolin-2-yl)-2-methyl-4-oxo-butanoate (0.061 g, 195.9494 μmol, 32.0481% yield) obtained as a white solid. LCMS: (ESI, m/z): [M+H]⁺=312.120.

### Step c: Ethyl (2S)-4-[5-(3-bromopropoxy)-4-fluoro-6-methoxy-benzothiophen-2-yl]-2-methyl-4-oxo-butanoate

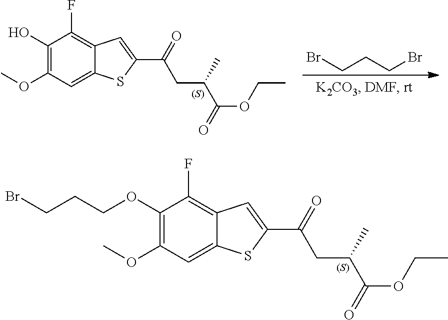

1,3-Dibromopropane (0.255 g, 1.2631 mmol) was added to a mixture of ethyl (S)-4-(4-fluoro-5-hydroxy-6-methoxybenzo[b]thiophen-2-yl)-2-methyl-4-oxobutanoate (0.061 g, 179.2189 mol) and Potassium carbonate (0.129 g, 933.3927 μmol) in DMF (3 mL) at 20° C. under N₂ atmosphere. The reaction mixture was stirred for 3 h at 20° C. The mixture was purified on C18 column ACN/H₂O (0.1% FA)(0-70%). The pure fractions was concentrated and dried under vacuo. There was ethyl (2S)-4-[5-(3-bromopropoxy)-4-fluoro-6-methoxy-benzothiophen-2-yl]-2-methyl-4-oxo-butanoate (0.66 g, 1.4306 mmol, 798.2476% yield) obtained as a white solid. LCMS: (ESI, m/z): [M+H]⁺=461.040.

### Step d: Ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo[b]thiophen-2-yl)-2-methyl-4-oxobutanoate

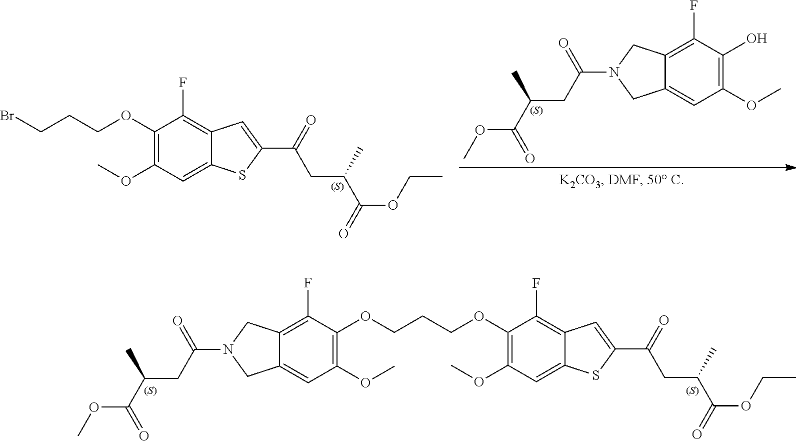

Potassium carbonate (0.074 g, 535.4346 μmol) was added to a solution of ethyl (2S)-4-[5-(3-bromopropoxy)-4-fluoro-6-methoxy-benzothiophen-2-yl]-2-methyl-4-oxo-butanoate (0.071 g, 153.8990 μmol) and methyl (2S)-4-(4-fluoro-5-hydroxy-6-methoxy-isoindolin-2-yl)-2-methyl-4-oxo-butanoate (0.056 g, 179.8880 μmol) in DMF (3 mL) at 20° C. The reaction mixture was stirred overnight at 50° C. under N₂ atmosphere. The reaction mixture was purified on C18 column ACN/H₂O (0.1% FA)(0-50%). The pure fractions was concentrated and dried under vacuo. There was ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo[b]thiophen-2-yl)-2-methyl-4-oxobutanoate (61 mg, 88.1841 μmol, 57.3000% yield) obtained as a white solid. LCMS: (ESI, m/z): [M+H]⁺=692.230. ¹H NMR (400 MHz, DMSO-d₆) δ 8.31 (s, 1H), 7.56 (s, 1H), 6.90 (s, 0.5H), 6.85 (s, 0.5H), 4.77 (s, 2H), 4.56 (s, 1H), 4.54 (s, 1H), 4.25 (t, J=6.1 Hz, 2H), 4.19 (t, J=6.1 Hz, 2H), 4.05 (q, J=7.0 Hz, 2H), 3.88 (s, 3H), 3.77 (d, J=2.1 Hz, 3H), 3.59 (s, 3H), 3.55-3.43 (m, 1H), 3.26-3.15 (m, 1H), 3.00-2.90 (m, 1H), 2.90-2.79 (m, 1H), 2.76-2.66 (m, 1H), 2.50-2.44 (m, 1H), 2.09-1.99 (m, 2H), 1.21-1.10 (m, 9H).

### Step e: (S)-4-(5-(3-((2-((S)-3-carboxybutanoyl)-4-fluoro-6-methoxybenzo[b]thiophen-5-yl)oxy)propoxy)-4-fluoro-6-methoxyisoindolin-2-yl)-2-methyl-4-oxobutanoic Acid

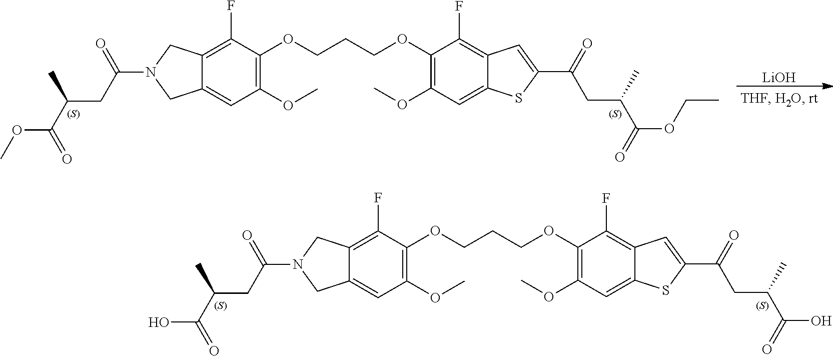

LiOH (0.038 g, 1.5868 mmol) was added to a mixture of ethyl (S)-4-(4-fluoro-5-(3-((4-fluoro-6-methoxy-2-((S)-4-methoxy-3-methyl-4-oxobutanoyl)isoindolin-5-yl)oxy)propoxy)-6-methoxybenzo[b]thiophen-2-yl)-2-methyl-4-oxobutanoate (0.038 g, 54.9344 μmol) in THF (2 mL) and H₂O (2 mL) at 20° C. The reaction mixture was stirred for 2 h at 20° C. The reaction mixture was adjusted to pH=3 with HCl (1 mol/L). The reaction mixture was evaporated under reduced pressure. The residue was purified on C18 column ACN/H₂O (0.1% FA)(0-45%). The pure fractions was concentrated and dried by lyophilization. There was (S)-4-(5-(3-((2-((S)-3-carboxybutanoyl)-4-fluoro-6-methoxybenzo[b]thiophen-5-yl)oxy)propoxy)-4-fluoro-6-methoxyisoindolin-2-yl)-2-methyl-4-oxobutanoic acid (18 mg, 27.7070 μmol, 50.4365% yield) obtained as a white solid. LCMS: (ESI, m/z): [M+H]⁺=650.18. ¹H NMR (400 MHz, DMSO-d₆) δ 12.15 (s, 2H), 8.30 (s, 1H), 7.56 (s, 1H), 6.90 (s, 0.5H), 6.85 (s, 0.5H), 4.76 (s, 2H), 4.56 (s, 1H), 4.54 (s, 1H), 4.26 (t, J=6.5 Hz, 2H), 4.19 (t, J=6.1 Hz, 2H), 3.88 (s, 3H), 3.77 (s, 3H), 3.53-3.44 (m, 1H), 3.17-3.05 (m, 1H), 2.93-2.83 (m, 1H), 2.79-2.61 (m, 2H), 2.45-2.35 (m, 1H), 2.11-1.98 (m, 2H), 1.22-1.10 (m, 6H).

### Example 18

### 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic Acid

### Step a: Ethyl 3-[[6-methoxy-5-(methoxymethoxy)benzothiophene-2-carbonyl]amino]propanoate

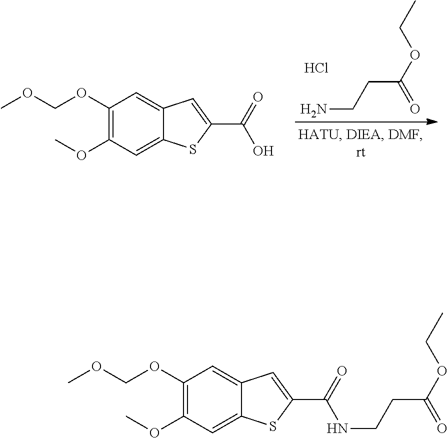

Ethyl 3-aminopropanoate hydrochloride (0.34 g, 2.2134 mmol) was added to a solution of HATU (0.85 g, 2.2355 mmol), DIEA (0.77 g, 5.9578 mmol) and 6-methoxy-5-(methoxymethoxy)benzo[b]thiophene-2-carboxylic acid (0.29 g, 1.0809 mmol) in DMF (15 mL) at 20° C. The reaction mixture was stirred for 2 h at 20° C. The mixture was purified on C18 column ACN/H₂O (0.1% FA)(0-50%). The pure fractions was concentrated and dried under vacuo. There was ethyl 3-[[6-methoxy-5-(methoxymethoxy)benzothiophene-2-carbonyl]amino]propanoate (0.35 g, 952.5987 μmol, 88.1269% yield) obtained as a yellow oil. LCMS: (ESI, m/z): [M+H]⁺=368.110.

### Step b: Ethyl 3-[(5-hydroxy-6-methoxy-benzothiophene-2-carbonyl)amino]propanoate

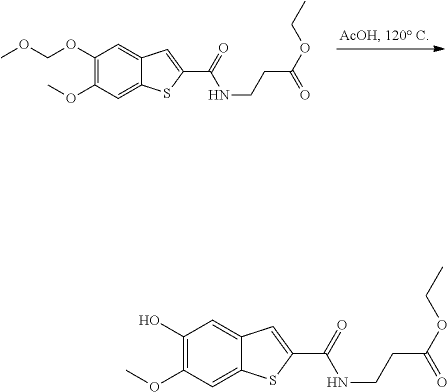

Ethyl 3-[[6-methoxy-5-(methoxymethoxy)benzothiophene-2-carbonyl]amino]propanoate (0.29 g, 789.2963 μmol) was added to Acetic acid (10 mL) at 20° C. The reaction mixture was heated to 120° C. and stirred for 4 h. The mixture was purified on C18 column ACN/H₂O (0.1% FA)(0-40%). The pure fractions was concentrated and dried under vacuo. There was ethyl 3-[(5-hydroxy-6-methoxy-benzothiophene-2-carbonyl)amino]propanoate (0.23 g, 711.2738 μmol, 90.1149% yield) obtained as a white solid. LCMS: (ESI, m/z): [M+H]⁺=324.080.

### Step c: Ethyl 4-[5-[3-[2-[(3-ethoxy-3-oxo-propyl)carbamoyl]-6-methoxy-benzothiophen-5-yl]oxypropoxy]-6-methoxy-isoindolin-2-yl]-4-oxo-butanoate Ethyl

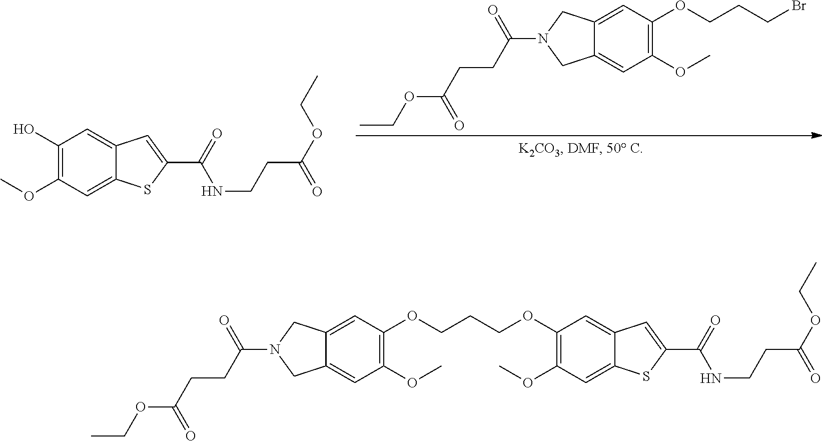

Potassium carbonate (0.25 g, 1.8089 mmol) was added to a solution of ethyl 3-[(5-hydroxy-6-methoxy-benzothiophene-2-carbonyl)amino]propanoate (0.21 g, 649.4249 μmol) and ethyl 4-(5-(3-bromopropoxy)-6-methoxyisoindolin-2-yl)-4-oxobutanoate (0.21 g, 506.8913 μmol) in DMF (5 mL) at 20° C. under N₂ atmosphere. The reaction mixture was heated to 50° C. and stirred for 8 h. The reaction mixture was purified on C18 column ACN/H₂O (0.1% FA)(0-65%). The pure fractions was concentrated and dried under vacuo. There was ethyl 4-[5-[3-[2-[(3-ethoxy-3-oxo-propyl)carbamoyl]-6-methoxy-benzothiophen-5-yl]oxypropoxy]-6-methoxy-iso indolin-2-yl]-4-oxo-butanoate (0.147 g, 223.8324 μmol, 34.4662% yield) obtained as a white solid. LCMS: (ESI, m/z): [M+H]⁺=657.240.

### Step d: 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic Acid

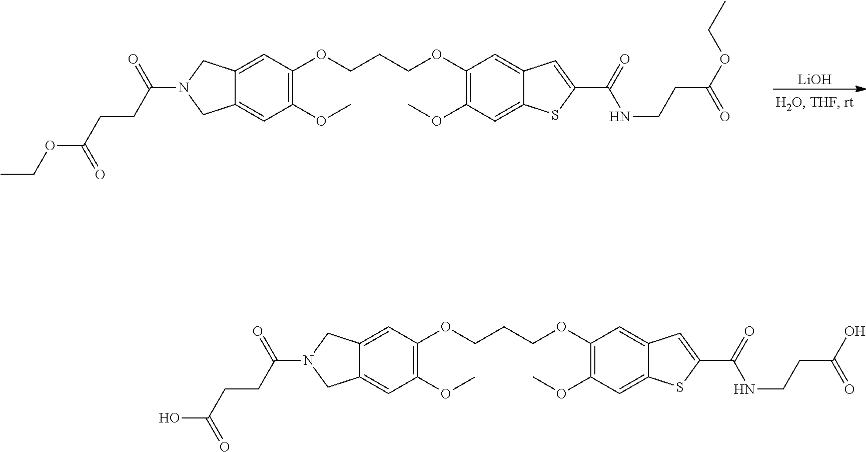 [SMILES: CCOC(=O)CCNC(=O)c1cc2cc(OCCCOc3cc4c(cc3OC)CN(C(=O)CCC(=O)OCC)C4)c(OC)cc2s1 ; "LiOH" ; COc1cc2c(cc1OCCCOc1cc3cc(C(=O)NCCC(=O)O)sc3cc1OC)CN(C(=O)CCC(=O)O)C2]

LiOH (0.101 g, 4.2174 mmol) was added to a solution of ethyl 4-[5-[3-[2-[(3-ethoxy-3-oxo-propyl)carbamoyl]-6-methoxy-benzothiophen-5-yl]oxypropoxy]-6-methoxy-iso indolin-2-yl]-4-oxo-butanoate (0.082 g, 124.8589 μmol) in THF (8 mL) and H₂O (8 mL) at 20° C. The reaction mixture was stirred for 2 h at 20° C. The reaction mixture was adjusted to pH=3 with HCl (1 mol/L). The mixture was evaporated under reduced pressure. The residue was purified on C18 column ACN/H₂O (0.1% FA)(0-45%). The pure fractions was concentrated and dried by lyophilization. There was 4-(5-(3-((2-((2-carboxyethyl)carbamoyl)-6-methoxybenzo[b]thiophen-5-yl)oxy)propoxy)-6-methoxyisoindolin-2-yl)-4-oxobutanoic acid (49 mg, 81.5803 μmol, 65.3380% yield) obtained as a white solid. LCMS: (ESI, m/z): [M+H]⁺=601.180. ¹H NMR (400 MHz, DMSO-d₆) δ 12.20 (s, 2H), 8.69 (t, J=5.5 Hz, 1H), 7.88 (s, 1H), 7.56 (s, 1H), 7.44 (d, J=2.4 Hz, 1H), 7.08-6.90 (m, 2H), 4.74 (s, 1H), 4.71 (s, 1H), 4.53 (s, 2H), 4.25-4.16 (m, 2H), 4.16-4.09 (m, 2H), 3.84 (d, J=1.5 Hz, 3H), 3.74 (d, J=2.1 Hz, 3H), 3.50-3.42 (m, 2H), 2.60-2.52 (m, 4H), 2.50-2.47 (m, 2H), 2.27-2.17 (m, 2H).

### Example 19

### 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic Acid

### Step a: Ethyl 4-[5-[2-(chloromethyl)allyloxy]-6-methoxy-isoindolin-2-yl]-4-oxo-butanoate

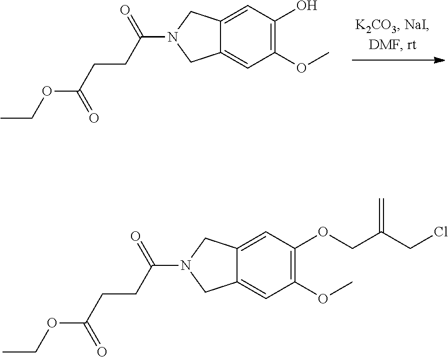

3-Chloro-2-(chloromethyl)prop-1-ene (1.108 g, 8.8643 mmol) was added to a mixture of ethyl 4-(5-hydroxy-6-methoxyisoindolin-2-yl)-4-oxobutanoate (0.507 g, 1.7285 mmol) and Potassium carbonate (0.816 g, 5.9043 mmol) in DMF (20 mL) at 20° C. The reaction mixture was stirred for 8 h at 20° C. The reaction mixture was quenched with adding of water (100 mL) at 20° C., extracted with EA (3×50 mL) and washed with brine (50 mL). The organics dried over Na₂SO₄, filtered and evaporated under reduced pressure. The residue was purified on silica gel column EA/n-Hex (0-70%). The pure fractions was concentrated and dried under vacuo. There was ethyl 4-[5-[2-(chloromethyl)allyloxy]-6-methoxy-isoindolin-2-yl]-4-oxo-butanoate (0.27 g, 707.0847 μmol, 40.9069% yield) obtained as a yellow oil. LCMS: (ESI, m/z): [M+H]⁺=382.130.

### Step b: Ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate

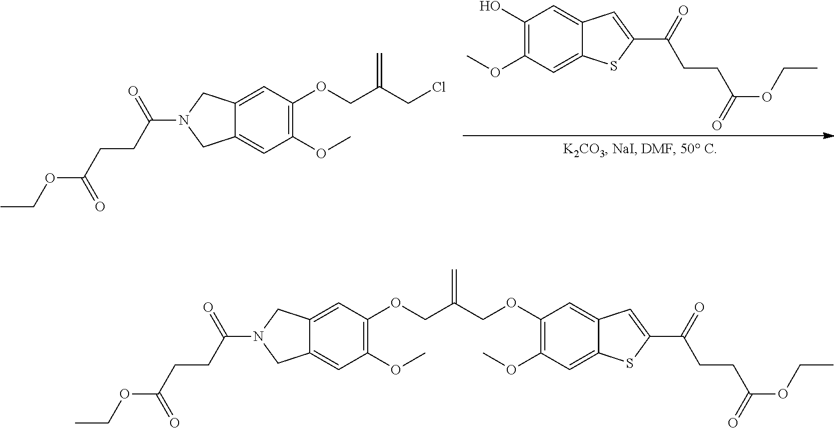

Sodium iodide (0.179 g, 1.1942 mmol) was added to a mixture of ethyl 4-[5-[2-(chloromethyl)allyloxy]-6-methoxy-isoindolin-2-yl]-4-oxo-butanoate (0.216 g, 55.6671 μmol), ethyl 4-(5-hydroxy-6-methoxybenzo[b]thiophen-2-yl)-4-oxobutanoate (0.186 g, 603.2128 μmol) and Potassium carbonate (0.238 g, 1.7221 mmol) in DMF (5 mL) at 20° C. The reaction mixture was stirred for 5 h at 20° C. under N₂ atmosphere. The mixture was purified on C18 column ACN/H₂O (0.1% FA)(0-70%). The pure fractions was concentrated and dried under vacuo. There was ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate (0.169 g, 258.5135 μmol, 45.7006% yield) obtained as a white solid. LCMS: (ESI, m/z): [M+H]⁺=654.230. ¹H NMR (400 MHz, DMSO-d₆) δ 8.16 (s, 1H), 7.63 (d, J=4.9 Hz, 1H), 7.53 (s, 1H), 7.05-6.87 (m, 2H), 5.45-5.32 (m, 2H), 4.81-4.72 (m, 3H), 4.66 (s, 3H), 4.52 (s, 1H), 4.50 (s, 1H), 4.10-4.00 (m, 4H), 3.87 (d, J=1.9 Hz, 3H), 3.76 (d, J=2.8 Hz, 3H), 3.32-3.26 (m, 2H), 2.72-2.52 (m, 6H), 1.21-1.13 (m, 6H).

### Step c: 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic Acid

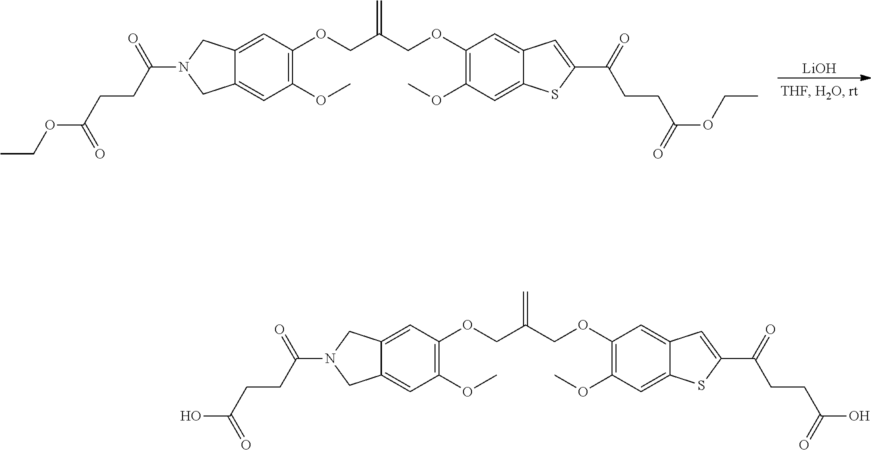

LiOH (0.073 g, 3.0482 mmol) was added to a mixture of ethyl 4-[5-[2-[[2-(4-ethoxy-4-oxo-butanoyl)-6-methoxy-isoindolin-5-yl]oxymethyl]allyloxy]-6-methoxy-benzothiophen-2-yl]-4-oxo-butanoate (0.075 g, 114.7249 μmol) in THF (4 mL) and H₂O (2 mL) at 20° C. The reaction mixture was stirred for 2 h at 20° C. The reaction mixture was adjusted to pH=3 with HCl (1 mol/L). The mixture was evaporated under reduced pressure. The residue was purified on C18 column ACN/H₂O (0.1% FA)(0-45%). The pure fractions was concentrated and dried by lyophilization. There was 4-(5-((2-(((2-(3-carboxypropanoyl)-6-methoxybenzo[b]thiophen-5-yl)oxy)methyl)allyl)oxy)-6-methoxyisoindolin-2-yl)-4-oxobutanoic acid (0.0423 g, 70.7794 μmol, 61.6949% yield) obtained as a white solid. LCMS: (ESI, m/z): [M+H]⁺=598.170. ¹H NMR (400 MHz, DMSO-d₆) δ 12.18 (s, 2H), 8.15 (s, 1H), 7.63 (d, J=4.0 Hz, 1H), 7.53 (s, 1H), 7.06-6.90 (m, 2H), 5.45-5.32 (m, 2H), 4.78-4.70 (m, 3H), 4.66 (t, J=3.3 Hz, 3H), 4.53 (s, 1H), 4.50 (s, 1H), 3.87 (d, J=1.6 Hz, 3H), 3.76 (d, J=2.6 Hz, 3H), 3.29-3.24 (m, 2H), 2.65-2.50 (m, 6H).

### Example 20

### 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic Acid

### Step a: Ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate

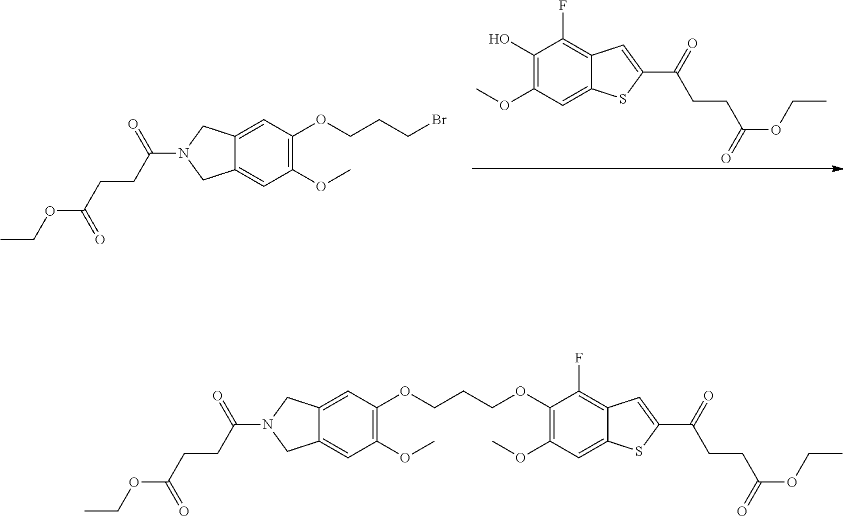 [SMILES: CCOC(=O)CCC(=O)N1Cc2cc(OC)c(OCCCBr)cc2C1 ; CCOC(=O)CCC(=O)c1cc2c(F)c(O)c(OC)cc2s1 ; CCOC(=O)CCC(=O)c1cc2c(F)c(OCCCOc3cc4c(cc3OC)CN(C(=O)CCC(=O)OCC)C4)c(OC)cc2s1]

Ethyl 4-(4-fluoro-5-hydroxy-6-methoxybenzo[b]thiophen-2-yl)-4-oxobutanoate (0.081 g, 248.2078 μmol) and Potassium carbonate (0.136 g, 984.0419 μmol) was added to a solution of ethyl 4-(5-(3-bromopropoxy)-6-methoxyisoindolin-2-yl)-4-oxobutanoate (0.103 g, 248.6182 μmol) in N,N-Dimethylformamide (10 mL) at 20° C. The reaction mixture was stirred overnight at 50° C. The reaction mixture was diluted with Ethyl acetate (100 mL), washed sequentially with water (100 mL) and brine (100 mL). The organic layer was dried over Na₂SO₄, filtered and evaporated under reduced pressure. The residue was purified on C-18 column MeCN/water (0-100%). The pure fractions was concentrated and dried under vacuo. There was ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate (66 mg, 100.0428 μmol, 40.2396% yield) obtained as a white solid. LCMS: (ESI, m/z): [M+H]⁺=432.070. ¹H NMR (400 MHz, DMSO-d₆) δ 8.32 (s, 1H), 7.57 (d, J=3.2 Hz, 1H), 6.98 (s, 0.5H), 6.95 (s, 1H), 6.92 (s, 0.5H), 4.74 (s, 2H), 4.53 (s, 2H), 4.31-4.09 (m, 4H), 4.09-3.98 (m, 4H), 3.87 (s, 3H), 3.73 (s, 3H), 3.41-3.34 (m, 2H), 2.70-2.53 (m, 6H), 2.17-2.04 (m, 2H), 1.21-1.12 (m, 6H).

### Step b: 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic Acid

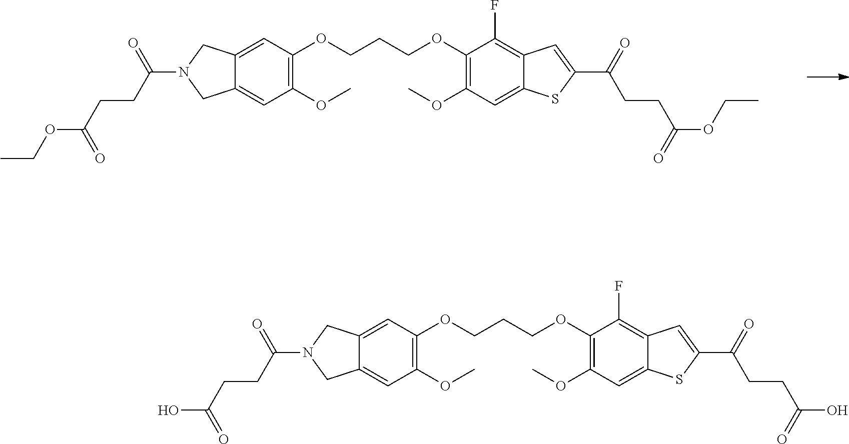 [SMILES: CCOC(=O)CCC(=O)c1cc2c(F)c(OCCCOc3cc4c(cc3OC)CN(C(=O)CCC(=O)OCC)C4)c(OC)cc2s1 ; COc1cc2c(cc1OCCCOc1c(OC)cc3sc(C(=O)CCC(=O)O)cc3c1F)CN(C(=O)CCC(=O)O)C2]

LiOH (0.010 g, 417.5662 μmol) was added to a solution of ethyl 4-(5-(3-((2-(4-ethoxy-4-oxobutanoyl)-4-fluoro-6-methoxybenzo[b]thiophen-5-yl)oxy)propoxy)-6-methoxyisoindolin-2-yl)-4-oxobutanoate (0.052 g, 78.8217 μmol) in Tetrahydrofuran (10 mL) and Water (2 mL). The reaction mixture was stirred for 2 hours at 50° C. The reaction mixture was acidified pH=4 with HCl (1 M). The mixture was evaporated under reduced pressure. The residue was purified on C-18 column MeCN/water (0-100%). The pure fractions was concentrated and dried under vacuo. There was 4-(5-(3-((2-(3-carboxypropanoyl)-4-fluoro-6-methoxybenzo[b]thiophen-5-yl)oxy)propoxy)-6-methoxyisoindolin-2-yl)-4-oxobutanoic acid (22 mg, 36.4473 μmol, 46.2402% yield) obtained as a white solid. LCMS: (ESI, m/z): [M+H]⁺=604.157. ¹H NMR (400 MHz, DMSO-d₆) δ 12.15 (s, 2H), 8.30 (s, 1H), 7.57 (s, 1H), 6.98 (s, 0.5H), 6.95 (s, 1H), 6.92 (s, 0.5H), 4.74 (s, 2H), 4.53 (s, 2H), 4.33-4.06 (m, 4H), 3.91 (s, 3H), 3.68 (s, 3H), 3.30-3.38 (m, 2H), 2.71-2.53 (m, 6H), 2.18-2.10 (m, 2H).

### Example 21

### trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic Acid

### Step a: Methyl trans-2-[5-(5,5-dimethyl-1,3,2-dioxaborinan-2-yl)-6-methoxy-benzothiophene-2-carbonyl]cyclopropanecarboxylate

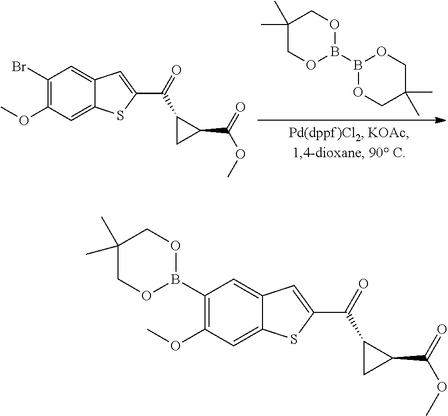

[1,1′-Bis(diphenylphosphino)ferrocene]dichloropalladium(II) (0.203 g, 277.4347 μmol) was added to a mixture of methyl trans-2-(5-bromo-6-methoxybenzo[b]thiophene-2-carbonyl)cyclopropane-1-carboxylate (0.510 g, 1.3813 mmol), bis(neopentyl glycolato)diboron (0.953 g, 4.2190 mmol) and KOAc (0.437 g, 4.4527 mmol) in 1,4-Dioxane (20 mL) at 20° C. The reaction mixture was heated to 90° C. and stirred overnight under N₂ atmosphere. The reaction mixture was filtered through a celite pad. The filtrate was evaporated under reduced pressure. There was a crude methyl trans-2-[5-(5,5-dimethyl-1,3,2-dioxaborinan-2-yl)-6-methoxy-benzothiophene-2-carbonyl]cyclopropanecarboxylate (1.61 g, 2.0012 mmol, 144.8795% yield) obtained as a black oil, which was directly used in the next step. LCMS: (ESI, m/z): [M+H]⁺=403.13.

### Step b: Methyl trans-2-(5-hydroxy-6-methoxy-benzothiophene-2-carbonyl)cyclopropanecarboxylate

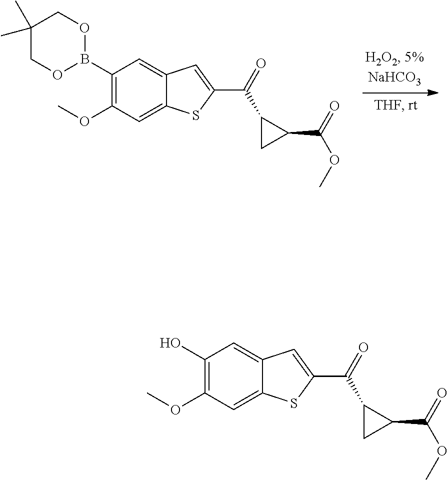 [SMILES: COC(=O)[C@H]1C[C@@H]1C(=O)c1cc2cc(B3OCC(C)(C)CO3)c(OC)cc2s1 ; COC(=O)[C@H]1C[C@@H]1C(=O)c1cc2cc(O)c(OC)cc2s1]

H₂O₂ (3.0 mL, 24.7468 mmol, 30% aqueous solution) was added to a mixture of methyl trans-2-[5-(5,5-dimethyl-1,3,2-dioxaborinan-2-yl)-6-methoxy-benzothiophene-2-carbonyl]cyclopropanecarboxylate (1.56 g, 1.9390 mmol) and NaHCO₃ (3.0 mL, 1.6722 mmol, 5% aqueous solution) in THF (30 mL) at 20° C. The reaction mixture was stirred for 1 h at 20° C. The resulting reaction mixture was diluted with brine (150 mL), extracted with EA (150 mL) and washed with brine (150 mL). The organic layer was collected and dried over Na₂SO₄, filtered and evaporated under reduced pressure. The residue was purified by silica gel column chromatography, eluting with ethyl acetate/n-hexane (0-100%). The pure fractions was concentrated and dried under reduced pressure. There was methyl trans-2-(5-hydroxy-6-methoxy-benzothiophene-2-carbonyl)cyclopropanecarboxylate (0.30 g, 979.3261 μmol, 71.43% yield) obtained as a reddish brown solid. LCMS: (ESI, m/z): [M+H]⁺=307.05. ¹H NMR (400 MHz, CDCl₃-d₆) δ 7.98 (s, 1H), 7.38 (s, 1H), 7.26 (s, 1H), 4.02 (s, 3H), 3.76 (s, 3H), 3.23-3.11 (m, 1H), 2.49-2.38 (m, 1H), 1.73-1.66 (m, 1H), 1.65-1.58 (m, 1H).

### Step c: Tert-butyl 4-fluoro-6-methoxy-5-[3-[6-methoxy-2-[trans-2-methoxycarbonylcyclopropanecarbonyl]benzothiophen-5-yl]oxypropoxy]isoindoline-2-carboxylate

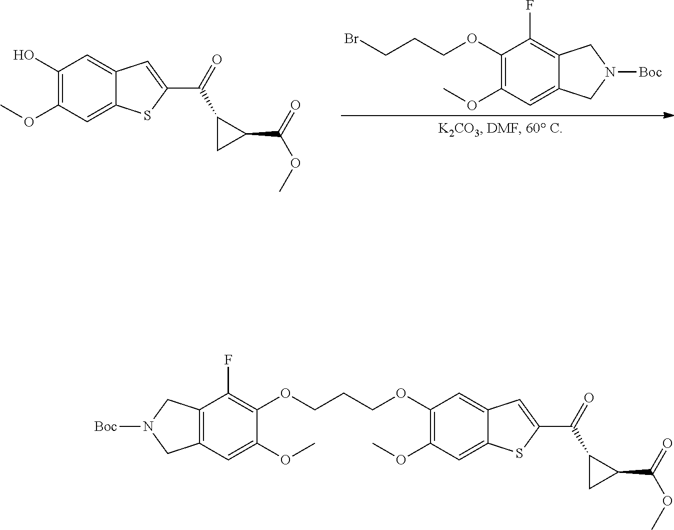 [SMILES: COC(=O)[C@H]1C[C@@H]1C(=O)c1cc2cc(O)c(OC)cc2s1 ; COc1cc2c(c(F)c1OCCCBr)CN(C(=O)OC(C)(C)C)C2 ; COC(=O)[C@H]1C[C@@H]1C(=O)c1cc2cc(OCCCOc3c(OC)cc4c(c3F)CN(C(=O)OC(C)(C)C)C4)c(OC)cc2s1]

K₂CO₃ (0.501 g, 3.6250 mmol) was added to a solution of methyl trans-2-(5-hydroxy-6-methoxy-benzothiophene-2-carbonyl)cyclopropanecarboxylate (0.300 g, 979.3264 mol) and tert-butyl 5-(3-bromopropoxy)-4-fluoro-6-methoxyisoindoline-2-carboxylate (0.406 g, 1.0043 mmol) in DMF (10 mL) at 20° C. The reaction mixture was heated to 60° C. and stirred overnight. The resulting reaction mixture was diluted with H₂O (150 mL), extracted with EA (150 mL) and washed with brine (2×150 mL). The organic layer was collected and dried over Na₂SO₄, filtered and evaporated under reduced pressure. The residue was purified by silica gel column chromatography, eluting with ethyl acetate/n-hexane (0-50%). The pure fractions was concentrated and dried under reduced pressure. There was tert-butyl 4-fluoro-6-methoxy-5-[3-[6-methoxy-2-[trans-2-methoxycarbonylcyclopropanecarbonyl]benzothiophen-5-yl]oxypropoxy]isoindoline-2-carboxylate (0.45 g, 714.6357 μmol, 72.9722% yield) obtained as a yellowish solid. LCMS: (ESI, m/z): [M+H-Boc]⁺=530.20. ¹H NMR (400 MHz, CDCl₃-d₆) δ 7.92 (s, 1H), 7.29 (s, 1H), 7.18 (s, 1H), 6.50 (s, 1H), 4.55 (s, 4H), 4.28 (t, J=6.4 Hz, 2H), 4.20 (t, J=5.8 Hz, 2H), 3.87 (s, 3H), 3.71 (s, 3H), 3.67 (s, 3H), 3.11-3.04 (m, 1H), 2.38-2.31 (m, 1H), 2.30-2.20 (m, 2H), 1.65-1.58 (m, 1H), 1.57-1.50 (m, 1H), 1.44 (s, 9H).

### Step d: Methyl trans-2-[5-[3-(4-fluoro-6-methoxy-isoindolin-5-yl)oxypropoxy]-6-methoxy-benzothiophene-2-carbonyl]cyclopropanecarboxylate

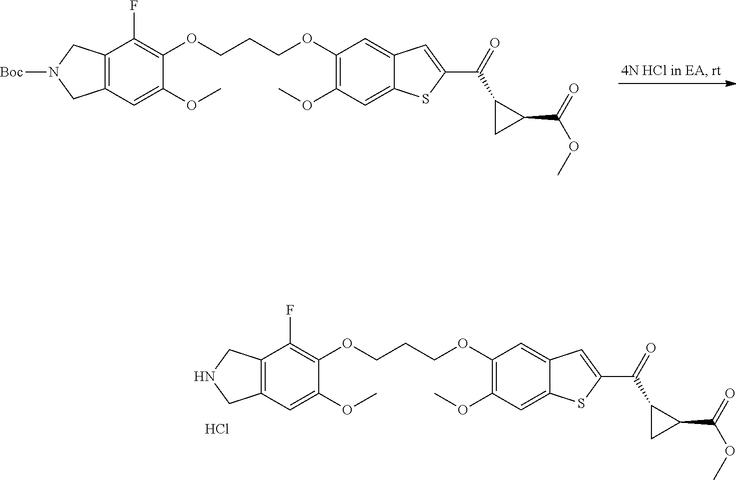 [SMILES: COC(=O)[C@H]1C[C@@H]1C(=O)c1cc2cc(OCCCOc3c(OC)cc4c(c3F)CN(C(=O)OC(C)(C)C)C4)c(OC)cc2s1 ; COC(=O)[C@H]1C[C@@H]1C(=O)c1cc2cc(OCCCOc3c(OC)cc4c(c3F)CNC4)c(OC)cc2s1 ; Cl]

HCl (4 N) in EA (15 mL, 60 mmol) was added to a solution of tert-butyl 4-fluoro-6-methoxy-5-[3-[6-methoxy-2-[trans-2-methoxycarbonylcyclopropanecarbonyl]benzothiophen-5-yl]oxypropoxy]isoindoline-2-carboxylate (0.45 g, 714.6351 μmol) in EA (5 mL) at 20° C. The reaction mixture was stirred for 2 h at 20° C. The resulting reaction mixture was concentrated under reduced pressure. There was methyl trans-2-[5-[3-(4-fluoro-6-methoxy-isoindolin-5-yl)oxypropoxy]-6-methoxy-benzothiophene-2-carbonyl]cyclopropanecarboxylate (0.42 g, 667.8009 μmol, 93.4464% yield, HCl salt) obtained as a gray solid. LCMS: (ESI, m/z): [M+H]⁺=530.20. ¹H NMR (400 MHz, DMSO-d₆) δ 10.17 (s, 2H), 8.49 (s, 1H), 7.63 (s, 1H), 7.52 (s, 1H), 6.98 (s, 1H), 4.50 (s, 2H), 4.46 (s, 2H), 4.23 (t, J=6.0 Hz, 2H), 4.17 (t, J=5.8 Hz, 2H), 3.86 (s, 3H), 3.77 (s, 3H), 3.68 (s, 3H), 3.30-3.26 (m, 1H), 2.27-2.20 (m, 1H), 2.19-2.11 (m, 2H), 1.54 (t, J=7.1 Hz, 2H).

### Step e: Methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate

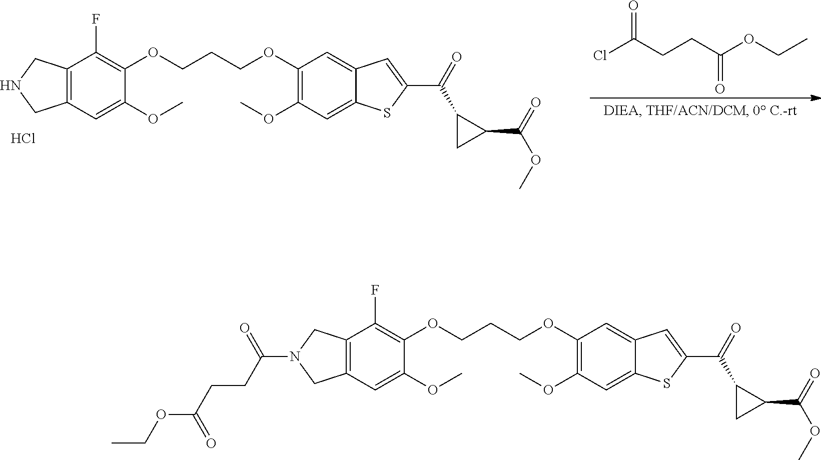

Ethyl 4-chloro-4-oxobutanoate (0.055 mL, 385.9673 μmol) in DCM (2 mL) was added dropwise to a solution of methyl trans-2-[5-[3-(4-fluoro-6-methoxy-isoindolin-5-yl)oxypropoxy]-6-methoxy-benzothiophene-2-carbonyl]cyclopropanecarboxylate (0.202 g, 356.8671 μmol, HCl salt) and N,N-Diisopropylethylamine (0.185 mL, 1.0621 mmol) in THF (4 mL) and ACN (4 mL) at 0° C. The reaction mixture was stirred for 2 h at 20° C. The reaction mixture was concentrated under reduced pressure. The residue was purified by Prep-TLC with the developing solvent of DCM/MeOH (25:1). There was methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate (0.221 g, 336.0184 μmol, 94.1579% yield) obtained as an off-white semi-solid. LCMS: (ESI, m/z): [M+H]⁺=658.25. ¹H NMR (400 MHz, CDCl₃-d₆) δ 7.93 (s, 1H), 7.19 (s, 2H), 6.54 (s, 0.5H), 6.49 (s, 0.5H), 4.74 (d, J=7.7 Hz, 2H), 4.68 (d, J=5.6 Hz, 2H), 4.29 (s, 2H), 4.21 (s, 2H), 4.09 (q, J=7.1 Hz, 2H), 3.88 (s, 3H), 3.73 (s, 3H), 3.68 (s, 3H), 3.15-3.02 (m, 1H), 2.72-2.54 (m, 4H), 2.40-2.31 (m, 1H), 2.30-2.18 (m, 2H), 1.65-1.51 (m, 2H), 1.20 (t, J=7.0 Hz, 3H).

### Step f: trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic Acid

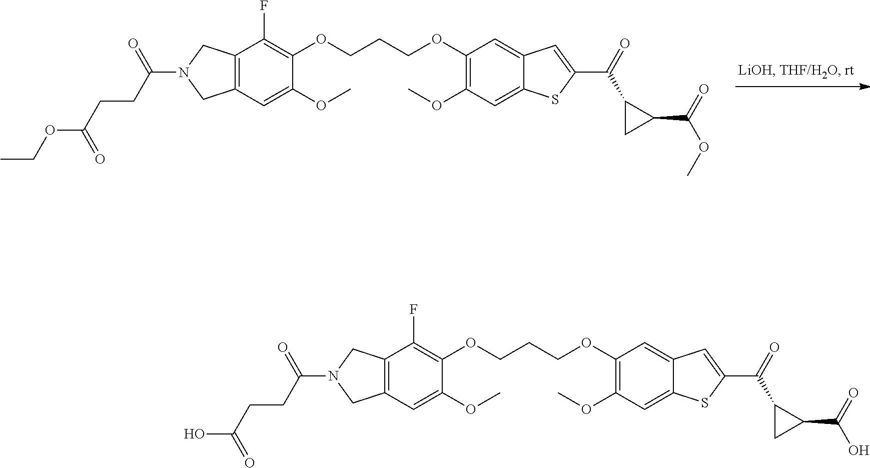 [SMILES: CCOC(=O)CCC(=O)N1Cc2cc(OC)c(OCCCOc3cc4cc(C(=O)[C@H]5C[C@@H]5C(=O)OC)sc4cc3OC)c(F)c2C1 ; COc1cc2sc(C(=O)[C@H]3C[C@@H]3C(=O)O)cc2cc1OCCCOc1c(OC)cc2c(c1F)CN(C(=O)CCC(=O)O)C2]

LiOH (0.027 g, 1.1274 mmol) was added to a solution of methyl trans-2-[5-[3-[2-(4-ethoxy-4-oxo-butanoyl)-4-fluoro-6-methoxy-isoindolin-5-yl]oxypropoxy]-6-methoxy-benzothiophene-2-carbonyl]cyclopropanecarboxylate (0.165 g, 250.8735 μmol) in THF (6 mL) and H₂O (2 mL) at 20° C. The reaction mixture was stirred for 2 h at 20° C. The resulting reaction mixture was adjusted to pH=2-3 with HCl (6 N) and evaporated under reduced pressure. The residue was purified by reverse C18 column chromatography, eluting with the mobile phase ACN/H₂O (0.1% FA)(0-100%). The pure fractions was concentrated and lyophilized overnight. There was trans-2-(5-(3-((2-(3-carboxypropanoyl)-4-fluoro-6-methoxyisoindolin-5-yl)oxy)propoxy)-6-methoxybenzo[b]thiophene-2-carbonyl)cyclopropane-1-carboxylic acid (0.052 g, 84.4674 μmol, 33.6693% yield) obtained as a white solid. LCMS: (ESI, m/z): [M+H]⁺=616.20. ¹H NMR (400 MHz, DMSO-d₆) δ 12.45 (s, 2H), 8.48 (s, 1H), 7.61 (s, 1H), 7.51 (d, J=2.2 Hz, 1H), 6.91 (s, 0.5H), 6.88 (s, 0.5H), 4.78 (d, J=5.5 Hz, 2H), 4.56 (s, 2H), 4.23 (t, J=5.0 Hz, 2H), 4.17 (t, J=5.9 Hz, 2H), 3.86 (s, 3H), 3.76 (s, 3H), 3.27-3.19 (m, 1H), 2.60-2.53 (m, 2H), 2.49-2.44 (m, 2H), 2.21-2.07 (m, 3H), 1.49 (t, J=7.1 Hz, 2H).

The following example shown in table 2 was synthesized using the above procedure with the corresponding starting materials.

TABLE 2

| | | | MS: |
|---|---|---|---|
| | | | (M + H)⁺ |
| EX | | | & |
| No. | Chemical name | Structure | ¹HNMR |
| 22. | 4-(5-(3-((2-(3-carboxy- butanoyl)-6-methoxybenzo [b]thiophen-5-yl)oxy) propoxy)-6-methoxy- isoindolin-2-yl)-2-methyl- 4-oxobutanoic acid | 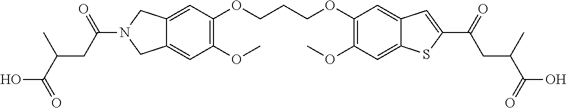 [SMILES: COc1cc2c(cc1OCCCOc1cc3cc(C(=O)CC(C)C(=O)O)sc3cc1OC)CN(C(=O)CC(C)C(=O)O)C2] | 614 |
| 23. | (S)-4-(5-(3-((2-((S)-3- carboxybutanoyl)-6- methoxybenzo[b]thiophen- 5-yl)oxy)propoxy)-6- methoxyisoindolin-2-yl)- 2-methyl-4-oxobutanoic acid | 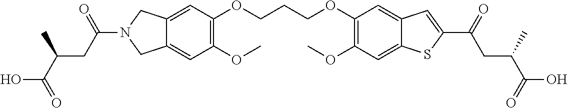 [SMILES: COc1cc2c(cc1OCCCOc1cc3cc(C(=O)C[C@H](C)C(=O)O)sc3cc1OC)CN(C(=O)C[C@H](C)C(=O)O)C2] | 614 |
| 24. | (S)-4-(5-(3-((2-(3-carboxy- propanoyl)-6-methoxy- isoindolin-5-yl)oxy) propoxy)-6-methoxy- benzo[b]thiophen-2-yl)-2- methyl-4-oxobutanoic acid | 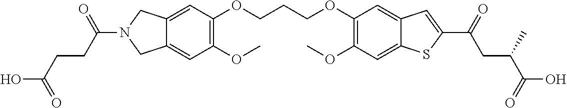 [SMILES: COc1cc2c(cc1OCCCOc1cc3cc(C(=O)C[C@H](C)C(=O)O)sc3cc1OC)CN(C(=O)CCC(=O)O)C2] | 600 |
| 25. | (S)-4-(5-(3-((2-(3-carboxy- propanoyl)-6-methoxy- benzo[b]thiophen-5-yl) oxy)propoxy)-6-methoxy- isoindolin-2-yl)-2- methyl-4-oxobutanoic acid | 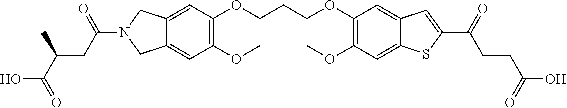 [SMILES: COc1cc2c(cc1OCCCOc1cc3cc(C(=O)CCC(=O)O)sc3cc1OC)CN(C(=O)C[C@H](C)C(=O)O)C2] | 600 |
| 26. | (S)-4-(5-(2-((2-((S)-3- carboxybutanoyl)-6- methoxybenzo[b]thiophen- 5-yl)oxy)ethoxy)-6- methoxyisoindolin-2-yl)- 2-methyl-4-oxobutanoic acid | 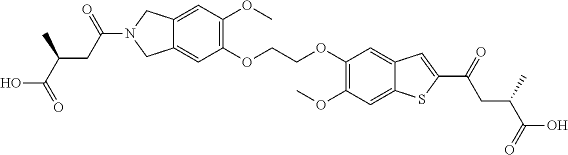 [SMILES: COc1cc2c(cc1OCCOc1cc3cc(C(=O)C[C@H](C)C(=O)O)sc3cc1OC)CN(C(=O)C[C@H](C)C(=O)O)C2] | 600 |
| 27. | (S)-4-(5-(4-((2-((S)-3- carboxybutanoyl)-6- methoxybenzo[b]thiophen- 5-yl)oxy)butoxy)-6- methoxyisoindolin-2-yl)- 2-methyl-4-oxobutanoic acid | 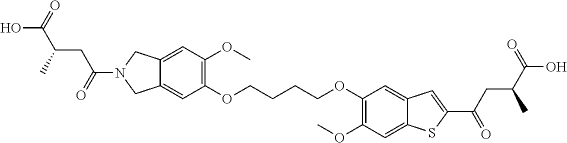 [SMILES: COc1cc2c(cc1OCCCCOc1cc3cc(C(=O)C[C@H](C)C(=O)O)sc3cc1OC)CN(C(=O)C[C@H](C)C(=O)O)C2] | 628 |
| 28. | (S)-4-(5-((5-((2-((S)-3- carboxybutanoyl)-6- methoxybenzo[b]thiophen- 5-yl)oxy)pentyl)oxy)-6- methoxyisoindolin-2-yl)- 2-methyl-4-oxobutanoic acid | 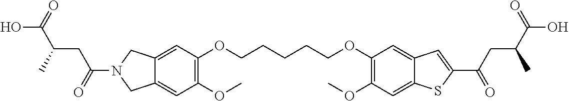 [SMILES: COc1cc2c(cc1OCCCCCOc1cc3cc(C(=O)C[C@H](C)C(=O)O)sc3cc1OC)CN(C(=O)C[C@H](C)C(=O)O)C2] | 642 |
| 29. | (S)-4-(5-(4-(2-((S)-3- carboxybutanoyl)-6- methoxybenzo[b]thiophen- 5-yl)butoxy)-6-methoxy- isoindolin-2-yl)-2-methyl- 4-oxobutanoic acid | 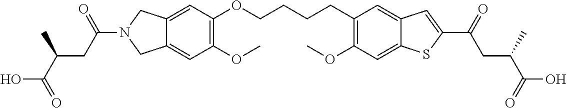 [SMILES: COc1cc2sc(C(=O)C[C@H](C)C(=O)O)cc2cc1CCCCOc1cc2c(cc1OC)CN(C(=O)C[C@H](C)C(=O)O)C2] | 612 |
| 30. | (S)-4-(5-(3-((2-((S)-3- carboxybutanoyl)-6- methoxybenzo[b]thiophen- 5-yl)amino)propoxy)-6- methoxyisoindolin-2-yl)- 2-methyl-4-oxobutanoic acid | 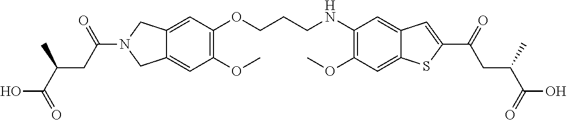 [SMILES: COc1cc2sc(C(=O)C[C@H](C)C(=O)O)cc2cc1NCCCOc1cc2c(cc1OC)CN(C(=O)C[C@H](C)C(=O)O)C2] | 613 |
| 31. | (S)-4-(5-(4-((2-((S)-3- carboxybutanoyl)-6- methoxybenzo[b]thiophen- 5-yl)amino)butyl)-6- methoxyisoindolin-2-yl)- 2-methyl-4-oxobutanoic acid | 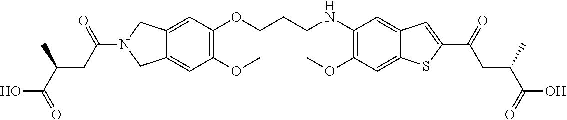 [SMILES: COc1cc2sc(C(=O)C[C@H](C)C(=O)O)cc2cc1NCCCOc1cc2c(cc1OC)CN(C(=O)C[C@H](C)C(=O)O)C2] | 611 |
| 32. | (S)-4-(5-(4-(2-((S)-3- carboxybutanoyl)-6- methoxybenzo[b]thiophen- 5-yl)butyl)-6-methoxy- isoindolin-2-yl)-2- methyl-4-oxobutanoic acid | 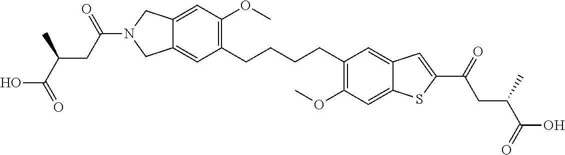 [SMILES: COc1cc2c(cc1CCCCc1cc3cc(C(=O)C[C@H](C)C(=O)O)sc3cc1OC)CN(C(=O)C[C@H](C)C(=O)O)C2] | 596 |
| 33. | (S)-4-(5-(5-(2-((S)-3- carboxybutanoyl)-6- methoxybenzo[b]thiophen- 5-yl)pentyl)-6-methoxy- isoindolin-2-yl)-2-methyl- 4-oxobutanoic acid | 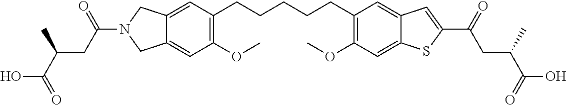 [SMILES: COc1cc2c(cc1CCCCCc1cc3cc(C(=O)C[C@H](C)C(=O)O)sc3cc1OC)CN(C(=O)C[C@H](C)C(=O)O)C2] | 610 |
| 34. | (2S)-4-(5-((4-((2-((S)-3- carboxybutanoyl)-6- methoxybenzo[b] thiophen-5-yl)oxy)pentan- 2-yl)oxy)-6-methoxy- isoindolin-2-yl)-2-methyl- 4-oxobutanoic acid | 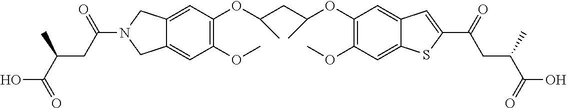 [SMILES: COc1cc2c(cc1OC(C)CC(C)Oc1cc3cc(C(=O)C[C@H](C)C(=O)O)sc3cc1OC)CN(C(=O)C[C@H](C)C(=O)O)C2] | 642 |
| 35. | (S)-4-(5-(3-((2-((S)-3- carboxybutanoyl)-6- methoxybenzo[b]thiophen- 5-yl)oxy)-2,2-dimethyl- propoxy)-6-methoxy- isoindolin-2-yl)-2-methyl- 4-oxobutanoic acid | 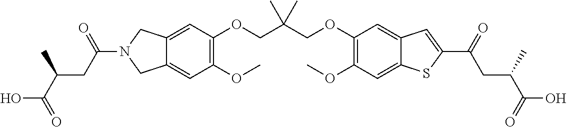 [SMILES: COc1cc2c(cc1OCC(C)(C)COc1cc3cc(C(=O)C[C@H](C)C(=O)O)sc3cc1OC)CN(C(=O)C[C@H](C)C(=O)O)C2] | 642 |
| 36. | (S)-4-(5-((1-(((2-((S)-3- carboxybutanoyl)-6- methoxybenzo[b]thiophen- 5-yl)oxy)methyl)cyclo- propyl)methoxy)-6- methoxyisoindolin-2-yl)- 2-methyl-4-oxobutanoic acid | 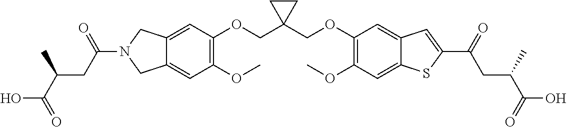 [SMILES: COc1cc2c(cc1OCC1(COc3cc4cc(C(=O)C[C@H](C)C(=O)O)sc4cc3OC)CC1)CN(C(=O)C[C@H](C)C(=O)O)C2] | 640 |
| 37. | 4-(5-(3-((2-(3-carboxy- propanoyl)-6-methoxy- isoindolin-5-yl)oxy)pro- poxy)-6-methoxybenzo [b]thiophen-2-yl)-2,2- dimethyl-4-oxobutanoic acid | 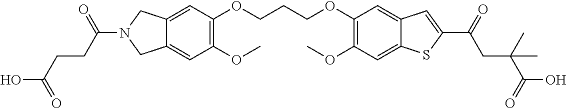 [SMILES: COc1cc2c(cc1OCCCOc1cc3cc(C(=O)CC(C)(C)C(=O)O)sc3cc1OC)CN(C(=O)CCC(=O)O)C2] | 614 |
| 38. | 4-(5-(3-((2-(3-carboxy- propanoyl)-6-methoxy- benzo[b]thiophen-5-yl) oxy)propoxy)-6- methoxyisoindolin-2-yl)- 2,2-dimethyl-4- oxobutanoic acid | 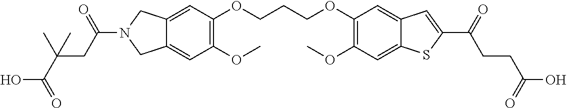 [SMILES: COc1cc2c(cc1OCCCOc1cc3cc(C(=O)CCC(=O)O)sc3cc1OC)CN(C(=O)CC(C)(C)C(=O)O)C2] | 614 |
| 39. | 4-(5-(3-((2-(3-carboxy-3- methylbutanoyl)-6- methoxybenzo[b]thiophen- 5-yl)oxy)propoxy)-6- methoxyisoindolin-2- yl)-2,2-dimethyl-4- oxobutanoic acid | 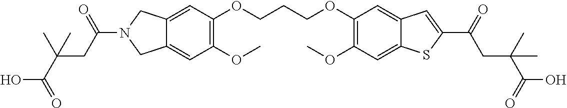 [SMILES: COc1cc2c(cc1OCCCOc1cc3cc(C(=O)CC(C)(C)C(=O)O)sc3cc1OC)CN(C(=O)CC(C)(C)C(=O)O)C2] | 642 |
| 40. | (S)-4-(5-((4-(2-((S)-3- carboxybutanoyl)-6- methoxybenzo[b]thiophen- 5-yl)butyl)amino)-6- methoxyisoindolin-2-yl)-2- methyl-4-oxobutanoic acid | 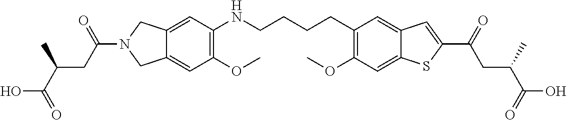 [SMILES: COc1cc2sc(C(=O)C[C@H](C)C(=O)O)cc2cc1CCCCNc1cc2c(cc1OC)CN(C(=O)C[C@H](C)C(=O)O)C2] | 611 |
| 41. | (S)-4-(5-((3-((2-((S)-3- carboxybutanoyl)-6- methoxybenzo[b]thiophen- 5-yl)oxy)propyl)amino)- 6-methoxyisoindolin-2- yl)-2-methyl-4-oxo- butanoic acid | 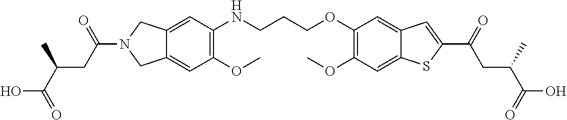 [SMILES: COc1cc2c(cc1NCCCOc1cc3cc(C(=O)C[C@H](C)C(=O)O)sc3cc1OC)CN(C(=O)C[C@H](C)C(=O)O)C2] | 613 |
| 42. | (S)-4-(5-(4-((2-((S)-3- carboxybutanoyl)-6- methoxybenzo[b] thiophen-5-yl)oxy)butyl)- 6-methoxyisoindolin-2-yl)- 2-methyl-4-oxobutanoic acid | 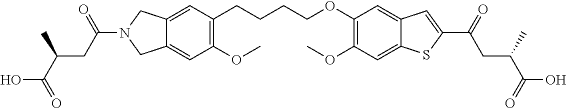 [SMILES: COc1cc2c(cc1CCCCOc1cc3cc(C(=O)C[C@H](C)C(=O)O)sc3cc1OC)CN(C(=O)C[C@H](C)C(=O)O)C2] | 612 |
| 43. | (S)-4-(5-(3-((2-((S)-3- carboxybutanoyl)-4- fluoro-6-methoxybenzo [b]thiophen-5-yl)oxy) propoxy)-6-methoxy- isoindolin-2-yl)-2-methyl- 4-oxobutanoic acid | 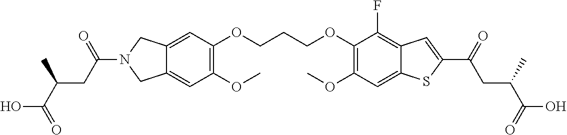 [SMILES: COc1cc2c(cc1OCCCOc1c(OC)cc3sc(C(=O)C[C@H](C)C(=O)O)cc3c1F)CN(C(=O)C[C@H](C)C(=O)O)C2] | 632 |
| 44. | (S)-4-(5-(3-((2-((S)-3- carboxybutanoyl)-4- chloro-6-methoxybenzo [b]thiophen-5-yl)oxy) propoxy)-6-methoxyiso- indolin-2-yl)-2-methyl-4- oxobutanoic acid | 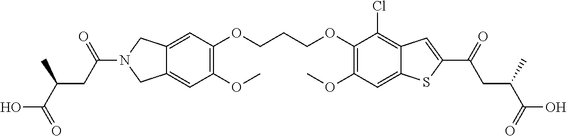 [SMILES: COc1cc2c(cc1OCCCOc1c(OC)cc3sc(C(=O)C[C@H](C)C(=O)O)cc3c1Cl)CN(C(=O)C[C@H](C)C(=O)O)C2] | 648 |
| 45. | (S)-4-(5-(3-((4-bromo-2- ((S)-3-carboxybutanoyl)- 6-methoxybenzo[b] thiophen-5-yl)oxy) propoxy)-6-methoxyiso- indolin-2-yl)-2-methyl-4- oxobutanoic acid | 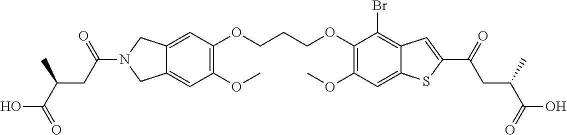 [SMILES: COc1cc2c(cc1OCCCOc1c(OC)cc3sc(C(=O)C[C@H](C)C(=O)O)cc3c1Br)CN(C(=O)C[C@H](C)C(=O)O)C2] | 692 |
| 46. | (S)-4-(5-(3-((2-((S)-3- carboxybutanoyl)-6- methoxybenzo[d]thiazol- 5-yl)oxy)propoxy)-6- methoxyisoindolin-2-yl)- 2-methyl-4-oxobutanoic acid | 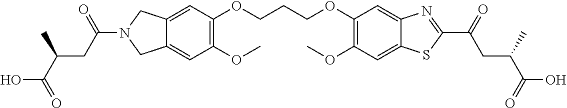 [SMILES: COc1cc2c(cc1OCCCOc1cc3nc(C(=O)C[C@H](C)C(=O)O)sc3cc1OC)CN(C(=O)C[C@H](C)C(=O)O)C2] | 615 |
| 47. | (S)-4-(5-(3-((2-((S)-3- carboxybutanoyl)-6- hydroxybenzo[b]thiophen- 5-yl)oxy)propoxy)-6- methoxyisoindolin-2-yl)- 2-methyl-4-oxobutanoic acid | 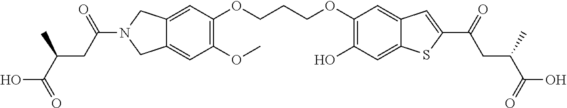 [SMILES: COc1cc2c(cc1OCCCOc1cc3cc(C(=O)C[C@H](C)C(=O)O)sc3cc1O)CN(C(=O)C[C@H](C)C(=O)O)C2] | 600 |
| 48. | (S)-4-(5-(3-((2-((S)-3- carboxybutanoyl)-4,7- difluoro-6-methoxy- benzo[b]thiophen-5-yl) oxy)propoxy)-6- methoxyisoindolin-2- yl)-2-methyl-4- oxobutanoic acid | 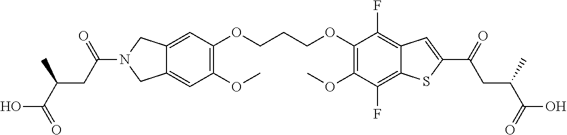 [SMILES: COc1cc2c(cc1OCCCOc1c(OC)c(F)c3sc(C(=O)C[C@H](C)C(=O)O)cc3c1F)CN(C(=O)C[C@H](C)C(=O)O)C2] | 650 |
| 49. | (S)-4-(5-(3-((2-((S)-3- carboxybutanoyl)-4,7- dichloro-6-methoxy- benzo[b]thiophen-5-yl) oxy)propoxy)-6-methoxy- isoindolin-2-yl)-2- methyl-4-oxobutanoic acid | 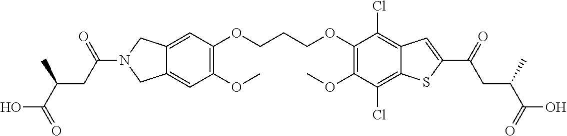 [SMILES: COc1cc2c(cc1OCCCOc1c(OC)c(Cl)c3sc(C(=O)C[C@H](C)C(=O)O)cc3c1Cl)CN(C(=O)C[C@H](C)C(=O)O)C2] | 682 |
| 50. | (2S)-4-(5-(3-((2-((S)-3- carboxybutanoyl)-6- methoxy-3-methyl- isoindolin-5-yl)oxy) propoxy)-6- methoxybenzo[b]thiophen- 2-yl)-2-methyl-4- oxobutanoic acid | 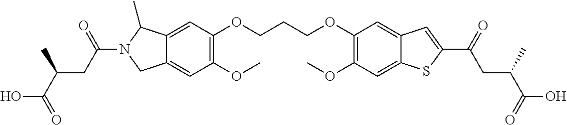 [SMILES: COc1cc2c(cc1OCCCOc1cc3cc(C(=O)C[C@H](C)C(=O)O)sc3cc1OC)C(C)N(C(=O)C[C@H](C)C(=O)O)C2] | 628 |
| 51. | (S)-4-(5-(3-((2-((S)-3- carboxybutanoyl)-4- fluoro-6-methoxy- isoindolin-5-yl)oxy) propoxy)-6-methoxy- benzo[b]thiophen-2- yl)-2-methyl-4- oxobutanoic acid | 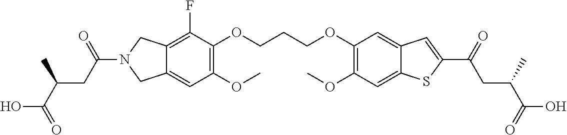 [SMILES: COc1cc2sc(C(=O)C[C@H](C)C(=O)O)cc2cc1OCCCOc1c(OC)cc2c(c1F)CN(C(=O)C[C@H](C)C(=O)O)C2] | 632 |
| 52. | (S)-4-(5-(3-((2-((S)-3- carboxybutanoyl)-4- chloro-6-methoxy- isoindolin-5-yl)oxy) propoxy)-6-methoxy- benzo[b]thiophen-2-yl)- 2-methyl-4-oxobutanoic acid | 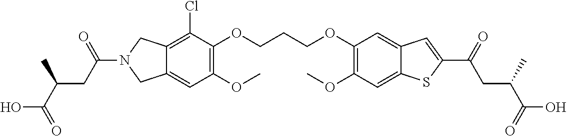 [SMILES: COc1cc2sc(C(=O)C[C@H](C)C(=O)O)cc2cc1OCCCOc1c(OC)cc2c(c1Cl)CN(C(=O)C[C@H](C)C(=O)O)C2] | 648 |
| 53. | (S)-4-(5-(3-((4-bromo-2- ((S)-3-carboxybutanoyl)- 6-methoxyisoindolin-5- yl)oxy)propoxy)-6- methoxybenzo[b]thiophen- 2-yl)-2-methyl-4- oxobutanoic acid | 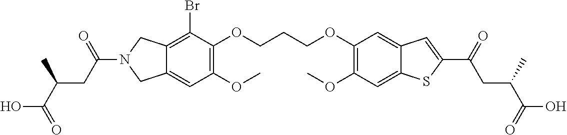 [SMILES: COc1cc2sc(C(=O)C[C@H](C)C(=O)O)cc2cc1OCCCOc1c(OC)cc2c(c1Br)CN(C(=O)C[C@H](C)C(=O)O)C2] | 692 |
| 54. | (S)-4-(5-(3-((2-((S)-3- carboxybutanoyl)-6- hydroxyisoindolin-5-yl) oxy)propoxy)-6- methoxybenzo[b] thiophen-2-yl)-2-methyl- 4-oxobutanoic acid | 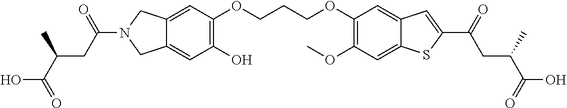 [SMILES: COc1cc2sc(C(=O)C[C@H](C)C(=O)O)cc2cc1OCCCOc1cc2c(cc1O)CN(C(=O)C[C@H](C)C(=O)O)C2] | 600 |
| 55. | (S)-4-(6-(3-((2-((S)-3- carboxybutanoyl)-6- methoxybenzo[b]thiophen- 5-yl)oxy)propoxy)-4- fluoro-5-methoxy- isoindolin-2-yl)-2-methyl- 4-oxobutanoic acid | 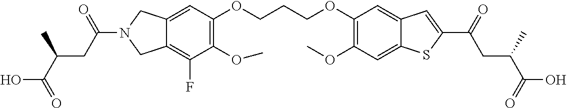 [SMILES: COc1cc2sc(C(=O)C[C@H](C)C(=O)O)cc2cc1OCCCOc1cc2c(c(F)c1OC)CN(C(=O)C[C@H](C)C(=O)O)C2] | 632 |
| 56. | (S)-4-(6-(3-((2-((S)-3- carboxybutanoyl)-6- methoxybenzo[b]thiophen- 5-yl)oxy)propoxy)-4- chloro-5-methoxy- isoindolin-2-yl)-2-methyl- 4-oxobutanoic acid | 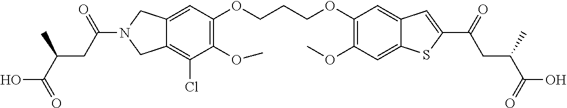 [SMILES: COc1cc2sc(C(=O)C[C@H](C)C(=O)O)cc2cc1OCCCOc1cc2c(c(Cl)c1OC)CN(C(=O)C[C@H](C)C(=O)O)C2] | 648 |
| 57. | (S)-4-(5-(3-((7-bromo-2- ((S)-3-carboxybutanoyl)- 6-methoxyisoindolin-5- yl)oxy)propoxy)-6- methoxybenzo[b]thiophen- 2-yl)-2-methyl-4- oxobutanoic acid | 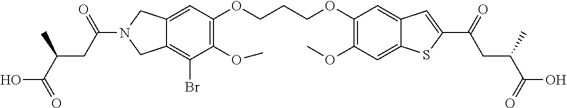 [SMILES: COc1cc2sc(C(=O)C[C@H](C)C(=O)O)cc2cc1OCCCOc1cc2c(c(Br)c1OC)CN(C(=O)C[C@H](C)C(=O)O)C2] | 692 |
| 58. | (S)-4-(5-(3-((2-((S)-3- carboxybutanoyl)-4,7- difluoro-6-methoxy- isoindolin-5-yl)oxy) propoxy)-6-methoxy- benzo[b]thiophen-2-yl)-2- methyl-4-oxobutanoic acid | 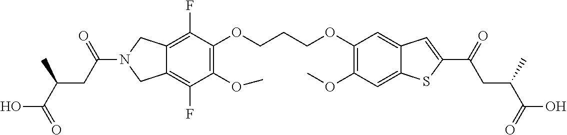 [SMILES: COc1cc2sc(C(=O)C[C@H](C)C(=O)O)cc2cc1OCCCOc1c(F)c2c(c(F)c1OC)CN(C(=O)C[C@H](C)C(=O)O)C2] | 650 |
| 59. | (S)-4-(5-(3-((2-((S)-3- carboxybutanoyl)-6- methoxybenzo[b]thiophen- 5-yl)oxy)propoxy)-7- chloro-4-fluoro-6- methoxyisoindolin-2-yl)- 2-methyl-4-oxobutanoic acid | 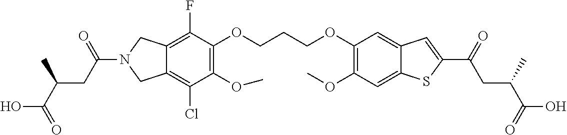 [SMILES: COc1cc2sc(C(=O)C[C@H](C)C(=O)O)cc2cc1OCCCOc1c(F)c2c(c(Cl)c1OC)CN(C(=O)C[C@H](C)C(=O)O)C2] | 666 |
| 60. | (S)-4-(5-(3-((2-((S)-3- carboxybutanoyl)-4,7- dichloro-6-methoxy- isoindolin-5-yl)oxy) propoxy)-6-methoxy- benzo[b]thiophen-2-yl)-2- methyl-4-oxobutanoic acid | 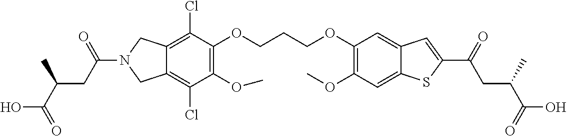 [SMILES: COc1cc2sc(C(=O)C[C@H](C)C(=O)O)cc2cc1OCCCOc1c(Cl)c2c(c(Cl)c1OC)CN(C(=O)C[C@H](C)C(=O)O)C2] | 682 |
| 61. | (S)-4-(5-(3-((2-((S)-3- carboxybutanoyl)-4- chloro-6-methoxybenzo[b] thiophen-5-yl)oxy) propoxy)-4-fluoro-6- methoxyisoindolin-2-yl)- 2-methyl-4-oxobutanoic acid | 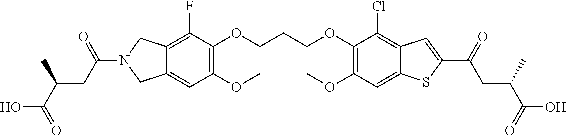 [SMILES: COc1cc2c(c(F)c1OCCCOc1c(OC)cc3sc(C(=O)C[C@H](C)C(=O)O)cc3c1Cl)CN(C(=O)C[C@H](C)C(=O)O)C2] | 666 |
| 62. | (S)-4-(5-(3-((2-((S)-3- carboxybutanoyl)-4- chloro-6-methoxyiso- indolin-5-yl)oxy)propoxy)- fluoro-6-methoxybenzo[b] thiophen-2-yl)-2-methyl- 4-oxobutanoic acid | 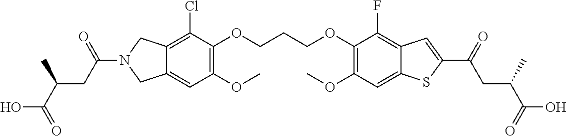 [SMILES: COc1cc2c(c(Cl)c1OCCCOc1c(OC)cc3sc(C(=O)C[C@H](C)C(=O)O)cc3c1F)CN(C(=O)C[C@H](C)C(=O)O)C2] | 666 |
| 63. | (S)-4-(5-(3-((2-((S)-3- carboxybutanoyl)-4- chloro-6-methoxybenzo[b] thiophen-5-yl)oxy) propoxy)-4-chloro-6- methoxyisoindolin-2-yl)- 2-methyl-4-oxobutanoic acid | 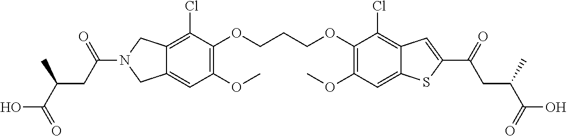 [SMILES: COc1cc2c(c(Cl)c1OCCCOc1c(OC)cc3sc(C(=O)C[C@H](C)C(=O)O)cc3c1Cl)CN(C(=O)C[C@H](C)C(=O)O)C2] | 682 |
| 64. | (S)-4-(6-(3-((2-((S)-3- carboxybutanoyl)-7- fluoro-6-methoxybenzo[b] thiophen-5-yl)oxy) propoxy)-4-fluoro-5- methoxyisoindolin-2-yl)- 2-methyl-4-oxobutanoic acid | 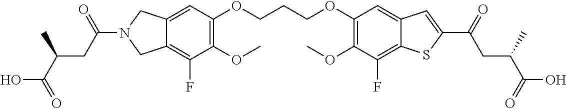 [SMILES: COc1c(OCCCOc2cc3cc(C(=O)C[C@H](C)C(=O)O)sc3c(F)c2OC)cc2c(c1F)CN(C(=O)C[C@H](C)C(=O)O)C2] | 650 |
| 65. | (S)-4-(6-(3-((2-((S)-3- carboxybutanoyl)-7- chloro-6-methoxybenzo[b] thiophen-5-yl)oxy) propoxy)-4-fluoro-5- methoxyisoindolin-2- yl)-2-methyl-4- oxobutanoic acid | 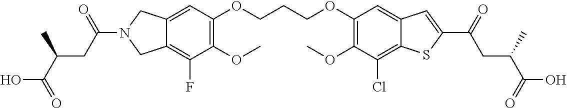 [SMILES: COc1c(OCCCOc2cc3cc(C(=O)C[C@H](C)C(=O)O)sc3c(Cl)c2OC)cc2c(c1F)CN(C(=O)C[C@H](C)C(=O)O)C2] | 666 |
| 66. | (S)-4-(5-(3-((2-((S)-3- carboxybutanoyl)-7- chloro-6-methoxyiso- indolin-5-yl)oxy)propoxy)- 7-fluoro-6-methoxybenzo [b]thiophen-2-yl)-2- methyl-4-oxobutanoic acid | 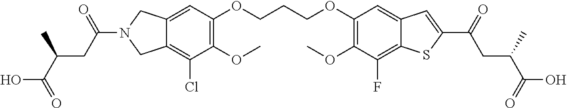 [SMILES: COc1c(OCCCOc2cc3cc(C(=O)C[C@H](C)C(=O)O)sc3c(F)c2OC)cc2c(c1Cl)CN(C(=O)C[C@H](C)C(=O)O)C2] | 666 |
| 67. | (S)-4-(5-(3-((2-((S)-3- carboxybutanoyl)-4,7- difluoro-6-methoxy- isoindolin-5-yl)oxy) propoxy)-7-chloro-6- methoxybenzo[b]thiophen- 2-yl)-2-methyl-4- oxobutanoic acid | 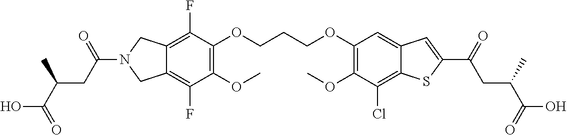 [SMILES: COc1c(F)c2c(c(F)c1OCCCOc1cc3cc(C(=O)C[C@H](C)C(=O)O)sc3c(Cl)c1OC)CN(C(=O)C[C@H](C)C(=O)O)C2] | 684 |
| 68. | (S)-4-(5-(3-((2-((S)-3- carboxybutanoyl)-4,7- difluoro-6-methoxy- isoindolin-5-yl)oxy) propoxy)-7-fluoro-6- methoxybenzo[b]thiophen- 2-yl)-2-methyl-4- oxobutanoic acid | 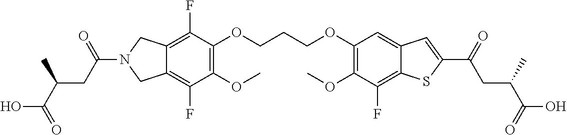 [SMILES: COc1c(F)c2c(c(F)c1OCCCOc1cc3cc(C(=O)C[C@H](C)C(=O)O)sc3c(F)c1OC)CN(C(=O)C[C@H](C)C(=O)O)C2] | 668 |
| 69. | (S)-4-(5-(3-((2-((S)-3- carboxybutanoyl)-4- chloro-7-fluoro-6- methoxyisoindolin-5-yl) oxy)propoxy)-7-fluoro- 6-methoxybenzo[b] thiophen-2-yl)-2-methyl- 4-oxobutanoic acid | 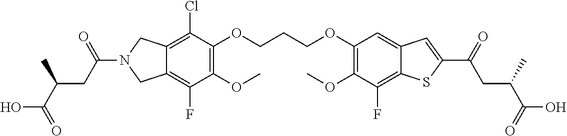 [SMILES: COc1c(F)c2c(c(Cl)c1OCCCOc1cc3cc(C(=O)C[C@H](C)C(=O)O)sc3c(F)c1OC)CN(C(=O)C[C@H](C)C(=O)O)C2] | 684 |
| 70. | (S)-4-(6-(3-((2-((S)-3- carboxybutanoyl)-4,7- difluoro-6-methoxy- benzo[b]thiophen-5-yl) oxy)propoxy)-4-fluoro- 5-methoxyisoindolin-2-yl)- 2-methyl-4-oxobutanoic acid | 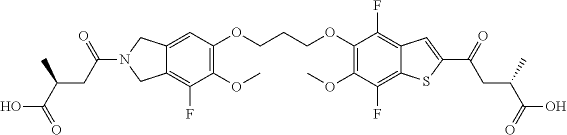 [SMILES: COc1c(OCCCOc2c(OC)c(F)c3sc(C(=O)C[C@H](C)C(=O)O)cc3c2F)cc2c(c1F)CN(C(=O)C[C@H](C)C(=O)O)C2] | 668 |
| 71. | (S)-4-(6-(3-((2-((S)-3- carboxybutanoyl)-7- chloro-4-fluoro-6- methoxybenzo[b]thiophen- 5-yl)oxy)propoxy)-4- fluoro-5-methoxy- isoindolin-2-yl)-2- methyl-4-oxobutanoic aic | 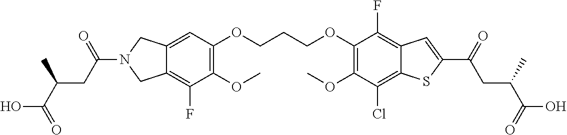 [SMILES: COc1c(OCCCOc2c(OC)c(Cl)c3sc(C(=O)C[C@H](C)C(=O)O)cc3c2F)cc2c(c1F)CN(C(=O)C[C@H](C)C(=O)O)C2] | 684 |
| 72. | (S)-4-(6-(3-((2-((S)-3- carboxybutanoyl)-4,7- dichloro-6-methoxybenzo [b]thiophen-5-yl)oxy) propoxy)-4-fluoro-5- methoxyisoindolin-2-yl)- 2-methyl-4-oxobutanoic acid | 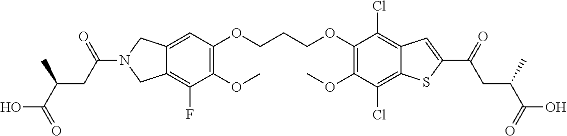 [SMILES: COc1c(OCCCOc2c(OC)c(Cl)c3sc(C(=O)C[C@H](C)C(=O)O)cc3c2Cl)cc2c(c1F)CN(C(=O)C[C@H](C)C(=O)O)C2] | 700 |
| 73. | (S)-4-(5-(3-((2-((S)-3- carboxybutanoyl)-4- chloro-7-fluoro-6- methoxyisoindolin-5-yl) oxy)propoxy)-7-chloro- 6-methoxybenzo[b] thiophen-2-yl)-2-methyl- 4-oxobutanoic acid | 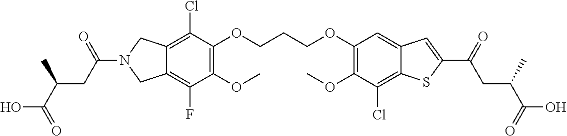 [SMILES: COc1c(F)c2c(c(Cl)c1OCCCOc1cc3cc(C(=O)C[C@H](C)C(=O)O)sc3c(Cl)c1OC)CN(C(=O)C[C@H](C)C(=O)O)C2] | 700 |
| 74. | (S)-4-(5-(3-((2-((S)-3- carboxybutanoyl)-4,7- dichloro-6-methoxy- isoindolin-5-yl)oxy) propoxy)-7-fluoro-6- methoxybenzo[b] thiophen-2-yl)-2-methyl 4-oxobutanoic acid | 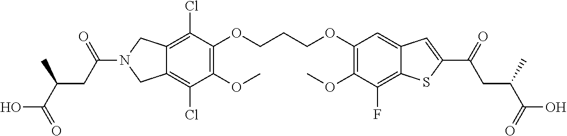 [SMILES: COc1c(Cl)c2c(c(Cl)c1OCCCOc1cc3cc(C(=O)C[C@H](C)C(=O)O)sc3c(F)c1OC)CN(C(=O)C[C@H](C)C(=O)O)C2] | 700 |
| 75. | (S)-4-(5-(3-((2-((S)-3- carboxybutanoyl)-7- chloro-4-fluoro-6- methoxyisoindolin-5-yl) oxy)propoxy)-7-fluoro- 6-methoxybenzo[b] thiophen-2-yl)-2-methyl- 4-oxobutanoic acid | 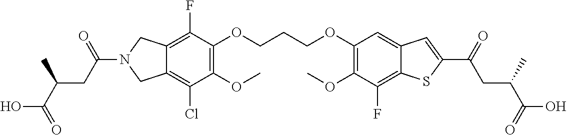 [SMILES: COc1c(Cl)c2c(c(F)c1OCCCOc1cc3cc(C(=O)C[C@H](C)C(=O)O)sc3c(F)c1OC)CN(C(=O)C[C@H](C)C(=O)O)C2] | 684 |
| 76. | (S)-4-(6-(3-((2-((S)-3- carboxybutanoyl)-4,7- difluoro-6-methoxybenzo [b]thiophen-5-yl)oxy) propoxy)-4-chloro-5- methoxyisoindolin-2-yl)- 2-methyl-4-oxobutanoic acid | 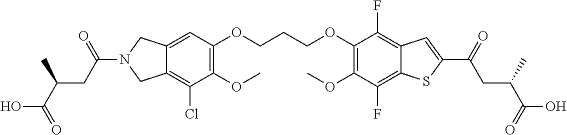 [SMILES: COc1c(OCCCOc2c(OC)c(F)c3sc(C(=O)C[C@H](C)C(=O)O)cc3c2F)cc2c(c1Cl)CN(C(=O)C[C@H](C)C(=O)O)C2] | 684 |
| 77. | (S)-4-(6-(3-((2-((S)-3- carboxybutanoyl)-4- chloro-7-fluoro-6- methoxybenzo[b]thiophen- 5-yl)oxy)propoxy)-4- chloro-5-methoxy- isoindolin-2-yl)-2-methyl- 4-oxobutanoic acid | 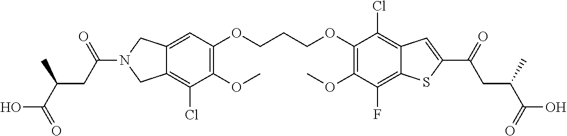 [SMILES: COc1c(OCCCOc2c(OC)c(F)c3sc(C(=O)C[C@H](C)C(=O)O)cc3c2Cl)cc2c(c1Cl)CN(C(=O)C[C@H](C)C(=O)O)C2] | 700 |
| 78. | (S)-4-(5-(3-((2-((S)-3- carboxybutanoyl)-4- fluoro-6-hydroxyiso- indolin-5-yl)oxy)propoxy)- 4-fluoro-6-methoxybenzo [b]thiophen-2-yl)-2- methyl-4-oxobutanoic acid | 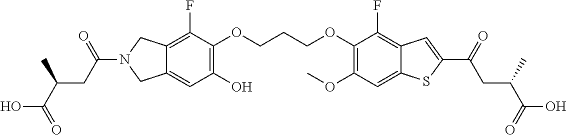 [SMILES: COc1cc2sc(C(=O)C[C@H](C)C(=O)O)cc2c(F)c1OCCCOc1c(O)cc2c(c1F)CN(C(=O)C[C@H](C)C(=O)O)C2] | 636 |
| 79. | (S)-4-(5-(3-((2-((S)-3- carboxybutanoyl)-4- chloro-6-methoxybenzo [b]thiophen-5-yl)oxy) propoxy)-4-fluoro-6- hydroxyisoindolin-2-yl)- 2-methyl-4-oxobutanoic acid | 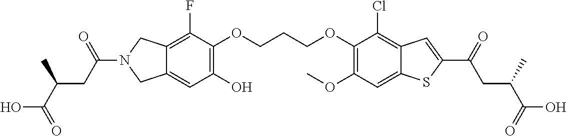 [SMILES: COc1cc2sc(C(=O)C[C@H](C)C(=O)O)cc2c(Cl)c1OCCCOc1c(O)cc2c(c1F)CN(C(=O)C[C@H](C)C(=O)O)C2] | 652 |
| 80. | (S)-4-(5-(3-((2-((S)-3- carboxybutanoyl)-4- chloro-6-hydroxyiso- indolin-5-yl)oxy) propoxy)-4-fluoro-6- methoxybenzo[b]thiophen- 2-yl)-2-methyl-4- oxobutanoic acid | 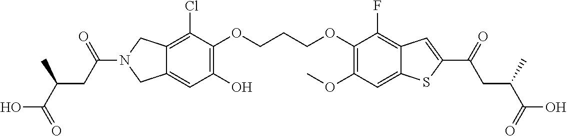 [SMILES: COc1cc2sc(C(=O)C[C@H](C)C(=O)O)cc2c(F)c1OCCCOc1c(O)cc2c(c1Cl)CN(C(=O)C[C@H](C)C(=O)O)C2] | 652 |
| 81. | (S)-4-(5-(3-((2-((S)-3- carboxybutanoyl)-4- chloro-6-hydroxyiso- indolin-5-yl)oxy) propoxy)-4-chloro-6- methoxybenzo[b] thiophen-2-yl)-2-methyl- 4-oxobutanoic acid | 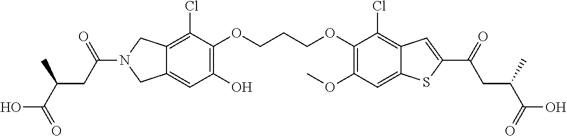 [SMILES: COc1cc2sc(C(=O)C[C@H](C)C(=O)O)cc2c(Cl)c1OCCCOc1c(O)cc2c(c1Cl)CN(C(=O)C[C@H](C)C(=O)O)C2] | 668 |
| 82. | (S)-4-(6-(3-((2-((S)-3- carboxybutanoyl)-6- methoxybenzo[b]thiophen- 5-yl)oxy)propoxy)-4- fluoro-5-hydroxyiso- indolin-2-yl)-2-methyl- 4-oxobutanoic acid | 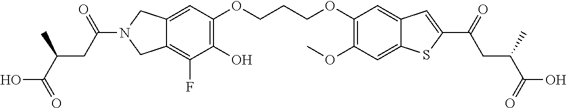 [SMILES: COc1cc2sc(C(=O)C[C@H](C)C(=O)O)cc2cc1OCCCOc1cc2c(c(F)c1O)CN(C(=O)C[C@H](C)C(=O)O)C2] | 618 |
| 83. | (S)-4-(5-(3-((2-((S)-3- carboxybutanoyl)-7- fluoro-6-hydroxyiso- indolin-5-yl)oxy)propoxy)- 7-fluoro-6-methoxybenzo [b]thiophen-2-yl)-2- methyl-4-oxobutanoic acid | 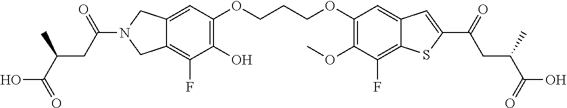 [SMILES: COc1c(OCCCOc2cc3c(c(F)c2O)CN(C(=O)C[C@H](C)C(=O)O)C3)cc2cc(C(=O)C[C@H](C)C(=O)O)sc2c1F] | 636 |
| 84. | (S)-4-(6-(3-((2-((S)-3- carboxybutanoyl)-7- chloro-6-methoxybenzo [b]thiophen-5-yl)oxy) propoxy)-4-fluoro-5- hydroxyisoindolin-2-yl)- 2-methyl-4-oxobutanoic acid | 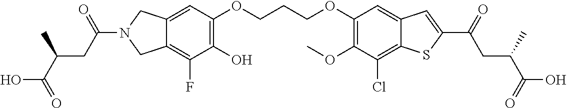 [SMILES: COc1c(OCCCOc2cc3c(c(F)c2O)CN(C(=O)C[C@H](C)C(=O)O)C3)cc2cc(C(=O)C[C@H](C)C(=O)O)sc2c1Cl] | 652 |
| 85. | (S)-4-(5-(3-((2-((S)-3- carboxybutanoyl)-7- chloro-6-hydroxy- isoindolin-5-yl)oxy) propoxy)-7-fluoro-6- methoxybenzo[b] thiophen-2-yl)-2-methyl- 4-oxobutanoic acid | 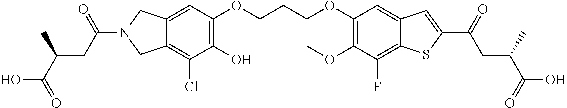 [SMILES: COc1c(OCCCOc2cc3c(c(Cl)c2O)CN(C(=O)C[C@H](C)C(=O)O)C3)cc2cc(C(=O)C[C@H](C)C(=O)O)sc2c1F] | 652 |
| 86. | (S)-4-(5-(3-((2-((S)-3- carboxybutanoyl)-4,7- difluoro-6-methoxy- isoindolin-5-yl)oxy) propoxy)-4-chloro-7- fluoro-6-methoxybenzo [b]thiophen-2-yl)-2- methyl-4-oxobutanoic acid | 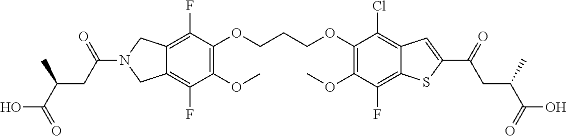 [SMILES: COc1c(F)c2c(c(F)c1OCCCOc1c(OC)c(F)c3sc(C(=O)C[C@H](C)C(=O)O)cc3c1Cl)CN(C(=O)C[C@H](C)C(=O)O)C2] | 702 |
| 87. | (S)-4-(5-(3-((2-((S)-3- carboxybutanoyl)-4- chloro-7-fluoro-6- methoxybenzo[b]thiophen- 5-yl)oxy)propoxy)-4- chloro-7-fluoro-6- methoxyisoindolin-2-yl)- 2-methyl-4-oxobutanoic acid | 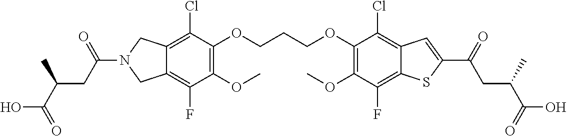 [SMILES: COc1c(F)c2c(c(Cl)c1OCCCOc1c(OC)c(F)c3sc(C(=O)C[C@H](C)C(=O)O)cc3c1Cl)CN(C(=O)C[C@H](C)C(=O)O)C2] | 718 |
| 88. | (S)-4-(5-(3-((2-((S)-3- carboxybutanoyl)-4,7- difluoro-6-methoxy- benzo[b]thiophen-5-yl) oxy)propoxy)-4,7- difluoro-6-methoxy- isoindolin-2-yl)-2- methyl-4-oxobutanoic acid | 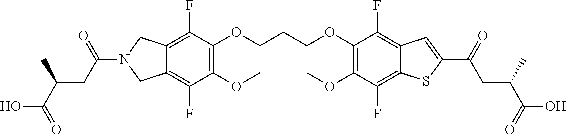 [SMILES: COc1c(F)c2c(c(F)c1OCCCOc1c(OC)c(F)c3sc(C(=O)C[C@H](C)C(=O)O)cc3c1F)CN(C(=O)C[C@H](C)C(=O)O)C2] | 686 |
| 89. | (S)-4-(5-(3-((2-((S)-3- carboxybutanoyl)-4,7- difluoro-6-methoxy- isoindolin-5-yl)oxy) propoxy)-7-chloro-4- fluoro-6-methoxybenzo [b]thiophen-2-yl)-2- methyl-4-oxobutanoic acid | 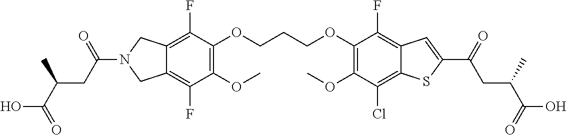 [SMILES: COc1c(F)c2c(c(F)c1OCCCOc1c(OC)c(Cl)c3sc(C(=O)C[C@H](C)C(=O)O)cc3c1F)CN(C(=O)C[C@H](C)C(=O)O)C2] | 702 |
| 90. | (S)-4-(5-(3-((2-((S)-3- carboxybutanoyl)-4,7- dichloro-6-methoxybenzo [b]thiophen-5-yl)oxy) propoxy)-4,7-difluoro-6- methoxyisoindolin-2- yl)-2-yl)-2-methyl-4- oxobutanoic acid | 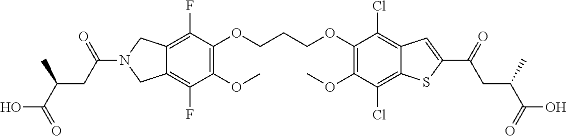 [SMILES: COc1c(F)c2c(c(F)c1OCCCOc1c(OC)c(Cl)c3sc(C(=O)C[C@H](C)C(=O)O)cc3c1Cl)CN(C(=O)C[C@H](C)C(=O)O)C2] | 718 |
| 91. | (S)-4-(5-(3-((2-((S)-3- carboxybutanoyl)-4,7- dichloro-6-methoxybenzo [b]thiophen-5-yl)oxy) propoxy)-4-chloro-7- fluoro-6-methoxy- isoindolin-2-yl)-2-methyl- 4-oxobutanoic acid | 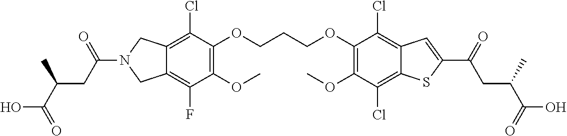 [SMILES: COc1c(F)c2c(c(Cl)c1OCCCOc1c(OC)c(Cl)c3sc(C(=O)C[C@H](C)C(=O)O)cc3c1Cl)CN(C(=O)C[C@H](C)C(=O)O)C2] | 734 |
| 92. | (S)-4-(5-(3-((2-((S)-3- carboxybutanoyl)-4- chloro-7-fluoro-6- methoxyisoindolin-5-yl) oxy)propoxy)-7-chloro- 4-fluoro-6-methoxybenzo [b]thiophen-2-yl)-2- methyl-4-oxobutanoic acid | 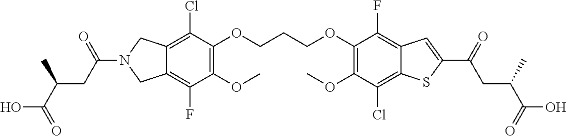 [SMILES: COc1c(F)c2c(c(Cl)c1OCCCOc1c(OC)c(Cl)c3sc(C(=O)C[C@H](C)C(=O)O)cc3c1F)CN(C(=O)C[C@H](C)C(=O)O)C2] | 718 |
| 93. | (S)-4-(5-(3-((2-((S)-3- carboxybutanoyl)-4,7- dichloro-6-methoxy- isoindolin-5-yl)oxy) propoxy)-4-chloro-7- fluoro-6-methoxybenzo [b]thiophen-2-yl)-2- methyl-4-oxobutanoic acid | 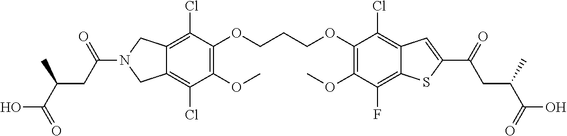 [SMILES: COc1c(Cl)c2c(c(Cl)c1OCCCOc1c(OC)c(F)c3sc(C(=O)C[C@H](C)C(=O)O)cc3c1Cl)CN(C(=O)C[C@H](C)C(=O)O)C2] | 734 |
| 94. | (S)-4-(5-(3-((2-((S)-3- carboxybutanoyl)-4-chloro- 7-fluoro-6-methoxybenzo [b]thiophen-5-yl)oxy) propoxy)-7-chloro-4- fluoro-6-methoxy- isoindolin-2-yl)-2-methyl- 4-oxobutanoic acid | 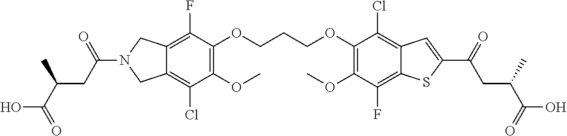 [SMILES: COc1c(Cl)c2c(c(F)c1OCCCOc1c(OC)c(F)c3sc(C(=O)C[C@H](C)C(=O)O)cc3c1Cl)CN(C(=O)C[C@H](C)C(=O)O)C2] | 718 |
| 95. | (2S,2′S)-4,4′-(7,8,11,13, 17,18-hexahydro-6H,12H, 16H-thieno[2″,3″:4′,5′] benzo[1′,2′:9,10][1,4,8,11] tetraoxacyclotetradecino [2,3-f]isoindoline-2,12- diyl)bis(2-methyl-4-oxo- butanoic acid) | 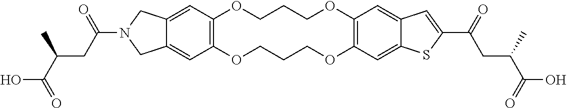 [SMILES: C[C@@H](CC(=O)c1cc2cc3c(cc2s1)OCCCOc1cc2c(cc1OCCCO3)CN(C(=O)C[C@H](C)C(=O)O)C2)C(=O)O] | 626 |
| 96. | (S)-4-(6-methoxy-5-(3- (6-methoxy-2-((S)-3- methyl-4-(methyl- sulfonamido)-4- oxobutanoyl)isoindolin- 5-yl)oxy)propoxy) benzo[b]thiophen-2-yl)- 2-methyl-4-oxobutanoic acid | 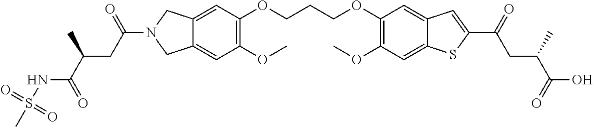 [SMILES: COc1cc2c(cc1OCCCOc1cc3cc(C(=O)C[C@H](C)C(=O)O)sc3cc1OC)CN(C(=O)C[C@H](C)C(=O)NS(C)(=O)=O)C2] | 691 |
| 97. | (S)-4-(5-(3-((2-((S)-4- ((N,N-dimethylsulfamoyl) amino)-3-methyl-4-oxo- butanoyl)-6-methoxy- isoindolin-5-yl)oxy) propoxy)-6-methoxy- benzo[b]thiophen-2-yl)-2- methyl-4-oxobutanoic acid | 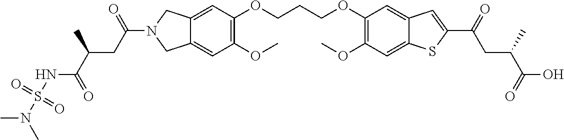 [SMILES: COc1cc2c(cc1OCCCOc1cc3cc(C(=O)C[C@H](C)C(=O)O)sc3cc1OC)CN(C(=O)C[C@H](C)C(=O)NS(=O)(=O)N(C)C)C2] | 720 |
| 98. | (S)-4-(5-(3-((2-(3-cyano- propanoyl)-6-methoxy- isoindolin-5-yl)oxy) propoxy)-6-methoxy- benzo[b]thiophen-2-yl)-2- methyl-4-oxobutanoic acid | 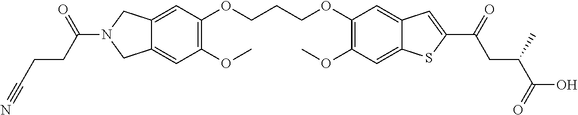 [SMILES: COc1cc2c(cc1OCCCOc1cc3cc(C(=O)C[C@H](C)C(=O)O)sc3cc1OC)CN(C(=O)CCC#N)C2] | 581 |
| 99. | 4-(5-(3-((2-(3-cyano- propanoyl)-6-methoxy- benzo[b]thiophen-5-yl) oxy)propoxy)-6-methoxy- isoindolin-2-yl)-4- oxobutanenitrile | 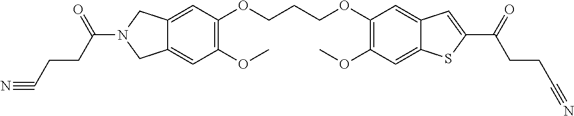 [SMILES: COc1cc2c(cc1OCCCOc1cc3cc(C(=O)CCC#N)sc3cc1OC)CN(C(=O)CCC#N)C2] | 548 |
| 100. | (S)-4-(5-(3-((2-(3-cyano- propanoyl)-6-methoxy- benzo[b]thiophen-5-yl) oxy)propoxy)-6-methoxy- isoindolin-2-yl)-2-methyl- 4-oxobutanoic acid | 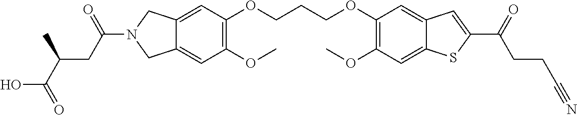 [SMILES: COc1cc2c(cc1OCCCOc1cc3cc(C(=O)CCC#N)sc3cc1OC)CN(C(=O)C[C@H](C)C(=O)O)C2] | 581 |
| 101. | (1R,2R)-2-(5-(3-((2-((S)- 3-carboxybutanoyl)-6- methoxyisoindolin-5-yl) oxy)propoxy)-6-methoxy- benzo[b]thiophene-2- carbonyl)cyclobutane-1- carboxylic acid | 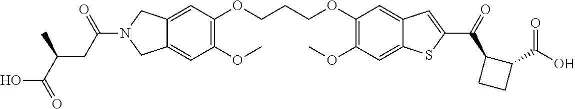 [SMILES: COc1cc2c(cc1OCCCOc1cc3cc(C(=O)[C@@H]4CC[C@H]4C(=O)O)sc3cc1OC)CN(C(=O)C[C@H](C)C(=O)O)C2] | 626 |
| 102. | (1R,2R)-2-(5-(3-((2-((1R, 2R)-2-carboxycyclo- butane-1-carbonyl)-6- methoxybenzo[b]thiophen- 5-yl)oxy)propoxy)-6- methoxyisoindoline-2- carbonyl)cyclobutane-1- carboxylic acid | 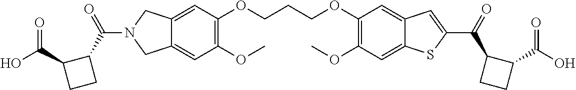 [SMILES: COc1cc2c(cc1OCCCOc1cc3cc(C(=O)[C@@H]4CC[C@H]4C(=O)O)sc3cc1OC)CN(C(=O)[C@@H]1CC[C@H]1C(=O)O)C2] | 638 |
| 103. | (1R,2R)-2-(5-(3-((2-((S)- 3-carboxybutanoyl)-6- methoxybenzo[b]thiophen- 5-yl)oxy)propoxy)-6- methoxyisoindoline-2- carbonyl)cyclobutane-1- carboxylic acid | 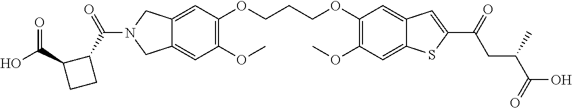 [SMILES: COc1cc2c(cc1OCCCOc1cc3cc(C(=O)C[C@H](C)C(=O)O)sc3cc1OC)CN(C(=O)[C@@H]1CC[C@H]1C(=O)O)C2] | 626 |
| 104. | (1R,2R)-2-(5-(3-((2-((S)- 3-carboxybutanoyl)-6- methoxyisoindolin-5-yl) oxy)propoxy)-6-methoxy- benzo[b]thiophene-2- carbonyl)cyclopropane-1- carboxylic acid | 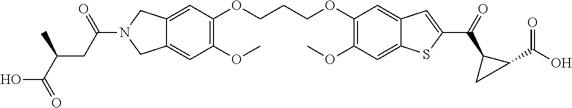 [SMILES: COc1cc2c(cc1OCCCOc1cc3cc(C(=O)[C@@H]4C[C@H]4C(=O)O)sc3cc1OC)CN(C(=O)C[C@H](C)C(=O)O)C2] | 612 |
| 105. | (1R,2R)-2-(5-(3-((2-((1R, 2R)-2-carboxycyclo- propane-1-carbonyl)-6- methoxybenzo[b]thiophen- 5-yl)oxy)propoxy)-6- methoxyisoindoline-2- carbonyl)cyclopropane-1- carboxylic acid | 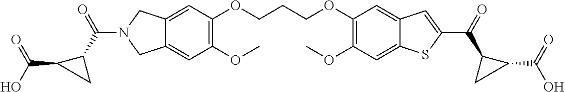 [SMILES: COc1cc2c(cc1OCCCOc1cc3cc(C(=O)[C@@H]4C[C@H]4C(=O)O)sc3cc1OC)CN(C(=O)[C@@H]1C[C@H]1C(=O)O)C2] | 610 |
| 106. | (1R,2R)-2-(5-(3-((2-((S)- 3-carboxybutanoyl)-6- methoxybenzo[b]thiophen- 5-yl)oxy)propoxy)-6- methoxyisoindoline-2- carbonyl)cyclopropane-1- carboxylic acid | 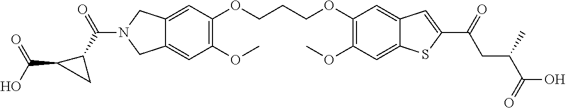 [SMILES: COc1cc2c(cc1OCCCOc1cc3cc(C(=O)C[C@H](C)C(=O)O)sc3cc1OC)CN(C(=O)[C@@H]1C[C@H]1C(=O)O)C2] | 612 |
| 107. | (S)-N-(N,N-dimethyl- sulfamoyl)-4-(5-(3-((2-((S)- 4-((N,N-dimethyl- sulfamoyl)amino)-3-methyl- 4-oxobutanoyl)-6- methoxybenzo[b]thiophen- 5-yl)oxy)propoxy)-6- methoxyisoindolin-2-yl)-2- methyl-4-oxobutanamide | 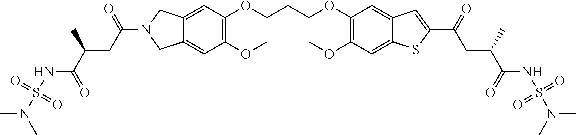 | 826 |
| 108. | (S)-4-(5-(3-((2-((S)-4- ((N,N-dimethylsulfamoyl) amino)-3-methyl-4-oxo- butanoyl)-6-methoxybenzo [b]thiophen-5-yl)oxy) propoxy)-6-methoxyiso- indolin-2-yl)-2-methyl-4- oxobutanoic acid | 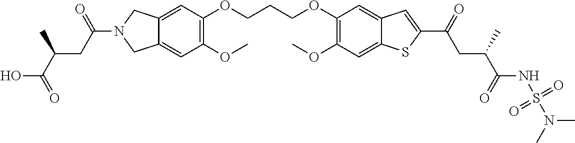 [SMILES: COc1cc2c(cc1OCCCOc1cc3cc(C(=O)C[C@H](C)C(=O)NS(=O)(=O)N(C)C)sc3cc1OC)CN(C(=O)C[C@H](C)C(=O)O)C2] | 720 |
| 109. | (S)-4-(6-methoxy-5-(3- ((6-methoxy-2-((S)-3- methyl-4-(methyl- sulfonamido)-4-oxobutanoyl) isoindolin-5-yl)oxy) propoxy)benzo[b]thiophen- 2-yl)-2-methyl-N-(methyl- sulfonyl)-4-oxobutanamide | 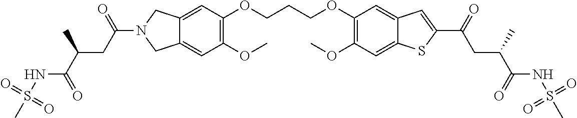 [SMILES: COc1cc2c(cc1OCCCOc1cc3cc(C(=O)C[C@H](C)C(=O)NS(C)(=O)=O)sc3cc1OC)CN(C(=O)C[C@H](C)C(=O)NS(C)(=O)=O)C2] | 768 |
| 110. | (S)-4-(5-methoxy-6-(3- ((6-methoxy-2-((S)-3-methyl- 4-(methylsulfonamido)-4- oxobutanoyl)benzo[b] thiophen-5-yl)oxy) propoxy)isoindolin-2-yl)- 2-methyl-4-oxobutanoic acid | 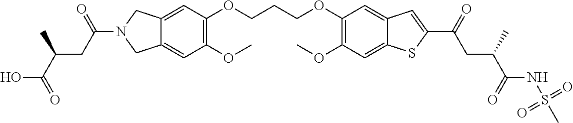 [SMILES: COc1cc2c(cc1OCCCOc1cc3cc(C(=O)C[C@H](C)C(=O)NS(C)(=O)=O)sc3cc1OC)CN(C(=O)C[C@H](C)C(=O)O)C2] | 691 |
| 111. | (S)-4-(4-(3-((2-((S)-3- carboxybutanoyl)-6- methoxybenzo[b]thiophen- 5-yl)oxy)propoxy)-6- methoxyisoindolin-2-yl)-2- methyl-4-oxobutanoic acid | 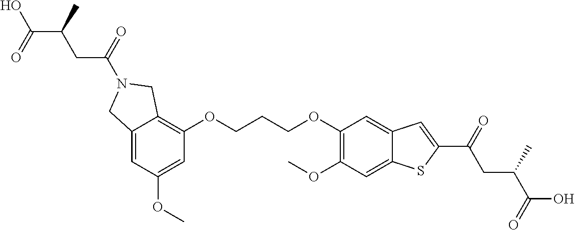 [SMILES: COc1cc2c(c(OCCCOc3cc4cc(C(=O)C[C@H](C)C(=O)O)sc4cc3OC)c1)CN(C(=O)C[C@H](C)C(=O)O)C2] | 614 |
| 112. | (S)-4-(5-(3-((2-((S)-3- carboxybutanoyl)-6- methoxybenzo[b]thiophen- 4-yl)oxy)propoxy)-6- methoxyisoindolin-2-yl)-2- methyl-4-oxobutanoic acid | 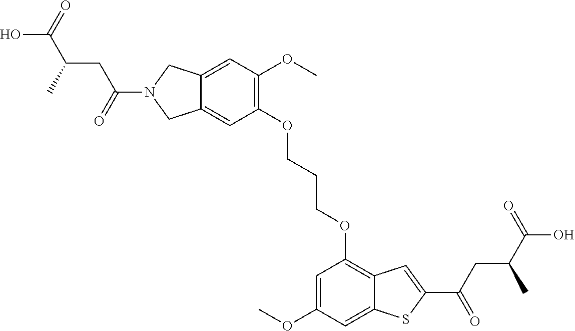 [SMILES: COc1cc(OCCCOc2cc3c(cc2OC)CN(C(=O)C[C@H](C)C(=O)O)C3)c2cc(C(=O)C[C@H](C)C(=O)O)sc2c1] | 614 |
| 113. | (S)-4-(4-(4-((2-((S)-3- carboxybutanoyl)-6- methoxybenzo[b]thiophen- 5-yl)oxy)butyl)-6- methoxyisoindolin-2-yl)-2- methyl-4-oxobutanoic acid | 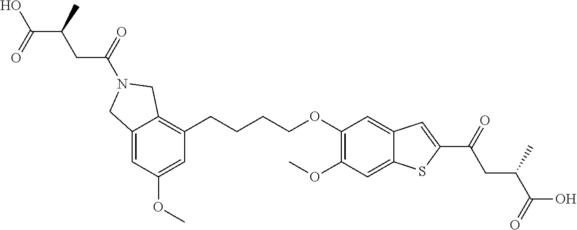 [SMILES: COc1cc(CCCCOc2cc3cc(C(=O)C[C@H](C)C(=O)O)sc3cc2OC)c2c(c1)CN(C(=O)C[C@H](C)C(=O)O)C2] | 612 |
| 114. | (S)-4-(4-(4-(2-((S)-3- carboxybutanoyl)-6- methoxybenzo[b]thiophen- 5-yl)butoxy)-6-methoxy- isoindolin-2-yl)-2-methyl-4- oxobutanoic acid | 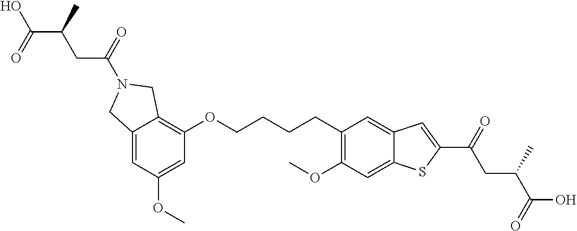 [SMILES: COc1cc2c(c(OCCCCc3cc4cc(C(=O)C[C@H](C)C(=O)O)sc4cc3OC)c1)CN(C(=O)C[C@H](C)C(=O)O)C2] | 612 |
| 115. | (S)-4-(5-(4-((2-((S)-3- carboxybutanoyl)-6- methoxybenzo[b]thiophen- 4-yl)oxy)butyl)-6- methoxyisoindolin-2-yl)-2- methyl-4-oxobutanoic acid | 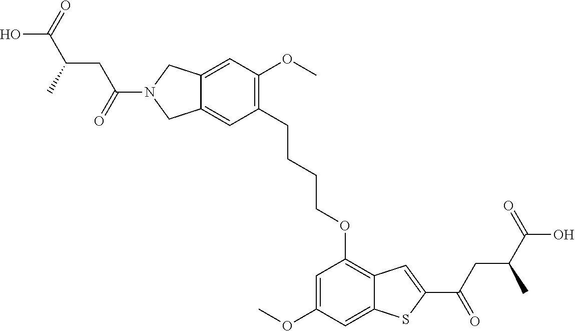 [SMILES: COc1cc(OCCCCc2cc3c(cc2OC)CN(C(=O)C[C@H](C)C(=O)O)C3)c2cc(C(=O)C[C@H](C)C(=O)O)sc2c1] | 612 |
| 116. | (S)-4-(5-(4-(2-((S)-3- carboxybutanoyl)-6- methoxybenzo[b]thiophen- 4-yl)butoxy)-6-methoxy- isoindolin-2-yl)-2-methyl-4- oxobutanoic acid | 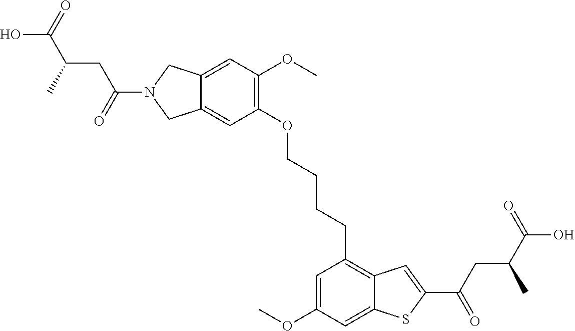 [SMILES: COc1cc(CCCCOc2cc3c(cc2OC)CN(C(=O)C[C@H](C)C(=O)O)C3)c2cc(C(=O)C[C@H](C)C(=O)O)sc2c1] | 612 |
| 117. | (S)-4-(5-(3-((2-((S)-3- carboxybutanoyl)-6- methoxyisoindolin-5-yl)oxy) propoxy)-6-methylbenzo[b] thiophen-2-yl)-2-methyl-4- oxobutanoic acid | 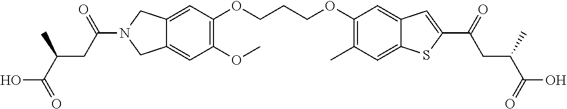 [SMILES: COc1cc2c(cc1OCCCOc1cc3cc(C(=O)C[C@H](C)C(=O)O)sc3cc1C)CN(C(=O)C[C@H](C)C(=O)O)C2] | 598 |
| 118. | (S)-4-(5-(3-((2-((S)-3- carboxybutanoyl)-6-ethyl- benzo[b]thiophen-5-yl) oxy)propoxy)-6-methoxy- isoindolin-2-yl)-2-methyl- 4-oxobutanoic acid | 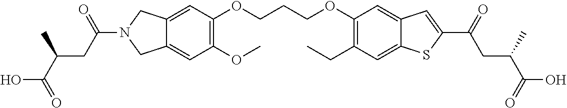 [SMILES: CCc1cc2sc(C(=O)C[C@H](C)C(=O)O)cc2cc1OCCCOc1cc2c(cc1OC)CN(C(=O)C[C@H](C)C(=O)O)C2] | 612 |
| 119. | (S)-4-(5-(3-((2-((S)-3- carboxybutanoyl)-6- methoxyisoindolin-5-yl)oxy) propoxy)-6-vinylbenzo[b] thiophen-2-yl)-2-methyl- 4-oxobutanoic acid | 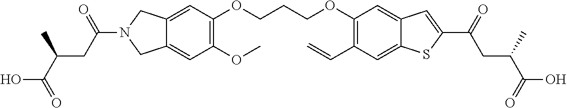 [SMILES: C=Cc1cc2sc(C(=O)C[C@H](C)C(=O)O)cc2cc1OCCCOc1cc2c(cc1OC)CN(C(=O)C[C@H](C)C(=O)O)C2] | 610 |
| 120. | (S)-4-(5-(3-((2-((S)-3- carboxybutanoyl)-6-ethynyl- benzo[b]thiophen-5-yl)oxy) propoxy)-6-methoxy- isoindolin-2-yl)-2-methyl-4- oxobutanoic acid | 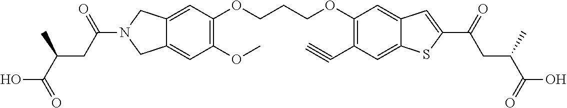 [SMILES: C#Cc1cc2sc(C(=O)C[C@H](C)C(=O)O)cc2cc1OCCCOc1cc2c(cc1OC)CN(C(=O)C[C@H](C)C(=O)O)C2] | 608 |
| 121. | (S)-4-(5-(3-((6-amino-2- ((S)-3-carboxybutanoyl) benzo[b]thiophen-5-yl)oxy) propoxy)-6-methoxy- isoindolin-2-yl)-2-methyl- 4-oxobutanoic acid | 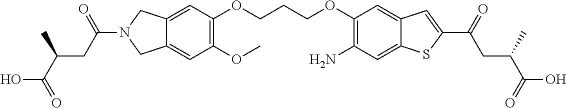 [SMILES: COc1cc2c(cc1OCCCOc1cc3cc(C(=O)C[C@H](C)C(=O)O)sc3cc1N)CN(C(=O)C[C@H](C)C(=O)O)C2] | 599 |
| 122. | (S)-4-(5-(3-((2-((S)-3- carboxybutanoyl)-6- (methylamino)benzo[b] thiophen-5-yl)oxy)propoxy)- 6-methoxyisoindolin-2-yl)- 2-methyl-4-oxobutanoic acid | 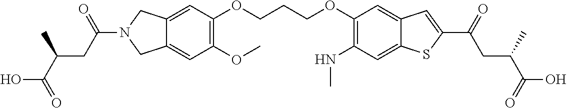 [SMILES: CNc1cc2sc(C(=O)C[C@H](C)C(=O)O)cc2cc1OCCCOc1cc2c(cc1OC)CN(C(=O)C[C@H](C)C(=O)O)C2] | 613 |
| 123. | (S)-4-(5-(3-((6-bromo-2- ((S)-3-carboxybutanoyl) benzo[b]thiophen-5-yl)oxy) propoxy)-6-methoxy- isoindolin-2-yl)-2-methyl- 4-oxobutanoic acid | 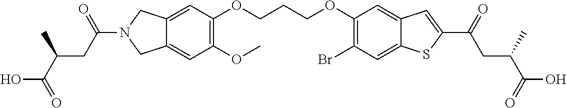 [SMILES: COc1cc2c(cc1OCCCOc1cc3cc(C(=O)C[C@H](C)C(=O)O)sc3cc1Br)CN(C(=O)C[C@H](C)C(=O)O)C2] | 662 |
| 124. | (S)-4-(5-(3-((2-((S)-3- carboxybutanoyl)-6- methoxybenzo[b]thiophen- 5-yl)oxy)propoxy)-6- methylisoindolin-2-yl)-2- methyl-4-oxobutanoic acid | 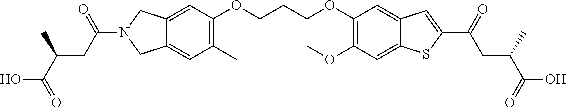 [SMILES: COc1cc2sc(C(=O)C[C@H](C)C(=O)O)cc2cc1OCCCOc1cc2c(cc1C)CN(C(=O)C[C@H](C)C(=O)O)C2] | 598 |
| 125. | (S)-4-(5-(3-((2-((S)-3- carboxybutanoyl)-6-ethyl- isoindolin-5-yl)oxy) propoxy)-6-methoxybenzo[b] thiophen-2-yl)-2-methyl-4- oxobutanoic acid | 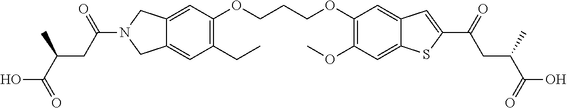 [SMILES: CCc1cc2c(cc1OCCCOc1cc3cc(C(=O)C[C@H](C)C(=O)O)sc3cc1OC)CN(C(=O)C[C@H](C)C(=O)O)C2] | 612 |
| 126. | (S)-4-(5-(3-((2-((S)-3- carboxybutanoyl)-6- methoxybenzo[b]thiophen- 5-yl)oxy)propoxy)-6-vinyl- isoindolin-2-yl)-2-methyl-4- oxobutanoic acid | 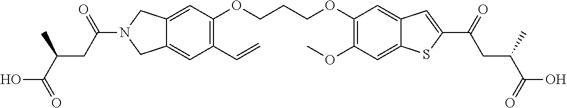 [SMILES: C=Cc1cc2c(cc1OCCCOc1cc3cc(C(=O)C[C@H](C)C(=O)O)sc3cc1OC)CN(C(=O)C[C@H](C)C(=O)O)C2] | 610 |
| 127. | (S)-4-(5-(3-((2-((S)-3- carboxybutanoyl)-6-ethynyl- isoindolin-5-yl)oxy) propoxy)-6-methoxybenzo[b] thiophen-2-yl)-2-methyl-4- oxobutanoic acid | 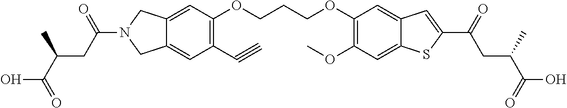 [SMILES: C#Cc1cc2c(cc1OCCCOc1cc3cc(C(=O)C[C@H](C)C(=O)O)sc3cc1OC)CN(C(=O)C[C@H](C)C(=O)O)C2] | 608 |
| 128. | (S)-4-(5-(3-((6-amino-2- ((S)-3-carboxybutanoyl) isoindolin-5-yl)oxy) propoxy)-6-methoxybenzo [b]thiophen-2-yl)-2- methyl-4-oxobutanoic acid | 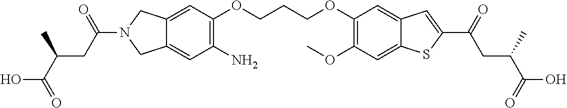 [SMILES: COc1cc2sc(C(=O)C[C@H](C)C(=O)O)cc2cc1OCCCOc1cc2c(cc1N)CN(C(=O)C[C@H](C)C(=O)O)C2] | 599 |
| 129. | (S)-4-(5-(3-((2-((S)-3- carboxybutanoyl)-6-(methyl- amino)isoindolin-5-yl) oxy)propoxy)-6-methoxy- benzo[b]thiophen-2-yl)-2- methyl-4-oxobutanoic acid | 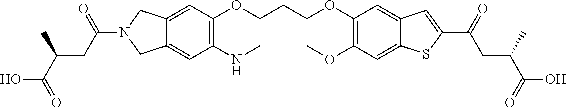 [SMILES: CNc1cc2c(cc1OCCCOc1cc3cc(C(=O)C[C@H](C)C(=O)O)sc3cc1OC)CN(C(=O)C[C@H](C)C(=O)O)C2] | 613 |
| 130. | (S)-4-(5-(3-((6-bromo-2- ((S)-3-carboxybutanoyl) isoindolin-5-yl)oxy) propoxy)-6-methoxybenzo [b]thiophen-2-yl)-2-methyl- 4-oxobutanoic acid | 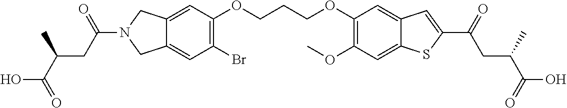 [SMILES: COc1cc2sc(C(=O)C[C@H](C)C(=O)O)cc2cc1OCCCOc1cc2c(cc1Br)CN(C(=O)C[C@H](C)C(=O)O)C2] | 662 |
| 131. | (S)-4-(5-(3-((6-((S)-3- carboxybutanoyl)-3- methoxy-6,7-dihydro-5H- pyrrolo[3,4-b]pyridin-2-yl) oxy)propoxy)-6-methoxy- benzo[b]thiophen-2-yl)-2- methyl-4-oxobutanoic acid | 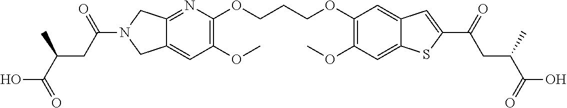 [SMILES: COc1cc2sc(C(=O)C[C@H](C)C(=O)O)cc2cc1OCCCOc1nc2c(cc1OC)CN(C(=O)C[C@H](C)C(=O)O)C2] | 615 |
| 132. | (S)-4-(5-(3-((6-((S)-3- carboxybutanoyl)-3- methoxy-6,7-dihydro-5H- pyrrolo[3,4-b]pyridin-2-yl) oxy)propoxy)-4-fluoro-6- methoxybenzo[b]thiophen- 2-yl)-2-methyl-4- oxobutanoic acid | 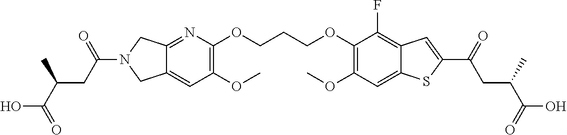 [SMILES: COc1cc2c(nc1OCCCOc1c(OC)cc3sc(C(=O)C[C@H](C)C(=O)O)cc3c1F)CN(C(=O)C[C@H](C)C(=O)O)C2] | 633 |
| 133. | (S)-4-(5-(3-((6-((S)-3- carboxybutanoyl)-3- methoxy-6,7-dihydro-5H- pyrrolo[3,4-b]pyridin-2-yl) oxy)propoxy)-4-chloro-6- methoxybenzo[b]thiophen- 2-yl)-2-methyl-4- oxobutanoic acid | 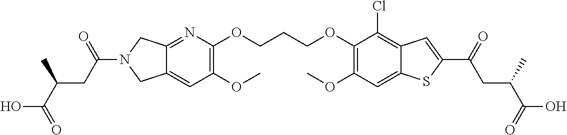 [SMILES: COc1cc2c(nc1OCCCOc1c(OC)cc3sc(C(=O)C[C@H](C)C(=O)O)cc3c1Cl)CN(C(=O)C[C@H](C)C(=O)O)C2] | 649 |
| 134. | (S)-4-(5-(3-((6-((S)-3- carboxybutanoyl)-3- hydroxy-6,7-dihydro-5H- pyrrolo[3,4-b]pyridin-2-yl) oxy)propoxy)-6-methoxy- thieno[3,2-b]pyridin-2-yl)- 2-methyl-4-oxobutanoic acid | 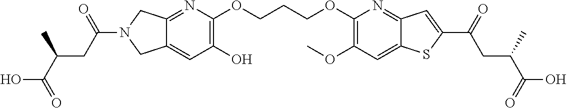 [SMILES: COc1cc2sc(C(=O)C[C@H](C)C(=O)O)cc2nc1OCCCOc1nc2c(cc1O)CN(C(=O)C[C@H](C)C(=O)O)C2] | 602 |
| 135. | (S)-4-(5-(3-((6-((S)-3- carboxybutanoyl)-3-methoxy- 6,7-dihydro-5H-pyrrolo [3,4-b]pyridin-2-yl)oxy) propoxy)-6-hydroxy- thieno[3,2-b]pyridin-2-yl)-2- methyl-4-oxobutanoic acid | 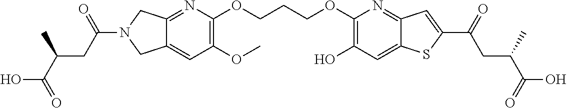 [SMILES: COc1cc2c(nc1OCCCOc1nc3cc(C(=O)C[C@H](C)C(=O)O)sc3cc1O)CN(C(=O)C[C@H](C)C(=O)O)C2] | 602 |
| 136. | (S)-4-(5-(3-((6-((S)-3- carboxybutanoyl)-3- methoxy-6,7-dihydro-5H- pyrrolo[3,4-b]pyridin-2-yl) oxy)propoxy)-6-methoxy- thieno[3,2-b]pyridin-2-yl)- 2-methyl-4-oxobutanoic acid | 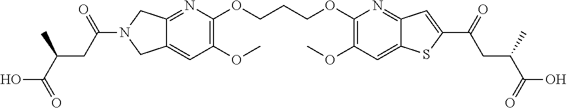 [SMILES: COc1cc2c(nc1OCCCOc1nc3cc(C(=O)C[C@H](C)C(=O)O)sc3cc1OC)CN(C(=O)C[C@H](C)C(=O)O)C2] | 616 |
| 137. | (S)-4-(5-(3-((6-((S)-3- carboxybutanoyl)-4-fluoro- 3-methoxy-6,7-dihydro- 5H-pyrrolo[3,4-b]pyridin-2- yl)oxy)propoxy)-6-methoxy- thieno[3,2-b]pyridin-2-yl)-2- methyl-4-oxobutanoic acid | 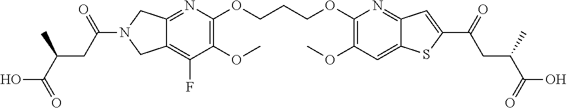 [SMILES: COc1cc2sc(C(=O)C[C@H](C)C(=O)O)cc2nc1OCCCOc1nc2c(c(F)c1OC)CN(C(=O)C[C@H](C)C(=O)O)C2] | 634 |
| 138. | (S)-4-(5-(3-((6-((S)-3- carboxybutanoyl)-4-chloro- 3-methoxy-6,7-dihydro- 5H-pyrrolo[3,4-b]pyridin-2- yl)oxy)propoxy)-6-methoxy- thieno[3,2-b]pyridin-2-yl)-2- methyl-4-oxobutanoic acid | 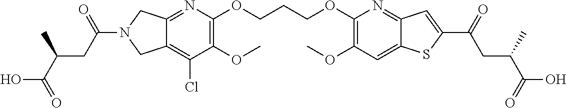 [SMILES: COc1cc2sc(C(=O)C[C@H](C)C(=O)O)cc2nc1OCCCOc1nc2c(c(Cl)c1OC)CN(C(=O)C[C@H](C)C(=O)O)C2] | 650 |
| 139. | (S)-4-(5-(3-((2-((S)-3- carboxybutanoyl)-6- methoxyisoindolin-5-yl)oxy) propoxy)-6-methoxythieno [3,2-b]pyridin-2-yl)-2- methyl-4-oxobutanoic acid | 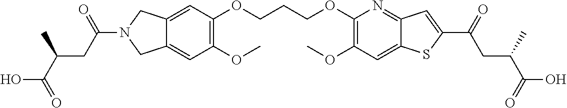 [SMILES: COc1cc2c(cc1OCCCOc1nc3cc(C(=O)C[C@H](C)C(=O)O)sc3cc1OC)CN(C(=O)C[C@H](C)C(=O)O)C2] | 615 |
| 140. | (S)-4-(5-(3-((2-((S)-3- carboxybutanoyl)-4-fluoro- 6-methoxyisoindolin-5- yl)oxy)propoxy)-6- methoxythieno[3,2-b]pyridin- 2-yl)-2-methyl-4-oxobutanoic acid | 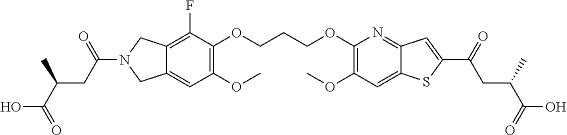 [SMILES: COc1cc2sc(C(=O)C[C@H](C)C(=O)O)cc2nc1OCCCOc1c(OC)cc2c(c1F)CN(C(=O)C[C@H](C)C(=O)O)C2] | 633 |
| 141. | (S)-4-(5-(3-((2-((S)-3- carboxybutanoyl)-4-chloro- 6-methoxyisoindolin-5- yl)oxy)propoxy)-6- methoxythieno[3,2-b]pyridin- 2-yl)-2-methyl-4- oxobutanoic acid | 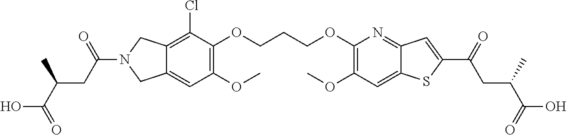 [SMILES: COc1cc2sc(C(=O)C[C@H](C)C(=O)O)cc2nc1OCCCOc1c(OC)cc2c(c1Cl)CN(C(=O)C[C@H](C)C(=O)O)C2] | 649 |
| 142. | (S)-4-(5-(3-((2-((S)-3- carboxybutanoyl)-4,7- dichloro-6-methoxyiso- indolin-5-yl)oxy)propoxy)- 6-methoxythieno[3,2-b] pyridin-2-yl)-2-methyl-4- oxobutanoic acid | 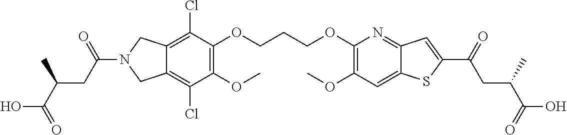 [SMILES: COc1cc2sc(C(=O)C[C@H](C)C(=O)O)cc2nc1OCCCOc1c(Cl)c2c(c(Cl)c1OC)CN(C(=O)C[C@H](C)C(=O)O)C2] | 683 |
| 143. | (S)-4-(5-(3-((2-((S)-3- carboxybutanoyl)-4-chloro- 7-fluoro-6-methoxy- isoindolin-5-yl)oxy) propoxy)-6-methoxythieno [3,2-b]pyridin-2-yl)-2- methyl-4-oxobutanoic acid | 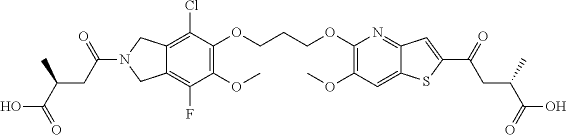 [SMILES: COc1cc2sc(C(=O)C[C@H](C)C(=O)O)cc2nc1OCCCOc1c(Cl)c2c(c(F)c1OC)CN(C(=O)C[C@H](C)C(=O)O)C2] | 667 |
| 144. | (S)-4-(5-(3-((2-((S)-3- carboxybutanoyl)-4,7- difluoro-6-methoxy- isoindolin-5-yl)oxy) propoxy)-6-methoxy- thieno[3,2-b]pyridin-2-yl)- 2-methyl-4-oxobutanoic acid | 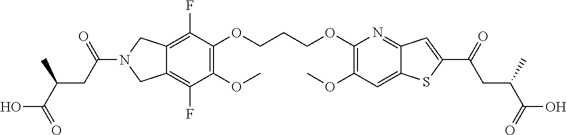 [SMILES: COc1cc2sc(C(=O)C[C@H](C)C(=O)O)cc2nc1OCCCOc1c(F)c2c(c(F)c1OC)CN(C(=O)C[C@H](C)C(=O)O)C2] | 651 |
| 145. | (S)-4-(5-(3-((2-((S)-3- carboxybutanoyl)-4,7- difluoro-6-hydroxyiso- indolin-5-yl)oxy)propoxy)- 6-methoxythieno[3,2-b] pyridin-2-yl)-2-methyl-4- oxobutanoic acid | 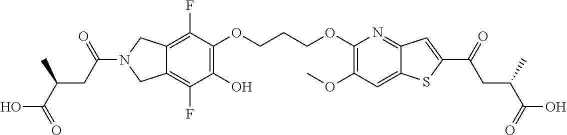 [SMILES: COc1cc2sc(C(=O)C[C@H](C)C(=O)O)cc2nc1OCCCOc1c(O)c(F)c2c(c1F)CN(C(=O)C[C@H](C)C(=O)O)C2] | 637 |
| 146. | (S)-4-(5-(3-((2-((S)-3- carboxybutanoyl)-4-fluoro- 6-hydroxybenzo[b]thiophen- 5-yl)oxy)propoxy)-4-fluoro- 6-methoxyisoindolin-2-yl)- 2-methyl-4-oxobutanoic acid | 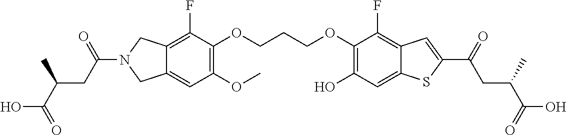 [SMILES: COc1cc2c(c(F)c1OCCCOc1c(O)cc3sc(C(=O)C[C@H](C)C(=O)O)cc3c1F)CN(C(=O)C[C@H](C)C(=O)O)C2] | 636 |
| 147. | (S)-4-(5-(3-((2-((S)-3- carboxybutanoyl)-4-chloro- 6-hydroxybenzo[b]thiophen- 5-yl)oxy)propoxy)-4-fluoro- 6-methoxyisoindolin-2-yl)- 2-methyl-4-oxobutanoic acid | 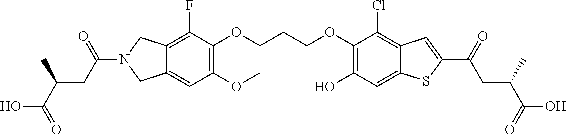 [SMILES: COc1cc2c(c(F)c1OCCCOc1c(O)cc3sc(C(=O)C[C@H](C)C(=O)O)cc3c1Cl)CN(C(=O)C[C@H](C)C(=O)O)C2] | 652 |
| 148. | (S)-4-(5-(3-((2-((S)-3- carboxybutanoyl)-4-chloro- 6-methoxyisoindolin-5-yl) oxy)propoxy)-4-fluoro-6- hydroxybenzo[b]thiophen- 2-yl)-2-methyl-4- oxobutanoic acid | 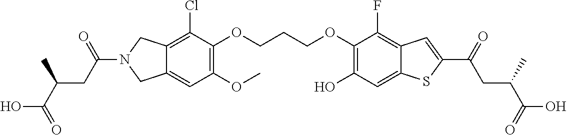 [SMILES: COc1cc2c(c(Cl)c1OCCCOc1c(O)cc3sc(C(=O)C[C@H](C)C(=O)O)cc3c1F)CN(C(=O)C[C@H](C)C(=O)O)C2] | 652 |
| 149. | (S)-4-(5-(3-((2-((S)-3- carboxybutanoyl)-4-chloro- 6-hydroxybenzo[b]thiophen- 5-yl)oxy)propoxy)-4-chloro- 6-methoxyisoindolin-2-yl)-2- methyl-4-oxobutanoic acid | 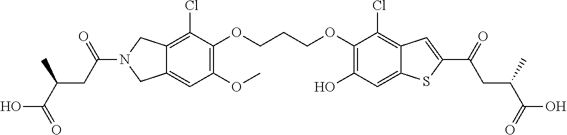 [SMILES: COc1cc2c(c(Cl)c1OCCCOc1c(O)cc3sc(C(=O)C[C@H](C)C(=O)O)cc3c1Cl)CN(C(=O)C[C@H](C)C(=O)O)C2] | 668 |
| 150. | (S)-4-(6-(3-((2-((S)-3- carboxybutanoyl)-6-hydroxy- benzo[b]thiophen-5-yl)oxy) propoxy)-4-fluoro-5- methoxyisoindolin-2- yl)-2-methyl-4-oxobutanoic acid | 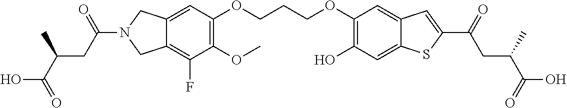 [SMILES: COc1c(OCCCOc2cc3cc(C(=O)C[C@H](C)C(=O)O)sc3cc2O)cc2c(c1F)CN(C(=O)C[C@H](C)C(=O)O)C2] | 618 |
| 151. | (S)-4-(6-(3-((2-((S)-3- carboxybutanoyl)-7-fluoro- 6-hydroxybenzo[b]thiophen- 5-yl)oxy)propoxy)-4-fluoro- 5-methoxyisoindolin-2-yl)-2- methyl-4-oxobutanoic acid | 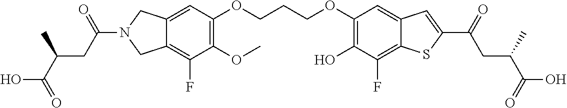 [SMILES: COc1c(OCCCOc2cc3cc(C(=O)C[C@H](C)C(=O)O)sc3c(F)c2O)cc2c(c1F)CN(C(=O)C[C@H](C)C(=O)O)C2] | 636 |
| 152. | (S)-4-(6-(3-((2-((S)-3- carboxybutanoyl)-7-chloro- 6-hydroxybenzo[b]thiophen- 5-yl)oxy)propoxy)-4-fluoro- 5-methoxyisoindolin-2-yl)-2- methyl-4-oxobutanoic acid | 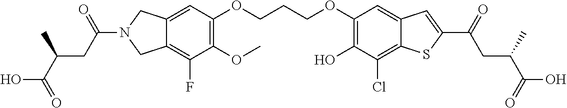 [SMILES: COc1c(OCCCOc2cc3cc(C(=O)C[C@H](C)C(=O)O)sc3c(Cl)c2O)cc2c(c1F)CN(C(=O)C[C@H](C)C(=O)O)C2] | 652 |
| 153. | (S)-4-(5-(3-((2-((S)-3- carboxybutanoyl)-7-chloro- 6-methoxyisoindolin-5- yl)oxy)propoxy)-7-fluoro- 6-hydroxybenzo[b] thiophen-2-yl)-2-methyl-4- oxobutanoic acid | 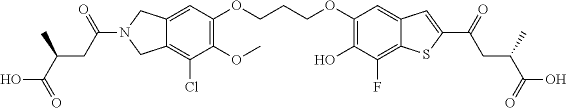 [SMILES: COc1c(OCCCOc2cc3cc(C(=O)C[C@H](C)C(=O)O)sc3c(F)c2O)cc2c(c1Cl)CN(C(=O)C[C@H](C)C(=O)O)C2] | 652 |
| 154. | (S)-4-(5-(3-((2-((S)-3- carboxybutanoyl)-4,7- difluoro-6-methoxyiso- indolin-5-yl)oxy)propoxy)- 7-chloro-6-hydroxybenzo[b] thiophen-2-yl)-2-methyl-4- oxobutanoic acid | 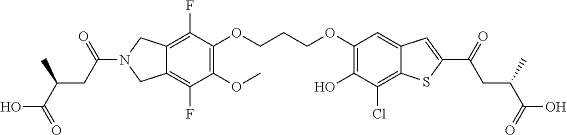 [SMILES: COc1c(F)c2c(c(F)c1OCCCOc1cc3cc(C(=O)C[C@H](C)C(=O)O)sc3c(Cl)c1O)CN(C(=O)C[C@H](C)C(=O)O)C2] | 670 |
| 155. | (S)-4-(5-(3-((2-((S)-3- carboxybutanoyl)-4,7- difluoro-6-methoxyiso- indolin-5-yl)oxy)propoxy)- 7-fluoro-6-hydroxybenzo[b] thiophen-2-yl)-2-methyl- 4-oxobutanoic acid | 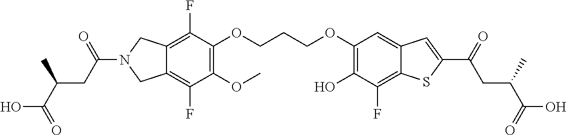 [SMILES: COc1c(F)c2c(c(F)c1OCCCOc1cc3cc(C(=O)C[C@H](C)C(=O)O)sc3c(F)c1O)CN(C(=O)C[C@H](C)C(=O)O)C2] | 654 |
| 156. | (S)-4-(5-(3-((2-((S)-3- carboxybutanoyl)-4-chloro- 7-fluoro-6-methoxy- isoindolin-5-yl)oxy) propoxy)-7-fluoro-6- hydroxybenzo[b]thiophen-2- yl)-2-methyl-4-oxobutanoic acid | 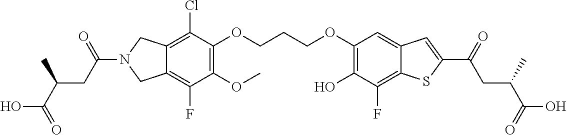 [SMILES: COc1c(F)c2c(c(Cl)c1OCCCOc1cc3cc(C(=O)C[C@H](C)C(=O)O)sc3c(F)c1O)CN(C(=O)C[C@H](C)C(=O)O)C2] | 670 |
| 157. | (S)-4-(6-(3-((2-((S)-3- carboxybutanoyl)-4,7- difluoro-6-hydroxybenzo[b] thiophen-5-yl)oxy) propoxy)-4-fluoro-5- methoxyisoindolin-2-yl)-2- methyl-4-oxobutanoic acid | 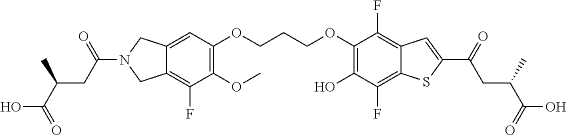 [SMILES: COc1c(OCCCOc2c(O)c(F)c3sc(C(=O)C[C@H](C)C(=O)O)cc3c2F)cc2c(c1F)CN(C(=O)C[C@H](C)C(=O)O)C2] | 654 |
| 158. | (S)-4-(6-(3-((2-((S)-3- carboxybutanoyl)-7-chloro- 4-fluoro-6-hydroxybenzo[b] thiophen-5-yl)oxy) propoxy)-4-fluoro-5- methoxyisoindolin-2-yl)-2- methyl-4-oxobutanoic acid | 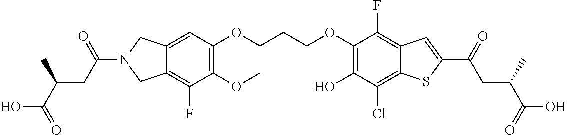 [SMILES: COc1c(OCCCOc2c(O)c(Cl)c3sc(C(=O)C[C@H](C)C(=O)O)cc3c2F)cc2c(c1F)CN(C(=O)C[C@H](C)C(=O)O)C2] | 670 |
| 159. | (S)-4-(6-(3-((2-((S)-3- carboxybutanoyl)-4,7- dichloro-6-hydroxybenzo[b] thiophen-5-yl)oxy) propoxy)-4-fluoro-5- methoxyisoindolin-2-yl)-2- methyl-4-oxobutanoic acid | 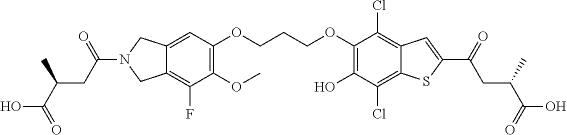 [SMILES: COc1c(OCCCOc2c(O)c(Cl)c3sc(C(=O)C[C@H](C)C(=O)O)cc3c2Cl)cc2c(c1F)CN(C(=O)C[C@H](C)C(=O)O)C2] | 686 |
| 160. | (S)-4-(5-(3-((2-((S)-3- carboxybutanoyl)-5- methoxybenzo[b]thiophen- 6-yl)oxy)propoxy)-6- methoxyisoindolin-2-yl)-2- methyl-4-oxobutanoic acid | 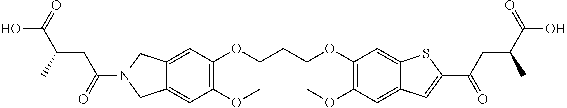 [SMILES: COc1cc2c(cc1OCCCOc1cc3sc(C(=O)C[C@H](C)C(=O)O)cc3cc1OC)CN(C(=O)C[C@H](C)C(=O)O)C2] | 614 |
| 161. | 4-(5-(3-((2-(3-carboxy- propanoyl)-6-methoxy- isoindolin-5-yl)oxy) propoxy)-6-methoxythieno [2,3-b]pyridin-2-yl)-4- oxobutanoic acid | 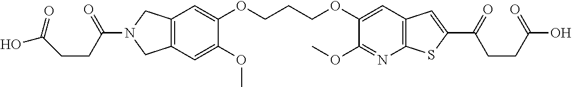 | 587 |
| 162. | 4-(5-(3-((2-(4-(((4R,5R)- 5-hydroxy-1,2-dithian-4- yl)oxy)-4-oxobutanoyl)-6- methoxythieno[2,3-b] pyridin-5-yl)oxy)propoxy)-6- methoxyisoindolin-2-yl)-4- oxobutanoic acid | 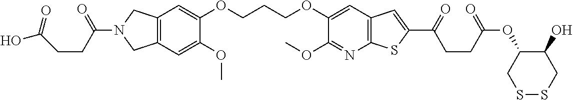 [SMILES: COc1cc2c(cc1OCCCOc1cc3cc(C(=O)CCC(=O)O[C@H]4CSSC[C@@H]4O)sc3nc1OC)CN(C(=O)CCC(=O)O)C2] | 721 |
| 163. | 4-(5-(3-((6-(3-carboxy- propanoyl)-3-methoxy-6,7- dihydro-5H-pyrrolo[3,4-b] pyridin-2-yl)oxy)propoxy)- 6-methoxybenzo[b]thiophen- 2-yl)-4-oxobutanoic acid | 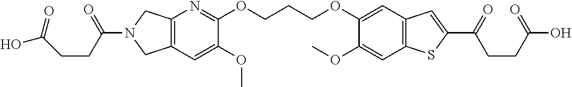 | 587 |
| 164. | 4-(2-(3-((2-(4-(((4R,5R)- 5-hydroxy-1,2-dithian- 4-yl)oxy)-4-oxobutanoyl)-6- methoxybenzo[b]thiophen- 5-yl)oxy)propoxy)-3- methoxy-5,7-dihydro-6H- pyrrolo[3,4-b]pyridin-6-yl)-4- oxobutanoic acid | 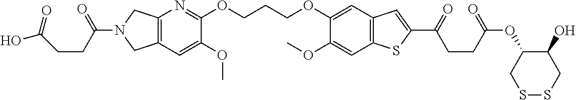 [SMILES: COc1cc2sc(C(=O)CCC(=O)O[C@H]3CSSC[C@@H]3O)cc2cc1OCCCOc1nc2c(cc1OC)CN(C(=O)CCC(=O)O)C2] | 721 |
| 165. | 4-(5-(3-((6-(3-carboxy- propanoyl)-2-methoxy-6,7- dihydro-5H-pyrrolo[3,4-b] pyridin-3-yl)oxy)propoxy)-6- methoxybenzo[b]thiophen-2- yl)-4-oxobutanoic acid | 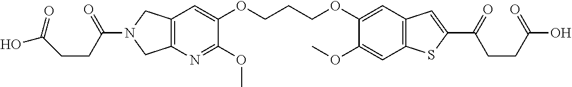 | 587 |
| 166. | 4-(3-(3-((2-(4-(((4R,5R)- 5-hydroxy-1,2-dithian-4- yl)oxy)-4-oxobutanoyl)-6- methoxybenzo[b]thiophen-5- yl)oxy)propoxy)-2-methoxy- 5,7-dihydro-6H-pyrrolo[3,4- b]pyridin-6-yl)-4-oxobutanoic acid | 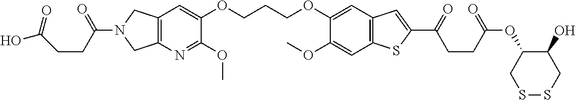 [SMILES: COc1cc2sc(C(=O)CCC(=O)O[C@H]3CSSC[C@@H]3O)cc2cc1OCCCOc1cc2c(nc1OC)CN(C(=O)CCC(=O)O)C2] | 721 |
| 167. | 4-(2-(3-((2-(4-(((4R,5R)- 5-hydroxy-1,2-dithian-4-yl) oxy)-4-oxobutanoyl)-6- methoxythieno[3,2-b]pyridin- 5-yl)oxy)propoxy)-3- methoxy-5,7-dihydro-6H- pyrrolo[3,4-b]pyridin-6-yl)- 4-oxobutanoic acid | 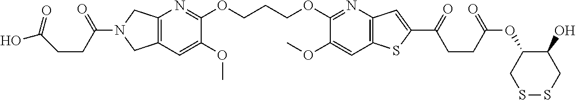 [SMILES: COc1cc2c(nc1OCCCOc1nc3cc(C(=O)CCC(=O)O[C@H]4CSSC[C@@H]4O)sc3cc1OC)CN(C(=O)CCC(=O)O)C2] | 722 |
| 168. | 4-(5-(3-((2-(3-carboxy- propanoyl)-5-methoxythieno [2,3-b]pyridin-6-yl)oxy) propoxy)-6-methoxy- isoindolin-2-yl)-4- oxobutanoic acid | 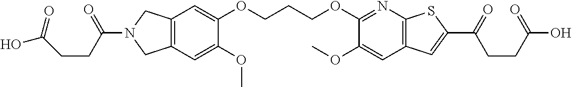 | 587 |
| 169. | (S)-4-(5-(3-((2-(3-carboxy- propanoyl)-5-methoxy- thieno[2,3-b]pyridin-6-yl) oxy)propoxy)-6-methoxy- isoindolin-2-yl)-2-methyl-4- oxobutanoic acid | 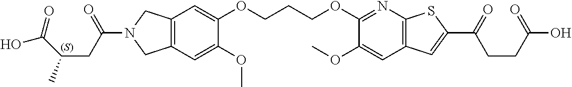 | 601 |
| 170. | (S)-4-(6-(3-((2-(3-carboxy- propanoyl)-6-methoxy- isoindolin-5-yl)oxy) propoxy)-5-methoxythieno [2,3-b]pyridin-2-yl)-2- methyl-4-oxobutanoic acid | 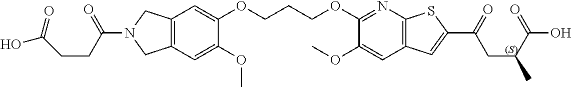 | 601 |
| 171. | (S)-4-(5-(3-((2-((S)-3- carboxybutanoyl)-5- methoxythieno[2,3-b]pyridin- 6-yl)oxy)propoxy)-6- methoxyisoindolin-2-yl)-2- methyl-4-oxobutanoic acid | 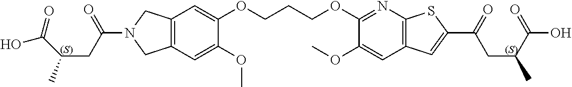 | 615 |
| 172. | 4-(5-(3-((2-(4-(((4R,5R)- 5-hydroxy-1,2-dithian-4- yl)oxy)-4-oxobutanoyl)-5- methoxythieno[2,3-b] pyridin-6-yl)oxy)propoxy)- 6-methoxyisoindolin-2-yl)- 4-oxobutanoic acid | 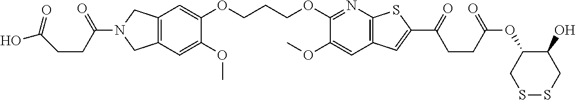 [SMILES: COc1cc2c(cc1OCCCOc1nc3sc(C(=O)CCC(=O)O[C@H]4CSSC[C@@H]4O)cc3cc1OC)CN(C(=O)CCC(=O)O)C2] | 721 |

### Example A: Human STING WT Binding Assay

Cisbio Bioassays' human STING WT binding assay (#64BDSTGPEG & 64BDSTGPEH, Cisbio) is for quantitative measurement of human STING WT ligand using HTRF® technology.

1. Adding Compounds

Negative control: Dispense 5 μL of diluent into each negative control well. Standard: Dispense 5 μL of each Human STING WT Standard 2′3′-cGAMP (Std 0-Std 7) into each standard well. Compound: Dispense 5 μL of compound into each compound well.

2. Adding Proteins

Negative control: Add 5 μL of detection buffer to all wells. Other wells: Add 5 μL of human STING WT protein 6His-tagged protein to all wells.

3. Adding Antibodies

Add 10 μL of premixed STING WT ligand d2 reagent and 6His Tb antibody working solution to all wells.

4. RT Incubation

Seal the plate and incubate 3 hours at RT or at Over Night if necessary.

5. Reading Plate

Remove the plate sealer and read on an HTRF® compatible reader (PerkinElmer, USA). Results were analyzed with a two-wavelength signal ratio: intensity (665 nm)/intensity (620 nm).

6. Curve Fitting

Calculate HTRF Ratio:

$$Ratio = \frac{Signal 665 nm}{Signal 620 nm} \times {10}^{4}$$

Fit the data in GraphPad to obtain IC₅₀ values using equation (2)

Y=Bottom+(Top−Bottom)/(1+10\hat{ }((Log IC₅₀−X)*Hill Slope)) Equation (2):

Y is HTRF Ratio and X is compound concentration.

IC₅₀ value of binding assay for human STING WT:

| | hSTING |
|---|---|
| | WT binding |
| Example | IC₅₀/nM |
| 1 | 0.06 |
| 2 | 11.37 |
| 3 | 8.40 |
| 4 | 0.18 |
| 5 | 0.32 |
| 6 | 0.36 |
| 7 | 1.04 |
| 8 | 0.65 |
| 9 | 2.23 |
| 10 | 0.28 |
| 11 | 2.54 |
| 13 | 12.84 |
| 14 | 46.60 |
| 15 | 92.57 |
| 16 | >150 |
| 17 | 0.19 |
| 18 | 4.59 |
| 19 | 54.62 |
| 20 | 0.19 |
| 21 | 158.60 |

The compounds of the present invention are preferably formulated as pharmaceutical compositions administered by a variety of routes. Most preferably, such compositions are for oral administration. Such pharmaceutical compositions and processes for preparing the same are well known in the art. See, e.g., REMINGTON: THE SCIENCE AND PRACTICE OF PHARMACY (A. Gennaro, et al, eds., 19^{th} ed., Mack Publishing Co., 1995). The compounds of Formula I are generally effective over a wide dosage range.

For example, dosages per day normally fall within the range of about 0.2 mg to about 100 mg total daily dose, preferably 0.2 mg to 50 mg total daily dose, more preferably 0.2 mg to 20 mg total daily dose. In some instances dosage levels below the lower limit of the aforesaid range may be more than adequate, while in other cases still larger doses may be employed. The above dosage range is not intended to limit the scope of the invention in any way. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

What is claimed is:

1. A compound of Formula I or a pharmaceutically acceptable salt thereof: 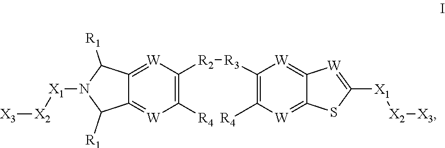
wherein
each W is independently selected from CR₁ or N;
each R₁ is independently selected from H, deuterium, halogen, OR⁶, N(R⁶)₂, COOR⁶, or C(O)N(R⁶)₂, CN or C₁-C₆ alkyl; wherein the C₁-C₆ alkyl is optionally substituted with one or more deuterium, halogen, OR⁶, N(R⁶)₂, COOR⁶, or C(O)N(R⁶)₂;
R₂ and R₃ are independently selected from the group consisting of O—(C₁-C₄ alkylene or haloalkylene), C₁-C₅ alkylene or haloalkylene, N(R⁶)—(C₁-C₄ alkylene or haloalkylene), -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and
each Rs independently is H, deuterium or C₁-C₆ alkyl optionally substituted with one or more halogen;
each R⁴ is independently selected from the group consisting of H, deuterium, halogen, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶);
each R⁶ is independently selected from the group consisting of —H, deuterium, halogen, —NH₂, —CN, —OH, —N₃, —NO₂, carboxyl, C₁₋₆alkyl, C₁₋₆alkoxy, C₂₋₆alkenyl, C₂₋₆alkynyl, —C₆₋₁₀aryl, —C₅₋₁₀heteroaryl, C₃₋₁₀heterocyclic ring or C₃₋₁₀carbocyclic ring, and each of which is independently optionally substituted with deuterium, halogen, —NH₂, —CN, —OH, —NO₂, carbonyl, ═O, oxo, carboxyl, C₁₋₆alkoxy, or C₁₋₆alkyl; and each of the heteroaryl and heterocyclic ring contains at least one heteroatoms selected from N, O or S;
each X₁ is independently selected from the group consisting of C═O, —CH₂—, —CHF—, and —CF₂—;
each X₂ is independently selected from —(C(R⁸)₂)₍₁₋₃₎, —NR⁸(C(R⁸)₂)₍₁₋₃₎, —NH(C(R⁸)₂)₍₁₋₃₎, —N(C₁₋₆alkyl) (C(R⁸)₂)₍₁₋₃₎ or —N(haloC₁₋₆alkyl) (C(R⁸)₂)₍₁₋₃₎; wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle;
each X₃ is independently selected from the group consisting of COOR⁶, C(O)SR⁶, C(S)OR⁶, SO₂R⁶, C(O)N(R⁹)₂, 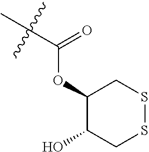 [SMILES: *C(=O)O[C@H]1CSSC[C@@H]1O]
and CN; and each R⁹ is independently selected from H, deuterium, COOR⁶, SO₂R⁶, (CH₂)₁₋₃—C(═O)OR⁶, OR⁶, SR⁶, NH₂, NH(C₁-C₆ alkyl), N(C₁-C₆ alkyl)₂, O(C₁-C₆ alkyl), O(C₆-C₁₀ aryl), O(C₁-C₆ alkyl)-OR⁶, S(C₁-C₆alkyl), S(C₆-C₁₀ aryl), S(═O)₂R⁶, S(═O)₂OR⁶, P(═O)(R⁶)₂, C₁-C₆alkyl, C₂-C₆ alkenyl, C₂-C₆ alkynyl, C₃-C₈ cycloalkyl, C₆-C₁₀aryl, 3-8 membered heterocycloalkyl, or 3-10 membered heteroaryl.

2. The compound or pharmaceutically acceptable salt thereof of claim 1, wherein
wherein 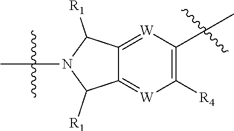
is independently selected from the group consisting of 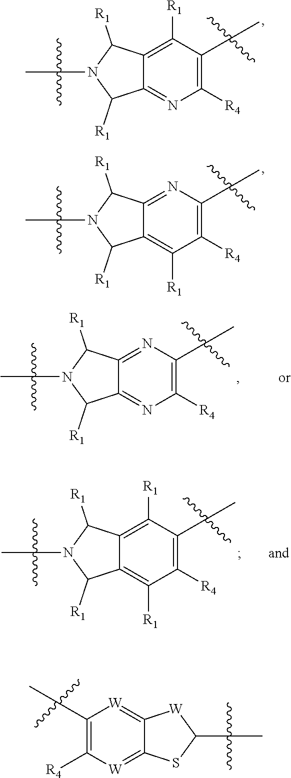
is independently selected from the group consisting of

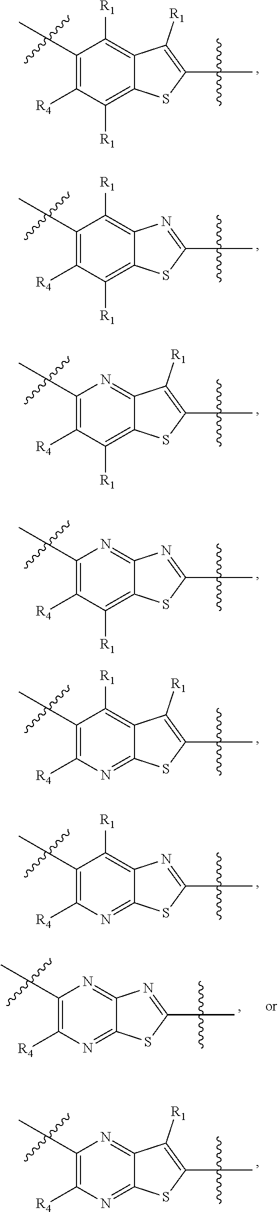

3. The compound or pharmaceutically acceptable salt thereof of claim 1 or 2, wherein each R₁ is independently selected from the group consisting of H, deuterium, halogen, OR⁶, N(R⁶)₂, C₁-C₆ alkyl; wherein the C₁-C₆ alkyl is optionally substituted with one or more deuterium, halogen, OR⁶, N(R⁶)₂, COOR⁶, or C(O)N(R⁶)₂.

4. The compound or pharmaceutically acceptable salt thereof of any one of claims 1-3, wherein each R₁ is independently selected from the group consisting of H, deuterium, halogen, C₁-C₃ alkyl, and C₁-C₃ haloalkyl.

5. The compound or pharmaceutically acceptable salt thereof of any one of claims 1-4, wherein each R₁ is independently selected from the group consisting of H, deuterium, F, Cl, Br, CN and methyl.

6. The compound or pharmaceutically acceptable salt thereof of any one of claims 1-5, wherein R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

7. The compound or pharmaceutically acceptable salt thereof of any one of claims 1-6, wherein R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═O)—(C₁-C₆alkyl)-C(═O)-T_{b}-, -Tₐ-C(═O)—(C₃-C₁₂cycloalkyl)-C(═O)-T_{b}-, -Tₐ-C(═O)—(C₁-C₆alkyl)-(C₃-C₁₂cycloalkyl)-(C₁-C₆alkyl)-C(═O)-T_{b}-, -Tₐ-C(═O)-(3- to 12-membered heterocycloalkyl)-C(═O)-T_{b}-, -Tₐ-C(═O)—(C₁-C₆alkyl)-(3- to 12-membered heterocycloalkyl)-(C₁-C₆alkyl)-C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs; wherein in -Tₐ-(C₃-C₁₂ cycloalkyl)-T_{b}- or -Tₐ-(3- to 12-membered heterocycloalkyl)-T_{b}-, the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(Rs)-, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and each Rs independently is H, deuterium or C₁-C₆ alkyl optionally substituted with one or more halogen.

8. The compound or pharmaceutically acceptable salt thereof of any one of claims 1-7, wherein R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

9. The compound or pharmaceutically acceptable salt thereof of any one of claims 1-8, wherein R₂ and R₃ are independently selected from -Tₐ-C₁-C₆ alkyl-T_{b}-, -Tₐ-N(Rs)-T_{b}, -Tₐ-O-T_{b}, -Tₐ-PEGₙ-O-T_{b}, -Tₐ-S—S-T_{b}, -Tₐ-S—S—S-T_{b}, -Tₐ-N(Rₛ)—N(Rs)-T_{b}-, -Tₐ-C₂-C₆alkenyl-T_{b}-, -Tₐ-C₂-C₆alkynyl-T_{b}-, -Tₐ-C(═O)-T_{b}-, -Tₐ-C(═CH₂)-T_{b}-, -Tₐ-C(═O)—C(═O)-T_{b}-, -Tₐ-C(═S)-T_{b}-, -Tₐ-S(═O)₂-T_{b}-, -Tₐ-S(═O)-T_{b}-, -Tₐ-P(═O)(—ORs)-T_{b}-, -Tₐ-(C₃-C₁₂cycloalkyl)-T_{b}-, -Tₐ-(C₆-C₁₂ aryl)-T_{b}-, -Tₐ-(3- to 12-membered heterocycloalkyl)-T_{b}-, or -Tₐ-(5- to 12-membered heteroaryl)-T_{b}-, wherein the C₁-C₆alkyl, C₂-C₆ alkenyl, C₂-C₆ alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs; and wherein the C₃-C₁₂ cycloalkyl or 3- to 12-membered heterocycloalkyl is attached to Tₐ and T_{b} respectively via two different atoms of the C₃-C₁₂ cycloalkyl or 3- to 12-membered heterocycloalkyl;
PEGₙ is (—OCH₂CH₂—)ₙ, n=1-8;
Tₐ and T_{b} each independently are absent, —N(Rs)-, —O—, C₁-C₆ alkyl, —N(Rs)-(C₁-C₆alkyl)-, —(C₁-C₆ alkyl)-N(R_{S})—, —N(Rs)-(C₁-C₆alkyl)-N(Rs)-, —O—(C₁-C₆ alkyl)-, —(C₁-C₆alkyl)-O—, or —O—(C₁-C₆ alkyl)-O—; wherein the C₁-C₆ alkyl is optionally substituted with one or more halogen; and each Rs independently is H, deuterium or C₁-C₆ alkyl optionally substituted with one or more halogen.

10. The compound or pharmaceutically acceptable salt thereof of any one of claims 1-9, wherein R₂ and R₃ are independently selected from 0-(C₁-C₄ alkylene or haloalkylene)-C₂-C₆alkenyl, C₁-C₅ alkylene or haloalkylene, (C₁-C₄ alkylene or haloalkylene)-N(R⁶), and N(R⁶)—(C₁-C₄ alkylene or haloalkylene)-C₂-C₆alkenyl, —C₀₋₆alkyl-NH—C₀₋₆alkyl-, —C₀₋₆alkyl-N(C₁₋₆alkyl)-C₀₋₆alkyl-, —C₀₋₆alkyl-O—C₀₋₆alkyl-, —C₀₋₆alkyl-PEGₙ-O—C₀₋₆alkyl, —C₀₋₆alkyl-S—S—C₀₋₆alkyl, —C₀₋₆alkyl-S—S—S—C₀₋₆alkyl, —C₀₋₆ alkyl-C₂-C₆alkenyl-C₀₋₆ alkyl-, —C₀₋₆ alkyl-C₂-C₆alkynyl-C₀₋₆ alkyl-, —C₀₋₆ alkyl-C(═O)—C₀₋₆ alkyl-, —C₀₋₆ alkyl-C(═CH₂)—C₀₋₆alkyl-, —C₀₋₆ alkyl-C(═O)—C(═O)—C₀₋₆alkyl-, —C₀₋₆ alkyl-C(═S)—C₀₋₆alkyl-, —C₀₋₆ alkyl-S(═O)₂—C₀₋₆alkyl-, —C₀₋₆alkyl-S(═O)—C₀₋₆alkyl-, —C₀₋₆ alkyl-P(═O)(—OH)—C₀₋₆alkyl-, —C₀₋₆ alkyl-C₃-C₁₂ cycloalkyl-C₀₋₆alkyl-, —C₀₋₆ alkyl-C₆-C₁₂ aryl-C₀₋₆alkyl-, —C₀₋₆ alkyl-(3- to 12-membered heterocyclyl)-C₀₋₆ alkyl-, —C₀₋₆ alkyl-(5- to 12-membered heteroaryl)-C₀₋₆ alkyl-, —C₀₋₆alkyl-O-(5- to 12-membered heteroaryl)-O—C₀₋₆alkyl-, —C₀₋₆alkyl-O—C(═O)—NH—C₀₋₆alkyl-, —C₀₋₆alkyl-O—C(═O)—C₀₋₆alkyl-, —C₀₋₆alkyl-NH—C(═O)—C₀₋₆alkyl-, —OC(═O)—O—, —NH—C(═O)—NH—, or —NH—C(═S)—NH—; wherein the C₂-C₆ alkenyl, C₂-C₆alkynyl, C₃-C₁₂ cycloalkyl, C₆-C₁₂ aryl, 3- to 12-membered heterocycloalkyl, or 5- to 12-membered heteroaryl is optionally substituted with one or more deuterium, halo, —ORs, —N(Rs)₂, or —C(═O)ORs.

11. The compound or pharmaceutically acceptable salt thereof of any one of claims 1-10, wherein R₂ and R₃ are independently selected from -Tₐ-C₂-C₆ alkenyl-T_{b}-, -Tₐ-C(═O)-T_{b}-, -Tₐ-PEGₙ-O-T_{b}, -Tₐ-S—S-T_{b}, -Tₐ-S—S—S-T_{b}, -Tₐ-C(═CH₂)-T_{b}-, or -Tₐ-(C₆-C₁₂ aryl)-T_{b}-, wherein the C₂-C₆alkenyl or C₆-C₁₂ aryl is optionally substituted with one or more deuterium, halo, —ORs, —N(Rs)₂, or —C(═O)ORs;
PEGₙ is (—OCH₂CH₂—)ₙ, n=1-8;
Tₐ and T_{b} each independently are —N(Rs)-, —O—, —(C₁-C₆ alkyl)-O—, or —O—(C₁-C₆ alkyl)-O—; wherein the C₁-C₆alkyl it optionally substituted with one or more halogen; each Rs independently is H, deuterium or C₁-C₆ alkyl optionally substituted with one or more halogen.

12. The compound or pharmaceutically acceptable salt thereof of any one of claims 1-11, wherein R₂-R₃ is selected from 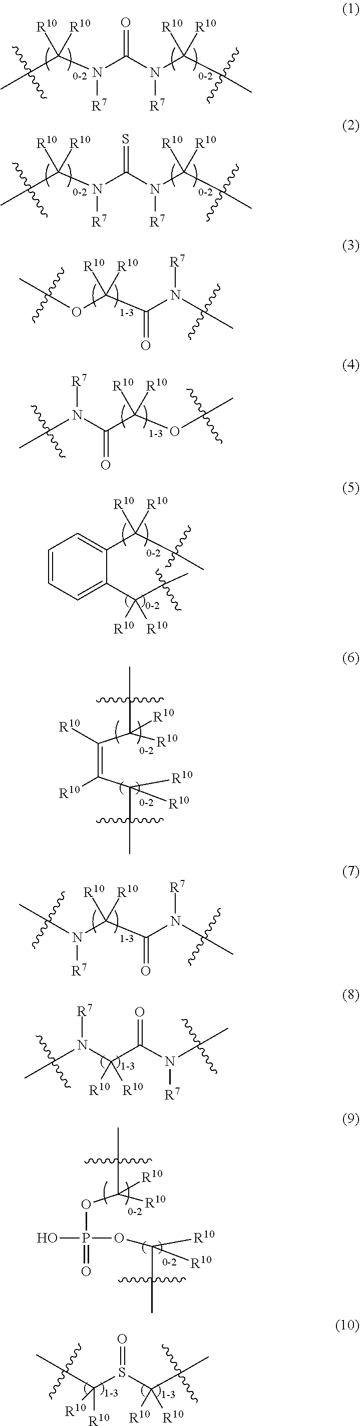 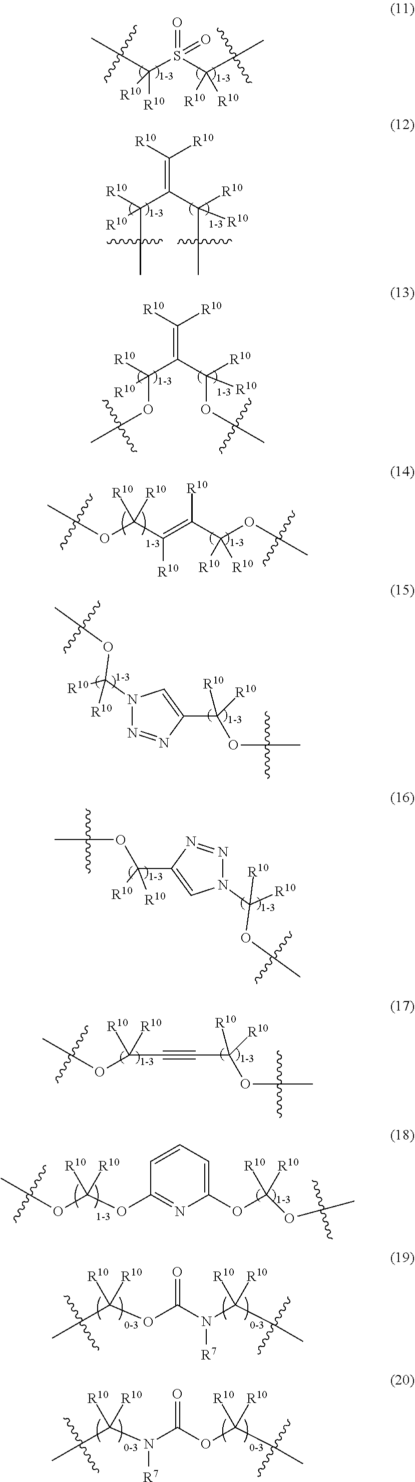 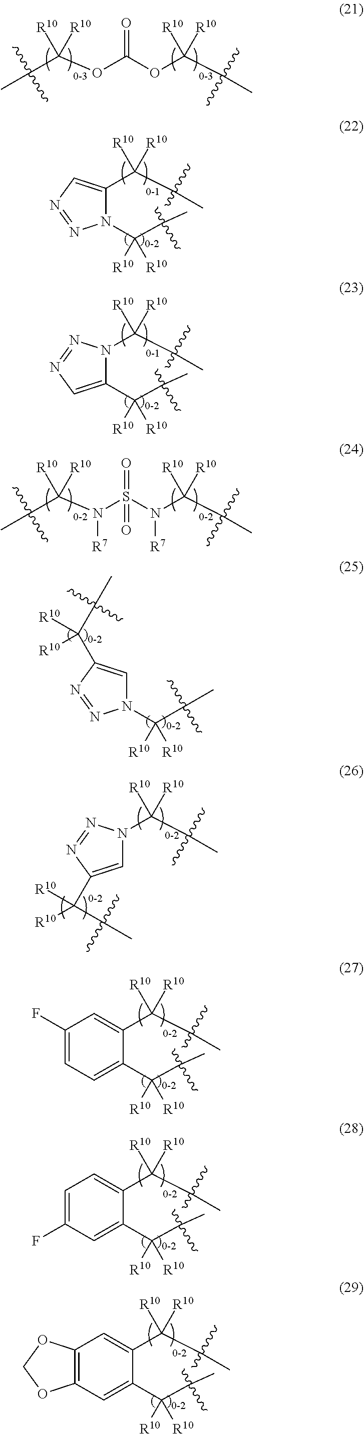 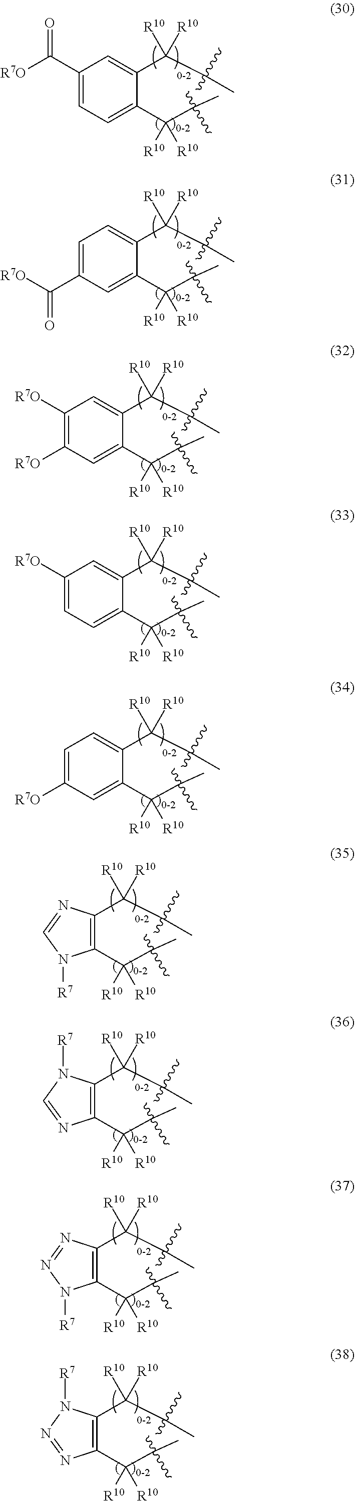 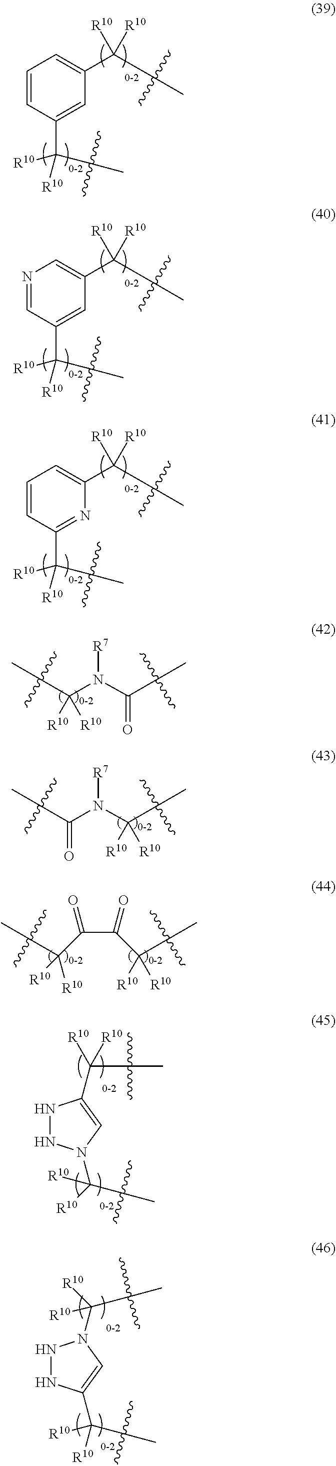 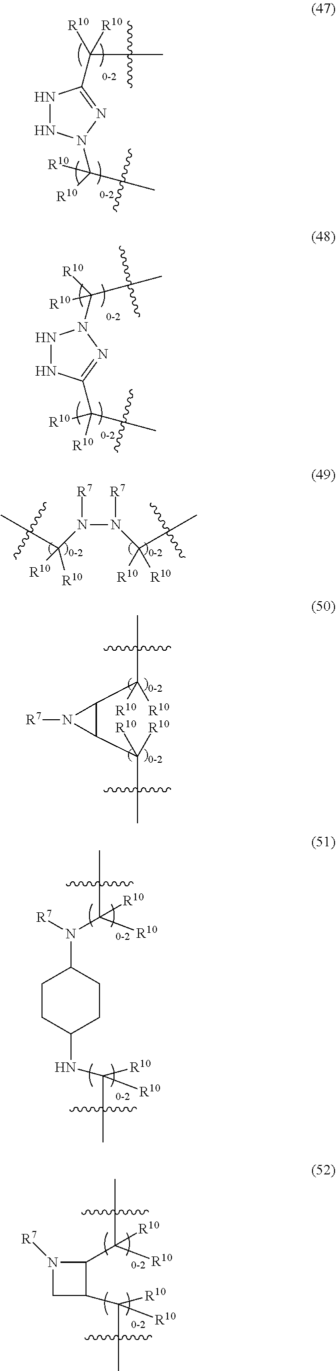 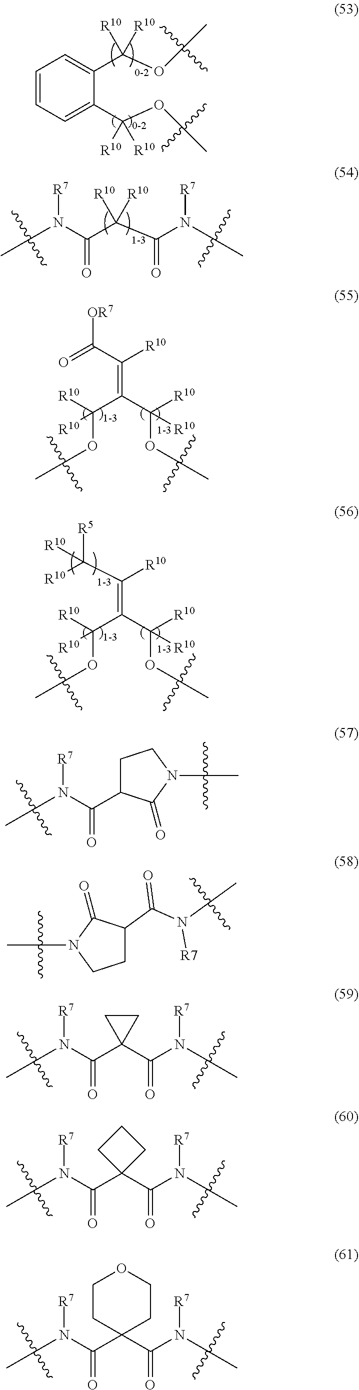 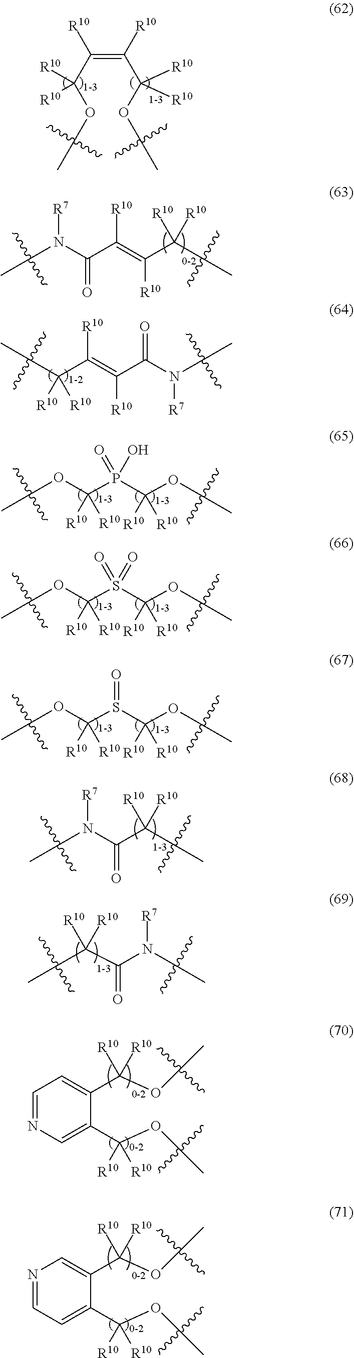 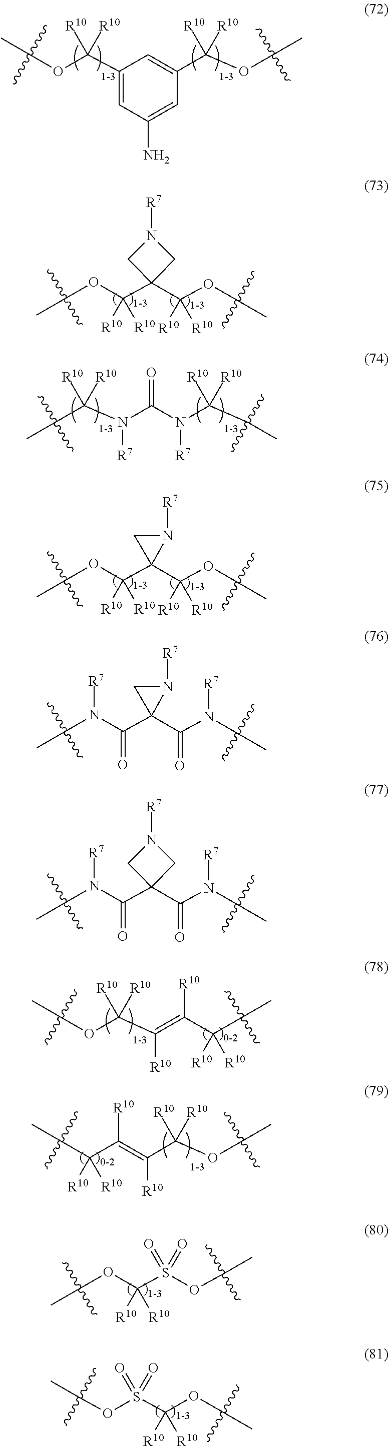 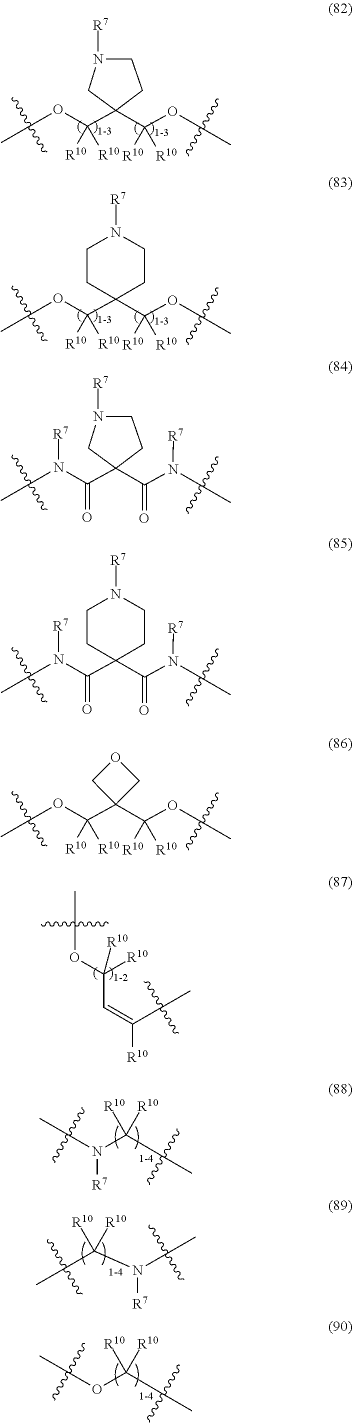 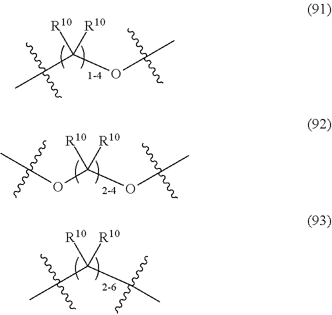
wherein:
each R₅ is independently —OR⁷, NR⁷ or —C(O)OR⁷;
each R⁷ is independently hydrogen or C₁₋₂ alkyl; and
each R¹⁰ is independently hydrogen, C₁₋₂ alkyl or halogen.

13. The compound or pharmaceutically acceptable salt thereof of any one of claims 1-12, wherein
each R⁷ is independently hydrogen, deuterium or methyl;
each R¹⁰ is independently hydrogen, deuterium, methyl or fluorine; or
one R¹⁰ is hydrogen, and the other R¹⁰ is methyl or fluorine.

14. The compound or pharmaceutically acceptable salt thereof of any one of claims 1-13, wherein R₂-R₃ is selected from —(CH₂)₂₋₈—, —O(CH₂)₁₋₇—, —O(CH₂)₁₋₆O—, —OCH₂CH(CH₃)CH₂O—, —OCH(CH₃)CH₂CH(CH₃)O—, 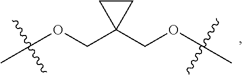 [SMILES: *OCC1(CO*)CC1]
—NH(CH₂)₁₋₇—, —(CH₂)₁₋₆NH(CH₂)₁₋₆—, —(CH₂)₁₋₆N(CH₃)(CH₂)₁₋₆—, —NH(CH₂)₁₋₆O—, —NH—CO—NH—, —N(CH₃)CO—NH—,

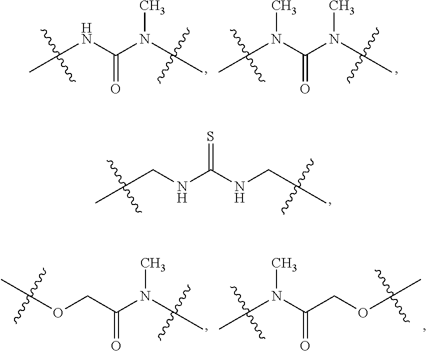

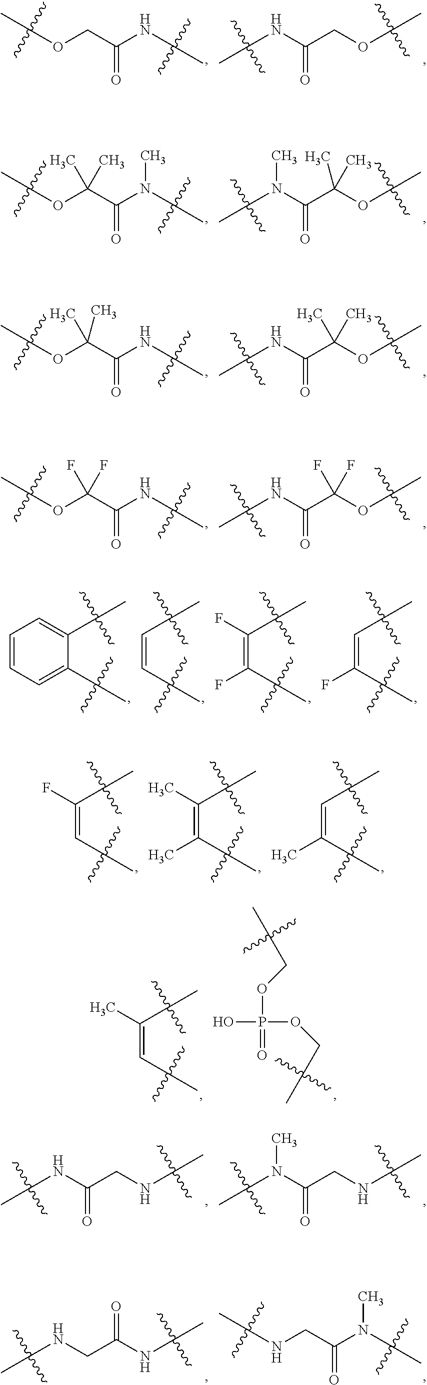

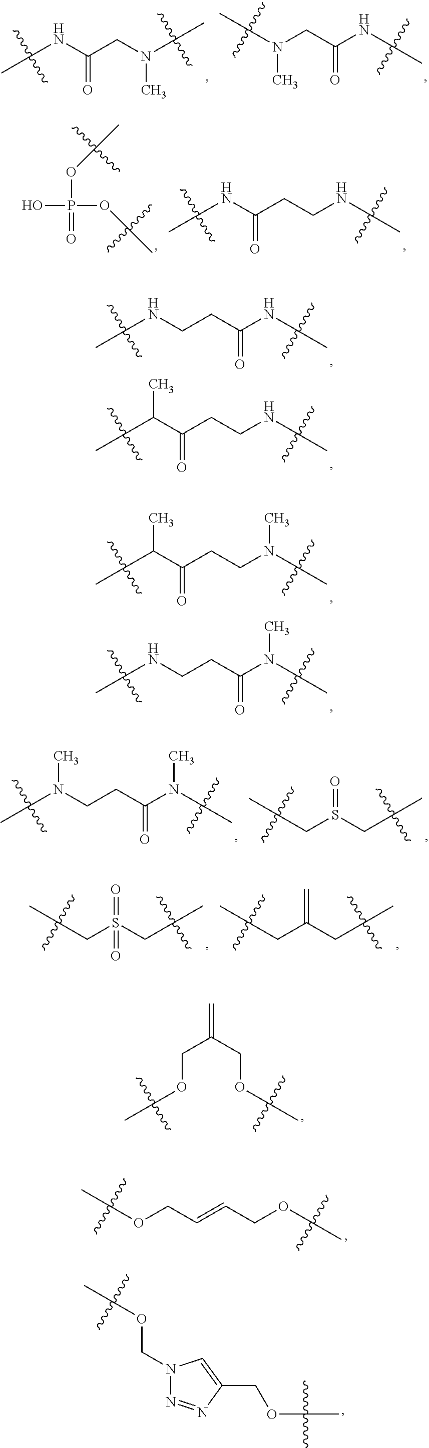

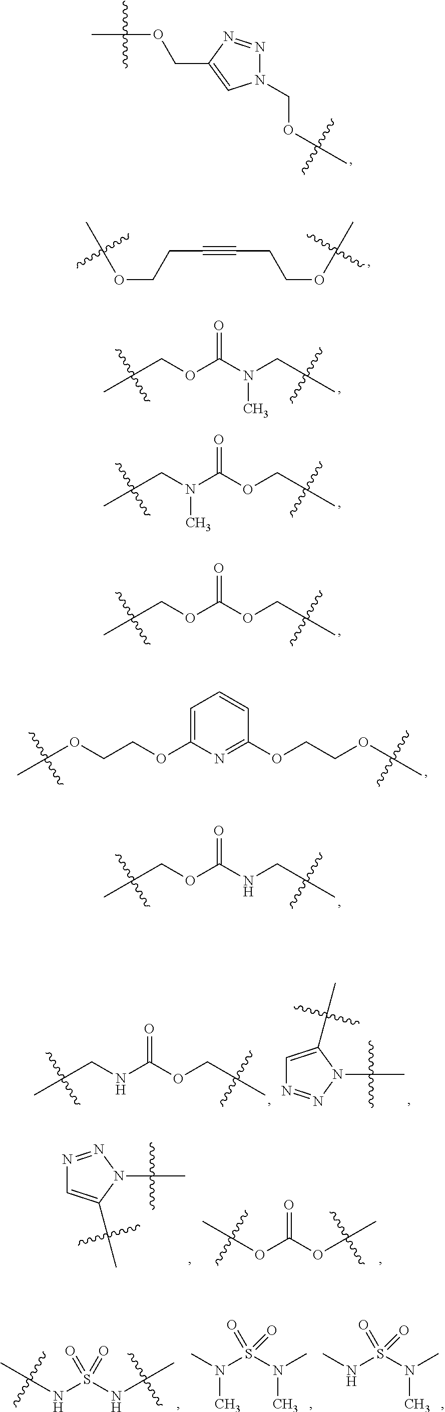

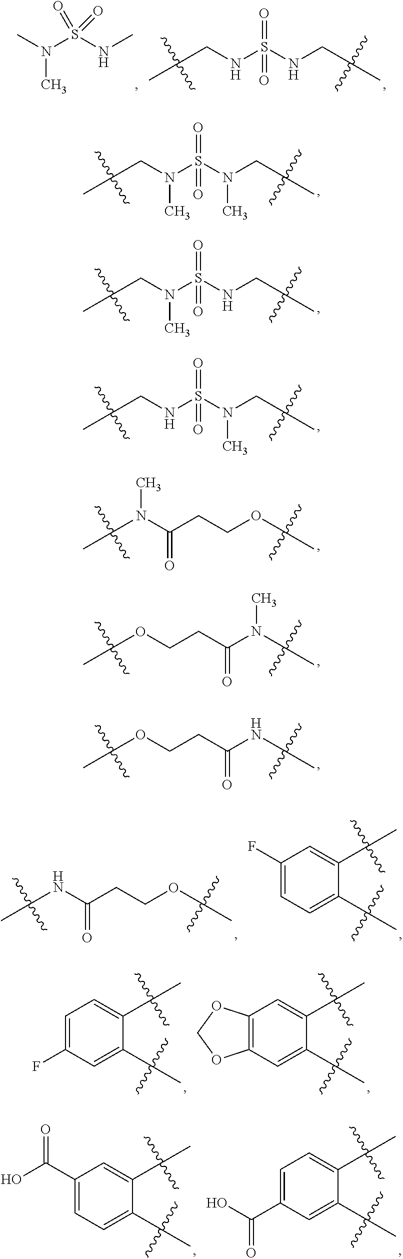

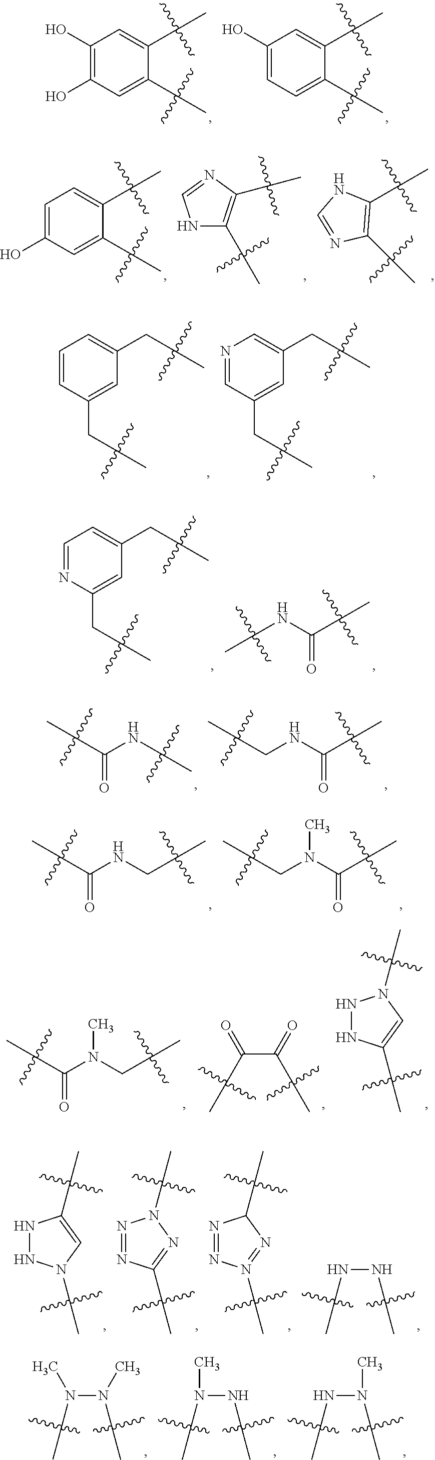

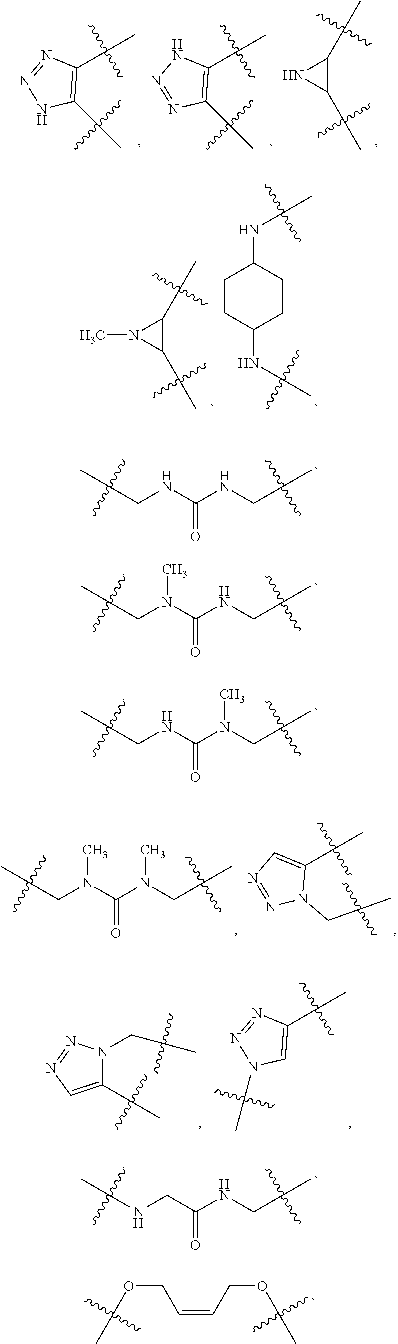

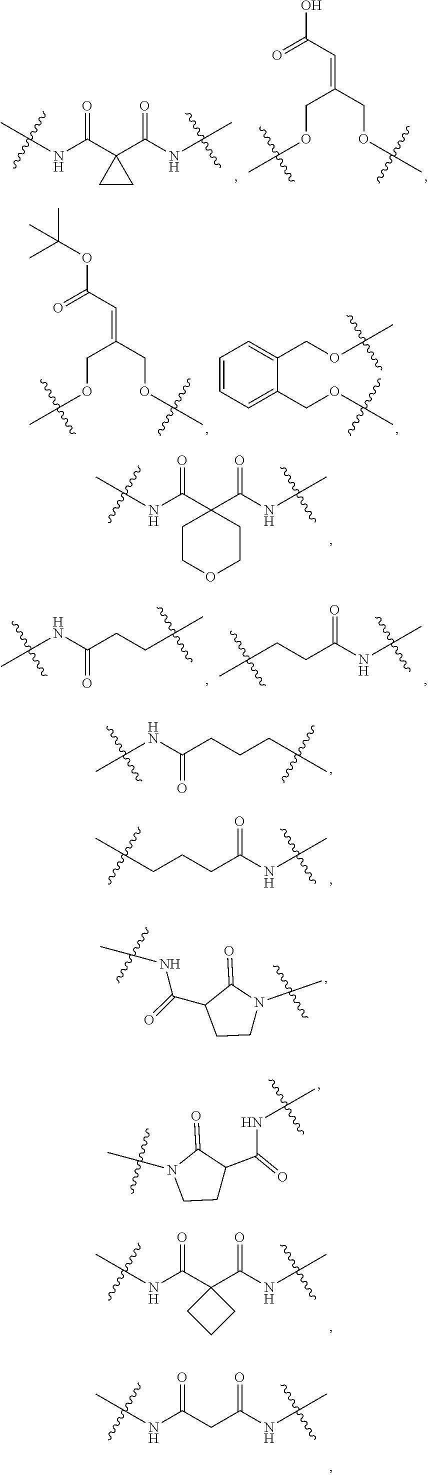

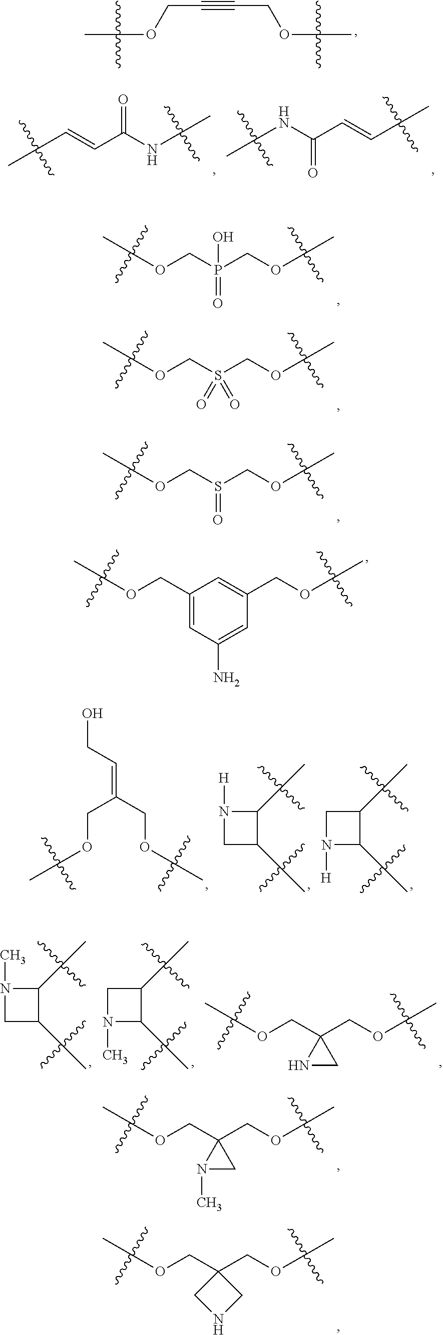

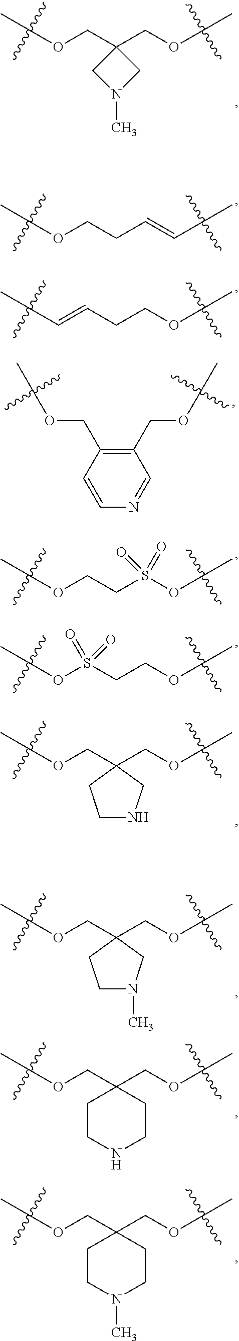

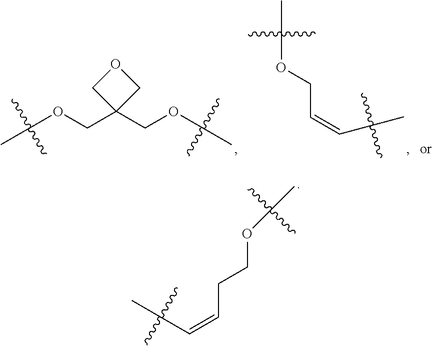 [SMILES: *OCC1(CO*)COC1 ; */C=C\CO* ; */C=C\CCO*]

15. The compound or pharmaceutically acceptable salt thereof of any one of claims 1-14, wherein R₂-R₃ is selected from —(CH₂)₂—, —(CH₂)₃—, —(CH₂)₄—, —(CH₂)₅—, —O(CH₂)₂—, —O(CH₂)₃—, —O(CH₂)₄—, —O(CH₂)₂O—, —O(CH₂)₃O—, —O(CH₂)₄O—, —OCH₂CH(CH₃)CH₂O—, —OCH(CH₃)CH₂CH(CH₃)O—, 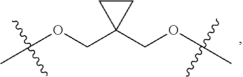 [SMILES: *OCC1(CO*)CC1]
—O(CH₂)₄O—, —O(CH₂)₅O—, —NH(CH₂)₂—, —NH(CH₂)₃—, —NH(CH₂)₄—, —(CH₂)₂NH—, —(CH₂)₃NH—, —(CH₂)₄NH—, —CH₂NHCH₂—, —CH₂N(CH₃)CH₂—, —NH(CH₂)₃O—, —NH—CO—NH—, 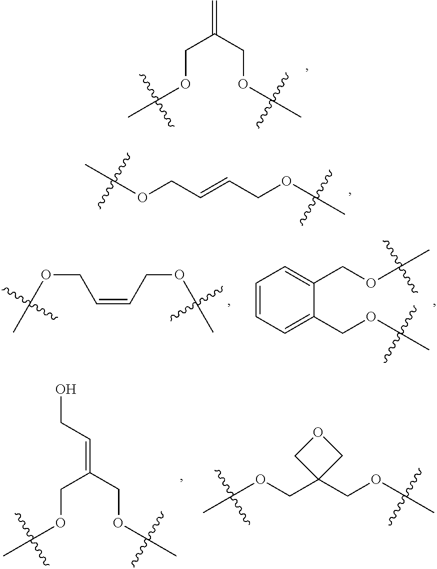
or —N(CH₃)CONH—.

16. The compound or pharmaceutically acceptable salt thereof of any one of claims 1-15, wherein each R₄ is independently selected from the group consisting of H, deuterium, F, Cl, Br, I, CN, OR⁶, N(R⁶)₂, COOR⁶, C(O)N(R⁶)₂, SO₂R⁶, C₁-C₆ alkyl, C₁-C₆ haloalkyl, C₁-C₆ alkyl substituted by OR⁶, C₂-C₆ alkenyl, C₂-C₆ haloalkenyl, C₂-C₆ alkenyl substituted by OR⁶, C₂-C₆ alkynyl, C₂-C₆ haloalkynyl, C₂-C₆ alkynyl substituted by OR⁶, C₃-C₆ cycloalkyl, and a 3- to 6-membered heterocyclic ring including 1 to 2 ring members selected from the group consisting of O, S, and N(R⁶).

17. The compound or pharmaceutically acceptable salt thereof of any one of claims 1-16, wherein, each R₄ is independently selected from the group consisting of H, deuterium, F, Cl, Br, I, OH, C₁-C₃ alkyl, C₁-C₃ haloalkyl, OC₁-C₃ alkyl, OC₁-C₃ haloalkyl, C₂-C₃alkenyl, C₂-C₃alkynyl, and N(R⁶)₂.

18. The compound or pharmaceutically acceptable salt thereof of any one of claims 1-17, wherein each R₄ independently is selected from the group consisting of H, deuterium, Br, Cl, OH, CH₃, CH₂CH₃, CH═CH₂, C≡CH, OCH₃, OCFH₂, OCF₂H, OCF₃, and N(R⁶)₂.

19. The compound or pharmaceutically acceptable salt thereof of any one of claims 1-18, wherein each R₄ independently is selected from the group consisting of H, deuterium, Br, OH, CH₃, CH₂CH₃, CH═CH₂, C≡CH, OCH₃, NH₂ and NHCH₃.

20. The compound or pharmaceutically acceptable salt thereof of any one of claims 1-19, wherein each R⁶ is independently selected from the group consisting of —H, deuterium, —F, —Cl, —Br, —I, —NH₂, —CN, —OH, —N₃, —NO₂, carboxyl, C₁-C₃alkyl, C₁-C₃alkoxy, C₂-C₄alkenyl, C₂-C₄alkynyl, 6-membered aryl, 7-membered aryl, 8-membered aryl, 5-membered heteroaryl, 6-membered heteroaryl, 7-membered heteroaryl, 8-membered heteroaryl, 5-membered heterocyclic ring, 6-membered heterocyclic ring, 7-membered heterocyclic ring, 8-membered heterocyclic ring, 5-membered carbocyclic ring, 6-membered carbocyclic ring, 7-membered carbocyclic ring, or 8-membered carbocyclic ring, and each of which is independently optionally substituted with deuterium, —F, —Cl, —Br, —I, —NH₂, —CN, —OH, —NO₂, carbonyl, ═O, oxo, carboxyl, C₁-C₃alkoxy, or C₁-C₃alkyl; and each of the heteroaryl and heterocyclic ring contains 1 or 2 heteroatoms selected from N, O or S.

21. The compound or pharmaceutically acceptable salt thereof of any one of claims 1-20, wherein each R⁶ is independently selected from the group consisting of —H, deuterium, —F, —Cl, —Br, —I, —NH₂, —CN, —OH, —N₃, —NO₂, carboxyl, methyl, ethyl, propyl, isopropyl, methoxy, ethoxy, propoxy, isopropoxy, ethylene, 6-membered aryl, 5-membered heteroaryl, 6-membered heteroaryl, 5-membered heterocyclic ring, 6-membered heterocyclic ring, 5-membered carbocyclic ring, or 6-membered carbocyclic ring, and each of which is independently optionally substituted with deuterium, —F, —Cl, —Br, —I, —NH₂, —CN, —OH, —NO₂, carbonyl, ═O, oxo, carboxyl, methyl, ethyl, propyl, isopropyl, methoxy, ethoxy, propoxy or isopropoxy; and each of the heteroaryl and heterocyclic ring contains 1 or 2 heteroatoms selected from N or O or S.

22. The compound or pharmaceutically acceptable salt thereof of any one of claims 1-21, wherein each R⁶ is independently selected from the group consisting of H, —F, —Cl, —Br, —I, —NH₂, —CN, —OH, methyl, ethyl, propyl, isopropyl, methoxy, ethoxy, propoxy, isopropoxy, CH₂F, —CHF₂, 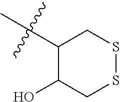 [SMILES: *C1CSSCC1O]
and —CF₃.

23. The compound or pharmaceutically acceptable salt thereof of any one of claims 1-22, wherein each X₁ is independently selected from the group consisting of C═O, —CH₂—, —CHF—, and —CF₂—.

24. The compound or pharmaceutically acceptable salt thereof of any one of claims 1-23, wherein each X₁ is selected from the group consisting of C═O and —CH₂—.

25. The compound or pharmaceutically acceptable salt thereof of any one of claims 1-24, wherein each X₁ is C═O.

26. The compound or pharmaceutically acceptable salt thereof of any one of claims 1-25, wherein each X₂ is independently selected from (C(R⁸)₂)₍₁₋₃₎, wherein each R⁸ is independently selected from the group consisting of H, deuterium, halogen, C₁-C₆ alkyl, CN, OR⁶, N(R⁶)₂, C₁-C₆ haloalkyl, C₃-C₆ cycloalkyl, C₁-C₆ alkyl substituted by OR⁶, and C₁-C₆ alkyl substituted by N(R⁶)₂; optionally 2 R⁸ on different carbon atoms may be taken together, along with the atoms to which they are attached, to form a 3- to 6-membered fused ring; and optionally 2 R⁸ on a single carbon atom may be taken together, along with the atom to which they are attached, to form a 3- to 6-membered spirocycle.

27. The compound or pharmaceutically acceptable salt thereof of any one of claims 1-26, wherein each X₂ is CH₂CHR⁸, where R⁸ is selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl.

28. The compound or pharmaceutically acceptable salt thereof of any one of claims 1-27, wherein each X₂ is CH₂CHR⁸, wherein R⁸ is selected from the group consisting of H, deuterium, CH₃, CH₂OH, CH₂CH₃, CH₂CH₂CH₃, CH(CH₃)₂, CH₂OCH₃, and cyclopropyl.

29. The compound or pharmaceutically acceptable salt thereof of any one of claims 1-28, wherein each X₂ is CHR⁸CHR⁸, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on different carbon atoms are taken together, along with the atoms to which they are attached, to form a 3- to 6-membered fused ring.

30. The compound or pharmaceutically acceptable salt thereof of any one of claims 1-29, wherein each X₂ is CHR⁸CHR⁸, where each R⁸ is independently selected from the group consisting of H, deuterium and C₁-C₃ alkyl, and optionally the 2 R⁸ on different carbon atoms are taken together, along with the atoms to which they are attached, to form a 3- to 6-membered fused ring.

31. The compound or pharmaceutically acceptable salt thereof of any one of claims 1-30, wherein each X₂ is CH₂C(R⁸)₂, where each R⁸ is independently selected from the group consisting of H, deuterium, C₁-C₃ alkyl, C₁-C₃ alkyl substituted by OH, C₁-C₃ alkyl substituted by OC₁-C₃ alkyl, and C₃-C₆ cycloalkyl, and optionally the 2 R⁸ on a single carbon atom are taken together, along with the atoms to which they are attached, to form a 3- to 6-membered spirocycle.

32. The compound or pharmaceutically acceptable salt thereof of any one of claims 1-31, wherein each X₂ is CH₂C(R⁸)₂, where each R⁸ is independently selected from the group consisting of H, deuterium and C₁-C₃ alkyl, and optionally the 2 R⁸ on a single carbon atom are taken together, along with the atoms to which they are attached, to form a 3- to 6-membered spirocycle.

33. The compound or pharmaceutically acceptable salt thereof of any one of claims 1-32, wherein each X³ is independently selected from the group consisting of COOR⁶, C(O)SR⁶, C(S)OR⁶, SO₂R⁶, C(O)N(R⁹)₂, 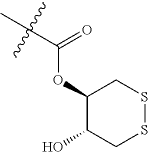 [SMILES: *C(=O)O[C@H]1CSSC[C@@H]1O]
and CN.

34. The compound or pharmaceutically acceptable salt thereof of any one of claims 1-33, wherein each X³ is independently selected from the group consisting of COOR⁶, SO₂R⁶, C(O)N(R⁹)₂, 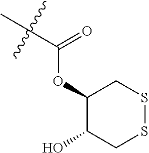 [SMILES: *C(=O)O[C@H]1CSSC[C@@H]1O]
and CN.

35. The compound or pharmaceutically acceptable salt thereof of any one of claims 1-34, wherein each X₃ is independently selected from the group consisting of COOR⁶, C(O)N(R⁹)₂, 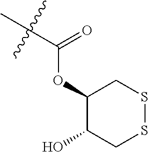 [SMILES: *C(=O)O[C@H]1CSSC[C@@H]1O]
and CN.

36. The compound or pharmaceutically acceptable salt thereof of any one of claims 1-35, wherein each X₃ is independently selected from the group consisting of COOH, COOCH₃, CONH₂, 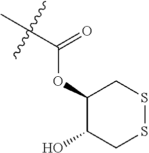 [SMILES: *C(=O)O[C@H]1CSSC[C@@H]1O]
and CN.

37. The compound or pharmaceutically acceptable salt thereof of any one of claims 1-36, wherein each R⁹ is independently selected from the group consisting of H, deuterium, COOR⁶, and SO₂R⁶.

38. The compound or pharmaceutically acceptable salt thereof of any one of claims 1-37, wherein each R⁹ is independently H or deuterium.

39. A compound of general formula I-1 or a pharmaceutically acceptable salt, 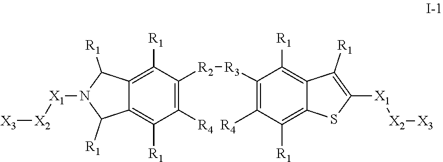
wherein each R₁, R₂, R₃, R₄, X₁, X₂ and X₃ are as defined in any one of claims 1-38.

40. A compound of general formula II or a pharmaceutically acceptable salt, 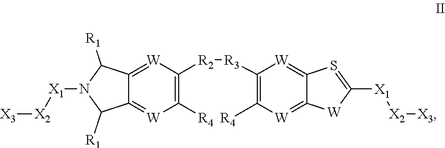
wherein each W, R₁, R₂, R₃, R₄, X₁, X₂ and X₃ are as defined in any one of claims 1-38.

41. The compound or pharmaceutically acceptable salt thereof of claim 40, wherein 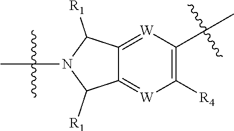
is independently selected from the group consisting of 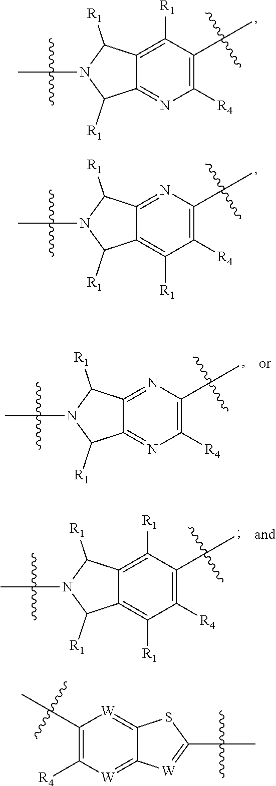
is independently selected from the group consisting of

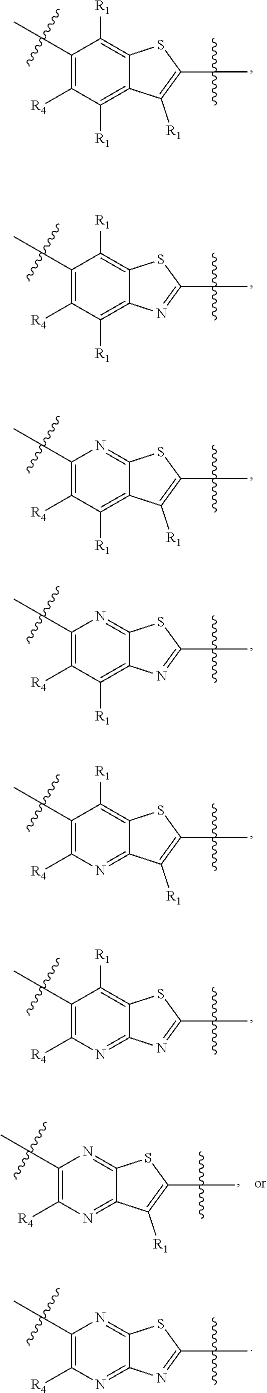

42. A compound of general formula III or a pharmaceutically acceptable salt, 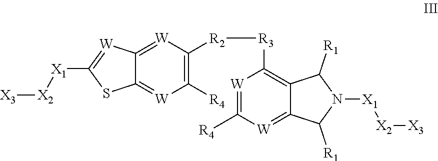
wherein each W, R₁, R₂, R₃, R₄, X₁, X₂ and X₃ are as defined in any one of claims 1-38.

43. The compound or pharmaceutically acceptable salt thereof of claim 42, wherein 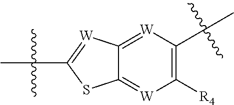
is independently selected from the group consisting of 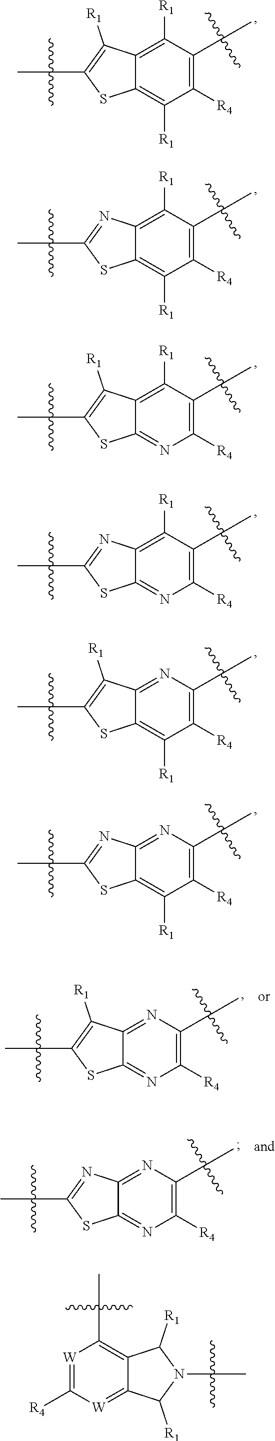
is independently selected from the group consisting of

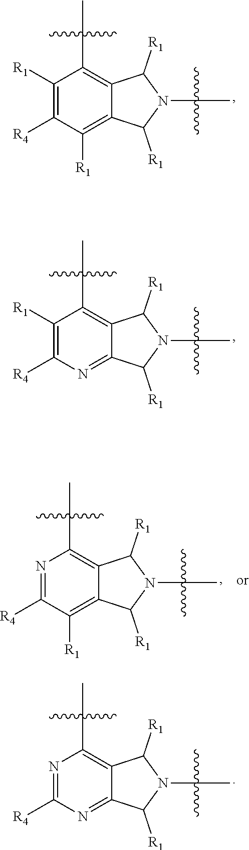

44. A compound of general formula IV or a pharmaceutically acceptable salt, 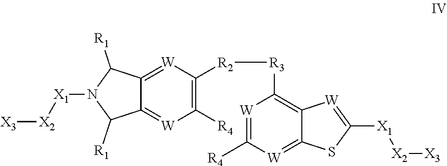
wherein each W, R₁, R₂, R₃, R₄, X₁, X₂ and X₃ are as defined in any one of claims 1-38.

45. The compound or pharmaceutically acceptable salt thereof of claim 44, wherein 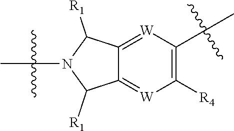
is independently selected from the group consisting of 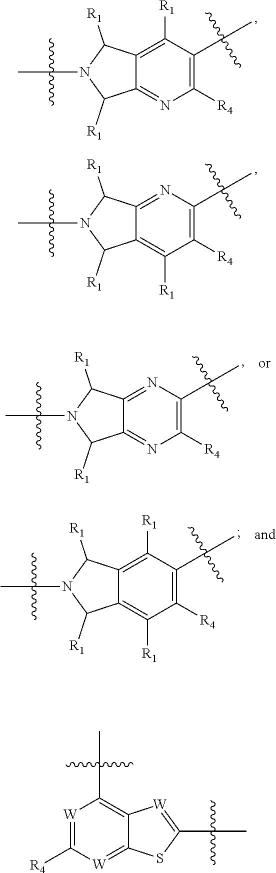
is independently selected from the group consisting of

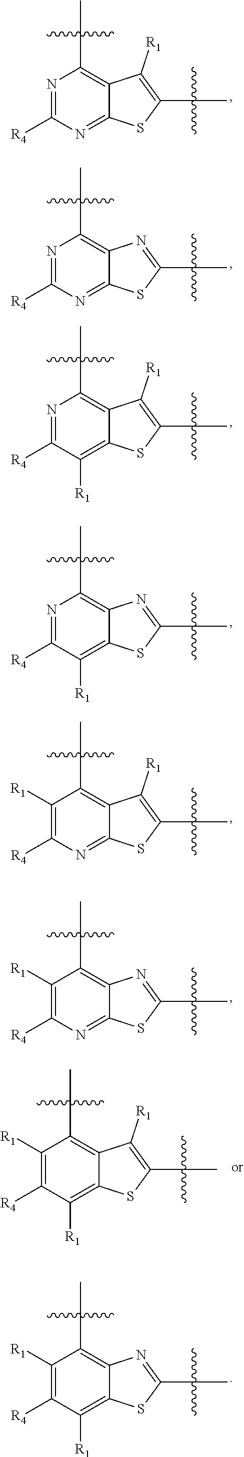

46. A compound of general formula V or a pharmaceutically acceptable salt, 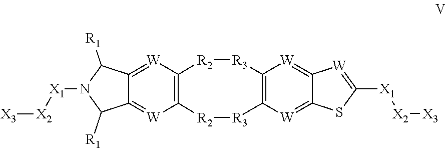
wherein each W, R₁, R₂, R₃, X₁, X₂ and X₃ are as defined in any one of claims 1-38.

47. The compound or pharmaceutically acceptable salt thereof of claim 46, wherein 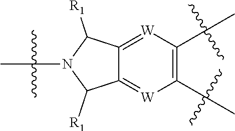
is independently selected from the group consisting of 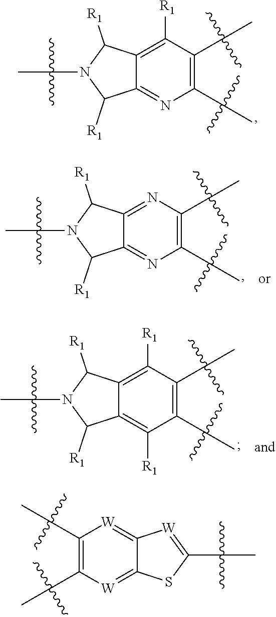
is independently selected from the group consisting of

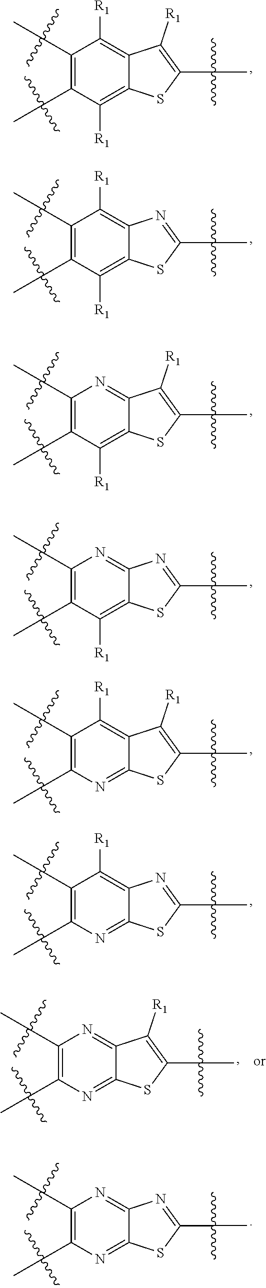

48. The compound or pharmaceutically acceptable salt thereof of any one of claims 1-47, wherein each heterocyclic ring group and each carbocyclic ring group includes single ring, spiral ring, bridge ring, fused ring and various combinations of spiral ring, bridge ring and/or fused ring.

49. The compound or pharmaceutically acceptable salt thereof of any one of claims 1-48, wherein the compound is

| | |
|---|---|
| 1. | 4-(5-(3-((2-(3-carboxypropanoyl)-6-methoxybenzo[b]thiophen-5-yl)oxy)propoxy)-6-metho |
| | xyisoindolin-2-yl)-4-oxobutanoic acid |
| 2. | 4-(5-(3-((2-(3-carboxypropanoyl)-4-chloro-6-methoxyisoindolin-5-yl)oxy)propoxy)-4-fluor |
| | 0-6-methoxybenzo[b]thiophen-2-yl)-4-oxobutanoic acid |
| 3. | 4-(5-(3-((2-(3-carboxypropanoyl)-4-chloro-6-methoxybenzo[b]thiophen-5-yl)oxy)propoxy) |
| | -4-fluoro-6-methoxyisoindolin-2-yl)-4-oxobutanoic acid |
| 4. | 4-(5-(3-((2-(3-carboxypropanoyl)-4-fluoro-6-methoxybenzo[b]thiophen-5-yl)oxy)propoxy)- |
| | 4-fluoro-6-methoxyisoindolin-2-yl)-4-oxobutanoic acid |
| 5. | sodium |
| | (S)-4-(5-(3-((2-((S)-3-carboxylatobutanoyl)-6-methoxybenzo[b]thiophen-5-yl)oxy)propoxy)-6- |
| | methoxyisoindolin-2-yl)-2-methyl-4-oxobutanoate |
| 6. | sodium |
| | (S)-4-(5-(3-((2-((S)-3-carboxylatobutanoyl)-4-fluoro-6-methoxybenzo[b]thiophen-5- |
| | yl)oxy)propoxy)-6-methoxyisoindolin-2-yl)-2-methyl-4-oxobutanoate |
| 7. | sodium |
| | 4-(5-(3-((2-(3-carboxylatopropanoyl)-6-hydroxybenzo[b]thiophen-5-yl)oxy)propoxy)-6- |
| | methoxyisoindolin-2-yl)-4-oxobutanoate |
| 8. | sodium |
| | 4-(5-(3-((2-(3-carboxylatopropanoyl)-6-methoxyisoindolin-5-yl)oxy)propoxy)-6-methoxythieno |
| | [3,2-b]pyridin-2-yl)-4-oxobutanoate |
| 9. | 4-(5-(3-((6-bromo-2-(3-carboxypropanoyl)benzo[b]thiophen-5-yl)oxy)propoxy)-6- |
| | methoxyisoindolin-2-yl)-4-oxobutanoic acid |
| 10. | 4-(5-(3-((2-(3-carboxypropanoyl)-4-fluoro-6-methoxyisoindolin-5-yl)oxy)propoxy)-6- |
| | methoxybenzo[b]thiophen-2-yl)-4-oxobutanoic acid |
| 11. | 4-(5-(3-((6-(3-carboxypropanoyl)-3-methoxy-6,7-dihydro-5H-pyrrolo[3,4-b]pyridin-2- |
| | yl)oxy)propoxy)-6-methoxythieno[3,2-b]pyridin-2-yl)-4-oxobutanoic acid |
| 12. | 4-(5-(3-((2-(4-ethoxy-4-oxobutanoyl)-6-methoxybenzo[b]thiophen-5-yl)oxy)propoxy)-6- |
| | methoxyisoindolin-2-yl)-4-oxobutanoic acid |
| 13. | sodium |
| | 4-(5-(3-((2-(3-carboxylatopropanoyl)-5-methoxythieno[2,3-b]pyridin-6-yl)oxy)propoxy)- |
| | 6-methoxyisoindolin-2-yl)-4-oxobutanoate |
| 14. | 4-(5-(3-((2-(3-carboxypropanoyl)-4-chloro-6-methoxyisoindolin-5-yl)oxy)propoxy)-6- |
| | methoxybenzo[b]thiophen-2-yl)-4-oxobutanoic acid |
| 15. | 4-(5-(3-((2-(3-carboxypropanoyl)-5-methoxythieno[3,2-b]pyridin-6-yl)oxy)propoxy)-6- |
| | methoxyisoindolin-2-yl)-4-oxobutanoic acid |
| 16. | 4-(5-((2-(((2-(3-carboxypropanoyl)-4-chloro-6-methoxyisoindolin-5-yl)oxy)methyl)allyl)oxy)- |
| | 4-fluoro-6-methoxybenzo[b]thiophen-2-yl)-4-oxobutanoic acid |
| 17. | (S)-4-(5-(3-((2-((S)-3-carboxybutanoyl)-4-fluoro-6-methoxybenzo[b]thiophen-5-yl)oxy)pro- |
| | poxy)-4-fluoro-6-methoxyisoindolin-2-yl)-2-methyl-4-oxobutanoic acid |
| 18. | 4-(5-(3-((2-((2-carboxyethyl)carbamoyl)-6-methoxybenzo[b]thiophen-5-yl)oxy)propoxy)-6- |
| | methoxyisoindolin-2-yl)-4-oxobutanoic acid |
| 19. | 4-(5-((2-(((2-(3-carboxypropanoyl)-6-methoxybenzo[b]thiophen-5-yl)oxy)methyl)allyl)oxy)-6- |
| | methoxyisoindolin-2-yl)-4-oxobutanoic acid |
| 20. | 4-(5-(3-((2-(3-carboxypropanoyl)-4-fluoro-6-methoxybenzo[b]thiophen-5-yl)oxy)propoxy)- |
| | 6-methoxyisoindolin-2-yl)-4-oxobutanoic acid |
| 21. | trans-2-(5-(3-((2-(3-carboxypropanoyl)-4-fluoro-6-methoxyisoindolin-5-yl)oxy)propoxy)-6- |
| | methoxybenzo[b]thiophene-2-carbonyl)cyclopropane-1-carboxylic acid |
| 22. | 4-(5-(3-((2-(3-carboxybutanoyl)-6-methoxybenzo[b]thiophen-5-yl)oxy)propoxy)-6- |
| | methoxyisoindolin-2-yl)-2-methyl-4-oxobutanoic acid |
| 23. | (S)-4-(5-(3-((2-((S)-3-carboxybutanoyl)-6-methoxybenzo[b]thiophen-5-yl)oxy)propoxy)-6- |
| | methoxyisoindolin-2-yl)-2-methyl-4-oxobutanoic acid |
| 24. | (S)-4-(5-(3-((2-(3-carboxypropanoyl)-6-methoxyisoindolin-5-yl)oxy)propoxy)-6-methoxy- |
| | benzo[b]thiophen-2-yl)-2-methyl-4-oxobutanoic acid |
| 25. | (S)-4-(5-(3-((2-(3-carboxypropanoyl)-6-methoxybenzo[b]thiophen-5-yl)oxy)propoxy)-6- |
| | methoxyisoindolin-2-yl)-2-methyl-4-oxobutanoic acid |
| 26. | (S)-4-(5-(2-((2-((S)-3-carboxybutanoyl)-6-methoxybenzo[b]thiophen-5-yl)oxy)ethoxy)-6- |
| | methoxyisoindolin-2-yl)-2-methyl-4-oxobutanoic acid |
| 27. | (S)-4-(5-(4-((2-((S)-3-carboxybutanoyl)-6-methoxybenzo[b]thiophen-5-yl)oxy)butoxy)-6- |
| | methoxyisoindolin-2-yl)-2-methyl-4-oxobutanoic acid |
| 28. | (S)-4-(5-((5-((2-((S)-3-carboxybutanoyl)-6-methoxybenzo[b]thiophen-5-yl)oxy)pentyl)oxy)- |
| | 6-methoxyisoindolin-2-yl)-2-methyl-4-oxobutanoic acid |
| 29. | (S)-4-(5-(4-(2-((S)-3-carboxybutanoyl)-6-methoxybenzo[b]thiophen-5-yl)butoxy)-6- |
| | methoxyisoindolin-2-yl)-2-methyl-4-oxobutanoic acid |
| 30. | (S)-4-(5-(3-((2-((S)-3-carboxybutanoyl)-6-methoxybenzo[b]thiophen-5-yl)amino)propoxy)- |
| | 6-methoxyisoindolin-2-yl)-2-methyl-4-oxobutanoic acid |
| 31. | (S)-4-(5-(4-((2-((S)-3-carboxybutanoyl)-6-methoxybenzo[b]thiophen-5-yl)amino)butyl)-6- |
| | methoxyisoindolin-2-yl)-2-methyl-4-oxobutanoic acid |
| 32. | (S)-4-(5-(4-(2-((S)-3-carboxybutanoyl)-6-methoxybenzo[b]thiophen-5-yl)butyl)-6- |
| | methoxyisoindolin-2-yl)-2-methyl-4-oxobutanoic acid |
| 33. | (S)-4-(5-(5-(2-((S)-3-carboxybutanoyl)-6-methoxybenzo[b]thiophen-5-yl)pentyl)-6- |
| | methoxyisoindolin-2-yl)-2-methyl-4-oxobutanoic acid |
| 34. | (2S)-4-(5-((4-((2-((S)-3-carboxybutanoyl)-6-methoxybenzo[b]thiophen-5-yl)oxy)pentan-2- |
| | yl)oxy)-6-methoxyisoindolin-2-yl)-2-methyl-4-oxobutanoic acid |
| 35. | (S)-4-(5-(3-((2-((S)-3-carboxybutanoyl)-6-methoxybenzo[b]thiophen-5-yl)oxy)-2,2- |
| | dimethylpropoxy)-6-methoxyisoindolin-2-yl)-2-methyl-4-oxobutanoic acid |
| 36. | (S)-4-(5-((1-(((2-((S)-3-carboxybutanoyl)-6-methoxybenzo[b]thiophen-5-yl)oxy)methyl) |
| | cyclopropyl)methoxy)-6-methoxyisoindolin-2-yl)-2-methyl-4-oxobutanoic acid |
| 37. | 4-(5-(3-((2-(3-carboxypropanoyl)-6-methoxyisoindolin-5-yl)oxy)propoxy)-6-methoxybenzo |
| | [b]thiophen-2-yl)-2,2-dimethyl-4-oxobutanoic acid |
| 38. | 4-(5-(3-((2-(3-carboxypropanoyl)-6-methoxybenzo[b]thiophen-5-yl)oxy)propoxy)-6- |
| | methoxyisoindolin-2-yl)-2,2-dimethyl-4-oxobutanoic acid |
| 39. | 4-(5-(3-((2-(3-carboxy-3-methylbutanoyl)-6-methoxybenzo[b]thiophen-5-yl)oxy)propoxy)- |
| | 6-methoxyisoindolin-2-yl)-2,2-dimethyl-4-oxobutanoic acid |
| 40. | (S)-4-(5-((4-(2-((S)-3-carboxybutanoyl)-6-methoxybenzo[b]thiophen-5-yl)butyl)amino)-6- |
| | methoxyisoindolin-2-yl)-2-methyl-4-oxobutanoic acid |
| 41. | (S)-4-(5-((3-((2-((S)-3-carboxybutanoyl)-6-methoxybenzo[b]thiophen-5-yl)oxy)propyl) |
| | amino)-6-methoxyisoindolin-2-yl)-2-methyl-4-oxobutanoic acid |
| 42. | (S)-4-(5-(4-((2-((S)-3-carboxybutanoyl)-6-methoxybenzo[b]thiophen-5-yl)oxy)butyl)-6- |
| | methoxyisoindolin-2-yl)-2-methyl-4-oxobutanoic acid |
| 43. | (S)-4-(5-(3-((2-((S)-3-carboxybutanoyl)-4-fluoro-6-methoxybenzo[b]thiophen-5-yl)oxy)pro- |
| | poxy)-6-methoxyisoindolin-2-yl)-2-methyl-4-oxobutanoic acid |
| 44. | (S)-4-(5-(3-((2-((S)-3-carboxybutanoyl)-4-chloro-6-methoxybenzo[b]thiophen-5-yl)oxy)pro- |
| | poxy)-6-methoxyisoindolin-2-yl)-2-methyl-4-oxobutanoic acid |
| 45. | (S)-4-(5-(3-((4-bromo-2-((S)-3-carboxybutanoyl)-6-methoxybenzo[b]thiophen-5-yl)oxy)pro- |
| | poxy)-6-methoxyisoindolin-2-yl)-2-methyl-4-oxobutanoic acid |
| 46. | (S)-4-(5-(3-((2-((S)-3-carboxybutanoyl)-6-methoxybenzo[d]thiazol-5-yl)oxy)propoxy)-6- |
| | methoxyisoindolin-2-yl)-2-methyl-4-oxobutanoic acid |
| 47. | (S)-4-(5-(3-((2-((S)-3-carboxybutanoyl)-6-hydroxybenzo[b]thiophen-5-yl)oxy)propoxy)-6- |
| | methoxyisoindolin-2-yl)-2-methyl-4-oxobutanoic acid |
| 48. | (S)-4-(5-(3-((2-((S)-3-carboxybutanoyl)-4,7-difluoro-6-methoxybenzo[b]thiophen-5- |
| | yl)oxy)propoxy)-6-methoxyisoindolin-2-yl)-2-methyl-4-oxobutanoic acid |
| 49. | (S)-4-(5-(3-((2-((S)-3-carboxybutanoyl)-4,7-dichloro-6-methoxybenzo[b]thiophen-5- |
| | yl)oxy)propoxy)-6-methoxyisoindolin-2-yl)-2-methyl-4-oxobutanoic acid |
| 50. | (2S)-4-(5-(3-((2-((S)-3-carboxybutanoyl)-6-methoxy-3-methylisoindolin-5-yl)oxy)propoxy)- |
| | 6-methoxybenzo[b]thiophen-2-yl)-2-methyl-4-oxobutanoic acid |
| 51. | (S)-4-(5-(3-((2-((S)-3-carboxybutanoyl)-4-fluoro-6-methoxyisoindolin-5-yl)oxy)propoxy)- |
| | 6-methoxybenzo[b]thiophen-2-yl)-2-methyl-4-oxobutanoic acid |
| 52. | (S)-4-(5-(3-((2-((S)-3-carboxybutanoyl)-4-chloro-6-methoxyisoindolin-5-yl)oxy)propoxy)- |
| | 6-methoxybenzo[b]thiophen-2-yl)-2-methyl-4-oxobutanoic acid |
| 53. | (S)-4-(5-(3-((4-bromo-2-((S)-3-carboxybutanoyl)-6-methoxyisoindolin-5-yl)oxy)propoxy)- |
| | 6-methoxybenzo[b]thiophen-2-yl)-2-methyl-4-oxobutanoic acid |
| 54. | (S)-4-(5-(3-((2-((S)-3-carboxybutanoyl)-6-hydroxyisoindolin-5-yl)oxy)propoxy)-6-methoxy- |
| | benzo[b]thiophen-2-yl)-2-methyl-4-oxobutanoic acid |
| 55. | (S)-4-(6-(3-((2-((S)-3-carboxybutanoyl)-6-methoxybenzo[b]thiophen-5-yl)oxy)propoxy)-4- |
| | fluoro-5-methoxyisoindolin-2-yl)-2-methyl-4-oxobutanoic acid |
| 56. | (S)-4-(6-(3-((2-((S)-3-carboxybutanoyl)-6-methoxybenzo[b]thiophen-5-yl)oxy)propoxy)-4- |
| | chloro-5-methoxyisoindolin-2-yl)-2-methyl-4-oxobutanoic acid |
| 57. | (S)-4-(5-(3-((7-bromo-2-((S)-3-carboxybutanoyl)-6-methoxyisoindolin-5-yl)oxy)propoxy)- |
| | 6-methoxybenzo[b]thiophen-2-yl)-2-methyl-4-oxobutanoic acid |
| 58. | (S)-4-(5-(3-((2-((S)-3-carboxybutanoyl)-4,7-difluoro-6-methoxyisoindolin-5-yl)oxy)propox |
| | y)-6-methoxybenzo[b]thiophen-2-yl)-2-methyl-4-oxobutanoic acid |
| 59. | (S)-4-(5-(3-((2-((S)-3-carboxybutanoyl)-6-methoxybenzo[b]thiophen-5-yl)oxy)propoxy)-7- |
| | chloro-4-fluoro-6-methoxyisoindolin-2-yl)-2-methyl-4-oxobutanoic acid |
| 60. | (S)-4-(5-(3-((2-((S)-3-carboxybutanoyl)-4,7-dichloro-6-methoxyisoindolin-5-yl)oxy)pro- |
| | poxy)-6-methoxybenzo[b]thiophen-2-yl)-2-methyl-4-oxobutanoic acid |
| 61. | (S)-4-(5-(3-((2-((S)-3-carboxybutanoyl)-4-chloro-6-methoxybenzo[b]thiophen-5-yl)oxy)pro- |
| | poxy)-4-fluoro-6-methoxyisoindolin-2-yl)-2-methyl-4-oxobutanoic acid |
| 62. | (S)-4-(5-(3-((2-((S)-3-carboxybutanoyl)-4-chloro-6-methoxyisoindolin-5-yl)oxy)propoxy)- |
| | 4-fluoro-6-methoxybenzo[b]thiophen-2-yl)-2-methyl-4-oxobutanoic acid |
| 63. | (S)-4-(5-(3-((2-((S)-3-carboxybutanoyl)-4-chloro-6-methoxybenzo[b]thiophen-5-yl)oxy)pro- |
| | poxy)-4-chloro-6-methoxyisoindolin-2-yl)-2-methyl-4-oxobutanoic acid |
| 64. | (S)-4-(6-(3-((2-((S)-3-carboxybutanoyl)-7-fluoro-6-methoxybenzo[b]thiophen-5-yl)oxy)pro- |
| | poxy)-4-fluoro-5-methoxyisoindolin-2-yl)-2-methyl-4-oxobutanoic acid |
| 65. | (S)-4-(6-(3-((2-((S)-3-carboxybutanoyl)-7-chloro-6-methoxybenzo[b]thiophen-5-yl)oxy)pro- |
| | poxy)-4-fluoro-5-methoxyisoindolin-2-yl)-2-methyl-4-oxobutanoic acid |
| 66. | (S)-4-(5-(3-((2-((S)-3-carboxybutanoyl)-7-chloro-6-methoxyisoindolin-5-yl)oxy)propoxy)- |
| | 7-fluoro-6-methoxybenzo[b]thiophen-2-yl)-2-methyl-4-oxobutanoic acid |
| 67. | (S)-4-(5-(3-((2-((S)-3-carboxybutanoyl)-4,7-difluoro-6-methoxyisoindolin-5-yl)oxy)pro- |
| | poxy)-7-chloro-6-methoxybenzo[b]thiophen-2-yl)-2-methyl-4-oxobutanoic acid |
| 68. | (S)-4-(5-(3-((2-((S)-3-carboxybutanoyl)-4,7-difluoro-6-methoxyisoindolin-5-yl)oxy)pro- |
| | poxy)-7-fluoro-6-methoxybenzo[b]thiophen-2-yl)-2-methyl-4-oxobutanoic acid |
| 69. | (S)-4-(5-(3-((2-((S)-3-carboxybutanoyl)-4-chloro-7-fluoro-6-methoxyisoindolin-5-yl)oxy) |
| | propoxy)-7-fluoro-6-methoxybenzo[b]thiophen-2-yl)-2-methyl-4-oxobutanoic acid |
| 70. | (S)-4-(6-(3-((2-((S)-3-carboxybutanoyl)-4,7-difluoro-6-methoxybenzo[b]thiophen-5- |
| | yl)oxy)propoxy)-4-fluoro-5-methoxyisoindolin-2-yl)-2-methyl-4-oxobutanoic acid |
| 71. | (S)-4-(6-(3-((2-((S)-3-carboxybutanoyl)-7-chloro-4-fluoro-6-methoxybenzo[b]thiophen-5- |
| | yl)oxy)propoxy)-4-fluoro-5-methoxyisoindolin-2-yl)-2-methyl-4-oxobutanoic acid |
| 72. | (S)-4-(6-(3-((2-((S)-3-carboxybutanoyl)-4,7-dichloro-6-methoxybenzo[b]thiophen-5- |
| | yl)oxy)propoxy)-4-fluoro-5-methoxyisoindolin-2-yl)-2-methyl-4-oxobutanoic acid |
| 73. | (S)-4-(5-(3-((2-((S)-3-carboxybutanoyl)-4-chloro-7-fluoro-6-methoxyisoindolin-5- |
| | yl)oxy)propoxy)-7-chloro-6-methoxybenzo[b]thiophen-2-yl)-2-methyl-4-oxobutanoic acid |
| 74. | (S)-4-(5-(3-((2-((S)-3-carboxybutanoyl)-4,7-dichloro-6-methoxyisoindolin-5-yl)oxy)pro- |
| | poxy)-7-fluoro-6-methoxybenzo[b]thiophen-2-yl)-2-methyl-4-oxobutanoic acid |
| 75. | (S)-4-(5-(3-((2-((S)-3-carboxybutanoyl)-7-chloro-4-fluoro-6-methoxyisoindolin-5- |
| | yl)oxy)propoxy)-7-fluoro-6-methoxybenzo[b]thiophen-2-yl)-2-methyl-4-oxobutanoic acid |
| 76. | (S)-4-(6-(3-((2-((S)-3-carboxybutanoyl)-4,7-difluoro-6-methoxybenzo[b]thiophen-5- |
| | yl)oxy)propoxy)-4-chloro-5-methoxyisoindolin-2-yl)-2-methyl-4-oxobutanoic acid |
| 77. | (S)-4-(6-(3-((2-((S)-3-carboxybutanoyl)-4-chloro-7-fluoro-6-methoxybenzo[b]thiophen- |
| | 5-yl)oxy)propoxy)-4-chloro-5-methoxyisoindolin-2-yl)-2-methyl-4-oxobutanoic acid |
| 78. | (S)-4-(5-(3-((2-((S)-3-carboxybutanoyl)-4-fluoro-6-hydroxyisoindolin-5-yl)oxy)propoxy)-4- |
| | fluoro-6-methoxybenzo[b]thiophen-2-yl)-2-methyl-4-oxobutanoic acid |
| 79. | (S)-4-(5-(3-((2-((S)-3-carboxybutanoyl)-4-chloro-6-methoxybenzo[b]thiophen-5- |
| | yl)oxy)propoxy)-4-fluoro-6-hydroxyisoindolin-2-yl)-2-methyl-4-oxobutanoic acid |
| 80. | (S)-4-(5-(3-((2-((S)-3-carboxybutanoyl)-4-chloro-6-hydroxyisoindolin-5-yl)oxy)propoxy)- |
| | 4-fluoro-6-methoxybenzo[b]thiophen-2-yl)-2-methyl-4-oxobutanoic acid |
| 81. | (S)-4-(5-(3-((2-((S)-3-carboxybutanoyl)-4-chloro-6-hydroxyisoindolin-5-yl)oxy)propoxy)- |
| | 4-chloro-6-methoxybenzo[b]thiophen-2-yl)-2-methyl-4-oxobutanoic acid |
| 82. | (S)-4-(6-(3-((2-((S)-3-carboxybutanoyl)-6-methoxybenzo[b]thiophen-5-yl)oxy)propoxy)- |
| | 4-fluoro-5-hydroxyisoindolin-2-yl)-2-methyl-4-oxobutanoic acid |
| 83. | (S)-4-(5-(3-((2-((S)-3-carboxybutanoyl)-7-fluoro-6-hydroxyisoindolin-5-yl)oxy)propoxy)- |
| | 7-fluoro-6-methoxybenzo[b]thiophen-2-yl)-2-methyl-4-oxobutanoic acid |
| 84. | (S)-4-(6-(3-((2-((S)-3-carboxybutanoyl)-7-chloro-6-methoxybenzo[b]thiophen-5- |
| | yl)oxy)propoxy)-4-fluoro-5-hydroxyisoindolin-2-yl)-2-methyl-4-oxobutanoic acid |
| 85. | (S)-4-(5-(3-((2-((S)-3-carboxybutanoyl)-7-chloro-6-hydroxyisoindolin-5-yl)oxy)pro |
| | poxy)-7-fluoro-6-methoxybenzo[b]thiophen-2-yl)-2-methyl-4-oxobutanoic acid |
| 86. | (S)-4-(5-(3-((2-((S)-3-carboxybutanoyl)-4,7-difluoro-6-methoxyisoindolin-5-yl)oxy)pro- |
| | poxy)-4-chloro-7-fluoro-6-methoxybenzo[b]thiophen-2-yl)-2-methyl-4-oxobutanoic acid |
| 87. | (S)-4-(5-(3-((2-((S)-3-carboxybutanoyl)-4-chloro-7-fluoro-6-methoxybenzo[b]thiophen-5- |
| | yl)oxy)propoxy)-4-chloro-7-fluoro-6-methoxyisoindolin-2-yl)-2-methyl-4-oxobutanoic acid |
| 88. | (S)-4-(5-(3-((2-((S)-3-carboxybutanoyl)-4,7-difluoro-6-methoxybenzo[b]thiophen-5- |
| | yl)oxy)propoxy)-4,7-difluoro-6-methoxyisoindolin-2-yl)-2-methyl-4-oxobutanoic acid |
| 89. | (S)-4-(5-(3-((2-((S)-3-carboxybutanoyl)-4,7-difluoro-6-methoxyisoindolin-5-yl)oxy)pro- |
| | poxy)-7-chloro-4-fluoro-6-methoxybenzo[b]thiophen-2-yl)-2-methyl-4-oxobutanoic acid |
| 90. | (S)-4-(5-(3-((2-((S)-3-carboxybutanoyl)-4,7-dichloro-6-methoxybenzo[b]thiophen-5- |
| | yl)oxy)propoxy)-4,7-difluoro-6-methoxyisoindolin-2-yl)-2-methyl-4-oxobutanoic acid |
| 91. | (S)-4-(5-(3-((2-((S)-3-carboxybutanoyl)-4,7-dichloro-6-methoxybenzo[b]thiophen-5- |
| | yl)oxy)propoxy)-4-chloro-7-fluoro-6-methoxyisoindolin-2-yl)-2-methyl-4-oxobutanoic acid |
| 92. | (S)-4-(5-(3-((2-((S)-3-carboxybutanoyl)-4-chloro-7-fluoro-6-methoxyisoindolin-5-yl)oxy)pro- |
| | poxy)-7-chloro-4-fluoro-6-methoxybenzo[b]thiophen-2-yl)-2-methyl-4-oxobutanoic acid |
| 93. | (S)-4-(5-(3-((2-((S)-3-carboxybutanoyl)-4,7-dichloro-6-methoxyisoindolin-5-yl)oxy)pro- |
| | poxy)-4-chloro-7-fluoro-6-methoxybenzo[b]thiophen-2-yl)-2-methyl-4-oxobutanoic acid |
| 94. | (S)-4-(5-(3-((2-((S)-3-carboxybutanoyl)-4-chloro-7-fluoro-6-methoxybenzo[b]thiophen-5- |
| | yl)oxy)propoxy)-7-chloro-4-fluoro-6-methoxyisoindolin-2-yl)-2-methyl-4-oxobutanoic acid |
| 95. | (2S,2′S)-4,4′-(7,8,11,13,17,18-hexahydro-6H,12H,16H-thieno[2″,3″:4′,5′]benzo[1′,2′:9,10] |
| | [1,4,8,11 ]tetraoxacyclotetradecino[2,3-f]isoindole-2,12-diyl)bis(2-methyl-4-oxobutanoic acid) |
| 96. | (S)-4-(6-methoxy-5-(3-((6-methoxy-2-((S)-3-methyl-4-(methylsulfonamido)-4-oxobutanoyl) |
| | isoindolin-5-yl)oxy)propoxy)benzo[b]thiophen-2-yl)-2-methyl-4-oxobutanoic acid |
| 97. | (S)-4-(5-(3-((2-((S)-4-((N,N-dimethylsulfamoyl)amino)-3-methyl-4-oxobutanoyl)-6- |
| | methoxyisoindolin-5-yl)oxy)propoxy)-6-methoxybenzo[b]thiophen-2-yl)-2-methyl-4- |
| | oxobutanoic acid |
| 98. | (S)-4-(5-(3-((2-(3-cyanopropanoyl)-6-methoxyisoindolin-5-yl)oxy)propoxy)-6-methoxy- |
| | benzo[b]thiophen-2-yl)-2-methyl-4-oxobutanoic acid |
| 99. | 4-(5-(3-((2-(3-cyanopropanoyl)-6-methoxybenzo[b]thiophen-5-yl)oxy)propoxy)-6- |
| | methoxyisoindolin-2-yl)-4-oxobutanenitrile |
| 100. | (S)-4-(5-(3-((2-(3-cyanopropanoyl)-6-methoxybenzo[b]thiophen-5-yl)oxy)propoxy)-6- |
| | methoxyisoindolin-2-yl)-2-methyl-4-oxobutanoic acid |
| 101. | (1R,2R)-2-(5-(3-((2-((S)-3-carboxybutanoyl)-6-methoxyisoindolin-5-yl)oxy)propoxy)- |
| | 6-methoxybenzo[b]thiophene-2-carbonyl)cyclobutane-1-carboxylic acid |
| 102. | (1R,2R)-2-(5-(3-((2-((1R,2R)-2-carboxycyclobutane-1-carbonyl)-6-methoxybenzo[b]thio- |
| | phen-5-yl)oxy)propoxy)-6-methoxyisoindoline-2-carbonyl)cyclobutane-1-carboxylic acid |
| 103. | (1R,2R)-2-(5-(3-((2-((S)-3-carboxybutanoyl)-6-methoxybenzo[b]thiophen-5-yl)oxy)pro |
| | poxy)-6-methoxyisoindoline-2-carbonyl)cyclobutane-1-carboxylic acid |
| 104. | (1R,2R)-2-(5-(3-((2-((S)-3-carboxybutanoyl)-6-methoxyisoindolin-5-yl)oxy)propoxy)-6- |
| | methoxybenzo[b]thiophene-2-carbonyl)cyclopropane-1-carboxylic acid |
| 105. | (1R,2R)-2-(5-(3-((2-((1R,2R)-2-carboxycyclopropane-1-carbonyl)-6-methoxybenzo[b]thio- |
| | phen-5-yl)oxy)propoxy)-6-methoxyisoindoline-2-carbonyl)cyclopropane-1-carboxylic acid |
| 106. | (1R,2R)-2-(5-(3-((2-((S)-3-carboxybutanoyl)-6-methoxybenzo[b]thiophen-5-yl)oxy)pro- |
| | poxy)-6-methoxyisoindoline-2-carbonyl)cyclopropane-1-carboxylic acid |
| 107. | (S)-N-(N,N-dimethylsulfamoyl)-4-(5-(3-((2-((S)-4-((N,N-dimethylsulfamoyl)amino)-3- |
| | methyl-4-oxobutanoyl)-6-methoxybenzo[b]thiophen-5-yl)oxy)propoxy)-6-methoxy- |
| | isoindolin-2-yl)-2-methyl-4-oxobutanamide |
| 108. | (S)-4-(5-(3-((2-((S)-4-((N,N-dimethylsulfamoyl)amino)-3-methyl-4-oxobutanoyl)-6- |
| | methoxybenzo[b]thiophen-5-yl)oxy)propoxy)-6-methoxyisoindolin-2-yl)-2-methyl- |
| | 4-oxobutanoic acid |
| 109. | (S)-4-(6-methoxy-5-(3-((6-methoxy-2-((S)-3-methyl-4-(methylsulfonamido)-4-oxo- |
| | butanoyl)isoindolin-5-yl)oxy)propoxy)benzo[b]thiophen-2-yl)-2-methyl-N-(methyl- |
| | sulfonyl)-4-oxobutanamide |
| 110. | (S)-4-(5-methoxy-6-(3-((6-methoxy-2-((S)-3-methyl-4-(methylsulfonamido)-4-oxobutanoyl |
| | )benzo[b]thiophen-5-yl)oxy)propoxy)isoindolin-2-yl)-2-methyl-4-oxobutanoic acid |
| 111. | (S)-4-(4-(3-((2-((S)-3-carboxybutanoyl)-6-methoxybenzo[b]thiophen-5-yl)oxy)propoxy)-6- |
| | methoxyisoindolin-2-yl)-2-methyl-4-oxobutanoic acid |
| 112. | (S)-4-(5-(3-((2-((S)-3-carboxybutanoyl)-6-methoxybenzo[b]thiophen-4-yl)oxy)propoxy)-6- |
| | methoxyisoindolin-2-yl)-2-methyl-4-oxobutanoic acid |
| 113. | (S)-4-(4-(4-((2-((S)-3-carboxybutanoyl)-6-methoxybenzo[b]thiophen-5-yl)oxy)butyl)-6- |
| | methoxyisoindolin-2-yl)-2-methyl-4-oxobutanoic acid |
| 114. | (S)-4-(4-(4-(2-((S)-3-carboxybutanoyl)-6-methoxybenzo[b]thiophen-5-yl)butoxy)-6- |
| | methoxyisoindolin-2-yl)-2-methyl-4-oxobutanoic acid |
| 115. | (S)-4-(5-(4-((2-((S)-3-carboxybutanoyl)-6-methoxybenzo[b]thiophen-4-yl)oxy)butyl)-6- |
| | methoxyisoindolin-2-yl)-2-methyl-4-oxobutanoic acid |
| 116. | (S)-4-(5-(4-(2-((S)-3-carboxybutanoyl)-6-methoxybenzo[b]thiophen-4-yl)butoxy)-6- |
| | methoxyisoindolin-2-yl)-2-methyl-4-oxobutanoic acid |
| 117. | (S)-4-(5-(3-((2-((S)-3-carboxybutanoyl)-6-methoxyisoindolin-5-yl)oxy)propoxy)-6- |
| | methylbenzo[b]thiophen-2-yl)-2-methyl-4-oxobutanoic acid |
| 118. | (S)-4-(5-(3-((2-((S)-3-carboxybutanoyl)-6-ethylbenzo[b]thiophen-5-yl)oxy)propoxy)-6- |
| | methoxyisoindolin-2-yl)-2-methyl-4-oxobutanoic acid |
| 119. | (S)-4-(5-(3-((2-((S)-3-carboxybutanoyl)-6-methoxyisoindolin-5-yl)oxy)propoxy)-6- |
| | vinylbenzo[b]thiophen-2-yl)-2-methyl-4-oxobutanoic acid |
| 120. | (S)-4-(5-(3-((2-((S)-3-carboxybutanoyl)-6-ethynylbenzo[b]thiophen-5-yl)oxy)propoxy)- |
| | 6-methoxyisoindolin-2-yl)-2-methyl-4-oxobutanoic acid |
| 121. | (S)-4-(5-(3-((6-amino-2-((S)-3-carboxybutanoyl)benzo[b]thiophen-5-yl)oxy)propoxy)- |
| | 6-methoxyisoindolin-2-yl)-2-methyl-4-oxobutanoic acid |
| 122. | (S)-4-(5-(3-((2-((S)-3-carboxybutanoyl)-6-(methylamino)benzo[b]thiophen-5- |
| | yl)oxy)propoxy)-6-methoxyisoindolin-2-yl)-2-methyl-4-oxobutanoic acid |
| 123. | (S)-4-(5-(3-((6-bromo-2-((S)-3-carboxybutanoyl)benzo[b]thiophen-5-yl)oxy)propoxy)- |
| | 6-methoxyisoindolin-2-yl)-2-methyl-4-oxobutanoic acid |
| 124. | (S)-4-(5-(3-((2-((S)-3-carboxybutanoyl)-6-methoxybenzo[b]thiophen-5-yl)oxy)propoxy)- |
| | 6-methylisoindolin-2-yl)-2-methyl-4-oxobutanoic acid |
| 125. | (S)-4-(5-(3-((2-((S)-3-carboxybutanoyl)-6-ethylisoindolin-5-yl)oxy)propoxy)-6- |
| | methoxybenzo[b]thiophen-2-yl)-2-methyl-4-oxobutanoic acid |
| 126. | (S)-4-(5-(3-((2-((S)-3-carboxybutanoyl)-6-methoxybenzo[b]thiophen-5-yl)oxy)propoxy)- |
| | 6-vinylisoindolin-2-yl)-2-methyl-4-oxobutanoic acid |
| 127. | (S)-4-(5-(3-((2-((S)-3-carboxybutanoyl)-6-ethynylisoindolin-5-yl)oxy)propoxy)-6- |
| | methoxybenzo[b]thiophen-2-yl)-2-methyl-4-oxobutanoic acid |
| 128. | (S)-4-(5-(3-((6-amino-2-((S)-3-carboxybutanoyl)isoindolin-5-yl)oxy)propoxy)-6- |
| | methoxybenzo[b]thiophen-2-yl)-2-methyl-4-oxobutanoic acid |
| 129. | (S)-4-(5-(3-((2-((S)-3-carboxybutanoyl)-6-(methylamino)isoindolin-5-yl)oxy)propoxy)- |
| | 6-methoxybenzo[b]thiophen-2-yl)-2-methyl-4-oxobutanoic acid |
| 130. | (S)-4-(5-(3-((6-bromo-2-((S)-3-carboxybutanoyl)isoindolin-5-yl)oxy)propoxy)-6- |
| | methoxybenzo[b]thiophen-2-yl)-2-methyl-4-oxobutanoic acid |
| 131. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-methoxybenzo[b]thiophen-2-yl)-2-methyl-4-oxobutanoic acid |
| 132. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-4-fluoro-6-methoxybenzo[b]thiophen-2-yl)-2-methyl-4-oxobutanoic |
| | acid |
| 133. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-4-chloro-6-methoxybenzo[b]thiophen-2-yl)-2-methyl-4-oxobutanoic |
| | acid |
| 134. | (S)-4-(5-(3-((6-((S)-3-carboxybutanoyl)-3-hydroxy-6,7-dihydro-5H-pyrrolo[3,4-b]pyridin- |
| | 2-yl)oxy)propoxy)-6-methoxythieno[3,2-b]pyridin-2-yl)-2-methyl-4-oxobutanoic acid |
| 135. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-hydroxythieno[3,2-b]pyridin-2-yl)-2-methyl-4-oxobutanoic acid |
| 136. | (S)-4-(5-(3-((6-((S)-3-carboxybutanoyl)-3-methoxy-6,7-dihydro-5H-pyrrolo[3,4-b]pyridin- |
| | 2-yl)oxy)propoxy)-6-methoxythieno[3,2-b]pyridin-2-yl)-2-methyl-4-oxobutanoic acid |
| 137. | (S)-4-(5-(3-((6-((S)-3-carboxybutanoyl)-4-fluoro-3-methoxy-6,7-dihydro-5H-pyrrolo[3,4-b] |
| | pyridin-2-yl)oxy)propoxy)-6-methoxythieno[3,2-b]pyridin-2-yl)-2-methyl-4-oxobutanoic |
| | acid |
| 138. | (S)-4-(5-(3-((6-((S)-3-carboxybutanoyl)-4-chloro-3-methoxy-6,7-dihydro-5H-pyrrolo[3,4- |
| | b]pyridin-2-yl)oxy)propoxy)-6-methoxythieno[3,2-b]pyridin-2-yl)-2-methyl-4-oxobutanoic |
| | acid |
| 139. | (S)-4-(5-(3-((2-((S)-3-carboxybutanoyl)-6-methoxyisoindolin-5-yl)oxy)propoxy)-6- |
| | methoxythieno[3,2-b]pyridin-2-yl)-2-methyl-4-oxobutanoic acid |
| 140. | (S)-4-(5-(3-((2-((S)-3-carboxybutanoyl)-4-fluoro-6-methoxyisoindolin-5-yl)oxy)propoxy)- |
| | 6-methoxythieno[3,2-b]pyridin-2-yl)-2-methyl-4-oxobutanoic acid |
| 141. | (S)-4-(5-(3-((2-((S)-3-carboxybutanoyl)-4-chloro-6-methoxyisoindolin-5-yl)oxy)propoxy)- |
| | 6-methoxythieno[3,2-b]pyridin-2-yl)-2-methyl-4-oxobutanoic acid |
| 142. | (S)-4-(5-(3-((2-((S)-3-carboxybutanoyl)-4,7-dichloro-6-methoxyisoindolin-5-yl)oxy)pro- |
| | poxy)-6-methoxythieno[3,2-b]pyridin-2-yl)-2-methyl-4-oxobutanoic acid |
| 143. | (S)-4-(5-(3-((2-((S)-3-carboxybutanoyl)-4-chloro-7-fluoro-6-methoxyisoindolin-5- |
| | yl)oxy)propoxy)-6-methoxythieno[3,2-b]pyridin-2-yl)-2-methyl-4-oxobutanoic acid |
| 144. | (S)-4-(5-(3-((2-((S)-3-carboxybutanoyl)-4,7-difluoro-6-methoxyisoindolin-5-yl)oxy)pro- |
| | poxy)-6-methoxythieno[3,2-b]pyridin-2-yl)-2-methyl-4-oxobutanoic acid |
| 145. | (S)-4-(5-(3-((2-((S)-3-carboxybutanoyl)-4,7-difluoro-6-hydroxyisoindolin-5-yl)oxy)pro- |
| | poxy)-6-methoxythieno[3,2-b]pyridin-2-yl)-2-methyl-4-oxobutanoic acid |
| 146. | (S)-4-(5-(3-((2-((S)-3-carboxybutanoyl)-4-fluoro-6-hydroxybenzo[b]thiophen-5- |
| | yl)oxy)propoxy)-4-fluoro-6-methoxyisoindolin-2-yl)-2-methyl-4-oxobutanoic acid |
| 147. | (S)-4-(5-(3-((2-((S)-3-carboxybutanoyl)-4-chloro-6-hydroxybenzo[b]thiophen-5- |
| | yl)oxy)propoxy)-4-fluoro-6-methoxyisoindolin-2-yl)-2-methyl-4-oxobutanoic acid |
| 148. | (S)-4-(5-(3-((2-((S)-3-carboxybutanoyl)-4-chloro-6-methoxyisoindolin-5-yl)oxy)pro |
| | poxy)-4-fluoro-6-hydroxybenzo[b]thiophen-2-yl)-2-methyl-4-oxobutanoic acid |
| 149. | (S)-4-(5-(3-((2-((S)-3-carboxybutanoyl)-4-chloro-6-hydroxybenzo[b]thiophen-5- |
| | yl)oxy)propoxy)-4-chloro-6-methoxyisoindolin-2-yl)-2-methyl-4-oxobutanoic acid |
| 150. | (S)-4-(6-(3-((2-((S)-3-carboxybutanoyl)-6-hydroxybenzo[b]thiophen-5-yl)oxy)propoxy)- |
| | 4-fluoro-5-methoxyisoindolin-2-yl)-2-methyl-4-oxobutanoic acid |
| 151. | (S)-4-(6-(3-((2-((S)-3-carboxybutanoyl)-7-fluoro-6-hydroxybenzo[b]thiophen-5- |
| | yl)oxy)propoxy)-4-fluoro-5-methoxyisoindolin-2-yl)-2-methyl-4-oxobutanoic acid |
| 152. | (S)-4-(6-(3-((2-((S)-3-carboxybutanoyl)-7-chloro-6-hydroxybenzo[b]thiophen-5- |
| | yl)oxy)propoxy)-4-fluoro-5-methoxyisoindolin-2-yl)-2-methyl-4-oxobutanoic acid |
| 153. | (S)-4-(5-(3-((2-((S)-3-carboxybutanoyl)-7-chloro-6-methoxyisoindolin-5-yl)oxy)pro- |
| | poxy)-7-fluoro-6-hydroxybenzo[b]thiophen-2-yl)-2-methyl-4-oxobutanoic acid |
| 154. | (S)-4-(5-(3-((2-((S)-3-carboxybutanoyl)-4,7-difluoro-6-methoxyisoindolin-5-yl)oxy)pro- |
| | poxy)-7-chloro-6-hydroxybenzo[b]thiophen-2-yl)-2-methyl-4-oxobutanoic acid |
| 155. | (S)-4-(5-(3-((2-((S)-3-carboxybutanoyl)-4,7-difluoro-6-methoxyisoindolin-5-yl)oxy)pro- |
| | poxy)-7-fluoro-6-hydroxybenzo[b]thiophen-2-yl)-2-methyl-4-oxobutanoic acid |
| 156. | (S)-4-(5-(3-((2-((S)-3-carboxybutanoyl)-4-chloro-7-fluoro-6-methoxyisoindolin-5- |
| | yl)oxy)propoxy)-7-fluoro-6-hydroxybenzo[b]thiophen-2-yl)-2-methyl-4-oxobutanoic acid |
| 157. | (S)-4-(6-(3-((2-((S)-3-carboxybutanoyl)-4,7-difluoro-6-hydroxybenzo[b]thiophen-5-yl)oxy) |
| | propoxy)-4-fluoro-5-methoxyisoindolin-2-yl)-2-methyl-4-oxobutanoic acid |
| 158. | (S)-4-(6-(3-((2-((S)-3-carboxybutanoyl)-7-chloro-4-fluoro-6-hydroxybenzo[b]thiophen- |
| | 5-yl)oxy)propoxy)-4-fluoro-5-methoxyisoindolin-2-yl)-2-methyl-4-oxobutanoic acid |
| 159. | (S)-4-(6-(3-((2-((S)-3-carboxybutanoyl)-4,7-dichloro-6-hydroxybenzo[b]thiophen-5-yl)oxy) |
| | propoxy)-4-fluoro-5-methoxyisoindolin-2-yl)-2-methyl-4-oxobutanoic acid |
| 160. | (S)-4-(5-(3-((2-((S)-3-carboxybutanoyl)-5-methoxybenzo[b]thiophen-6-yl)oxy)propoxy)-6- |
| | methoxyisoindolin-2-yl)-2-methyl-4-oxobutanoic acid |
| 161. | 4-(5-(3-((2-(3-carboxypropanoyl)-6-methoxyisoindolin-5-yl)oxy)propoxy)-6- |
| | methoxythieno[2,3-b]pyridin-2-yl)-4-oxobutanoic acid |
| 162. | 4-(5-(3-((2-(4-(((4R,5R)-5-hydroxy-1,2-dithian-4-yl)oxy)-4-oxobutanoyl)-6-methoxy- |
| | thieno[2,3-b]pyridin-5-yl)oxy)propoxy)-6-methoxyisoindolin-2-yl)-4-oxobutanoic acid |
| 163. | 4-(5-(3-((6-(3-carboxypropanoyl)-3-methoxy-6,7-dihydro-5H-pyrrolo[3,4-b]pyridin-2- |
| | yl)oxy)propoxy)-6-methoxybenzo[b]thiophen-2-yl)-4-oxobutanoic acid |
| 164. | 4-(2-(3-((2-(4-(((4R,5R)-5-hydroxy-1,2-dithian-4-yl)oxy)-4-oxobutanoyl)-6-methoxy- |
| | benzo[b]thiophen-5-yl)oxy)propoxy)-3-methoxy-5,7-dihydro-6H-pyrrolo[3,4-b]pyridin- |
| | 6-yl)-4-oxobutanoic acid |
| 165. | 4-(5-(3-((6-(3-carboxypropanoyl)-2-methoxy-6,7-dihydro-5H-pyrrolo[3,4-b]pyridin-3- |
| | yl)oxy)propoxy)-6-methoxybenzo[b]thiophen-2-yl)-4-oxobutanoic acid |
| 166. | 4-(3-(3-((2-(4-(((4R,5R)-5-hydroxy-1,2-dithian-4-yl)oxy)-4-oxobutanoyl)-6-methoxy- |
| | benzo[b]thiophen-5-yl)oxy)propoxy)-2-methoxy-5,7-dihydro-6H-pyrrolo[3,4-b]pyridin- |
| | 6-yl)-4-oxobutanoic acid |
| 167. | 4-(2-(3-((2-(4-(((4R,5R)-5-hydroxy-1,2-dithian-4-yl)oxy)-4-oxobutanoyl)-6-methoxy- |
| | thieno[3,2-b]pyridin-5-yl)oxy)propoxy)-3-methoxy-5, 7-dihydro-6H-pyrrolo[3,4-b]pyridin- |
| | 6-yl)-4-oxobutanoic acid |
| 168. | 4-(5-(3-((2-(3-carboxypropanoyl)-5-methoxythieno[2,3-b]pyridin-6-yl)oxy)propoxy)-6- |
| | methoxyisoindolin-2-yl)-4-oxobutanoic acid |
| 169. | (S)-4-(5-(3-((2-(3-carboxypropanoyl)-5-methoxythieno[2,3-b]pyridin-6-yl)oxy)propoxy)-6- |
| | methoxyisoindolin-2-yl)-2-methyl-4-oxobutanoic acid |
| 170. | (S)-4-(6-(3-((2-(3-carboxypropanoyl)-6-methoxyisoindolin-5-yl)oxy)propoxy)-5- |
| | methoxythieno[2,3-b]pyridin-2-yl)-2-methyl-4-oxobutanoic acid |
| 171. | (S)-4-(5-(3-((2-((S)-3-carboxybutanoyl)-5-methoxythieno[2,3-b]pyridin-6-yl)oxy)propoxy)- |
| | 6-methoxyisoindolin-2-yl)-2-methyl-4-oxobutanoic acid |
| 172. | 4-(5-(3-((2-(4-(((4R,5R)-5-hydroxy-1,2-dithian-4-yl)oxy)-4-oxobutanoyl)-5-methoxy- |
| | thieno[2,3-b]pyridin-6-yl)oxy)propoxy)-6-methoxyisoindolin-2-yl)-4-oxobutanoic acid. |

50. A pharmaceutical composition comprising a therapeutically effective amount of at least one compound or pharmaceutically acceptable salt thereof of any one of claims 1-49 and at least one pharmaceutically acceptable excipient.

51. The pharmaceutical composition of claim 50, wherein, the said compound or pharmaceutically acceptable salt thereof is in a weight ratio to the said excipient within the range from about 0.0001 to about 10.

52. The pharmaceutical composition of claim 50 or 51, further comprising at least one of additional active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents.

53. Use of at least one compound or pharmaceutically acceptable salt thereof of any one of claims 1-49 and/or a pharmaceutical composition of any one of claims 50-52 for the manufacture of a medicament.

54. The use of claim 53, wherein the medicament is used for inducing an immune response in a subject.

55. The use of claim 53, wherein the medicament is used for inducing STING-dependent type I interferon production in a subject.

56. The use of claim 53, wherein the medicament is used for inducing a STING-dependent cytokine production in a subject.

57. The use of claim 53, wherein the medicament is used for treating a cell proliferation disorder in a subject.

58. The use of claim 57, wherein the cell proliferation disorder is cancer.

59. At least one compound or pharmaceutically acceptable salt thereof for use as defined in any one of claims 1-49 and/or a pharmaceutical composition of any one of claims 50-52, which is for use in therapy.

60. At least one compound or pharmaceutically acceptable salt thereof for use as defined in any one of claims 1-49 and/or a pharmaceutical composition of any one of claims 50-52, which is for use in inducing an immune response in a subject.

61. At least one compound or pharmaceutically acceptable salt thereof for use as defined in any one of claims 1-49 and/or a pharmaceutical composition of any one of claims 50-52, which is for use in inducing STING-dependent type I interferon production in a subject.

62. At least one compound or pharmaceutically acceptable salt thereof for use as defined in any one of claims 1-49 and/or a pharmaceutical composition of any one of claims 50-52, which is for use in inducing a STING-dependent cytokine production in a subject.

63. At least one compound or pharmaceutically acceptable salt thereof for use as defined in any one of claims 1-49 and/or a pharmaceutical composition of any one of claims 50-52, which is for use in treating a cell proliferation disorder in a subject.

64. The use of claim 63, wherein the cell proliferation disorder is cancer.

65. At least one compound or pharmaceutically acceptable salt thereof as defined in any one of claims 1-49 and/or a pharmaceutical composition of any one of claims 50-52, which is for use as an STING agonists.

66. At least one compound or pharmaceutically acceptable salt thereof as defined in any one of claims 1-49 and/or a pharmaceutical composition of any one of claims 50-52, which is for use as a medicament.

67. A method of inducing an immune response in a subject, said method comprising administering a therapeutically effective amount of a compound according to any one of claims 1-49, or a pharmaceutically acceptable salt thereof, and/or a pharmaceutical composition of any one of claims 50-52, to the subject.

68. A method of inducing STING-dependent type I interferon production in a subject, said method comprising administering a therapeutically effective amount of a compound according to any one of claims 1-49, or a pharmaceutically acceptable salt thereof, and/or a pharmaceutical composition of any one of claims 50-52, to the subject.

69. A method of inducing a STING-dependent cytokine production in a subject, said method comprising administering a therapeutically effective amount of a compound according to any one of claims 1-49, or a pharmaceutically acceptable salt thereof, and/or a pharmaceutical composition of any one of claims 50-52, to the subject.

70. A method of treating a cell proliferation disorder in a subject, said method comprising administering a therapeutically effective amount of a compound according to any one of claims 1-49, and/or a pharmaceutical composition of any one of claims 50-52, to the subject.

71. The method of claim 70, wherein the cell proliferation disorder is cancer.
